(19) United States
(12) Patent Application Publication
(10) Pub. No.: US 2025/0084122 A1
(43) Pub. Date: Mar. 13, 2025

(54) VOLUMETRIC ABSORPTIVE MICROSAMPLING DEVICES AND METHODS OF USING THE SAME

(71) Applicants: Sangui Bio Pty Ltd, Manly, New South Wales (AU); The University of Sydney, Sydney, New South Wales (AU)

(72) Inventors: Cameron John Hill, St Leonards, New South Wales (AU); Ben Herbert, North Epping, New South Wales (AU); Mark Paul Molloy, West Pymble, New South Wales (AU); Natasha Lucas, Lilyfield, New South Wales (AU)

(73) Assignees: Sangui Bio Pty Ltd, Manly, New South Wales (AU); The University of Sydney, Sydney, New South Wales (AU)

(21) Appl. No.: 18/728,860

(22) PCT Filed: Jan. 6, 2023
(86) PCT No.: PCT/AU2023/050004

§ 371 Date: Jul. 12, 2024

Related U.S. Application Data
(60) Provisional application No. 63/299,281, filed on Jan. 13, 2022.
(60) Provisional application No. 63/392,336, filed on Jul. 26, 2022.

(51) Int. Cl. C07K 1/14; G01N 33/487; G01N 33/68

(52) U.S. Cl. CPC C07K 1/145; G01N 33/4875; G01N 33/6803; G01N 33/6842; G01N 33/6848; G01N 2570/00

(57) ABSTRACT

The object of the present invention is to provide a microsampling device that allows analysis of proteins in a biological sample, such as a whole blood sample, and provide methods of using the same.

### CROSS-REFERENCE TO RELATED APPLICATIONS

The present application claims priority to U.S. Provisional Application No. 63/299,281 filed 13 Jan. 2022 and U.S. Provisional Application No. 63/392,336 filed 26 Jul. 2022. The entire content of each of these applications is herein incorporated by reference.

### FIELD

The present invention relates to a microsampling device and analytic methods for detecting a target component in a biologic sample, such as a blood sample.

### BACKGROUND

In the last 20 years the concept of precision medicine, prevention and treatment strategies that take individual variability into account, has emerged as a way of transforming disease prediction, prognosis, and encouraging individuals to participate in their healthcare (Collins and Varmus, N Engl J Med. 2015; 372 (9): 793-5; Denny et al., N Engl J Med. 2019; 381 (7): 668-76; Franks et al., J Intern Med. 2021; Snyderman, Biotechnol J. 2012; 7 (8): 973-9). Driven by access to increasingly affordable genomic technologies, precision medicine genetic analyses are revolutionizing medicine.

Blood microsampling, which enables patients to provide longitudinal samples without attending a hospital or pathology lab, is developing rapidly and is an important innovation that links precision medicine with the growing trend of telehealth (Hollander and Sites, NEJM Catalyst. 2020). Blood volumes typically below 100 μL are collected in miniaturized devices as this is sufficient material for many molecular detection techniques (Lei and Prow, Biomed Microdevices. 2019; 21 (4): 81).

Microsampling of patient blood promises several benefits over conventional phlebotomy practices to facilitate precision medicine studies and overcomes the need for specialist phlebotomists, allowing point of care collection, at-home collection and remote sampling to occur frequently. This at-home patient blood collection supports telehealth monitoring, minimal post-collection processing, and compatibility with non-refrigerated transport and storage.

Microsampling may also increase patient accessibility for testing and overcome hesitancy in those that find venipuncture stressful (Chapman et al. Bioanalysis. 2014; 6 (22): 2965-8.). Concomitant with collection of small blood volumes is their direct analysis of pre-selected targets from dried blood spots (DBS), typified by newborn screening of small molecule metabolites for inherited metabolic disorders (Moat et al., Int J Neonatal Screen. 2020; 6 (2): 26).

The use of DBS for bottom-up, non-targeted discovery proteomic analysis of whole blood (WB), which would support precision medicine studies has not been thoroughly investigated. However, for proteomic biomarker studies, mass spectrometry of whole blood has generally been avoided in favor of using plasma or serum obtained from venipuncture.

While conventional DBS involves application of blood to paper, followed by drying, newer approaches have been developed. One such approach involves a microsampling device known as volumetric absorptive microsampling (VAMS) (Kok and Fillet, J Pharm Biomed Anal. 2018; 147:288-96). This device allows a precise volume of blood to be collected to account for variations in hematocrit levels, an important variable in diseased individuals.

Under traditional methods of blood separation, protein detection is hampered by the high relative concentrations of hemoglobin, other structural components of erythrocyte membranes as well as high concentrations of many plasma proteins. The present disclosure provides methods for analyzing proteins and cells in whole blood that are otherwise not detected by traditional blood separation methods due to dynamic range limitations. The present disclosure also provides devices and methods for utilizing said devices for analyzing proteins and cells in whole blood that are otherwise not detected by traditional blood separation methods due to dynamic range limitations.

### SUMMARY

In one aspect, provided herein is a method of fractionating a sample, comprising: a) introducing a sample into a porous material; b) optionally, centrifuging the porous material containing the sample; c) drying the sample in the porous material; d) extracting a first set of proteins from the dried sample-containing porous material, comprising: 1) incubating the dried sample-containing porous material in a first extraction solution; and 2) separating the first extraction solution from the first incubated porous material; e) detecting the first set of proteins in the separated extraction solution.

In one aspect, provided herein is a method of fractionating a sample, comprising: a) introducing a sample into a porous material; b) optionally, centrifuging the porous material containing the sample; c) drying the sample in the porous material; d) extracting a first set of proteins from the dried sample-containing porous material, comprising: 1) incubating the dried sample-containing porous material in a first extraction solution; 2) separating the first extraction solution from the first incubated porous material; and 3) optionally, washing the separated first extracted porous material; e) extracting a second set of proteins from the first extracted porous material, comprising: 1) incubating the first extracted porous material in a second extraction solution; 2) separating the second extraction solution from the second incubated porous material; and f) detecting the first or second set of proteins from the separated first or second extraction solution, respectively.

In one aspect, provided herein is a method further comprising: g) optionally, washing the separated second extracted porous material; h) extracting a third set of proteins from the second extracted porous material, comprising: 1) incubating the second extracted porous material in a third extraction solution; and 2) separating the third extraction solution from the third incubated porous material; and i) detecting the third set of proteins in the separated third extraction solution.

In some embodiments, the method further comprises sequentially extracting one or more further sets of proteins from the third extracted porous material comprising repeating the steps g)-i) with one or more further extraction solutions.

In one aspect, provided herein is a method of fractionating a sample, comprising: a) introducing a sample into a porous material; b) optionally, centrifuging the porous material containing the sample; c) drying the sample in the porous material; d) extracting a first set proteins from the dried sample-containing porous material, comprising: 1) incubating the dried sample-containing porous material in a first extraction solution; 2) separating the first extraction solution from the first incubated porous material; and 3) optionally, washing the separated first extracted porous material; e) digesting proteins remaining in the first extracted porous material, comprising: 1) incubating the first extracted porous material in a digestion solution; and 2) separating the digestion solution from the digestion incubated porous material; and f) detecting one or more proteins in the separated digestion solution.

In some embodiments, prior to the digestion step the method further comprises: A) extracting a second set of proteins from the first extracted porous material, comprising: 1) incubating the first extracted porous material in a second extraction solution; 2) separating the second extraction solution from the second incubated porous material; and g) optionally, washing the separated second extracted porous material; and B) optionally, detecting the second set of proteins in the separated second extraction solution.

In some embodiments, prior to the digestion step the method further comprises sequentially extracting one or more further sets of proteins from the second extracted porous material comprising repeating the steps A)-B) with one or more further extraction solutions.

In one aspect, provided herein is a method of fractionating a sample, comprising: a) introducing a sample into a porous material; b) optionally, centrifuging the porous material containing the sample; c) drying the sample in the porous material; d) extracting a first set molecules from the dried sample-containing porous material, comprising: 1) incubating the dried sample-containing porous material in a first extraction solution; 2) separating the first extraction solution from the porous material; and 3) optionally, washing the porous material; e) digesting proteins remaining in the porous material, comprising: 1) incubating the porous material in a digestion solution; and 2) separating the digestion solution from the porous material; and f) optionally, detecting one or more molecules in the separated first extraction solution; and g) detecting one or more peptides or proteins in the digestion solution.

In one aspect, provided herein is a method further comprising, following extraction of a first set of molecules and prior to the digestion step: A) extracting a second set of molecules from the first extracted porous material, comprising: 1) incubating the first extracted porous material in a second extraction solution; 2) separating the second extraction solution from the second incubated porous material; and h) optionally, washing the separated second extracted porous material; and B) optionally, detecting the second set of molecules in the separated second extraction solution.

In some embodiments, the method further comprises, prior to the digestion step, sequentially extracting one or more further sets of molecules from the second extracted porous material comprising repeating the steps A)-B) with one or more further extraction solutions.

In some embodiments, the set of molecules is selected from the group consisting of proteins, nucleic acids, and glycans. In another embodiment, the proteins are phosphoproteins.

In some embodiments, the sample is or comprises a body fluid sample. In some embodiments, the sample comprises cells or tissue. In some embodiments, the sample comprises cells suspended in a liquid. In some embodiments, the sample comprises cultured cells suspended in a culture media. In some embodiments, the sample is or comprises blood, blood fractions, plasma, a nucleic acid-containing stabilized sample, a stabilized blood sample (e.g., blood in a Streck tube sample), urine, tears, wound fluid, CSF, bronchoalveolar lavage, or ascites. In some embodiments, the sample is a blood sample. In some embodiments, the sample is or comprises a nucleic acid-containing stabilized sample or a stabilized blood sample, such as a nucleic acid-containing stabilized blood sample, for example a blood sample in a Streck tube. In some embodiments, the sample has a volume in the range of 100 μL to 2 μL, 100 μL to 5 μL, 100 μL to 10 μL, 99 μL to 2 μL, 90 μL to 2 μL, 80 μL to 2 μL, 70 μL to 2 μL, 60 μL to 2 μL, 50 μL to 2 μL, 40 μL to 2 μL, 30 μL to 2 μL, 20 μL to 2 μL, 10 μL to 2 μL, or 5 μL to 2 μL. In some embodiments, the sample has a volume of <100 μL, <50 μL, <30 μL, <10 μL, or <5 μL. In some embodiments, the sample has a volume of at least 2 μL.

In some embodiments, the method comprises centrifuging the porous material containing the sample prior to the drying step.

In some embodiments, the drying step, the extraction step, and the digesting step are conducted with said sample-containing porous material in said tube.

In some embodiments, the drying step comprises air drying the sample within the porous material, after which the sample is dried in the porous material. In some embodiments, the drying step comprises centrifuging the sample within the porous material, after which the sample is dried in the porous material. In some embodiments, the drying step comprises drying for a period of time to adhere the sample to the porous material. In some embodiments, the drying step comprises drying for a period of time to adhere the sample to the porous material after which the sample-containing porous material is stored for a period of time prior to the first extraction step. In some embodiments, the sample-containing porous material is frozen prior to the first extraction step. In some embodiments, the drying step comprises air drying the sample within the porous material. In some embodiments the drying step is for less than 30 mins. In some embodiments the drying step is for less than 20 minutes. In some embodiments the drying step is for less than 10 minutes. In some embodiments the drying step is for 5 minutes. In some embodiments the drying step is for less than 5 minutes. In some embodiments, the air drying is for at least 30 mins, 1, 2, 4, 6, 8, 10, 12, 16, 20 or 24 hours. In some embodiments, the drying step comprises centrifuging the sample within the porous material. In some embodiments, the sample is frozen immediately after the sample is collected into the porous material, or optionally, the sample is air dried for less than 30 minutes before being frozen.

In some embodiments, the method comprises storing the sample-containing porous material for a period of time prior to the first extraction step. In some embodiments, the method comprises storing the sample-containing porous material immediately following the drying step.

In some embodiments, the sample-containing porous material is stored frozen. In some embodiments, the sample-containing porous material is stored frozen at −20° C. In some embodiments, the sample-containing porous material is stored frozen at −80° C. In some embodiments, the the sample-containing porous material is thawed and re-frozen prior to the first extraction step. In some embodiments, the sample-containing porous material is stored at room temperature.

In some embodiments, the first, the second, the third, and the further extraction solution comprises an extracting agent comprising a salt, a mild detergent, a strong detergent, a chaotrope, a reducing agent, a thiol-containing reducing agent, or an alkylating agent, an acid, an organic solvent, or an enzyme, or combinations thereof.

In some embodiments, the first extraction solution and the second extraction solution are different. In some embodiments, the first, the second, and the third extraction solutions are different. In some embodiments, the first, the second, the third, and the further extraction solutions are different. In some embodiments, the selection of the first, the second, the third, and the further extraction solutions is to fractionate the proteins contained in the porous material via differential solubility. In some embodiments, each sequential extraction solution selected is characterized as having greater solubility of the proteins remaining in the porous material. In some embodiments, the first, the second, the third, and the further extraction solutions comprise identical extracting agents of increasing concentrations as progress from one extraction to the next extraction.

In some embodiments, the separated first, second, third, or further, extraction solution comprises one or more proteins. In some embodiments, the separated first extraction solution comprises the first set of proteins. In some embodiments, the separated second extraction solution comprises the second set of proteins. In some embodiments, the separated third extraction solution comprises the third set of proteins. In some embodiments, the separated further extraction solution comprises the further set of proteins. In some embodiments, the first, the second, the third, or the further, extraction solution has a volume of less than 100 μL. In some embodiments, the first, the second, the third, or the further, extraction solution has a volume of 1-5 times the volume of porous material.

In some embodiments, the incubating in the first, the second, the third, or the further, extraction solution is conducted for a period of time long enough to solubilize one or more proteins contained within or adhered to the porous material. In some embodiments, the incubating in the first, the second, the third, or the further, extraction solution is conducted for a period of time long enough to extract one or more proteins contained within or adhered to the porous material. In some embodiments, the said incubating is conducted for 1 min-48 hr. In some embodiments, the incubating is conducted at ambient temperature. In some embodiments, the incubating is conducted at a temperature elevated above ambient temperature. In some embodiments, the incubating is conducted at a temperature elevated below ambient temperature. In some embodiments, the incubating is conducted with agitation.

In some embodiments, the incubating in the digestion solution is conducted for a period of time long enough to digest one or more proteins contained within or adhered to the porous material. In some embodiments, the incubating is conducted for 1 min-48 hour. In some embodiments, the incubating is conducted for 1 min-24 hour. In some embodiments, the incubating is conducted for 24-48 hour. In some embodiments, the incubating is conducted at ambient temperature. In some embodiments, the incubating is conducted at a temperature elevated above ambient temperature. In some embodiments, the incubating is conducted at a temperature elevated below ambient temperature. In some embodiments, the incubating is conducted with agitation.

In some embodiments, the separating comprises removing the first, the second, the third, or the further, incubated porous material from the first, the second, the third, or the further, extraction solution, respectively. In some embodiments, the separating comprises removing the first, the second, the third, or the further, extraction solution from the first, the second, the third, or the further, incubated porous material, respectively. In some embodiments, the separating of the first, the second, the third, or the further, extraction solution from the first, the second, the third, or the further, incubated porous material is via centrifugation, filtration, or a combination of centrifugation and filtration, respectively. In some embodiments, the separating step is conducted via centrifugation. In some embodiments, the separating step is conducted via filtration. In some embodiments, the separating step is conducted via a combination of centrifugation and filtration.

In some embodiments, the separating comprises removing the digestion incubated porous material from the digestion solution. In some embodiments, the separating comprises removing the digestion solution from the digestion incubated porous material. In some embodiments, the separating of the digestion solution from the digestion incubated porous material is via centrifugation, filtration, or a combination of centrifugation and filtration. In some embodiments, the separating step is conducted via centrifugation. In some embodiments, the separating step is conducted via filtration. In some embodiments, the separating step is conducted via a combination of centrifugation and filtration.

In some embodiments, the method comprises washing the separated first, second, third, or further, extracted porous material. In some embodiments, the separated first, second, third, or further extracted porous material is washed with a washing volume of the first, second, third, or further extraction solution, respectively. In some embodiments, the separated first extracted porous material is washed with an extraction solution different from the first extraction solution. In some embodiments, selection of the different extraction solution is limited to solutions based on proteins remaining in the separated first extracted porous material have equivalent or less solubility in the different extraction solution than the first extraction solution. In some embodiments, the selection of the different extraction solution is based on the proteins remaining in the separated first extracted porous material having equivalent or less solubility in the different extraction solution than said first extraction solution.

In some embodiments, the washing volume of the first, the second, the third, or the further, extraction solution is 50 μL or more. In some embodiments, the washing volume of the first, the second, the third, or the further, extraction solution is 100 μL or more. In some embodiments, the washing volume of the first, the second, the third, or the further, extraction solution is between 5 times the volume of porous material and 3 mL. In some embodiments, the washing of the separated first, second, third, or further extracted porous material is repeated 1, 2, or 3 times.

In some embodiments, the digestion solution comprises a reducing agent, an alkylating agent, a buffer, a detergent, or combinations thereof. In some embodiments, the digestion solution further comprises a salt, a mild detergent, a strong detergent, a chaotrope, or a thiol-containing reducing agent, or combinations thereof. In some embodiments, the digestion solution further comprises a tryptic digestion solution. In some embodiments, the digestion solution comprises a protease or a combination of proteases. In some embodiments, the protease is trypsin or the combination of proteases comprises trypsin.

In some embodiments, the detecting step comprises detecting proteins in the separated first extraction solution. In some embodiments, the detecting step comprises detecting proteins in the separated second extraction solution. In some embodiments, the detecting step comprises detecting proteins in the separated third extraction solution. In some embodiments, the detecting step comprises detecting proteins in the further extraction solution. In some embodiments, the detecting of the first, second, third, or further sets of proteins from the first, second, third, or further extraction solutions, respectively, is via immunoassay. In some embodiments, the detecting of the first, second, third, or further sets of proteins from the first, second, third, or further extraction solutions, respectively, is via Mass Spectrometry (MS). In some embodiments, the detection via MS comprises subjecting said first, second, third, or further extraction solution to digestion prior to said detection via Mass Spectrometry (MS). In some embodiments, prior to detecting the first, second, third, or further sets of proteins in the first, second, third, or further extraction solutions, respectively, the method further comprises incubating said first, second, third, or further extraction solution, in a digestion solution.

In some embodiments, the porous material is a three-dimensional porous material. In some embodiments, the three-dimensional porous material is a tip of a volumetric absorptive microsampling (VAMS) device. In some embodiments, the porous material is a non-pre-loaded porous material. In some embodiments, the porous material is not pre-loaded with protease inhibitors. In some embodiments, the porous material is a pre-loaded porous material. In some embodiments, the porous material is pre-loaded with a protease inhibitor. In some embodiments, the porous material is pre-loaded with an anticoagulant. In some embodiments, the porous material is pre-loaded with an enzyme. In some embodiments, the porous material is pre-loaded with a detergent.

In some embodiments, the method further comprises placing the sample-containing porous material in a tube. In some embodiments, the method further comprises placing the dried sample-containing porous material in a tube. In some embodiments, the method uses a plurality of the porous materials. In some embodiments, the sample is introduced into the plurality of the porous materials. In some embodiments, the plurality of the porous materials comprises two or more porous materials.

In one aspect, provided herein is a method of producing a protein profile, comprising the use of the any one of the methods disclosed herein.

In one aspect, provided herein is a method of isolating one or more proteins, comprising the use of the any one of the methods disclosed herein.

In one aspect, provided herein is a method of portioning proteins in a sample by differential solubility, comprising the use of the any one of the methods disclosed herein.

In one aspect, provided herein is a method for preparing sequential samples using an absorptive device, comprising the use of the any one of the methods disclosed herein.

In one aspect, provided herein is a microsampling device, comprising: a) a first porous material; b) a second porous material; and c) a tube.

In one aspect, provided herein is a microsampling device, comprising: a) a first porous material, b) a second porous material; c) a third porous material; and d) a tube.

In some embodiments, the tube is an Eppendorf tube.

In one aspect, provided herein is a microsampling device, comprising: a) a first porous material; b) a second porous material; and c) a shaft; wherein the first porous material is positioned at one end of the shaft and the second porous material is located at a position along the shaft so that the first porous material and the second porous material are separated and not in physical contact with each other.

In one aspect, provided herein is a microsampling device, comprising: a) a first porous material; b) a second porous material; c) a third porous material; and d) a shaft; wherein the first porous material is positioned at one end of the shaft and the second porous material and the third porous material are located at positions on the shaft so that the first porous material, the second porous material, and the third porous material are separated and not in physical contact with each other.

In some embodiments, the microsampling device is a volumetric absorptive microsampling (VAMS) device.

In some embodiments, the first porous material and the second porous material are positioned on the shaft and separated from each other by a spacer such that said first porous material and said second porous material are separated and not in physical contact with each other.

In some embodiments, the first porous material, the second porous material, and the third porous material are positioned on the shaft and separated from each other by spacers such that said first porous material, said second porous material, and said third porous material are separated and not in physical contact with each other.

In some embodiments, the shaft is a threaded shaft. In some embodiments, the shaft is a non-linear shaft. In some embodiments, the shaft is a curved shaft. In some embodiments, the shaft has a length of 10 mm to 50 mm. In some embodiments, the shaft has a diameter of 0.5 mm to 3 mm. In some embodiments, the shaft fits in the tube. In some embodiments, the shaft fits in a tube having a volume of 5 mL, 2 mL, 1.5 mL, or 1 mL. In some embodiments, the shaft fits in an Eppendorf tube. In some embodiments, the shaft fits in a 96-well plate.

In some embodiments, the first porous material and second porous material are pre-loaded with anticoagulant. In some embodiments, the first porous material has an absorptive capacity in the range of 2.5 μL to 50 μL. In some embodiments, the first porous material and second porous material have the same absorptive capacity. In some embodiments, the first porous material and second porous material have a different absorptive capacity. In some embodiments, the first porous material, the second porous material, and the third porous material have the same absorptive capacity. In some embodiments, the first porous material, the second porous material, and the third porous material have different absorptive capacities. In some embodiments, the first porous material and the second porous material have the same absorptive capacity, and the third porous material has a different absorptive capacity relative to said first and second porous materials.

In one aspect, provided herein is a method of using any one of the devices disclosed herein according to any one of the methods disclosed herein.

In one aspect there is provided a method of producing a protein profile comprising: a) obtaining at least one protein profile produced according to any one of the methods disclosed herein from a sample that has been obtained from a subject having a disease or disorder; b) obtaining at least one protein profile produced according to any one of the methods disclosed herein from a sample that has been obtained from at least one subject not having the disease or disorder; c) comparing the protein profile of the subject having the disease or disorder to the protein profile of the at least one subject not having the disease or disorder, and d) producing the disease protein profile from the comparison, wherein said produced disease protein profile comprises one or more proteins that have a different presence or level in the protein profile from the subject having the disease or disorder compared to the protein profile of the at least one subject not having the disease or disorder.

In some embodiments the protein profile is obtained from proteins obtained from one or more proteins obtained from an extraction solution and from a digestion solution.

### BRIEF DESCRIPTION OF THE DRAWINGS

FIGS. 1A-1D show VAMS DBS washing procedure. FIG. 1A. VAMS DBS tip was removed from its spindle and placed in extraction solution for 24 hours. FIG. 1B. VAMS tips were then pulsed 0-10000 g 3 times. FIG. 1C. VAMS tips were then placed within a custom column insert within a waste collection microcentrifuge tube and spun at 3000 g for 3 minutes. Steps B and C were repeated 3 times. FIG. 1D. After the third wash the dry tip with the extraction solution removed was placed in a fresh microcentrifuge tube for tryptic digestion.

FIG. 2 shows an UpSet plot showing proteins identified from whole blood (WB) and plasma using VAMS devices.

FIGS. 3A-3B show varying DBS washing conditions. FIG. 3A. Upset plot showing effect of different DBS washing conditions on protein detection. FIG. 3B. Principal component analysis (PC1, PC2) demonstrating that urea washing of DBS produces a different subset of proteins.

FIGS. 4A-4B show LC-MS analysis of proteins recovered from VAMS. FIG. 4A. Upset plot showing proteins detected from whole blood using paper DBS (PBS paper and LiCl paper) or VAMS devices (LiCl fresh and LiCl frozen) being freshly applied or applied after being frozen. FIG. 4B. Principal component analysis (PC1, PC2) demonstrating differences in protein recovery in PBS washed DBS on paper.

FIG. 5 shows a volcano plot identifying differentially abundant proteins in frozen blood cell pellets from a group of patients with the same disease. FC>2, p<0.05.

FIGS. 6A-6C show cellular components of plasma and white blood cell (WBC) fractions. FIG. 6A shows a bar graph depicting three cellular components including cellular anatomical entities, intracellular proteins and protein containing complexes. FIG. 6B shows a bar graph further classifying the categories of non-membrane-associated protein complexes. FIG. 6C shows a bar graph depicting the categories of membrane-associated protein complexes.

FIGS. 7A-7C show cellular components of an RBC fraction. FIG. 7A shows a bar graph depicting three cellular components including cellular anatomical entities, intracellular proteins and protein containing complexes. FIG. 7B shows a bar graph further classifying the categories of non-membrane-associated protein complexes. FIG. 7C shows a bar graph depicting the categories of membrane-associated protein complexes.

FIGS. 8A-8H shows a 96 well scale up of the device provided herein. FIG. 8A shows Neoteryx tips, with a blood sample absorbed onto the tips, placed into extraction solution in a 96 well plate. FIG. 8B shows that the tips are removed from the extraction solution. FIG. 8C shows a new 96 well plate with holes in the bottom placed atop the first 96 well plate. FIG. 8D shows the tips are lowered into the new 96 well plate. FIG. 8E shows a tip remover plate is placed over the top of the 96 well plate. FIG. 8F shows that the tips are removed from the shaft by sliding the tip remover plate sideways and lifting the tip shafts. FIG. 8G shows the shafts and the tip remover plate are removed. FIG. 8H shows the empty tips are retained in the top plate and the extract is retained in the bottom 96 well plate.

FIG. 9 shows an UpSet plot depicting that washing VAM devices 3 times with 1 mL of 0.1 M Tris-HCl containing 0.5 M NaCl results in the identification of 1465 proteins that are common to frozen whole blood (WB), WB and white blood cells (WBC), followed by 1233 proteins that are unique to WBC.

FIG. 10 shows an UpSet plot demonstrating that washing Neoteryx tips 2 times with 1 mL of 2M urea results in the identification of a different protein profile, with ˜550 less proteins identified relative to FIG. 9.

FIG. 11 shows an UpSet plot depicting that washing Neoteryx tips or filter paper with LiCl or NaCl, identifies 1321 proteins present in filter paper washed with LiCl, filter paper washed with NaCl, a Neoteryx tip washed with LiCl and a Neoteryx tip washed with NaCl.

FIG. 12 shows a clustergram depicting samples or wash types that produce distinct protein sets. The x-axis depicts the different sample types or washes. The y-axis depicts the proteome groupings identified by Kyoto Encyclopedia of Genes and Genomes (KEGG) database. The legend shows statistical significance by grayscale, where darker gray reflects a lower p-value and thus more significant values. 2 M urea was produced a significantly different protein profile compared to other washes.

FIG. 13 shows an example of two pieces of Mitra® 3-D porous material from Neoteryx inside a plastic tube. The bottom of the tube was sealed to prevent sample loss.

FIG. 14 shows a tube containing 2 pieces of Mitra® 3-D porous material from Neoteryx after loading a whole blood sample (approximately 100 μL) and fractionating by centrifugation (1500 g for 5 minutes).

FIG. 15 shows the 2 pieces of Mitra® 3-D porous material from Neoteryx after loading a whole blood sample and fractionating by centrifugation (1500 g for 5 minutes). The 2 pieces of Mitra® 3-D porous material have been removed from the tube and placed back on their handles to enable storage and drying. The left piece of Mitra® 3-D porous material is the top (plasma and white blood cell rich) sub-sample and the right piece of Mitra® 3-D porous material is the bottom (RBC rich) sub-sample.

FIG. 16 shows filtered microcentrifuge tubes comprising a top portion and bottom portion of a tube that the extract was stored frozen in at −20° C. until required for analysis.

FIG. 17 shows an experimental flow chart of a method of using the device provided herein.

FIG. 18 shows a Venn diagram depicting the number of identified proteins unique to the plasma fraction (246 proteins) and RBC rich fraction (255 proteins), and the number of proteins identified in both fractions (206 proteins).

FIG. 19 shows a Venn diagram depicting the number of identified proteins unique to the plasma fraction (3 proteins) and the RBC rich fraction (300 proteins), and the number of proteins identified in both fractions (51 proteins).

FIGS. 20A-20B show an example of two pieces of Mitra® 3-D porous material from Neoteryx mounted on a metal shaft (FIG. 20A), of which the individual sections are identified schematically in FIG. 20B.

FIGS. 21A-21B shows device FIG. 21A (i.e., the device from FIG. 20) and after it is loaded with a whole blood sample and fractionated by centrifugation (1500 g for 5 minutes) in FIG. 21B.

FIG. 22 shows a Venn diagram depicting the number of identified proteins unique to the plasma fraction (1017 proteins) and the RBC rich fraction (80 proteins), and the number of proteins identified in both fractions (262 proteins).

FIG. 23 shows a Venn diagram depicting the number of identified proteins unique to the plasma/WBC fraction (791 proteins) and the RBC rich fraction (29 proteins), and the number of proteins identified in both fractions (40 proteins).

FIG. 24 shows an UpSet plot depicting the presence of various proteins in frozen whole blood cell pellets from 5 healthy and 5 Stage III cancer donors. Healthy and cancer samples were matched for both age and sex. Healthy samples are shown in odd numbers 1, 3, 5, 7 and 9, and cancer samples are shown in even numbers 2, 4, 6, 8 and 10.

FIGS. 25A-25C show box and whisker plots depicting the middle 50 percent of the data values, also known as the interquartile range, or IQR. The median of the values is depicted as a line splitting the box in half. The IQR illustrates the variability in a set of values. A large IQR indicates a large spread in values, while a smaller IQR indicates most values fall near the center. Box plots also illustrate the minimum and maximum data values through whiskers extending from the box, and optionally, outliers as points extending beyond the whiskers. FIG. 25A shows label-free quantification (LFQ) unfiltered. FIG. 25B shows LFQ filtered with zeros shifted. FIG. 25C shows median norm filtered.

FIGS. 26A-26C show box and whisker plots depicting the middle 50 percent of the data values, also known as the interquartile range, or IQR. The median of the values is depicted as a line splitting the box in half. The IQR illustrates the variability in a set of values. A large IQR indicates a large spread in values, while a smaller IQR indicates most values fall near the center. Box plots also illustrate the minimum and maximum data values through whiskers extending from the box, and optionally, outliers as points extending beyond the whiskers. FIG. 26A shows missing values imputed by sample. FIG. 26B shows median norm filtered. FIG. 26C shows median norm LFQ with zeros imputed.

FIGS. 27A-27B show volcano plots depicting differentially expressed (DE) proteins/genes that were significantly different between cancer and control. FIG. 27A shows plain t-tests. FIG. 27B shows Limma moderated t-tests.

FIG. 28 shows a heat map depicting that the differentially expressed (DE) proteins/genes between cancer and controls show pronounced separation.

FIGS. 29A-29B show clustergrams depicting enrichment subcellular proteins between the cancer (frozen samples) to freshly prepared and frozen samples from healthy people.

FIGS. 30A-30C show a volumetric absorptive microsampling devices. FIG. 30A shows a tube containing two ‘plugs’ of sponge-like absorbent material that take up a defined volume of fluid. The absorbent plugs may also contain anti-coagulant material (wet or dry) to prevent blood clotting during the separation. FIG. 30B shows the introduction of blood to the top of the tube. FIG. 30C shows the tube after centrifugation. RBCs, WBCs and platelets are removed from the plasma and contained within absorbent plug 1. Absorbent plug 2 contains plasma.

FIGS. 31A-31D show a volumetric absorptive microsampling device. FIG. 31A shows a tube containing two ‘plugs’ of sponge-like absorbent material connected to each other by a shaft. FIG. 31B shows the introduction of blood to the top of the tube. FIG. 31C shows the tube after centrifugation. RBCs, WBCs and platelets are removed from the plasma and contained within absorbent plug 1. Absorbent plug 2 contains plasma. FIG. 31D shows the removal of the absorbent plugs after centrifugation.

FIGS. 32A-32D show a volumetric absorptive microsampling device. FIG. 32A shows tubes provided with a lid to prevent spillage and suppress the release of aerosols during centrifugation. FIG. 32B shows the introduction of blood to the top of the tube. FIG. 32C shows the tube after centrifugation. RBCs, WBCs and platelets are removed from the plasma and contained within absorbent plug 1. Absorbent plug 2 contains plasma. FIG. 32D shows the removal of the absorbent plugs after centrifugation.

FIGS. 33A-33B show a volumetric absorptive microsampling device. FIG. 33A shows tube that may have a wider opening at the top to facilitate blood collection by wiping or scraping blood drops from a fingertip. FIG. 33B shows a long and narrow tube, with variable spacing between the absorbent plugs to ensure that the plasma sample is not contaminated by cells or platelets.

FIG. 34 shows a squeeze tube shaft, as indicated by arrows, to create suction and aspirate blood drops.

FIG. 35 shows a wire to create the shaft (handle). This was shaped to keep the absorbent plugs separate. The blood sample was EDTA anti-coagulated venous blood. The fractionation device shown was placed in an empty 1,500 μL Eppendorf tube. Blood was added using a pipette, until the top absorbent plug was covered. The tube was closed and centrifuged at 1,500 g for 4 minutes. The wire shaft was used to remove the device from the tube and the absorbent plugs allowed to air dry. The cellular component, particularly RBCs, have been substantially removed from the top absorbent plug—by visual assessment.

FIGS. 36A-36C show 2 mm threaded bolts to create the shaft (handle). The thread on the shaft prevented the upper (plasma) absorbent plug from moving down during centrifugation. The blood sample was finger prick capillary blood. The absorbent plugs were wetted with 10× EDTA anti-coagulant. The fractionation devices shown were placed in empty 500 μL Eppendorf tubes. Blood was collected from finger prick drops using a disposable pipette and added-approximately 50 μL. The tube was closed and centrifuged for 2 minutes using a modified battery powered fan. The shaft with the absorbent plugs was removed using tweezers and the absorbent plugs allowed to air dry. The cellular component, particularly RBCs, have been substantially removed from the top absorbent plug by visual assessment.

FIG. 37 shows an experimental work flow of a method for sequential sample preparation using absorptive devices. In this basic work flow, a low volume extraction is performed with PBS followed by a high volume wash. These steps are repeated prior to trypsin digestion of the tip.

FIG. 38 shows an experimental work flow of a method for sequential sample preparation using absorptive devices. In this work flow, there are two extractions and two analyses. The first extraction is a low volume extraction with PBS or Tween (or PBS and Tween), followed by a high volume wash with NaCl or LiCl. The wash can be repeated 1, 2 or 3 times prior to trypsin digestion of the tip.

FIG. 39 shows an experimental work flow of a method for sequential sample preparation using absorptive devices. In this work flow, there are two extractions and analyses, with stringent washing to remove non-membrane proteins. This work flow modifies the basic work flow. First, a low volume extraction is performed with PBS or Tween (or PBS and Tween), followed by a high volume wash with urea, urea/thiourea or detergent. The wash is performed 1, 2 or 3 times prior to trypsin digestion of the tip.

FIG. 40 shows an experimental work flow of a method for sequential sample preparation using absorptive devices. In this work flow, three extractions and analyses are performed, with a stringent extraction as the middle step. First, a low volume extraction is performed with PBS or Tween (or PBS and Tween) followed by a low volume extraction with urea, urea/thiourea or detergent, prior to trypsin digestion of the tip.

FIG. 41 shows an experimental work flow of a method for sequential sample preparation using absorptive devices. In this work flow, 2 porous materials were used in the device. The device was centrifuged and two extractions and two analyses were performed. A low volume extraction was performed with Tween or PBS (or PBS and Tween) and a high volume extraction (1 mL) with NaCl or LiCl was performed 1, 2 or 3 times prior to trypsin digestion.

FIG. 42 shows total peptide yields recovered according to Example V.

FIG. 43 shows number of protein and peptide identifications (IDs) observed according to Example V.

FIG. 44 shows percentage of missed cleavages after trypsin digestion according to Example V.

FIG. 45 shows total protein yields recovered according to Example VI.

FIG. 46 shows number of protein and peptide identifications (IDs) observed according to Example VI.

FIG. 47 shows percentage of missed cleavages after trypsin digestion according to Example VI, and in Hela cells as a control (Hela Standard).

FIG. 48 shows percentage of alkylated peptides (containing carbamidomethyl modification (CAM)) according to Example VI.

FIG. 49 shows Venn diagram depicting number of identified proteins unique to peripheral blood mononuclear cells (PBMCs) samples and whole blood (WB) samples, and number of proteins identified in both fractions, according to Example VI.

FIG. 50 shows protein classes of those proteins found exclusively in PBMCs, according to Example VI.

FIG. 51 shows protein classes of those proteins found exclusively in WB, according to Example VI.

FIG. 52 shows total protein yields recovered according to Example VII.

FIG. 53 shows number of protein IDs observed according to Example VII.

FIG. 54 shows number of peptide IDs observed according to Example VII.

FIG. 55 shows percentage of missed cleavages after trypsin digestion according to Example VII.

FIG. 56 shows a Venn diagram depicting a number of identified proteins unique to WB samples and WB Streck tube samples, and a number of proteins identified in both fractions, according to Example VII.

FIG. 57 shows a Venn diagram depicting a number of identified proteins unique to PBMCs samples and WB samples, and a number of proteins identified in both fractions, according to Example VII.

FIG. 58 shows a Venn diagram depicting a number of identified proteins unique to PBMCs samples, WB samples, WB Streck tube samples, and a number of proteins identified in all three fractions, according to Example VII.

FIG. 59 shows protein classes of those proteins found exclusively in PBMCs samples, according to Example VII.

FIG. 60 shows protein classes of those proteins found exclusively in WB and WB-Streck tube samples, according to Example VII.

FIG. 61 shows protein yields recovered according to Example VIII.

FIG. 62 shows number of protein IDs observed according to Example VIII.

FIG. 63 shows number of peptide IDs observed according to Example VIII.

FIG. 64 shows percentage of missed cleavages after trypsin digestion according to Example VIII.

FIG. 65 shows a Venn diagram depicting a number of identified proteins unique to Buffy Coat samples washed with LiCl (BC LiCl), Buffy Coat samples washed Urea/Thio Urea (BC Urea/ThioU), and a number of proteins identified in both fractions, according to Example VIII.

FIG. 66 shows a Venn diagram depicting a number of identified proteins unique to WB samples washed with LiCl (WB LiCl), WB samples washed with Urea/Thio Urea (WB Urea/ThioU), and a number of proteins identified in both fractions, according to Example VIII.

FIG. 67 shows the top protein IDs observed from the Buffy Coat with LiCl wash samples, according to Example VIII.

FIG. 68 shows the top protein IDs observed from the Buffy Coat with Urea/ThioUrea wash samples, according to Example VIII.

FIG. 69 shows a Venn diagram depicting a number of identified proteins unique to WB samples (WB LiCl) and Buffy Coat samples (BC LiCL) each washed with LiCl, and a number of proteins identified in both fractions, according to Example VIII.

FIG. 70 shows a Venn diagram depicting a number of identified proteins unique to PBMC samples and Buffy Coat samples, and a number of proteins identified in both fractions, according to Example VIII.

FIG. 71 shows total protein yields recovered according to Example IX.

FIG. 72 shows number of protein IDs observed according to Example IX.

FIG. 73 shows a Venn diagram depicting overlap of Protein ID's for Buffy Coat (BC) tips washed with LiCl; Urea/Thiourea (UT); Urea/Thiourea/SDC; Urea/Thiourea/SDC/TBP according to Example IX.

FIG. 74 shows protein classes of those proteins found exclusively in TBP, according to Example IX.

FIG. 75 shows protein classes of those proteins found exclusively in LiCl, according to Example IX.

FIG. 76 shows overlap of Protein IDs for RBC, RBC pellet and supernatant, according to Example IX.

FIG. 77 shows Proteins found exclusively in RBC pellet, according to Example IX.

FIG. 78 shows Proteins found exclusively in RBC supernatant, according to Example IX.

FIG. 79 shows a Venn diagram showing the overlap of proteins for PBMCs and whole blood, according to Example IX.

FIG. 80 shows the top abundant proteins in whole blood, according to Example IX.

FIG. 81 shows the top abundant proteins in PBMCs, according to Example IX.

FIG. 82 shows total protein yields recovered according to Example X.

FIG. 83 shows number of protein IDs observed according to Example X.

FIG. 84 shows protein and peptide IDs from DIA-NN for PBMCs digested directly in solution or digested in-tip following sequential extraction, according to Example X.

FIG. 85 shows a summary of proteins down-regulated from PBMCs in VAMS, according to Example X.

FIG. 86 Shows a summary of proteins up-regulated from PBMCs in VAMS, according to Example X.

FIG. 87 shows protein and peptide IDs from DIA-NN for samples subjected to different washing conditions, according to Example X.

FIG. 88 shows a Venn diagram displaying the overlap of upregulated proteins in TBP and CLA, according to Example X.

FIG. 89 shows a Venn diagram displaying the overlap of down regulated proteins, respectively, in TBP and CLA, according to Example X.

FIG. 90 shows protein quantitation views for the various samples, according to Example XI.

FIG. 91 shows volcano plot depicting differential expression for the various samples, according to Example XI.

FIG. 92 shows a heatmap of selected 13 differentially expressed proteins that clustered well with each participant group, according to Example XI.

FIG. 93 shows protein yield for individual tip replicates, according to Example XII.

FIG. 94 shows total yield between batches, according to Example XII.

FIG. 95 shows total protein and peptide IDs from DIA-NN, according to Example XIII.

FIG. 96 shows average missed cleavages for individual tip replicates, according to Example XI.

FIG. 97. Shows the number of proteins with % CV of <20% (blue) and % CV of ≥20% (orange) from analytical replicates using the same tip (intra-tip MS1), tips from each batch (inter-tip Batch 1 or 2), tips from Batch 1 re-analysed when Batch 2 were analysed (intra-tip MS2), all tips on each instrument (inter-instrument) and finally, all tips together (inter-tip all), according to Example XII.

FIG. 98 shows a box and whiskers plot of % CV from analytical replicates using the same tip (intra-tip MS1), tips from each batch (inter-tip Batch 1 or 2), tips from Batch 1 re-analysed when Batch 2 were analysed (intra-tip MS2), all tips on each instrument (inter-instrument) and finally, all tips together (inter-tip all), according to Example XII.

FIG. 99 shows violin plot of % CV for each batch (B1 and B2) and both batches together (intra-batch) left to right. Dotted line marks 20% CV. Dot marks the median % CV, according to Example XII.

FIG. 100 shows PCA plot of tip and batch reproducibility, according to Example XII.

FIG. 101 shows total yield from DBS allowed to dry at room temperature for a week (dried) and from DBS prevented from drying by freezing and cycling through 3× freeze thaws, according to Example XIII.

FIG. 102 protein IDs from DIA-NN for dried vs frozen DBS, according to Example XIII.

FIG. 103 is a Venn diagram showing the overlap of protein IDs from dried vs frozen VAMS, according to Example XIII.

FIG. 104 shows % CVs of replicates in dried DBS vs frozen DBS, according to Example XIII.

FIG. 105 shows the most abundant proteins in dried (A) vs frozen samples (B), according to Example XII.

FIG. 106 shows the most abundant blood proteins in dried vs frozen samples, according to Example XIII.

FIG. 107 shows dynamic range defined as the area difference between the highest and lowest peak from both DBS dried at room temperature and DBS frozen, according to Example XIII.

FIG. 108 shows total protein yield for whole blood DBS after storage at room temperature (dried), 37° C. (dried 37° C.), frozen at −80° C. (frozen) or lysed and then frozen at −80° C. (lysed-frozen), according to Example XIV.

FIG. 109 shows total protein yield for plasma DBS after storage at room temperature (dried) or frozen at −80° C. (frozen), according to Example XIV.

FIG. 110 is a Venn diagram showing overlap of dried vs frozen whole blood DBS that were extracted with DUTRA wash, according to Example XIV.

FIG. 111 shows proteins found exclusively in dried samples, according to Example XIV.

FIG. 112 shows proteins found exclusively in frozen samples, according to Example XIV.

FIG. 113 is a Venn diagram showing overlap of up- and down-regulated proteins in LiCl extraction and DUTRA extraction, according to Example XIV.

FIG. 114 shows the dynamic range defined as the area difference between the highest and lowest peak from both DBS dried at room temperature and DBS frozen followed by DUTRA extraction, according to Example XIV.

FIG. 115 shows overlap of down-regulated and up-regulated protein IDs from VAMS stored at room temperature (RT), 37° C. (37) or frozen at −80° C. (Fz), according to Example XIV.

FIG. 116 shows overlap of proteins identified in whole blood (un-lysed) tips (un-lysed frozen) and whole blood lysed prior to loading into tip (lysed frozen). Both sets of tips were stored frozen at −80° C., according to Example XIV.

FIG. 117 shows overlap of protein IDs from whole blood and plasma VAMS that have been dried at room temperature or else frozen immediately, according to Example XIV.

FIG. 118 shows protein classes up-regulated in plasma VAMS dried at room temperature vs frozen at −80° C., according to Example XIV.

FIG. 119 and down-regulated, respectively in plasma VAMS dried at room temperature vs frozen at −80° C., according to Example XIV.

FIG. 120 shows total protein yield from all samples, according to Example XV.

FIG. 121 shows the number of protein IDs from a variety of processing methods, according to Example XV.

FIG. 122 is a Venn diagram showing overlap of protein IDs from VAMS dried and stored at a variety of temperatures, room temperature (control), 37° C. (37), or 99° C. (100), according to Example XV.

FIG. 123 is a Venn diagram showing overlap in protein IDs from venous blood vs finger prick blood, according to Example XV.

FIG. 124 shows protein classes unique to venous blood samples, according to Example XV.

FIG. 125 shows protein classes unique to fingerprick blood samples, according to Example XV.

FIG. 126 is a Venn diagram showing overlap of protein IDs from samples processed using the standard methods and samples initially extracted for DNA then extracted for proteins, according to Example XV.

FIG. 127 is a Venn diagram showing overlap of protein IDs after using various washing buffers for extraction, according to Example XV.

FIG. 128 is an upset Upset plot of protein ID overlap from various washing and extraction methods including sonication and benzonase during extraction, according to Example XV.

FIG. 129 shows a PCA plot comparing each of the washing and drying methods of whole blood VAMS, according to Example XV.

FIG. 130 shows a summary of the protein peak areas for the most abundant proteins using various washing methods (1-20 most abundant proteins), according to Example XV.

FIG. 131 shows a summary of the protein peak areas for the most abundant proteins using various washing methods (21-30 most abundant proteins), according to Example XV.

FIG. 132 is a Venn diagram showing overlap in protein IDs for samples treated with benzonase at different stages of the extraction compared to an untreated control, according to Example XV.

FIG. 133 shows total protein yield from all digested samples, according to Example XVI.

FIG. 134 Shows protein IDs from liquid samples (digest) or in-tip processed samples (tip) for MBCC and yeast cells, according to Example XVI.

FIG. 135 shows the overlap of protein IDs between liquid (digest) and in-tip samples for MBCC and yeast cells, according to Example XVI.

FIG. 136 shows proteins found exclusively in MBCC liquid samples, according to Example XVI.

FIG. 137 shows proteins found exclusively in MBCC in-tip samples, according to Example XVI.

FIG. 138 shows proteins found exclusively in yeast liquid samples, according to Example XVI.

FIG. 139 shows proteins found exclusively in yeast in-tip samples, according to Example XVI.

FIG. 140 shows abundant mouse proteins across serially diluted MBCC in whole blood, according to Example XVI.

FIG. 141 shows abundant human proteins across serially diluted MBCC in whole blood, according to Example XVI.

FIG. 142 shows protein IDs from liquid samples in-tip processed samples that underwent a variety of washing, digesting, or processing methods, according to Example XVII.

FIG. 143 shows Protein IDs from liquid samples in-tip processed samples that underwent a variety of washing, digesting, or processing methods, according to Example XVII.

FIG. 144 shows PCA plot comparing each of the washing methods of whole blood VAMS as well as sample collection in a smaller volume VAMS as well as collection into PVDF membrane, according to Example XVII.

FIG. 145 shows Overlap of protein IDs from whole blood VAMS samples that were extracted according to the standard methods (LiCl) or using PBS for extraction of DNA or immunoassay analysis prior to mass spectrometry extraction and analysis, according to Example XVII.

FIG. 146 shows Relative abundance of top ranked abundant proteins for whole blood VAMS extracted with the standard method (WB Ctrl-LiCl), DTT, 2M Urea & CHAPS, Citric acid, or a combination wash containing benzonase, LiCl, CHAPS, and sonication, according to Example XVII.

FIG. 147 shows Protein classes found exclusively in 2M Urea & 2% CHAPS extraction, according to Example XVII.

FIG. 148 shows Protein classes found exclusively with DTT reduction during extraction, according to Example XVII.

FIG. 149 shows Protein classes found exclusively with combination wash containing benzonase, LiCl, CHAPS, and sonication, according to Example XVII.

FIG. 150 shows Overlap of (A) peptides and (B) proteins from whole blood samples digested with trypsin, Asp-N, or Glu-C, according to Example XVII.

FIG. 151 shows Overlap of proteins extracted from 30 μL VAMS and 10 μL VAMS using the same protocol, according to Example XVII.

FIG. 152 shows Relative abundance of top ranked abundant proteins for whole blood samples collected either in 30 μL VAMS (Mitra®) or onto PVDF membrane, according to Example XVII.

FIG. 153 shows Relative abundance of top ranked abundant proteins for plasma samples collected into VAMS pre-loaded with nothing (control), BSA, or SDS, according to Example XVII.

FIG. 154 shows Overlap of phosphorylated peptides identified across three analytical platforms, Fragger, DIANN, and Max Quant, according to Example XVII.

FIG. 155 shows Overlap of protein identifications from various biological fluids including whole blood, plasma, and saliva, according to Example XVII.

FIG. 156 shows Protein IDs from whole blood VAMS or filter paper samples that underwent a variety of washing methods, according to Example XVIII.

FIG. 157 shows a Venn diagram comparison of protein profiles from a single tip that was extracted sequentially three times (A: first extraction; B: second extraction; and C: third extraction). Mass spectrometry was performed on all three extractions, according to Example XVIII, according to Example XVIII.

FIG. 158 shows a Heatmap and Venn diagram comparison of protein profiles from samples washed with 250 mM, 500 mM, or 1M LiCl, according to Example XVIII.

FIG. 159 shows a Venn diagram of protein IDs from VAMS washed with CaCl₂) or with LiCl, according to Example XVIII.

FIG. 160 shows Venn diagrams of the protein IDs from VAMS (M) vs filter paper (FP) for (A) samples collected and frozen or (B) samples collected and dried, according to Example XVIII.

FIG. 161 shows a PCA plot of the differences between whole blood samples collected into filter paper or VAMS (Mitra®), and samples immediately frozen or dried overnight, according to Example XVIII.

FIG. 162 shows (A) Protein and (B) peptide IDs from samples digested in trypsin overnight (Tryp Ctrl), trypsin overnight followed by an additional 2 hours (Tryp-Tryp), or trypsin overnight followed by 2 hours digestion with Glu-C (Tryp-GluC), according to Example XVIII.

FIG. 163 shows Total protein yield from whole blood or plasma VAMS that underwent a variety of sample treatment and washing methods, according to Example XIX.

FIG. 164 is a Venn diagram comparison of protein profiles from (A) plasma samples or (B) whole blood samples that have been either untreated (Ctrl), adjusted to pH 8 (TEAB), or adjusted to pH 6 (Citric Acid), according to Example XIX.

FIG. 165 shows Protein class assessment of unique IDs from whole blood samples adjusted to (A) pH 8 or (B) pH 6. Each coloured bar is indicative of a different protein class, according to Example XIX.

FIG. 166 is a Venn diagram comparison of protein profiles from (A) plasma samples or (B) whole blood samples that have been loaded into standard VAMS (Ctrl) or long shelf-life VAMS (NM), according to Example XIX.

FIG. 167 shows a Venn diagram comparison of protein IDs from whole blood samples washed with LiCl at 500 mM, 125 mM, or 50 mM or with CaCl₂) at 50 mM, according to Example XIX.

FIG. 168 shows a Venn diagram comparison of protein IDs from (A) plasma or (B) whole blood samples sequentially extracted for glycan analysis followed by proteomic analysis. Samples were either prepared according to the standard methods (Ctrl), washed then prepared for glycomics (SB161B and SB159B Glyco), prepared directly for glycomics (SB162 Glyco and SB160 Glyco) or the reduction/alkylation fraction prior to glycomic digestion was analysed (R/A), according to Example XX.

FIG. 169 shows a PCA plot of protein IDs from (A) plasma or (B) whole blood samples sequentially extracted for glycan analysis followed by proteomic analysis. Samples were either prepared according to the standard methods (Ctrl), prepared for glycomics (Glyco), or the reduction/alkylation fraction prior to glycomic digestion was analysed (Red/Alk), according to Example XX.

FIG. 170. Venn diagram comparison of protein IDs from whole blood samples extracted with 100 mM Tris, H₂O, or 500 mM LiCl+100 mM Tris (Ctrl), according to Example XXI.

FIG. 171 shows Total protein yield from bovine liver tissue samples either digested in VAMS (Tip) or in-tube as a liquid digest (Digest) after solubilisation with either SDC or PBS, according to Example XXII.

FIG. 172 shows Protein IDs from bovine liver tissue samples either digested in VAMS (Tip) or in-tube as a liquid digest (Digest) after solubilisation with either SDC or PBS, according to Example XXII.

FIG. 173 is a Venn diagram comparison of bovine liver tissue samples either digested in VAMS (Tip) or in-tube as a liquid digest (Digest) after solubilisation with either SDC or PBS, according to Example XXII.

FIG. 174 shows an experimental work flow of a method for sequential sample preparation using absorptive devices. In this work flow, 3 and optionally four, extractions and analyses are performed. First, a low volume extraction is performed with PBS or Tween (or PBS and Tween) followed by a series of washes to target specific protein groups. The porous material is then treated to remove glycans, prior to trypsin digestion of the porous material. Analyses can be performed on the first PBS-based extraction, optionally on washes collected (which may be regarded as a second extraction), the removed glycans, and the trypsin digest of the porous material.

### DETAILED DESCRIPTION

As used herein, the terms “Mitra®”, “Neoteryx tip(s)”, and “absorbent plug” are used interchangeably and refer to the porous material disclosed herein.

As used herein, the terms “VAMS” and “microsampling device” are used interchangeably.

Provided herein is a volumetric absorptive microsampling (VAMS) device for use in precision medicine genetic analyses. Specifically, provided herein is a VAMs device comprising a first porous material, a second porous material and a shaft. Provided herein is a VAMS device comprising a first porous material, a second porous material and a tube. Provided herein is a VAMS device comprising a first porous material, a second porous material, a third porous material and a shaft. Provided herein is a VAMS device comprising a first porous material, a second porous material, a third porous material and a tube. Also provided herein are methods of using a VAMS device to produce a protein profile, fractionate a blood sample, or isolate blood cells.

Cost-effective single nucleotide polymorphism (SNP) arrays that contain ˜800,000 variants, enable a wide range of polymorphisms to be rapidly assessed. Recently, the cost of SNP arrays has decreased substantially, driven by the very large sample sizes needed to perform genome-wide association studies (GWAS) (Suratannon N et al., Frontiers in Immunology. 2020; 11 (614)). Various genome sequencing modalities including whole genome sequencing, whole exome sequencing, targeted gene panels and transcriptomics are decreasing in costs and increasingly being applied in clinical settings (Pleasance et al., Nature Cancer. 2020; 1 (4): 452-68; Lièvre et al., J Clin Oncol. 2008; 26 (3): 374-9; Mosele et al., Ann Oncol. 2020; 31 (11): 1491-505).

At least in principle, a further level of personalization may be achieved by using other ‘omics technologies to obtain a quantitative readout of functional molecules such as proteins and metabolites (Geyer et al., Cell Syst. 2016; 2 (3): 185-95). In practice, proteomic technologies have not kept pace with genomics, particularly in the quantitative longitudinal analyses of patient blood. Although thousands of proteins may be quantified from highly fractionated plasma by using long LC-MS runtimes, such workflows are impractical for routine precision medicine use, reducing the reported studies to demonstration projects (Dey et al., Clinical Proteomics. 2019; 16 (1): 16; Wewer et al., Cell Syst. 2018; 7 (6): 601-12.e3). The issue, specifically, is that the time required for fractionating plasma samples by multi-dimensional chromatography (multiple chromatographic steps and/or types, such as reverse phase and size exclusion) is usually many hours for each sample. Thus it is completely impractical to run this strategy on hundreds or thousands of samples. In addition, doing proteomics on the liquid component (plasma or serum) does not enable intracellular proteins, membrane proteins or other cell-associated proteins (such as membrane bound) to be identified.

In order for blood proteomics to add significant value to precision medicine studies, repeat, longitudinal sampling is required using a system simple enough to engender use by the majority of participants. Furthermore, a streamlined analytical workflow compatible with high-throughput measurement technologies is required to quantify protein abundances in these blood samples.

### Methods

In one aspect, provided herein is a method of fractionating a sample, comprising: a) introducing a sample into a porous material; b) optionally, centrifuging the porous material containing the sample; c) drying the sample in the porous material; d) extracting a first set of proteins from the dried sample-containing porous material, comprising: 1) incubating the dried sample-containing porous material in a first extraction solution; and 2) separating the first extraction solution from the first incubated porous material; e) detecting the first set of proteins in the separated extraction solution.

In one aspect, provided herein is a method of fractionating a sample, comprising: a) introducing a sample into a porous material; b) optionally, centrifuging the porous material containing the sample; c) drying the sample in the porous material; d) extracting a first set of proteins from the dried sample-containing porous material, comprising: 1) incubating the dried sample-containing porous material in a first extraction solution; 2) separating the first extraction solution from the first incubated porous material; and 3) optionally, washing the separated first extracted porous material; e) extracting a second set of proteins from the first extracted porous material, comprising: 1) incubating the first extracted porous material in a second extraction solution; 2) separating the second extraction solution from the second incubated porous material; and f) detecting the first or second set of proteins from the separated first or second extraction solution, respectively.

In one aspect, provided herein is a method further comprising: g) optionally, washing the separated second extracted porous material; h) extracting a third set of proteins from the second extracted porous material, comprising: 1) incubating the second extracted porous material in a third extraction solution; and 2) separating the third extraction solution from the third incubated porous material; and i) detecting the third set of proteins in the separated third extraction solution.

In some embodiments, the method further comprises sequentially extracting one or more further sets of proteins from the third extracted porous material comprising repeating the steps g)-i) with one or more further extraction solutions.

In one aspect, provided herein is a method of fractionating a sample, comprising: a) introducing a sample into a porous material; b) optionally, centrifuging the porous material containing the sample; c) drying the sample in the porous material; d) extracting a first set molecules from the dried sample-containing porous material, comprising: 1) incubating the dried sample-containing porous material in a first extraction solution; 2) separating the first extraction solution from the first incubated porous material; and 3) optionally, washing the separated first extracted porous material; e) digesting proteins remaining in the first extracted porous material, comprising: 1) incubating the first extracted porous material in a digestion solution; and 2) separating the digestion solution from the digestion incubated porous material; and f) detecting one or more proteins in the separated digestion solution; and g) optionally detecting one or molecules in the first set of molecules.

In some embodiments, prior to the digestion step the method further comprises: A) extracting a second set of molecules from the first extracted porous material, comprising: 1) incubating the first extracted porous material in a second extraction solution; 2) separating the second extraction solution from the second incubated porous material; and g) optionally, washing the separated second extracted porous material; and B) optionally, detecting the second set of molecules in the separated second extraction solution.

In some embodiments, prior to the digestion step the method further comprises sequentially extracting one or more further sets of molecules from the second (and subsequent) extracted porous material comprising repeating the steps A)-B) with one or more further extraction solutions.

In some embodiments the molecules are selected from the group consisting of proteins, nucleic acids and glycans. In some embodiments the proteins are phosphorylated proteins.

In one aspect provided herein is a method of fractionating a sample, comprising: a) introducing a sample into a porous material; b) optionally, centrifuging the porous material containing the sample; c) drying the sample in the porous material; d) extracting a first set molecules from the dried sample-containing porous material, comprising: 1) incubating the dried sample-containing porous material in a first extraction solution; 2) separating the first extraction solution from the first incubated porous material; and 3) optionally, washing the separated first extracted porous material; and 4) optionally detecting one or more molecules in the first set of molecules; e) extracting a second set of molecules from the first extracted porous material, comprising: 1) incubating the first extracted porous material in a second extraction solution; 2) separating the second extraction solution from the second incubated porous material; and 3) optionally, washing the separated second extracted porous material; and 4) optionally, detecting the one or more molecules in the second set of molecules in the separated second extraction solution; and f) digesting proteins remaining in the first extracted porous material, comprising: 1) incubating the separated second extracted porous material in a digestion solution; and 2) separating the digestion solution from the digestion incubated porous material; and 3) detecting one or more proteins in the separated digestion solution.

In some embodiments, the first set of molecules is or comprises proteins. In some embodiments, the first set of molecules is or comprises nucleic acids. In some embodiments, the first set of molecules is or comprises glycans. In some embodiments, the first set of molecules is nucleic acids and the second first set of molecules is or comprises proteins. In some embodiments, the first set of molecules is or comprises proteins and the second set of molecules is or comprises glycans. In some embodiments the proteins are phosphorylated proteins.

In some embodiments, the proteins in the first, second, or one or more further sets of molecules, respectively, and/or the one or more peptides or proteins in the digestion solution are phosphoproteins or phosphopeptides. In some embodiments, the methods further comprise performing an enrichment step to enrich for phosphorylated proteins and/or peptides in the separated first, second, or one or more further extraction solutions and/or the digestion solution. In some embodiments the method involves pooling two or more separated first extraction solutions, two or more separated second extraction solutions, two or more of said separated one or more further extraction solutions, and/or pooling two or more digestion solutions obtained from a fractionation method as described herein performed on one or more replicate samples or one or more separate samples. In some embodiments, the enrichment step comprises an affinity-based technique, an immunoprecipitation technique, or a chemical modification technique. In one embodiment the enrichment step comprises an immobilized metal affinity chromatography (IMAC), and/or metal oxide affinity chromatography (MOAC) enrichment step. In another embodiment, the enrichment step utilizes zirconium based IMAC microparticles.

In one aspect, provided herein is a method of producing a protein profile, comprising utilizing the method of fractionating a sample as disclosed herein. In some embodiments, the protein profile is produced by immunoassay work flow. In some embodiments, the protein profile is produced by a proteomics work flow. In some embodiments, the protein profile is produced by a combination of an immunoassay work flow and a proteomics work flow. In some embodiments, the method produces a protein profile of non-membrane/soluble protein complexes. In some embodiments, the method produces a protein profile of cell-membrane associated proteins. In some embodiments the method produces a protein profile of phosphorylated proteins.

In one aspect, provided herein is a method of isolating one or more proteins, comprising utilizing the method of fractionating a sample as disclosed herein.

In one aspect, provided herein is a method of partitioning proteins in a sample by differential solubility, comprising utilizing the method of fractionating a sample as disclosed herein.

In one aspect, provided herein is a method of preparing sequential samples using an absorptive device, comprising utilizing the method of fractionating a sample as disclosed herein.

In one aspect, provided herein is a method of producing a protein profile, comprising: a) introducing a blood sample into a porous material; b) drying the blood sample in the porous material; c) detecting a first set of proteins extracted from the dried sample-containing porous material with a first extraction solution in a vessel; and d) detecting cell-membrane associated proteins digested from the first extracted sample-containing porous material with a digestion solution in the vessel; wherein the protein profile produced from the extracted first set of proteins and the digested cell-membrane associated proteins comprises one or more additional proteins, relative to a protein profile produced from detection of proteins in plasma from said blood sample.

In one aspect, provided herein is a method of fractionating a blood sample, comprising: a) introducing a blood sample into a porous material; b) drying the blood sample in the porous material; c) detecting a first set of proteins extracted from the dried sample-containing porous material with a first extraction solution in a vessel; and d) detecting cell-membrane associated proteins digested from the first extracted sample-containing porous material with a digestion solution in the vessel; wherein the protein profile produced from the extracted first set of proteins and the digested cell-membrane associated proteins comprises one or more additional proteins, relative to a protein profile produced from detection of proteins in plasma from said blood sample.

In one aspect, provided herein is a method of isolating blood cells, comprising: a) introducing a blood sample into a porous material; b) drying the blood sample in the porous material; c) detecting a first set of proteins extracted from the dried sample-containing porous material with a first extraction solution in a vessel; and d) detecting cell-membrane associated proteins digested from the first extracted sample-containing porous material with a digestion solution in the vessel; wherein the protein profile produced from the extracted first set of proteins and the digested cell-membrane associated proteins comprises one or more additional proteins, relative to a protein profile produced from detection of proteins in plasma from said blood sample.

In one aspect, provided herein is a method of producing a protein profile, comprising: a) introducing a sample into a porous material; b) drying the sample in the porous material; c) collecting a first set of proteins from the dried sample-containing porous material, comprising: i) incubating a first mixture of the dried sample-containing porous material in a first extraction solution in a vessel; ii) centrifuging the first mixture in the vessel; iii) separating the first extraction solution from the centrifuged first mixture in the vessel, wherein the separated first extraction solution comprises the first set of proteins; and iv) optionally, repeating steps i)-iii) of step c) one or more times with the separated first extracted sample-containing porous material in said vessel and said first extraction solution; d) collecting cell-membrane associated proteins from the separated first extracted sample-containing porous material, comprising: i) incubating a digestion mixture of the separated first extracted sample-containing porous material from step c) in a digestion solution in said vessel; ii) centrifuging the digestion mixture in said vessel; and iii) separating the digestion solution from the centrifuged digestion mixture in said vessel, wherein the separated digestion solution comprises the cell-membrane associated proteins; and e) detecting said first set of proteins and said cell-membrane associated proteins.

In one aspect, provided herein is a method of fractionating a sample, comprising: a) introducing a sample into a porous material; b) drying the sample in the porous material; c) collecting a first set of proteins from the dried sample-containing porous material, comprising: i) incubating a first mixture of the dried sample-containing porous material in a first extraction solution in a vessel; ii) centrifuging the first mixture in the vessel; iii) separating the first extraction solution from the centrifuged first mixture in the vessel, wherein the separated first extraction solution comprises the first set of proteins; and iv) optionally, repeating steps i)-iii) of step c) one or more times with the separated first extracted sample-containing porous material in said vessel and said first extraction solution; d) collecting cell-membrane associated proteins from the separated first extracted sample-containing porous material, comprising: i) incubating a digestion mixture of the separated sample-containing porous material from step c) in a digestion solution in said vessel; ii) centrifuging the digestion mixture in said vessel; and iii) separating the digestion solution from the centrifuged digestion mixture in said vessel, wherein the separated digestion solution comprises the cell-membrane associated proteins; and e) detecting said first set of proteins and said cell-membrane associated proteins.

In one aspect, provided herein is a method of isolating blood cells, comprising: a) introducing a sample into a porous material; b) drying the sample in the porous material; c) collecting a first set of proteins from the dried sample-containing porous material, comprising: i) incubating a first mixture of the dried sample-containing porous material in a first extraction solution in a vessel; ii) centrifuging the first mixture in the vessel; iii) separating the first extraction solution from the centrifuged first mixture in the vessel, wherein the separated first extraction solution comprises the first set of proteins; and iv) optionally, repeating steps i)-iii) of step c) one or more times with the separated sample-containing porous material in said vessel and said first extraction solution; d) collecting a set of cell-membrane associated proteins from the first extracted sample-containing porous material, comprising: i) incubating a digestion mixture of the separated sample-containing porous material from step c) in a digestion solution in said vessel; ii) centrifuging the digestion mixture in said vessel; and iii) separating the digestion solution from the centrifuged digestion mixture in said vessel, wherein the separated digestion solution comprises the cell-membrane associated proteins; and e) detecting said first set of proteins and said cell-membrane associated proteins.

In one aspect, provided herein is a method of producing a protein profile, comprising: a) introducing a sample into a porous material; b) drying the sample in the porous material; c) detecting a first set of proteins extracted from the dried sample-containing porous material with a first extraction solution in a vessel; and d) detecting cell-membrane associated proteins digested from the first extracted sample-containing porous material with a digestion solution in the vessel; wherein the protein profile produced from the extracted first set of proteins and the digested cell-membrane associated proteins comprises one or more additional proteins, relative to a protein profile produced from detection of proteins from said sample.

In one aspect, provided herein is a method of fractionating a sample, comprising: a) introducing a sample into a porous material; b) drying the sample in the porous material; c) detecting a first set of proteins extracted from the dried sample-containing porous material with a first extraction solution in a vessel; and d) detecting cell-membrane associated proteins digested from the first extracted sample-containing porous material with a digestion solution in the vessel; wherein the protein profile produced from the extracted first set of proteins and the digested cell-membrane associated proteins comprises one or more additional proteins, relative to a protein profile produced from detection of proteins from said sample.

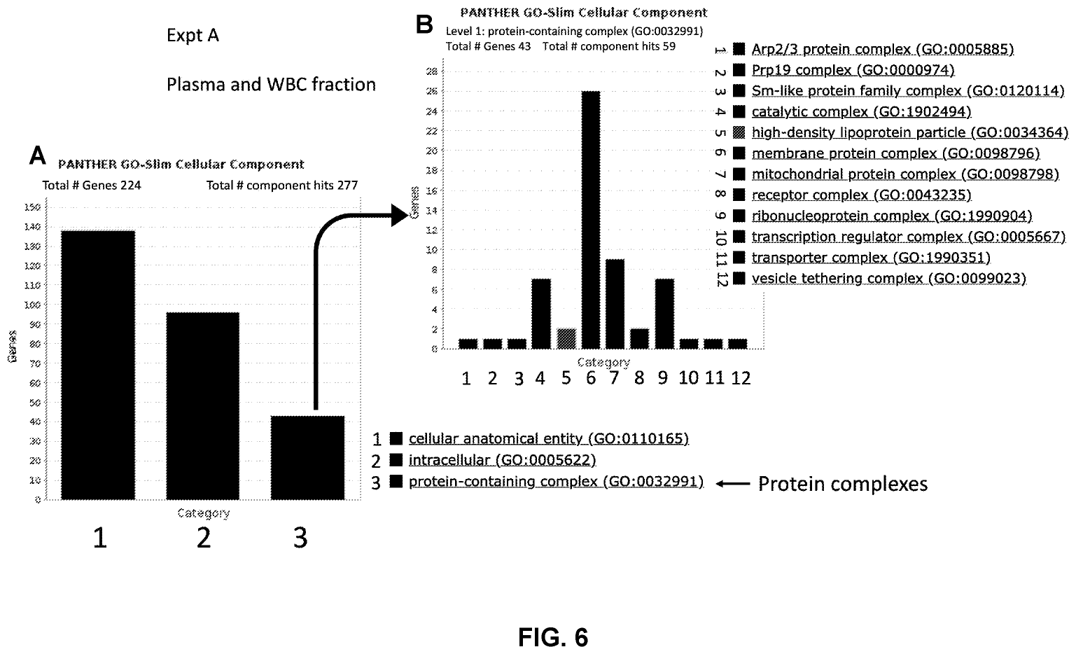

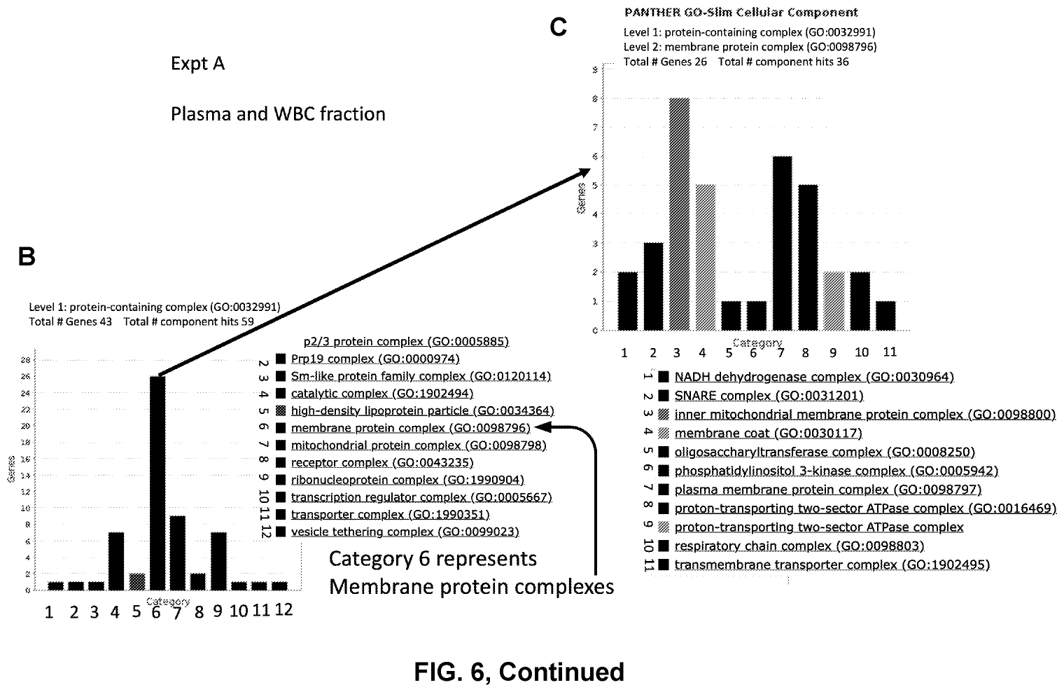

Table 1 shows a list of membrane-associated protein complexes and non-membrane associated protein complexes in FIGS. 6A-6C.

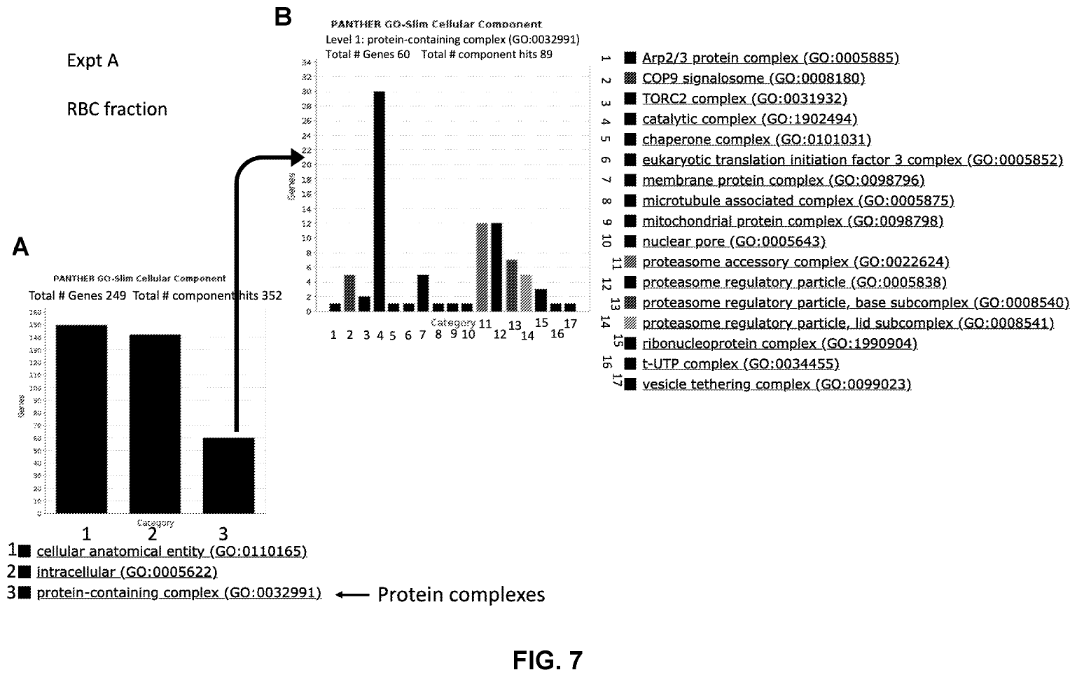

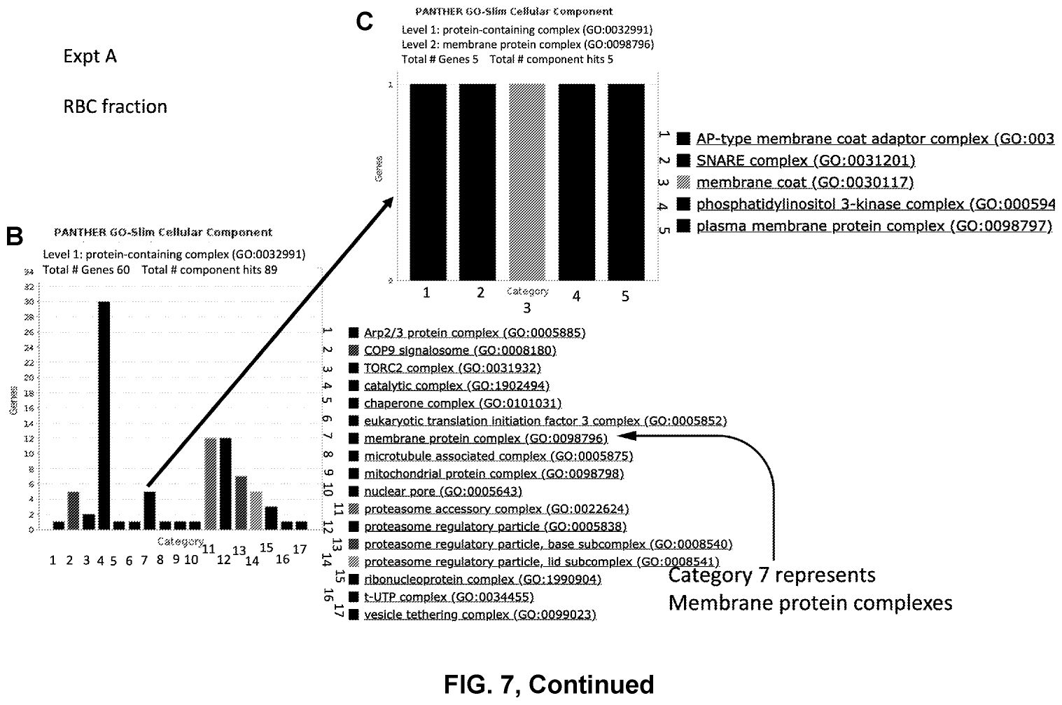

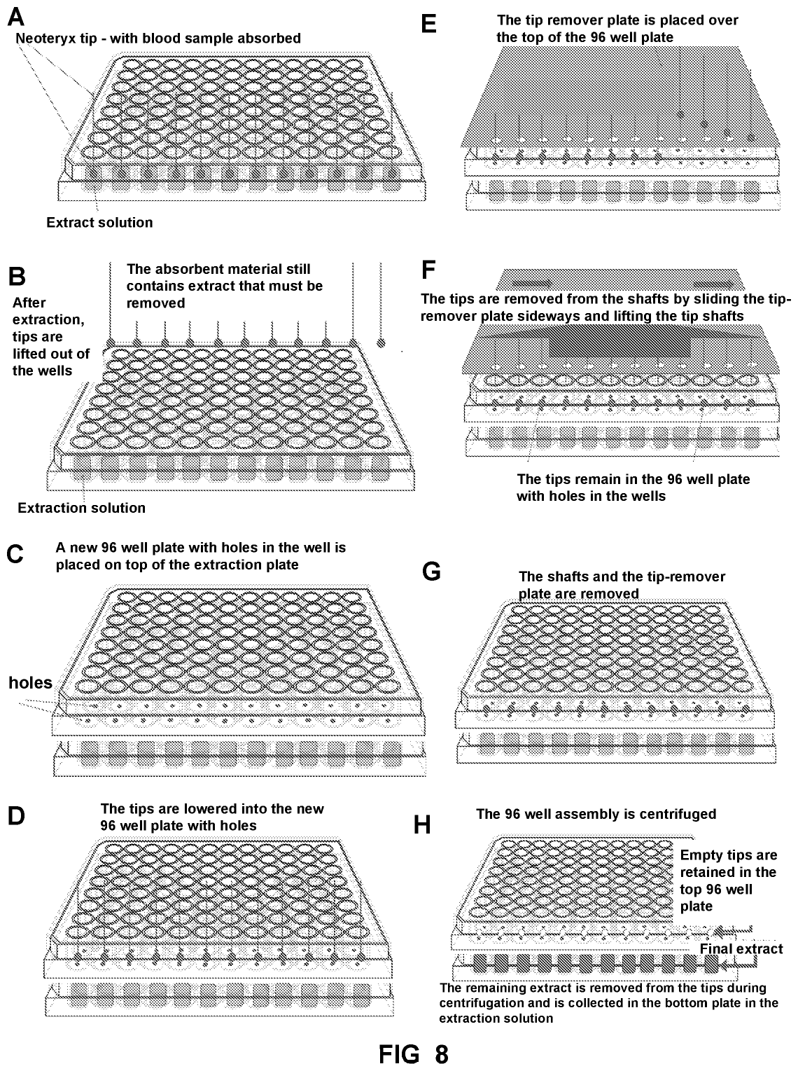

Table 2 shows a list of membrane-associated protein complexes and non-membrane associated protein complexes in FIGS. 7A-7C.

TABLE 1
List of membrane and non-membrane associated proteins.

| Gene | Protein Description | Protein Class |
|---|---|---|
| ATP6V0D1 | V-type proton ATPase subunit d 1; ATP6V0D1; ortholog | ATP synthase(PC00002) |
| ATP5H | ATP synthase subunit d, mitochondrial; ATP5PD; ortholog | ATP synthase(PC00002) |
| SARIA | GTP-binding protein SAR1a; SAR1A; ortholog | |
| COPB1 | Coatomer subunit beta; COPB1; ortholog | vesicle coat protein(PC00235) |
| PIK3CG | Phosphatidylinositol 4,5-bisphosphate 3-kinase catalytic | kinase(PC00137) |
| | subunit gamma isoform; PIK3CG; ortholog | |
| ATP5L | ATP synthase subunit g, mitochondrial; ATP5MG; ortholog | ATP synthase(PC00002) |
| NDUFB10 | NADH dehydrogenase [ubiquinone] 1 beta subcomplex | oxidoreductase(PC00176) |
| | subunit 10; NDUFB10; ortholog | |
| ITGB1 | Integrin beta-1; ITGB1; ortholog | integrin(PC00126) |
| STX11 | Syntaxin-11; STX11; ortholog | SNARE protein(PC00034) |
| NDUFB9 | NADH dehydrogenase [ubiquinone] 1 beta subcomplex | oxidoreductase(PC00176) |
| | subunit 9; NDUFB9; ortholog | |
| MCU | Calcium uniporter protein, mitochondrial; MCU; ortholog | |
| CHMP6 | Charged multivesicular body protein 6; CHMP6; ortholog | membrane traffic |
| | | protein(PC00150) |
| EPS15 | Epidermal growth factor receptor substrate 15; EPS15; | membrane traffic |
| | ortholog | protein(PC00150) |
| IMMT | MICOS complex subunit MIC60; IMMT; ortholog | |
| STXBP5 | Syntaxin-binding protein 5; STXBP5; ortholog | membrane trafficking |
| | | regulatory protein(PC00151) |
| TAP2 | Antigen peptide transporter 2; TAP2; ortholog | ATP-binding cassette (ABC) |
| | | transporter(PC00003) |
| ATP5I | ATP synthase subunit e, mitochondrial; ATP5ME; ortholog | ATP synthase(PC00002) |
| GNAZ | Guanine nucleotide-binding protein G(z) subunit alpha; | heterotrimeric G- |
| | GNAZ; ortholog | protein(PC00117) |
| SNTB1 | Beta-1-syntrophin; SNTB1; ortholog | |
| CAP1 | TNF receptor-associated factor 3; TRAF3; ortholog | scaffold/adaptor |
| | | protein(PC00226) |
| CLINT1 | Clathrin interactor 1; CLINT1; ortholog | |
| COPG1 | Coatomer subunit gamma-1; COPG1; ortholog | vesicle coat protein(PC00235) |
| DAD1 | Dolichyl-diphosphooligosaccharide--protein | glycosyltransferase(PC00111) |
| | glycosyltransferase subunit DAD1; DAD1; ortholog | |
| STX7 | Syntaxin-7; STX7; ortholog | SNARE protein(PC00034) |
| DNAJC19 | Mitochondrial import inner membrane translocase subunit | |
| | TIM14; DNAJC19; ortholog | |
| ATP6V0A1 | V-type proton ATPase 116 kDa subunit a1; ATP6V0A1; | ATP synthase(PC00002) |
| | ortholog | |

TABLE 2
List of membrane and non-membrane associated proteins.

| Gene | Protein Description | Protein Class |
|---|---|---|
| AP2A1 | AP-2 complex subunit alpha-1; AP2A1; ortholog | membrane traffic |
| | | protein(PC00150) |
| VAC14 | Protein VAC14 homolog; VAC14; ortholog | scaffold/adaptor |
| | | protein(PC00226) |
| NAPA | Alpha-soluble NSF attachment protein; NAPA; | membrane traffic |
| | ortholog | protein(PC00150) |
| SEC61A1 | Protein transport protein Sec61 subunit alpha isoform | transporter(PC00227) |
| | 1; SEC61A1; ortholog | |
| VPS25 | Vacuolar protein-sorting-associated protein 25; VPS25; | membrane traffic |
| | ortholog | protein(PC00150) |

In some embodiments, the method further comprises collecting a second set of proteins from the separated first extracted sample-containing porous material from step c), comprising: i) incubating a second mixture of the separated first extracted sample-containing porous material in a second extraction solution in a vessel; ii) centrifuging the second mixture in the vessel; iii) separating the second extraction solution from the centrifuged second mixture in the vessel, wherein the separated second extraction solution comprises the second set of proteins; and iv) optionally, repeating steps i)-iii) one or more times with the separated second extracted sample-containing porous material in said vessel and said second extraction solution.

In some embodiments, the collecting cell-membrane associated proteins step comprises collecting cell-membrane associated proteins from the separated second extracted sample-containing porous material, comprising: i) incubating a digestion mixture of the separated second extracted sample-containing porous material in a digestion solution in said vessel; ii) centrifuging the digestion mixture in said vessel; and iii) separating the digestion solution from the centrifuged digestion mixture in said vessel, wherein the separated digestion solution comprises the cell-membrane associated proteins.

In some embodiments, the detecting step comprises detecting said first set of proteins, said second set of proteins, and said cell-membrane associated proteins.

In some embodiments, the method further comprises collecting a third set of proteins from the separated second extracted sample-containing porous material, comprising: i) incubating a third mixture of the separated second extracted sample-containing porous material in a third extraction solution in a vessel; ii) centrifuging the third mixture in the vessel; iii) separating the third extraction solution from the centrifuged second mixture in the vessel, wherein the separated third extraction solution comprises the third set of proteins; and iv) optionally, repeating steps i)-iii) one or more times with the separated third extracted sample-containing porous material in said vessel and said third extraction solution.

In some embodiments, the collecting cell-membrane associated proteins step comprises collecting cell-membrane associated proteins from the separated third extracted sample-containing porous material, comprising: i) incubating a digestion mixture of the separated third extracted sample-containing porous material in a digestion solution in said vessel; ii) centrifuging the digestion mixture in said vessel; and iii) separating the digestion solution from the centrifuged digestion mixture in said vessel, wherein the separated digestion solution comprises the cell-membrane associated proteins.

In some embodiments, the detecting step comprises detecting said first set of proteins, said second set of proteins, said third set of proteins, and said cell-membrane associated proteins.

In some embodiments, the proteins in said first set of proteins, said second set of proteins, said third set of proteins, and said cell-membrane associated proteins are phosphoproteins or phosphopeptides. In some embodiments, the methods further comprise performing an enrichment step to enrich for phosphorylated proteins and/or peptides in the separated first, second, or third or one or more further extraction solutions and/or a digestion solution. In some embodiments the method involves pooling two or more separated first extraction solutions, two or more separated second extraction solutions, two or more of said separated one or more further extraction solutions, and/or pooling two or more digestion solutions obtained from a fractionation method as described herein performed on one or more replicate samples or one or more separate samples. In some embodiments, the enrichment step comprises an affinity-based technique, an immunoprecipitation technique, or a chemical modification technique. In one embodiment the enrichment step comprises an immobilized metal affinity chromatography (IMAC), and/or metal oxide affinity chromatography (MOAC) enrichment step. In another embodiment, the enrichment step utilizes zirconium based IMAC microparticles.

In some embodiments, the sample is or comprises a body fluid. In some embodiments, the sample comprises cells or tissue. In some embodiments, the sample comprises cells suspended in a liquid. In some embodiments, the sample comprises cultured cells suspended in a culture media. In some embodiments, the body fluid is or comprises blood, blood fractions, plasma, a nucleic acid-containing stabilized sample, a stabilized blood sample (e.g., blood in a Streck tube sample), urine, tears, wound fluid, CSF, bronchoalveolar lavage, or ascites. In some embodiments, the sample is or comprises plasma. In some embodiments, the sample is or comprises a blood sample. In some embodiments, the blood sample is a whole blood (WB) sample. In some embodiments, the blood sample is a red blood cell (RBC) sample. In some embodiments, the blood sample is a white blood cell (WBC) sample. In some embodiments, the blood sample is a frozen blood sample. In some embodiments, the blood sample is fresh. As used herein, the term “fresh” refers to a sample that has not been frozen. In some embodiments, the blood sample is frozen. In some embodiments, the sample is or comprises a nucleic acid-containing stabilized sample or a stabilized blood sample, such as a nucleic acid-containing stabilized blood sample, for example a blood sample in a Streck tube. In some embodiments the body fluid comprises blood or blood fractions. In some embodiments, the body fluid comprises frozen blood or frozen blood fractions. In some embodiments, the blood fractions comprise WBCs, platelets, or RBCs.

In other embodiments, the sample is or comprises a blood sample. In some embodiments, the sample is or comprises a whole blood sample. In some embodiments, the sample is a blood sample, and wherein the protein profile produced from the extracted at least first set of proteins and the digested cell-membrane associated proteins comprises one or more additional proteins, relative to a protein profile produced from detection of proteins in plasma from said blood sample. In some embodiments, the blood sample is a whole blood (WB) sample. In some embodiments, the blood sample is a red blood cell sample (RBC). In some embodiments, the blood sample is a fractionated blood sample. In some embodiments, the fractionated blood sample comprises white blood cells (WBCs), platelets and/or RBCs. In some embodiments, the blood sample is a frozen blood sample. In some embodiments, the blood sample is a fresh blood sample.

In some embodiments, the blood sample has a volume in the range of 100 μL to 2 μL, 100 μL to 5 μL, 100 μL to 10 μL, 99 μL to 2 μL, 90 μL to 2 μL, 80 μL to 2 μL, 70 μL to 2 μL, 60 μL to 2 μL, 50 μL to 2 μL, 40 μL to 2 μL, 30 μL to 2 μL, 20 μL to 2 μL, 10 μL to 2 μL, or 5 μL to 2 μL. In some embodiments, the blood sample has a volume of <100 μL, <50 μL, <30 μL, or <10 μL, or <5 μL. In some embodiments, the blood sample has a volume of at least 2 μL.

In some embodiments, the introducing step comprises absorbing the sample into the porous material. In some embodiments, the introducing step comprises absorbing the sample into the porous material via a finger prick. In some embodiments, the introducing step comprises dipping the porous material into the sample. In some embodiments, the introducing step comprises pipetting a known volume of the sample into the porous material.

In some embodiments, said method further comprises collecting the sample. In some embodiments, said method further comprises collecting of the sample with a microsampling device. In some embodiments, the introducing of the sample comprises absorbing the sample into the porous material.

In some embodiments, the blood sample is partitioned. In some embodiments, partitioning comprises centrifugation. In some embodiments, the method comprises centrifuging the porous material containing the sample prior to the drying step. In some embodiments, centrifugation is performed at a speed of 1,500 g for 5 min. In some embodiments, centrifugation is performed from 500 g to 10,000 g. In some embodiments, centrifugation is performed from 500 g to 3,000 g. In some embodiments, the centrifuging is for at least 1 to 15 minutes. In some embodiments, centrifugation is performed for no more than 15 minutes. In other embodiments, the blood samples has been partitioned via filtration.

In some embodiments, the method comprises sequential extraction. In some embodiments, sequential extraction is a fractionation of proteins based on solubility. In some embodiments, the method comprises one or non-sequential extraction.

In some embodiments, the method recovers 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or 2000 or more proteins compared to traditional microsampling. In some embodiments, the the method recovers a fraction of non-membrane/soluble protein complexes from a red blood cell (RBC) sample not obtained by traditional microsampling. In some embodiments, the method recovers a fraction of non-membrane/soluble protein complexes from a plasma sample not obtained by traditional microsampling. In some embodiments, the method recovers a fraction of non-membrane/soluble protein complexes from a whole blood sample not obtained by traditional microsampling. In some embodiments, the method recovers a fraction of membrane protein complexes from a red blood cell (RBC) sample not obtained by traditional microsampling. In some embodiments, the method recovers a fraction of membrane protein complexes from a plasma sample not obtained by traditional microsampling. In some embodiments, the method recovers a fraction of membrane protein complexes from a whole blood sample not obtained by traditional microsampling.

In some embodiments, the drying step, the extraction step, and the digesting step are conducted with said sample-containing porous material in said tube.

In some embodiments, the drying step comprises air drying the sample within the porous material, after which the sample is dried in the porous material. In some embodiments, the drying step comprises centrifuging the sample within the porous material after which, the sample is dried in the porous material. In some embodiments, the drying step comprises drying for a period of time to adhere the sample to the porous material. In some embodiments, the drying step comprises drying for a period of time to adhere the sample to the porous material after which the sample-containing porous material is stored for a period of time prior to the first extraction step. In some embodiments, the sample-containing porous material is frozen prior to the first extraction step. In some embodiments, the drying step comprises air drying. In some embodiments the drying step is for less than 30 mins. In some embodiments the drying step is for less than 20 minutes. In some embodiments the drying step is for less than 10 minutes. In some embodiments the drying step is for 5 minutes. In some embodiments the drying step is for less than 5 minutes. In some embodiments, air drying is for at least 30 mins, 1, 2, 4, 6, 8, 10, 12, 16, 20 or 24 hours. In some embodiments, air drying is for 24 hours. In some embodiments, air drying is for at least 1 day. In some embodiments, the drying step comprises vacuum drying. In some embodiments, the drying step comprises vacuuming the sample within the porous material. In some embodiments, the drying step is performed immediately after blood collection in the porous material. In some embodiments, the drying the porous material immediately after sample collection comprises air drying. In some embodiments, the drying step occurs after extraction and digestion. In some embodiments, the drying the porous material immediately after blood collection comprises centrifugation. In some embodiments, the drying step comprises centrifuging the sample within the porous material. In some embodiments, the centrifuging is at a speed of 500 g to 10,000 g. In some embodiments, the centrifuging is for at least 1 to 15 minutes. In some embodiments, the centrifuging is for no more than 15 minutes. In some embodiments, the sample is frozen immediately after the sample is collected into the porous material, or optionally, the sample is air dried for less than 30 minutes before being frozen.

In some embodiments, the first, the second, the third, and the further extraction solution comprises an extracting agent comprising a salt, a mild detergent, a strong detergent, a chaotrope, a reducing agent, a thiol-containing reducing agent, an alkylating agent, an acid, an organic solvent, or an enzyme, or combinations thereof. In some embodiments, the first extraction solution comprises an extracting agent comprising a salt, a mild detergent, a strong detergent, a chaotrope, a reducing agent, a thiol-containing reducing agent, an alkylating agent, an acid, an organic solvent, or an enzyme, or combinations thereof. In some embodiments, the second extraction solution comprises an extracting agent comprising a salt, a mild detergent, a strong detergent, a chaotrope, a reducing agent, a thiol-containing reducing agent, an alkylating agent, an acid, an organic solvent, or an enzyme, or combinations thereof. In some embodiments, the third extraction solution comprises an extracting agent comprising a salt, a mild detergent, a strong detergent, a chaotrope, a reducing agent, a thiol-containing reducing agent, an alkylating agent, an acid, an organic solvent, or an enzyme, or combinations thereof. In some embodiments, the further extraction solution comprises an extracting agent comprising a salt, a mild detergent, a strong detergent, a chaotrope, a reducing agent, a thiol-containing reducing agent, an alkylating agent, an acid, an organic solvent, or an enzyme, or combinations thereof.

In some embodiments, the first, the second, the third, and the further extraction solution comprises a salt. In some embodiments, the first, the second, the third, and the further extraction solution comprises a mild detergent. In some embodiments, the first, the second, the third, and the further extraction solution comprises a strong detergent. In some embodiments, the first, the second, the third, and the further extraction solution comprises a chaotrope. In some embodiments, the the first, the second, the third, and the further extraction solution comprises a reducing agent. In some embodiments, the first, the second, the third, and the further extraction solution comprises a thiol-containing reducing agent. In some embodiments, the first, the second, the third, and the further extraction solution comprises an alkylating agent. In some embodiments, the first, the second, the third, and the further extraction solution comprises an acid. In some embodiments, the first, the second, the third, and the further extraction solution comprises an organic solvent. In some embodiments, the first, the second, the third, and the further extraction solution comprises an enzyme. In some embodiments, the salt is or comprises NaCl, LiCl, or Tris-HCl. In some embodiments, the mild detergent is a non-ionic detergent, such as PBS or Tween. In some embodiments, the mild detergent is an ionic detergent, such as sodium deoxycholate. In some embodiments, the mild detergent is a zwitterionic detergent, such as a sulfobetaine or an amidosulfobetaine. In some embodiments, the mild detergent is or comprises PBS, Tween or sodium deoxycholate. In some embodiments, the mild detergent is or comprises Tween or sodium deoxycholate. In some embodiments, the strong detergent is or comprises cetyltrimethylammonium bromide (CTAB), (3-((3-cholamidopropyl) dimethylammonio)-1-propanesulfonate) (CHAPS) or sodium dodecyl sulfate (SDS). In some embodiments, the chaotrope is or comprises urea, thiourea, or guanidine. In some embodiments, the reducing agent is or comprises a phosphine. In some embodiments, the phosphine is or comprises tributyl phosphate (TBP) or tris-(2-carboxyethyl) phosphine (TCEP). In some embodiments, the thiol-containing reducing agent is or comprises betamercaptoethanol or dithiothreitol. In some embodiments, the alkylating agent is or comprises iodoacetamide or acrylamide. In some embodiments, the acid is or comprises citric acid or trifluoroacetic acid. In some embodiments, the organic solvent is methanol. In some embodiments, the enzyme is or comprises benzonase.

In some embodiments, the first extraction solution is a salt solution, a mild detergent-containing solution, or a salt and mild detergent-containing solution. In some embodiments, the first extraction solution is a salt solution, a chaotrope-containing solution, or a salt and chaotrope-containing solution. In some embodiments, the first extraction solution comprises NaCl, LiCl, Tris-HCl, PBS, or Tween, or combination thereof. In some embodiments, the first extraction solution comprises NaCl, LiCl, Tris-HCl, PBS, Tween, sodium deoxycholate, CTAB, CHAPS, SDS, urea, thiourea, or guanidine, or combination thereof. In some embodiments, the first extraction solution comprises NaCl, LiCl, Tris-HCl, PBS, Tween, sodium deoxycholate, CHAPS, SDS, urea, thiourea, guanidine, TBP, TCEP, betamercaptoethanol, dithiothreitol, iodoacetamide, or acrylamide, or combination thereof.

In some embodiments, where glycans present in the sample are to be assessed, the first, the second, the third, or the one or more further extraction solutions comprises an endoglycosidase. In some embodiments the endoglycosidase is Peptide-N-Glycosidase F (PNGase F) and/or O-glycosidase.

In some embodiments, the first extraction solution and the second extraction solution are different. In some embodiments, the first, the second, and the third extraction solutions are different. In some embodiments, the first, the second, the third, and the further extraction solutions are different. In some embodiments, the selection of the first, the second, the third, and the further extraction solutions is to fractionate the proteins contained in the porous material via differential solubility. In some embodiments, each sequential extraction solution selected is characterized as having greater solubility of the proteins remaining in the porous material. In some embodiments, the first, the second, the third, and the further extraction solutions comprise identical extracting agents of increasing concentrations as progress from one extraction to the next extraction.

In some embodiments, the selection of the different extraction solution is limited to solutions based on proteins remaining in the separated first extracted porous material have equivalent or less solubility in the different extraction solution than the first extraction solution. In some embodiments, the selection of the different extraction solution is based on the proteins remaining in the separated first extracted porous material having equivalent or less solubility in the different extraction solution than said first extraction solution.

In some embodiments, the method comprises extracting or washing the dried sample-containing porous material one or more times with the first extraction solution. In some embodiments, the dried sample-containing porous material extracted or washed 1, 2, or 3 times with the first extraction solution. In some embodiments, the dried sample-containing porous material is extracted or washed once with the first extraction solution. In some embodiments, the dried sample-containing porous material is extracted or washed twice with the first extraction solution. In some embodiments, the dried sample-containing porous material is extracted or washed 3 times with the first extraction solution.

In some embodiments, the concentration of the salt in the first, the second, the third, or the further extraction solution, is 0.01-1 M. In some embodiments, the concentration of the salt in the first, the second, the third, or the further extraction solution, is 0.5 M. In some embodiments, the concentration of the salt in the first, the second, the third, or the further extraction solution, is 0.1 M. In some embodiments, the salt is NaCl, LiCl, or Tris-HCl. In some embodiments, the concentration of the NaCl in the first, the second, the third, or the further extraction solution, is 0.5 M. In some embodiments, the concentration of the LiCl in the first, the second, the third, or the further extraction solution, is 0.5 M. In some embodiments, the concentration of the Tris-HCl in the first, the second, the third, or the further extraction solution, is 0.1 M. In some embodiments, the concentration of the chaotrope in the first, the second, the third, or the further extraction solution, is 0.01-8 M. In some embodiments, the concentration of the chaotrope in the first, the second, the third, or the further extraction solution, is 0.1, 0.5, 1, 1.5, 2, 2.5, 3, 3.5, 4, 4.5, 5, 5.5, 6, 6.5, 7, 7.5, or 8 M. In some embodiments, the concentration of the chaotrope in the first, the second, the third, or the further extraction solution, is 0.01-8 M. In some embodiments, the chaotrope is urea, thiourea, or guanidine. In some embodiments, the first, the second, the third, or the further extraction solution comprises urea, thiourea, or guanidine, or combinations thereof. In some embodiments, the first, the second, the third, or the further extraction solution comprises urea at a concentration of 2 M. In some embodiments, the first, the second, the third, or the further extraction solution comprises urea at a concentration of 5 M and thiourea at a concentration of 1 M. In some embodiments, the first, the second, the third, or the further extraction solution comprises urea at a concentration of 6 M and thiourea at a concentration of 1.5 M. In some embodiments, the first, the second, the third, or the further extraction solution comprises urea at a concentration of 7 M and thiourea at a concentration of 2 M.

In some embodiments, the first extraction solution is a salt solution or a urea-containing solution. In some embodiments, the salt solution is a Tris-HCl solution containing NaCl or LiCl. In some embodiments, the urea-containing solution is a Tris-HCl solution containing urea. In some embodiments, the first extraction solution is a salt solution, a urea containing solution or a salt and urea containing solution. In some embodiments, the first extraction solution comprises Tris-Cl, NaCl, LiCl or urea or combinations thereof. In some embodiments, the first extraction solution comprises Tris-HCl. In some embodiments, the first extraction solution comprises NaCl. In some embodiments, the first extraction solution comprises LiCl. In some embodiments, the first extraction solution comprises urea. In some embodiments, the Tris-HCl is present in the first extraction solution at a concentration of 0.1M. In some embodiments, the NaCl is present in the first extraction solution at a concentration of 0.5M. In some embodiments, the LiCl is present in the first extraction solution at a concentration of 0.5M. In some embodiments, the urea is present in the first extraction solution at a concentration of 2M. In some embodiments, the first extraction solution and the second extraction solution are different. In some embodiments, the first extraction solution and the second extraction solution are a salt solution or a urea containing solution. In some embodiments, the first extraction solution and the second extraction solution are different. In some embodiments, the second extraction solution comprises Tris-HCl, NaCl, LiCl or urea or combinations thereof. In some embodiments, the first extraction solution is a salt solution and the second extraction solution is a different salt solution from the first extraction solution or is a urea-containing solution. In some embodiments, the first extraction solution comprises Tris-HCl. In some embodiments, the first extraction solution comprises NaCl. In some embodiments, the first extraction solution comprises LiCl. In some embodiments, the first extraction solution comprises urea. In some embodiments, the second extraction solution comprises Tris-HCl. In some embodiments, the second extraction solution comprises NaCl. In some embodiments, the second extraction solution comprises LiCl. In some embodiments, the second extraction solution comprises urea. In some embodiments, the Tris-HCl is present in the extraction solution at a concentration of 0.1M. In some embodiments, the NaCl is present in the extraction solution at a concentration of 0.5M. In some embodiments, the LiCl is present in the extraction solution at a concentration of 0.5M. In some embodiments, the urea is present in the extraction solution at a concentration of 2M.

In some embodiments, the separated first, second, third, or further, extraction solution comprises one or more proteins. In some embodiments, the separated first extraction solution comprises the first set of proteins. In some embodiments, the separated first extraction solution comprises albumin, hemoglobin, IgG, or one or more additional proteins compared to conventionally isolated serum or plasma. In some embodiments, the separated second extraction solution comprises the second set of proteins. In some embodiments, the separated third extraction solution comprises the third set of proteins. In some embodiments, the separated further extraction solution comprises the further set of proteins.

In some embodiments, the first, the second, the third, or the further, extraction solution has a volume of less than 100 μL. In some embodiments, the first, the second, the third, or the further, extraction solution has a volume of 95 μL or less. In some embodiments, the first, the second, the third, or the further, extraction solution has a volume of 90 μL or less. In some embodiments, the first, the second, the third, or the further, extraction solution has a volume of 1-5 times the volume of porous material. In some embodiments, the first, the second, the third, or the further, extraction solution has a volume of 1, 2, 3, 4, or 5 times the volume of porous material. In some embodiments, the first, the second, the third, or the further, extraction solution has a volume of 3 times the volume of porous material.

In some embodiments, the incubating in the first, the second, the third, or the further, extraction solution is conducted for a period of time long enough to solubilize one or more proteins contained within or adhered to the porous material. In some embodiments, the incubating in the first, the second, the third, or the further, extraction solution is conducted for a period of time long enough to extract one or more proteins contained within or adhered to the porous material. In some embodiments, the incubating is conducted for 1 min-48 hour. In some embodiments, the incubating is conducted for 1, 5, 10, 15, 20, 25, 30, 35, 40, 45, 50, 55 or 60 min. In some embodiments, the incubating is conducted for 1, 2, 3, 4, 5, 6, 7, 8, 9 or 10 minutes. In some embodiments, the incubating is conducted for 1-48 hours, 1-40 hours, 1-30 hours, 1-20 hours, 1-10 hours, 1-5 hours, or 1-40 hours. In some embodiments, the incubating is conducted at ambient temperature. In some embodiments, the incubating is conducted at a temperature elevated above ambient temperature. In some embodiments, the incubating is conducted at a temperature elevated below ambient temperature. In some embodiments, the incubating is conducted with agitation.

In some embodiments, the separating comprises removing the first, the second, the third, or the further, incubated porous material from the first, the second, the third, or the further, extraction solution, respectively. In some embodiments, the separating comprises removing the first, the second, the third, or the further, extraction solution from the first, the second, the third, or the further, incubated porous material, respectively. In some embodiments, the separating of the first, the second, the third, or the further, extraction solution from the first, the second, the third, or the further, incubated porous material is via centrifugation, filtration, or a combination of centrifugation and filtration, respectively. In some embodiments, the separating step is conducted via centrifugation. In some embodiments, the separating step is conducted via filtration. In some embodiments, the separating step is conducted via a combination of centrifugation and filtration. In some embodiments, the centrifuging is at a speed of 500 g to 10,000 g. In some embodiments, the centrifuging is for at least 1 minute. In some embodiments, the centrifuging is for 1-15 minutes. In some embodiments, the centrifuging is for no more than 15 minutes.

In some embodiments, the digestion solution comprises a reducing agent, an alkylating agent, a buffer, a detergent, or combinations thereof. In some embodiments, the reducing agent is or comprises a phosphine. In some embodiments, the phosphine is or comprises TBP or TCEP. In some embodiments, the alkylating agent is or comprises iodoacetamide or acrylamide. In some embodiments, the digestion solution further comprises a salt, a mild detergent, a strong detergent, a chaotrope, or a thiol-containing reducing agent, or combinations thereof. In some embodiments, the the salt is or comprises NaCl, LiCl, or Tris-HCl. In some embodiments, the mild detergent is a non-ionic detergent. In some embodiments, the mild detergent is or comprises PBS or Tween. In some embodiments, the mild detergent is an ionic detergent, such as sodium deoxycholate. In some embodiments, the mild detergent is a zwitterionic detergent, such as a sulfobetaine or an amidosulfobetaine. In some embodiments, the mild detergent is or comprises PBS, or Tween or sodium deoxycholate. In some embodiments, the mild detergent is or comprises Tween or sodium deoxycholate. In some embodiments, the strong detergent is or comprises cetyltrimethylammonium bromide (CTAB), (3-((3-cholamidopropyl) dimethylammonio)-1-propanesulfonate) (CHAPS), or sodium dodecyl sulfate (SDS). In some embodiments, the chaotrope is or comprises urea, thiourea, or guanidine. In some embodiments, the thiol-containing reducing agent is or comprises betamercaptoethanol or dithiothreitol. In some embodiments, the digestion solution further comprises a tryptic digestion solution. In some embodiments, the tryptic digestion solution comprises triethylammonium bicarbonate, SDC, TCEP, chloroacetamide or combinations thereof. In some embodiments, the digestion solution comprises triethylammonium bicarbonate, sodium deoxycholate (SDC), TCEP, or chloracetamide, or combinations thereof. In some embodiments, the digestion solution comprises triethylammonium bicarbonate. In some embodiments, the triethylammonium bicarbonate is present in the digestion solution at a concentration of 0.1 M. In some embodiments, the digestion solution comprises sodium deoxycholate (SDC). In some embodiments, the sodium deoxycholate (SDC) is present in the digestion solution at a concentration of 0.05% to 10% (w/v). In some embodiments, the digestion solution comprises TCEP. In some embodiments, the TCEP is present in the digestion solution at a concentration of 1 mM to 100 mM. In some embodiments, the digestion solution comprises chloracetamide. In some embodiments, the chloroacetamide is present in the digestion solution at a concentration of 5 mM to 100 mM. In some embodiments, the digestion solution comprises a protease or a combination of proteases. In some embodiments, the protease is trypsin or the combination of proteases comprises trypsin.

In some embodiments, the digestion solution comprises triethylammonium bicarbonate, sodium deoxycholate (SDC), TCEP, or chloroacetamide, or combinations thereof. In some embodiments, the digestion solution comprises PBS. In some embodiments, the digestion solution comprises triethylammonium bicarbonate. In some embodiments, the digestion solution comprises sodium deoxycholate (SDC). In some embodiments, the digestion solution comprises TCEP. In some embodiments, the digestion solution comprises chloroacetamide. In some embodiments, the digestion solution comprises a protease or a combination of proteases. In some embodiments, the protease is trypsin or the combination of proteases comprises trypsin. In some embodiments, the PBS is present in the digestion solution at a concentration of 0.01 M. In some embodiments, the PBS may further comprise potassium chloride and sodium chloride. In some embodiments, the potassium chloride is at a concentration of 0.0027 M. In some embodiments, the sodium chloride is at a concentration of 0.137 M. In some embodiments, any PBS solution known in the art can be used. In some embodiments, the triethylammonium bicarbonate is present in the digestion solution at a concentration of 0.1M. In some embodiments, the sodium deoxycholate (SDC) is present in the digestion solution at a concentration of 1% (w/v). In some embodiments, the TCEP is present in the digestion solution at a concentration of 10 mM. In some embodiments, the chloroacetamide is present in the digestion solution at a concentration of 40 mM.

In some embodiments, the digestion solution comprises a protease or a combination of proteases. In some embodiments, the digestion solution comprises a protease or a combination of proteases, and further comprises triethylammonium bicarbonate, sodium deoxycholate (SDC), TCEP or chloroacetamide, or combinations thereof. In some embodiments, the digestion solution comprises trypsin.

In some embodiments, the incubating in the digestion solution is conducted for a period of time long enough to digest one or more proteins contained within or adhered to the porous material. In some embodiments, the incubating is conducted for 1 min-48 hour. In some embodiments, the incubating is conducted for 1 min-24 hour. In some embodiments, the incubating is conducted for 24-48 hour. In some embodiments, the incubating is conducted for 1, 5, 10, 15, 20, 25, 30, 35, 40, 45, 50, 55 or 60 min. In some embodiments, the incubating is conducted for 1, 2, 3, 4, 5, 6, 7, 8, 9, 10, 11, 12, 13, 14, 15, 16, 17, 18, 19, 20, 21, 22, 23 or 24 hours. In some embodiments, the incubating is conducted at ambient temperature. In some embodiments, the incubating is conducted at a temperature elevated above ambient temperature. In some embodiments, the incubating is conducted at a temperature elevated below ambient temperature. In some embodiments, the incubating is conducted with agitation.

In some embodiments, the separating comprises removing the digestion incubated porous material from the digestion solution. In some embodiments, the separating comprises removing the digestion solution from the digestion incubated porous material. In some embodiments, the separating of the digestion solution from the digestion incubated porous material is via centrifugation, filtration, or a combination of centrifugation and filtration. In some embodiments, the separating step is conducted via centrifugation. In some embodiments, the separating step is conducted via filtration. In some embodiments, the separating step is conducted via a combination of centrifugation and filtration. In some embodiments, the centrifuging is at a speed of 500 g to 10,000 g. In some embodiments, the centrifuging is for at least 1 minute. In some embodiments, the centrifuging is for 1-15 minutes. In some embodiments, the centrifuging is for no more than 15 minutes.

In some embodiments, the method comprises washing the separated first, second, third, or further, extracted porous material. In some embodiments, the method comprises washing the separated first extracted porous material. In some embodiments, the method comprises washing the separated second extracted porous material. In some embodiments, the method comprises washing the separated third extracted porous material. In some embodiments, the method comprises washing the separated further extracted porous material. In some embodiments, the separated first, second, third, or further extracted porous material is washed with a washing volume of the first, second, third, or further extraction solution, respectively. In some embodiments, the separated first extracted porous material is washed with a washing volume of the first extraction solution. In some embodiments, the separated second extracted porous material is washed with a washing volume of the first, the second, or a combination comprising the first and the second, extraction solution. In some embodiments, the separated third extracted porous material is washed with a washing volume of the first, the second, the third, or a combination comprising the first, the second, or the third, extraction solution. In some embodiments, the separated further extracted porous material is washed with a washing volume of the first, the second, the third, the further, or a combination comprising the first, the second, the third, or the further, extraction solution. In some embodiments, the separated first extracted porous material is washed with an extraction solution different from the first extraction solution.

In some embodiments, the washing volume of the first, the second, the third, or the further, extraction solution is 50 μL or more. In some embodiments, the washing volume of the first, the second, the third, or the further, extraction solution is 100 μL or more. In some embodiments, the washing volume of the first, the second, the third, or the further, extraction solution is between 5 times the volume of porous material and 3 mL. In some embodiments, the washing volume of the first, the second, the third, or the further, extraction solution is 0.05-3 mL. In some embodiments, the washing volume of the first, the second, the third, or the further, extraction solution is 0.1-3 mL. In some embodiments, the washing volume of the first, the second, the third, or the further, extraction solution is between 0.5-3 mL. In some embodiments, the washing volume of the first, the second, the third, or the further, extraction solution is 0.5, 1, 1.5, 2, 2.5, or 3 mL. In some embodiments, the washing of the separated first, second, third, or further extracted porous material is repeated 1, 2, or 3 times.

In some embodiments, the porous material is washed one or more times with Tris-HCl. In some embodiments, the porous material is washed once with Tris-HCl. In some embodiments, the porous material is washed twice with Tris-HCl. In some embodiments, the porous material is washed 3 times with Tris-HCl. In some embodiments, the Tris-HCl is at a concentration of 0.1M.

In some embodiments, the porous material is washed one or more times with Tris-HCl and NaCl. In some embodiments, the porous material is washed once with Tris-HCl and NaCl. In some embodiments, the porous material is washed twice with Tris-HCl and NaCl. In some embodiments, the porous material is washed 3 times with Tris-HCl and NaCl. In some embodiments, the Tris-HCl is at a concentration of 0.1M. In some embodiments, the NaCl is at a concentration of 0.5M.

In some embodiments, the porous material is washed one or more times with Tris-HCl and LiCl. In some embodiments, the porous material is washed once with Tris-HCl and LiCl. In some embodiments, the porous material is washed twice with Tris-HCl and LiCl. In some embodiments, the porous material is washed 3 times with Tris-HCl and LiCl. In some embodiments, the Tris-HCl is at a concentration of 0.1M. In some embodiments, the LiCl is at a concentration of 0.5M.

In some embodiments, the porous material is washed one or more with Tris-HCl and urea. In some embodiments, the porous material is washed once with Tris-HCl and urea. In some embodiments, the porous material is washed twice with Tris-HCl and urea. In some embodiments, the porous material is washed 3 times with Tris-HCl and urea. In some embodiments, the Tris-HCl is at a concentration of 0.1M. In some embodiments, the concentration of urea is 2M.

In some embodiments, the second extraction solution is a salt solution or a urea containing solution. In some embodiments, the first extraction solution and the second extraction solution are different. In some embodiments, the second extraction solution comprises Tris-HCl, NaCl, LiCl or urea or combinations thereof. In some embodiments, the first extraction solution is a salt solution and the second extraction solution is a different salt solution from the first extraction solution or is a urea-containing solution. In some embodiments, the third extraction solution is a salt solution or a urea-containing solution. In some embodiments, the third extraction solution comprises Tris-HCl, NaCl, LiCl, or urea, or combinations thereof. In some embodiments, the third extraction solution comprises Tris-HCl. In some embodiments, the third extraction solution comprises NaCl. In some embodiments, the third extraction solution comprises LiCl. In some embodiments, the third extraction solution comprises urea. In some embodiments, Tris-HCl is present in the third extraction solution at a concentration of 0.1M. In some embodiments, NaCl is present in the third extraction solution at a concentration of 0.5M. In some embodiments, LiCl is present in the third extraction solution is at a concentration of 0.5M. In some embodiments, urea is present in the third extraction solution at a concentration of 2M.

In some embodiments, the first extraction solution, the second extraction solution, and the third extraction solution are different. In some embodiments, the first extraction solution is a salt solution, the second extraction solution is a different salt solution from the first extraction solution and the third extraction solution is a different salt solution from the first extraction solution and the second extraction solution or is a urea-containing solution. In some embodiments, the second extraction solution comprises Tris-HCl. In some embodiments, the second extraction solution comprises NaCl. In some embodiments, the second extraction solution comprises LiCl. In some embodiments, the second extraction solution comprises urea. In some embodiments, the Tris-HCl is present in the second extraction solution at a concentration of 0.1M. In some embodiments, the NaCl is present in the second extraction solution at a concentration of 0.5M. In some embodiments, the LiCl is present in the second extraction solution at a concentration of 0.5M. In some embodiments, the urea is present in the second extraction solution at a concentration of 2M.

In some embodiments, the separated sample mixture is extracted with at least a second extraction solution.

In some embodiments, the first extraction solution or the second extraction solution comprises Tris-HCl. In some embodiments, the Tris-HCl is at a concentration of 0.1M. In some embodiments, the first extraction solution or the second extraction solution comprises Tris-HCl and NaCl. In some embodiments, the Tris-HCl is at a concentration of 0.1M. In some embodiments, the NaCl is at a concentration of 0.5M. In some embodiments, the first extraction solution or the second extraction solution comprises Tris-HCl and LiCl. In some embodiments, the Tris-HCl is at a concentration of 0.1M. In some embodiments, the LiCl is at a concentration of 0.5M. In some embodiments, the first extraction solution or the second extraction solution comprises Tris-HCl and urea. In some embodiments, the Tris-HCl is at a concentration of 0.1M. In some embodiments, the urea is at a concentration of 2M.

In some embodiments, the detecting step comprises detecting proteins in the separated first extraction solution. In some embodiments, the detecting step comprises detecting proteins in the separated second extraction solution. In some embodiments, the detecting step comprises detecting proteins in the separated third extraction solution. In some embodiments, the detecting step comprises detecting proteins in the further extraction solution. In some embodiments, the the detecting of the first, second, third, or further sets of proteins from the first, second, third, or further extraction solutions, respectively, is via immunoassay. In some embodiments, the detecting of the first, second, third, or further sets of proteins from the first, second, third, or further extraction solutions, respectively, is via Mass Spectrometry (MS).

In some embodiments, the detection via MS comprises subjecting said first, second, third, or further extraction solution to digestion prior to said detection via Mass Spectrometry (MS). In some embodiments, prior to detecting the first, second, third, or further sets of proteins in the first, second, third, or further extraction solutions, respectively, the method further comprises incubating said first, second, third, or further extraction solution, in a digestion solution.

In some embodiments, any one of the methods described herein further comprises producing a protein profile of the first set of proteins. In some embodiments, a first protein profile is produced from the collected first set of proteins. In some embodiments, the first protein profile is produced by a proteomics work flow. In some embodiments, the first protein profile is produced by immunoassay work flow.

In some embodiments, said detecting comprises an immunoassay work flow. In some embodiments, said immunoassay is or comprises an ELISA. In some embodiments, said immunoassay is or comprises a Western blot. In some embodiments, said immunoassay comprises a Luminex and Proximity Extension Assay. In some embodiments, the Luminex and Proximity Extension Assay further comprises Slow Offrate Modified Aptamer (SOMAmer) reagents.

In some embodiments, the detecting step comprises a proteomics work flow. In some embodiments, the proteomics work flow comprises mass spectrometry (MS). In some embodiments, the MS is LC-MS. In some embodiments, the MS is selected reaction monitoring mass spectrometry (SRM-MS). In some embodiments, the MS is data-dependent acquisition MS (DDA-MS). In some embodiments, the MS is data-independent acquisition MS (DIA-MS). In some embodiments, the MS is selected from the group consisting of matrix-assisted laser desorption/ionisation time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS; APCI-(MS)n; ion mobility spectrometry (IMS); inductively coupled plasma mass spectrometry (ICP-MS) atmospheric pressure photoionization mass spectrometry (APPI-MS), APPI-MS/MS; and APPI-(MS)n.

In some embodiments, any one of the methods described herein further comprises producing a protein profile of the cell-membrane associated proteins. In some embodiments, a cell-membrane associated protein profile is produced from the collected set of cell-membrane associated proteins. In some embodiments, the cell-membrane associated protein profile is produced by LC-MS. In some embodiments, the cell-membrane associated protein profile is produced by immunoassay. In some embodiments, the cell-membrane associated proteins are or comprise cell-membrane bound proteins or cell integral-membrane proteins. In some embodiments, the cell-membrane bound proteins comprise one or more membrane protein complexes.

In some embodiments, the porous material is a three-dimensional porous material. In some embodiments, the three-dimensional porous material comprises a plastic. In some embodiments, the three-dimensional porous material comprises a sponge. In some embodiments, the three-dimensional porous material is a tip of a volumetric absorptive microsampling (VAMS) device. In some embodiments, the porous material is a non-pre-loaded porous material. In some embodiments, the porous material is not pre-loaded with protease inhibitors. In some embodiments, the porous material is a pre-loaded porous material. In some embodiments, the porous material is pre-loaded with a protease inhibitor. In some embodiments, the porous material is pre-loaded with an anticoagulant. In some embodiments, the the anticoagulant is EDTA. In some embodiments, the anticoagulant is heparin.

In some embodiments, the method further comprises placing the sample-containing porous material in a tube. In some embodiments, the method further comprises placing the dried sample-containing porous material in a tube. In some embodiments, the tube is an Eppendorf tube.

In some embodiments, the method uses a plurality of the porous materials. In some embodiments, the sample is introduced into the plurality of the porous materials. In some embodiments, the plurality of the porous materials comprises two or more porous materials. In some embodiments, the plurality of the porous materials is two porous materials. In some embodiments, the plurality of the porous materials is three porous materials.

In some embodiments, one or more proteins detected in the first, second, third, or further set of proteins is or comprises a non-membrane/soluble protein complex. In some embodiments, one or more proteins detected in the separated digestion solution is or comprises a non-membrane/soluble protein complex. In some embodiments, one or more proteins detected in the first, second, third, or further extraction solution following incubating in a digestion solution is or comprises a non-membrane/soluble protein complex. In some embodiments, one or more proteins detected in the first, second, third, or further set of proteins is or comprises a cell-membrane associated protein. In some embodiments, one or more proteins detected in the separated digestion solution is or comprises a cell-membrane associated protein. In some embodiments, one or more proteins detected in the first, second, third, or further extraction solution following incubating in a digestion solution is or comprises a cell-membrane associated protein. In some embodiments, the cell-membrane associated protein is a cell-membrane bound protein or a cell integral-membrane protein. In some embodiments, the cell-membrane associated protein is a membrane protein complex.

In some embodiments, where glycans present in the sample are to be assessed, the method comprises a multi-glycomics workflow wherein different glycans can be sequentially released prior to the digestion step. Glycosaminoglycans (GAGs), Glycosphingolipids (GSLs) and N-glycans can all be released sequentially through enzymatic means while preserving the peptide backbone. In some embodiments, the method further comprises, prior to the digestion step, dividing the porous material, and analysing a portion of the portion of the porous material for remaining glycans. In one embodiment the remaining glycans are those which remain following an enzymatic treatment to release the glycans. In one embodiment the remaining glycans are analysed following release of O-glycans via reductive beta elimination.

### Devices

Dried blood spots (DBS) may be used as a sample collection system. However, while DBS provides a simple and reliable means for blood sample collection without the need for medical intervention, there are some drawbacks. For example, one drawback is the lack of separation in a DBS, which means the entire blood sample (plasma and cells) are collected in a single spot. This can be a concern where analytes are present in multiple compartments, plasma and cells for example, and the DBS extract returns a different answer to plasma separated from venous blood.

Provided herein is a device for obtaining defined volume fractions from small blood volumes.

In one aspect, provided herein is a microsampling device, comprising: a) a first porous material; b) a second porous material; and c) a tube.

In one aspect, provided herein is a microsampling device, comprising: a) a first porous material, b) a second porous material; c) a third porous material; and d) a tube.

In one aspect, provided herein is a microsampling device, comprising: a) a first porous material; b) a second porous material; and c) a shaft; wherein the first porous material is positioned at one end of the shaft and the second porous material is located at a position along the shaft so that the first porous material and the second porous material are separated and not in physical contact with each other.

In one aspect, provided herein is a microsampling device, comprising: a) a first porous material; b) a second porous material; c) a third porous material; and d) a shaft; wherein the first porous material is positioned at one end of the shaft and the second porous material and third porous material are located at positions on the shaft so that the first porous material, the second porous material, and the third porous material are separated and not in physical contact with each other.

In some embodiments, the microsampling device is a volumetric absorptive microsampling (VAMS) device.

In some embodiments, the tube is an Eppendorf tube.

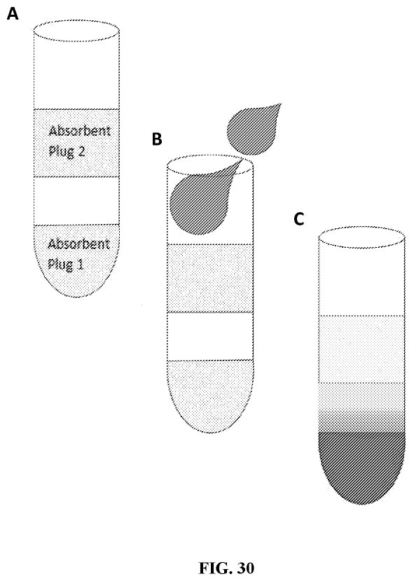

In some embodiments, a tube containing two absorbent materials (sometimes referred to as absorbent ‘plugs’ of sponge-like absorbent material) may take up a defined volume of fluid. In some embodiments, the absorbent plugs may also contain anti-coagulant material (wet or dry) to prevent blood clotting during the separation. In some embodiments, the device comprises the device of FIG. 30.

In some embodiments, blood is introduced to the top of the tube. In some embodiments, the blood is collected from a finger prick. In some embodiments, the tube comprises the tube of any one of FIGS. 30-33. In some embodiments, after centrifugation, the cells (RBCs, WBCs and platelets) are removed from the plasma, leaving plasma in absorbent plug 2 and RBCs and other cells in Absorbent Plug 1.

In some embodiments, the device provided here enable small volumes of blood, including from finger prick samples, to be immediately processed to obtain plasma and cellular fractions. The absorbent material used in the device disclosed herein may be a sponge-like material. In some embodiments, the absorbent material may be prepared from, or is, a Neoteryx tip.

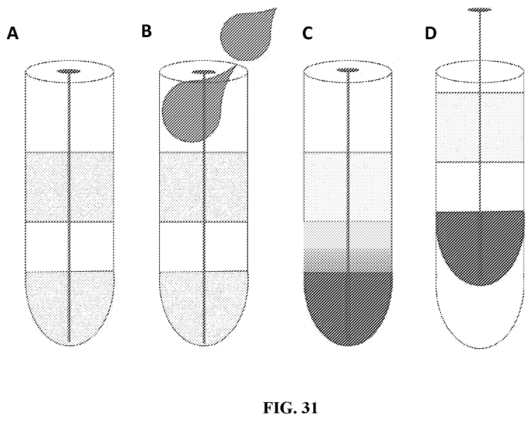

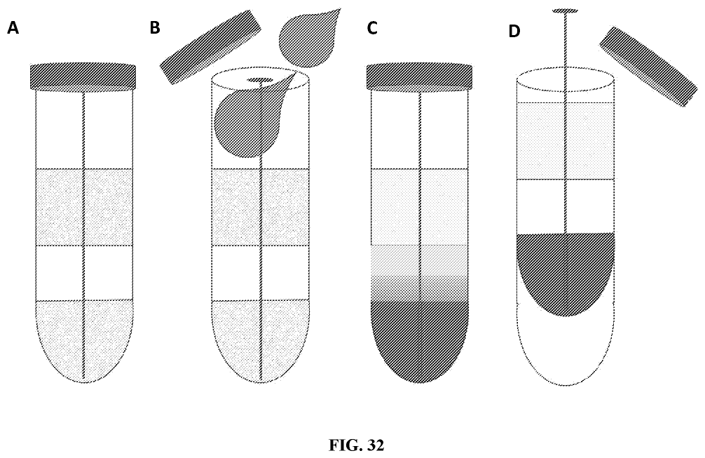

In one embodiment the two absorbent plugs are connected by a shaft, which also functions as a handle to remove the absorbent plugs (see FIG. 31).

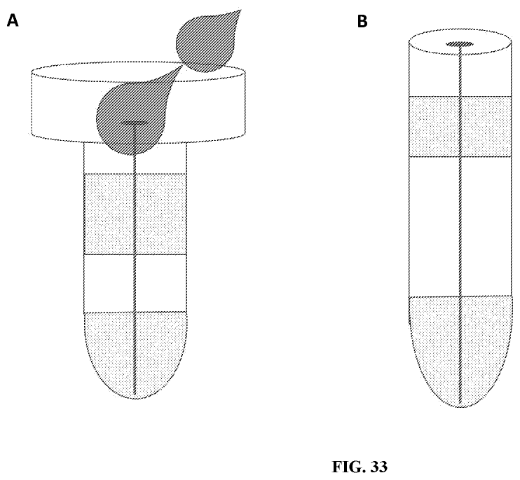

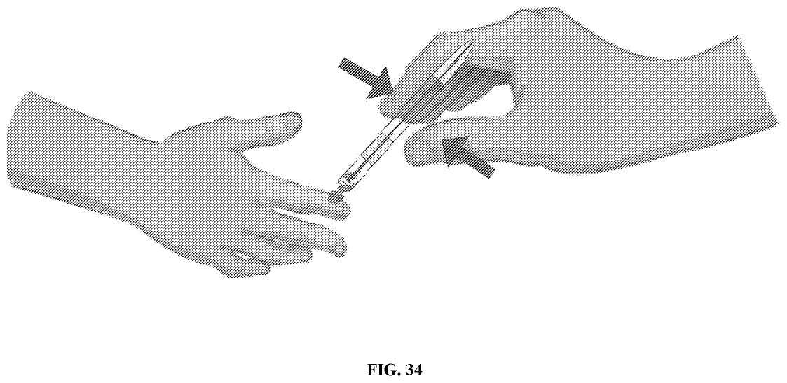

In some embodiments, the tubes may comprise a lid to prevent spillage and suppress the release of aerosols during centrifugation (see FIG. 32). In some embodiments, the tubes may have a wider opening at the top to facilitate blood collection by wiping or scraping blood drops from a fingertip (see FIG. 33). In some embodiments, the tubes may also be long and narrow, with variable spacing between the absorbent plugs to ensure that the plasma sample is not contaminated by cells or platelets. In some embodiments, the tubes may also be long and narrow, with flexible walls between the absorbent plugs to enable blood drops to be aspirated by squeezing the tube and releasing (see FIG. 34).

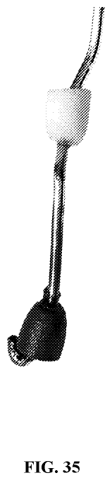

In some embodiments, the device may use a wire to create the shaft (handle). This was shaped to keep the absorbent plugs separate (see FIG. 35).

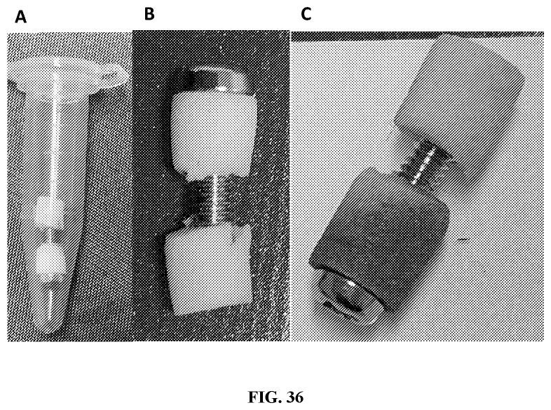

The device may use 2 mm threaded bolts to create the shaft (handle). The thread on the shaft prevented the upper (plasma) absorbent plug from moving down during centrifugation (see FIG. 36).

In some embodiments, the porous material is a pre-loaded porous material. In some embodiments, the porous material is pre-loaded with protease inhibitors. In some embodiments, the porous material is pre-loaded with anticoagulant. In some embodiments, the porous material is pre-loaded with enzymes. In some embodiments, the porous material is pre-loaded with detergent. In some embodiments, the anticoagulant is EDTA. In some embodiments, the anticoagulant is heparin. In some embodiments, the enzyme is benzonase. In some embodiments, the detergent is sodium dodecyl sulfate. In some embodiments, the porous material is a non-pre-loaded porous material. In some embodiments, the porous material is not pre-loaded with protease inhibitors. In some embodiments, the first porous material and second porous material are pre-loaded with anticoagulant.

In some embodiments, the first porous material and second porous material are equally spaced. In some embodiments, the first porous material, the second porous material and the third porous material are equally spaced.

In some embodiments, the first porous material and second porous material are air dried. In some embodiments, the first porous material and second porous material are air dried for at least 1, 2, 4, 6, 8, 10, 12, 16, 20 or 24 hours. In some embodiments, the first porous material and second porous material are air dried for 24 hours. In some embodiments, the first porous material and second porous material are air dried for at least 1 day. In some embodiments, the first porous material and second porous material are air dried for a period of time to adhere the sample to the porous material. In some embodiments, the first porous material and second porous material are vacuum dried.

In some embodiments, the first porous material has an absorptive capacity in the range of 2.5 μL to 50 μL. In some embodiments, the first porous material has an absorptive capacity of 2.5 μL, 5 μL, 10 μL, 20 μL, 30 μL, 40 μL, or 50 μL. In some embodiments, the second porous material has an absorptive capacity in the range of 2.5 μL to 50 μL. In some embodiments, the second porous material has an absorptive capacity in the range of 2.5 μL, 5 μL, 10 μL, 20 μL, 30 μL, 40 μL, or 50 μL. In some embodiments, the third porous material has an absorptive capacity in the range of 2.5 μL to 50 μL. In some embodiments, the third porous material has an absorptive capacity in the range of 2.5 μL, 5 μL, 10 μL, 20 μL, 30 μL, 40 μL, or 50 μL.

In some embodiments, the first porous material has an absorptive capacity of 5 μL. In some embodiments, the first porous material has an absorptive capacity of 10 μL. In some embodiments, the first porous material has an absorptive capacity of 20 μL. In some embodiments, the first porous material has an absorptive capacity of 30 μL. In some embodiments, the second porous material has an absorptive capacity of 5 μL. In some embodiments, the second porous material has an absorptive capacity of 10 μL. In some embodiments, the second porous material has an absorptive capacity of 20 μL. In some embodiments, the second porous material has an absorptive capacity of 30 μL.

In some embodiments, the first porous material and second porous material have the same absorptive capacity. In some embodiments, the first porous material, the second porous material, and the third porous material have the same absorptive capacity. In some embodiments, the first porous material and second porous material have different absorptive capacity. In some embodiments, the first porous material, the second porous material, and the third porous material have different absorptive capacities. In some embodiments, the first porous material and the second porous material have the same absorptive capacity, and the third porous material has a different absorptive capacity relative to said first and second porous materials. In some embodiments, the first porous material has an absorptive capacity of 5 μL and the second porous material has an absorptive capacity of 5 μL. In some embodiments, the first porous material has an absorptive capacity of 10 μL and the second porous material has an absorptive capacity of 10 μL. In some embodiments, the first porous material has an absorptive capacity of 20 μL and the second porous material has an absorptive capacity of 20 μL. In some embodiments, the first porous material has an absorptive capacity of 30 μL and the second porous material has an absorptive capacity of 30 μL. In some embodiments, the first porous material has an absorptive capacity of 30 μL and the second porous material has an absorptive capacity of 20 μL. In some embodiments, the first porous material has an absorptive capacity of 30 μL and the second porous material has an absorptive capacity of 10 μL. In some embodiments, the first porous material has an absorptive capacity of 30 μL and the second porous material has an absorptive capacity of 5 μL. In some embodiments, the first porous material has an absorptive capacity of 5 μL and the second porous material has an absorptive capacity of 30 μL. In some embodiments, the first porous material has an absorptive capacity of 10 μL and the second porous material has an absorptive capacity of 30 μL. In some embodiments, the first porous material has an absorptive capacity of 20 μL and the second porous material has an absorptive capacity of 30 μL.

In some embodiments, the first porous material, second porous material and third porous material have the same absorptive capacity. In some embodiments, the first porous material, second porous material and third porous material have a different absorptive capacity. In some embodiments, the first porous material and the second porous material have the same absorptive capacity and the third porous material has a different absorptive capacity relative to said first and second porous materials.

In some embodiments, the microsampling device further comprises a shaft. In some embodiments, the shaft is a threaded shaft. In some embodiments, the shaft is a non-linear shaft. In some embodiments, the shaft is a curved shaft. In some embodiments, the shaft has a length between 10 mm and 50 mm. In some embodiments, the shaft has a diameter between 0.5 mm and 3 mm. In some embodiments, the shaft has a diameter between 0.5 mm and 5 mm. In some embodiments, the shaft may be a hollow tube to enable airglow and prevent airlocks occurring after a sample is loaded. In some embodiments, the shaft fits in a tube. In some embodiments, the shaft fits in a tube having a volume of 5 mL, 2 mL, 1.5 mL, or 1 mL. In some embodiments, the shaft fits in an Eppendorf tube. In some embodiments, the shaft fits in an Eppendorf tube having a volume of 5 mL, 2 mL, 1.5 mL, or 1 mL. In some embodiments, the shaft fits in an Eppendorf tube having a volume of 5 mL. In some embodiments, the shaft fits in an Eppendorf tube having a volume of 2 mL. In some embodiments, the shaft fits in an Eppendorf tube having a volume of 1.5 mL. In some embodiments, the shaft fits in an Eppendorf tube having a volume of 1 mL. In some embodiments, shaft fits in a 96-well plate.

In some embodiments, the first porous material and the second porous material are positioned on the shaft and separated from each other by a spacer such that said first porous material and said second porous material are separated and not in physical contact with each other. In some embodiments, the first porous material, the second porous material, and the third porous material are positioned on the shaft and separated from each other by spacers such that said first porous material, said second porous material, and said third porous material are separated and not in physical contact with each other.

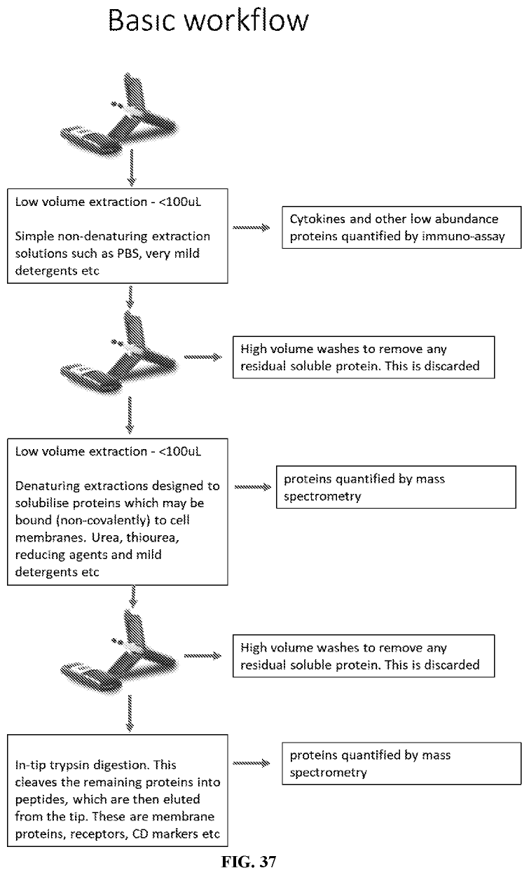

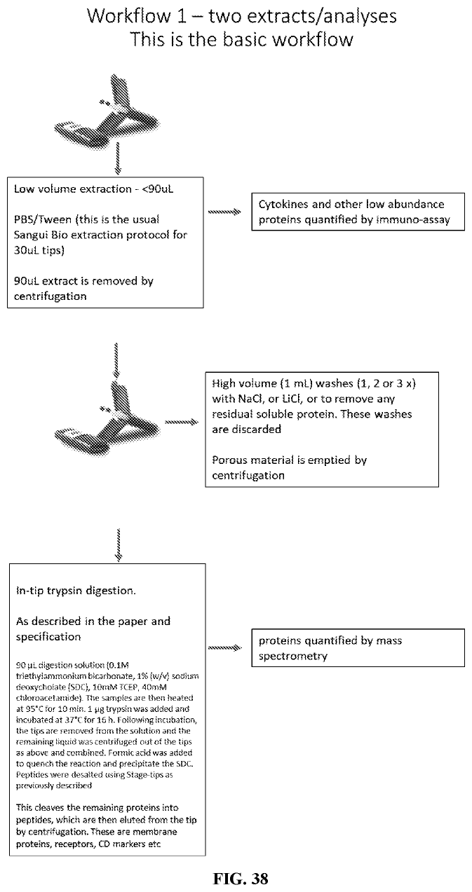

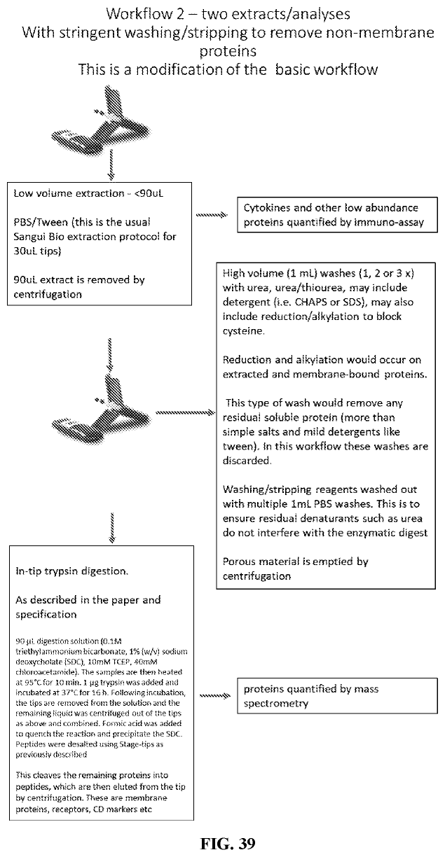

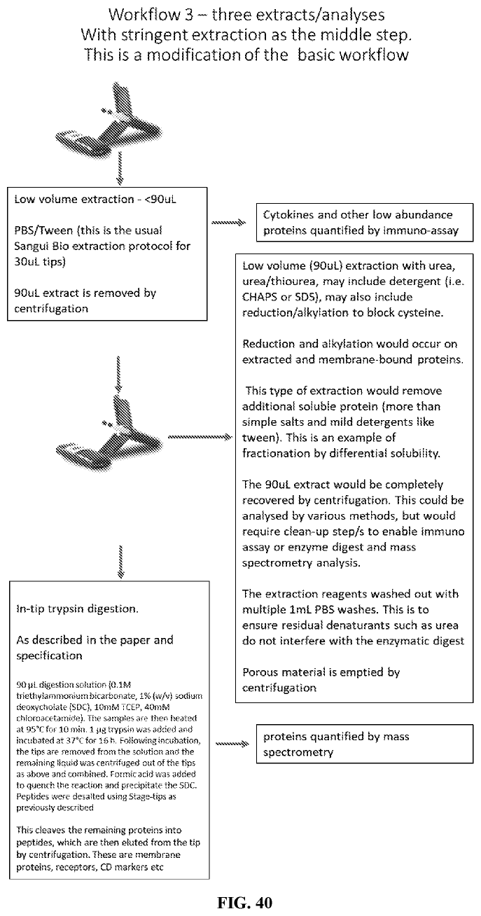

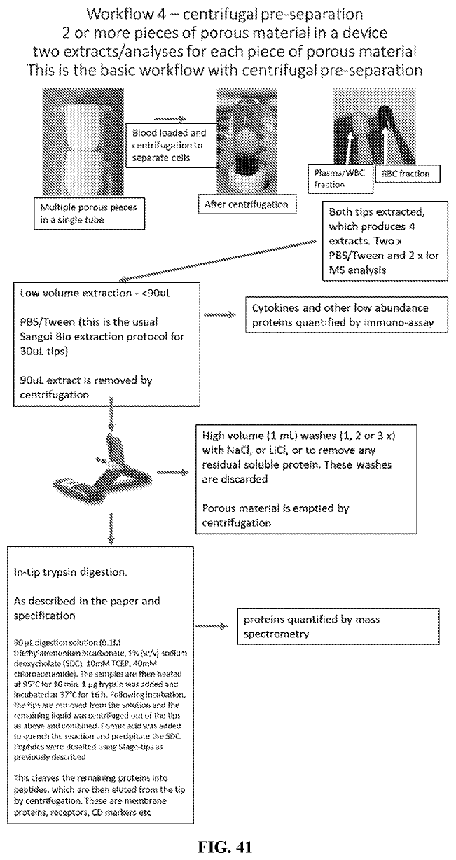

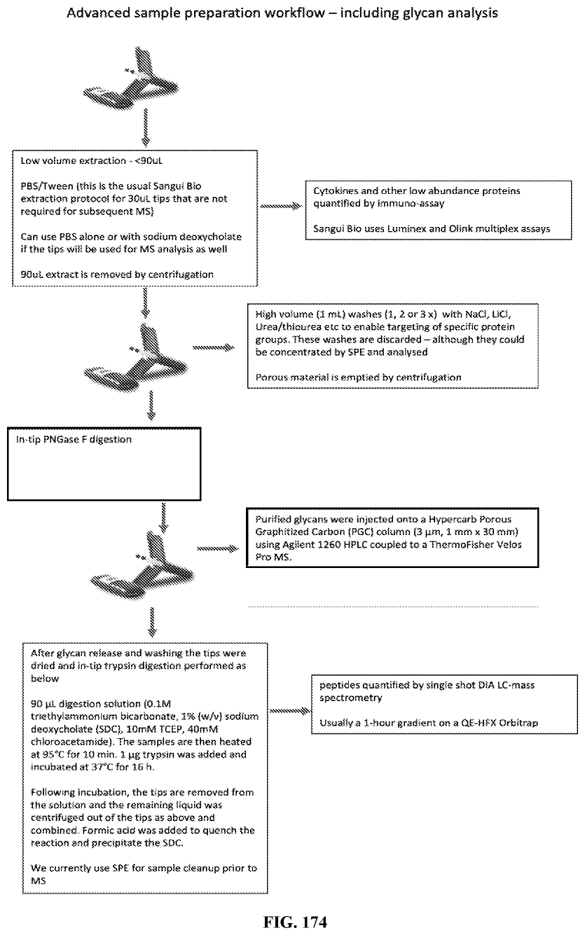

In some embodiments, any one of the methods provided herein may be performed using any one of the devices provided herein. In some embodiments, the method and device comprises the method and device of FIG. 37. In some embodiments, the method and device comprises the method and device of FIG. 38. In some embodiments, the method and device comprises the method and device of FIG. 39. In some embodiments, the method and device comprises the method and device of FIG. 40. In some embodiments, the method and device comprises the method and device of FIG. 41. In some embodiments, the method and device comprises the method and device of FIG. 174.

In some embodiments, provided herein is a kit comprising the microsampling device provided herein. In some embodiments, the kit further provides instructions for using the microsampling device.

### EXAMPLES

The following examples evaluate the use of a volumetric absorptive microsampling (VAMS) device as a substrate for a whole blood sample to enable untargeted proteomic analysis of dried whole blood. Particularly, the purpose of the following examples was to evaluate protein identification capabilities of the VAMS compared to traditional DBS methods.

The use of VAMS led to the detection and robust quantitation of up to 1,600 proteins from single-shot shotgun-LC-MS analysis of dried whole blood, greatly enhancing proteome depth compared with conventional single-shot LC-MS analyses of undepleted plasma. Some proteins not previously reported in blood were detected using this approach. Various washing reagents were used to demonstrate that proteins can be preferentially removed from VAMS devices prior to downstream analyses. Proof of concept experiments also show that archival of frozen blood cell pellets housed under long-term storage (exceeding 5 years) are also compatible with VAMS, enabling quantitation of potential biomarker proteins from biobank repositories for use in biomarker studies. These demonstrations are important steps in establishing viable analysis workflows to underpin large-scale precision medicine studies.

It has been demonstrated that VAMS are an ideal substrate to reduce the most highly abundant blood proteins through washing which enabled detection of over 1,600 proteins from single-shot analysis of dried whole blood, including those plasma proteins typically detected in LC-MS analyses of un-depleted plasma.

### Example I: Proteomic Analysis of Whole Blood and Plasma Using Volumetric Absorptive Microsampling (VAM) Device

Blood B Collection and Application of DBS Matrices. Written consent was obtained and blood was collected from healthy volunteers under a study approved by the Northern Sydney Local Health District ethics committee (2020/ETH01974). DBS samples were prepared using 30 μL Mitra® VAMS devices (Neoteryx, Torrance, CA) or Whatman FTA-DMPK C filter paper as specified.

Venous blood was collected from participants by venipuncture into EDTA vacutainers (BD, North Ryde, Australia). Whole blood was directly applied to the VAMS tips until filled completely. In some instances, frozen whole blood was compared to fresh whole blood. For this, an aliquot of EDTA blood was immediately frozen at −80° C. for 24 hrs. Blood was thawed and applied to the VAMS tip or filter paper. For preparation of plasma samples, the whole blood was centrifuged (10 min, 1000 g, brake at 50%) and the resulting plasma was collected and applied to the VAMS tips. The samples were left to dry for 5 minutes at room temperature before being transferred to foil zip-lock bags with desiccant. These samples were stored at 4° C. for a minimum of 24 hrs. before extraction. Whole blood was applied to Whatman FTA-DMPK C filter paper using a pipette, left to dry for 30 min at room temperature and stored at 4° C. for a minimum of 24 hrs. before extraction.

Frozen Whole Blood Cell Pellets from cancer Patients. Frozen whole blood cell pellets (plasma removed) from 5 healthy and 5 Stage III cancer donors, matched for both age and sex, were purchased from PrecisionMed Inc. (Solana Beach, CA, USA). Samples were collected between 2014-2018. The supplier indicated that samples from healthy participants were stored at −20° C. for between 35.6-263.0 weeks and 43.0-165.4 weeks for cancer patients. Upon receipt in our laboratory, samples were stored at −80° C. for 101 weeks prior to the analysis reported herein. The samples were thawed on ice and applied to the microsampling device until filled completely. The samples were then left to dry for 5 minutes at room temperature before being transferred to foil zip-lock bags with desiccant. These samples were all stored at 4° C. for a minimum of 24 hrs. before extraction.

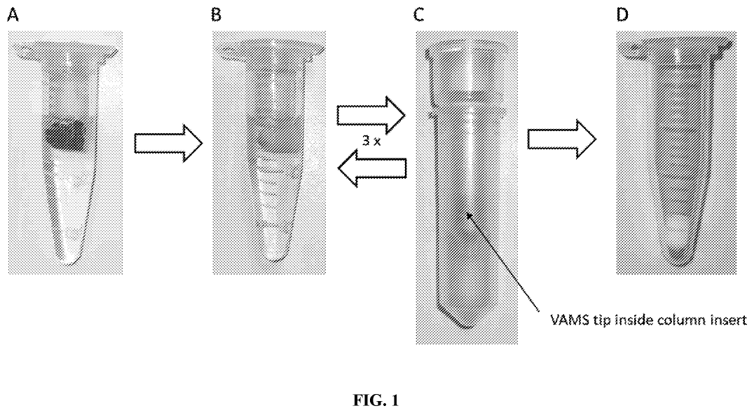

DBS Washing and Extraction. VAMS DBS tips were removed from their spindle and suspended in 1 mL of extraction solution in 1.5 mL Eppendorf tubes and left to incubate at room temperature for 24 hrs. whilst gently shaking (100 rpm). Extraction solution was 0.1M Tris-HCl containing either 0.5M NaCl, 0.5M LiCl or 2M urea as indicated. Following the incubation, the VAMS tips were washed three times in fresh extraction solution. This was performed by pulse centrifugation of the tip in the extraction solution (3× pulses 0-10,000 g) followed by centrifuging the extraction solution out of the tips with aid of a custom column insert device (Micro BioSpin P-30 gel column with column material and base filter removed, BioRad USA) and resuspending them in fresh extraction solution (3000 g, 3 min, full brake). After the final wash, the dry tip with the extraction solution removed, was placed into a fresh Eppendorf tube for tryptic digestion. An overview of the method is shown in FIG. 1.

Paper DBS disc was washed in 1 mL of PBS or 0.5M LiCl for 24 hours. The paper disc was then placed within a custom column insert device as described above, inside a waste collection microcentrifuge tube, and spun at 3000 g for 3 minutes. The wash solution was removed and the filter paper disc placed into a fresh microcentrifuge tube for tryptic digestion.

Trypsin Digestion. The washed DBS tips/paper were incubated with 90 μL digestion solution (0.1M triethylammonium bicarbonate, 1% (w/v) sodium deoxycholate (SDC), 10 mM TCEP, 40 mM chloroacetamide). The samples were then heated at 95° C. for 10 min. 1 μg trypsin was added and incubated at 37° C. for 16 h. Following incubation, the tips were removed from the solution and the remaining liquid was centrifuged out of the tips as above and combined. Formic acid was added to quench the reaction and precipitate the SDC. Peptides were desalted using Stage-tips as previously described (Rappsilber et al., Anal Chem. 2003; 75 (3): 663-70).

Mass Spectrometry acquisition. Peptides were loaded in 0.1% formic acid onto a 75 μm×40 cm 1.9 μm C18 (ReproSil-Pur C18-AQ) nanoLC column and eluted over 90 min gradient to a maximum of 30% solvent B (80% ACN, 0.1% FA).

Peptides were detected using a HF-X orbitrap mass spectrometer (Thermo, CA, USA). When operating in DDA-mode the following instrument settings were used: MS1-AGC 3e6, Resolution 60K, Scan range 300-1650 m/z. MS2-AGC 1e5, Resolution 15K, loop count 15.

When operating in DIA-mode the following instrument parameters were used: Full MS scan resolution 60K, AGC 3e6, maximum injection time 20 ms, scan range 350-1300 m/z, followed by two DIA events containing a total of 117×6 m/z mass windows to scan 400-1218 m/z with settings of Resolution 15 K, AGC 3e6, maximum injection time 32 ms. The normalized collision energy was set to 30, default charge state was 2.

Protein Identification. For DDA experiments, raw files were searched with MaxQuant V1.6.5 against the UniProt human proteome database (UP000005640, 20,350 reviewed protein entries) using the following settings: reverse-target decoy strategy to produce 1% FDR PSM and protein, min peptides 2, MSMS mass tolerance 20 ppm, missed cleavages set to max 2, variable oxidation of methionine and fixed carbamidomethyl modification of Cys, LFQ enabled requiring two peptides with default settings.

For DIA experiments analysis was performed using Spectronaut Pulsar X v12.0.2 (Biognosys, Switzerland) using a custom library produced from DDA experiments of the same specimens. A mutated decoy search method was applied with 1% precursor and protein FDR.

The mass spectrometry proteomics data have been deposited to the ProteomeXchange Consortium via the PRIDE partner repository with the dataset identifier PXD028605.

Quantitation and Statistical Analyses. For DDA experiments, Perseus software was used to filter out reverse hits and peptide matches to single peptides (two peptides was the minimum required for protein quantitation). Data was log 2 transformed and median normalization applied. Missing quantitation values were replaced by imputation with Perseus using default settings-values sampled from a Gaussian distribution down shifted by 1.8 standard deviations, with a width of 0.3. For DIA experiments peptides quantified by a minimum of three fragments, protein quantification by top n=5 peptides. Data was then exported as a “Q-value Sparse” pivot report matrix with normalized protein areas, missing values were represented with “NaN”. UpSet plots and principal component analysis plots were generated using R. Statistical analysis of sample group protein identification differences was conducted by ANOVA with Tukey post-hoc analysis, with Q<0.05. Gene ontology enrichment was conducted using the R package PloGO2 using a default set of 42 GO categories (Wu et al., Journal of Proteome Research. 2020; 19 (7): 2898-906). For the purpose of determining GO category enrichment, numbers of proteins in each category were compared to the baseline set of all proteins identified in the experiment using Fisher's exact test; counts, percentages and fdr-adjusted Fisher exact test p-values were generated and summarized. Differentially expressed proteins were identified using Limma moderated T-test, with FC>2 and P<0.05 considered significant.

LC-MS of Dried Whole Blood and Dried Plasma Applied to VAMS Devices. Capillary blood microsampling and deposition as dried blood spots (DBS) has important practical advantages over conventional venous blood phlebotomy. To date, however, proteomic biomarker discovery studies have typically avoided use of DBS, favoring shotgun LC/MS analysis of blood plasma or serum as this avoids the high concentrations of erythrocyte proteins which would otherwise dominate in DDA-MS studies to severely limit proteome depth. Nevertheless, it is widely accepted that LC-MS analysis of plasma will not solve these problems, due to the presence of highly abundant protein species including immunoglobulins, serum albumin, acute phase reactants, apolipoproteins, etc. that dominate MS sequencing time (Anderson and Anderson, Mol Cell Proteomics. 2002; 1 (11): 845-67; Hortin et al., Clin Chem. 2008; 54 (10): 1608-16; Chiu et al., Bioanalysis. 2009; 1 (4): 847-55). For most laboratories, a typical sampling of neat human plasma using single shot DDA LC-MS yields 200-300 proteins depending on instrument configuration and detection sensitivity. Efficacy of proteomic analysis of DBS collection and processing was determined using a VAMS device, as it was rationalized that signal saturation due to abundant blood proteins could be manipulated by chemical washing of proteins from the VAMS device prior to LC/MS. This would enable access to less abundant proteins associated with cells in circulation (i.e. leukocytes, platelets, circulating tumor cells, shed somatic cells) which are commonly missing from single-shot LC-MS proteomic analysis of blood plasma and they may provide important precision medicine biomarkers.

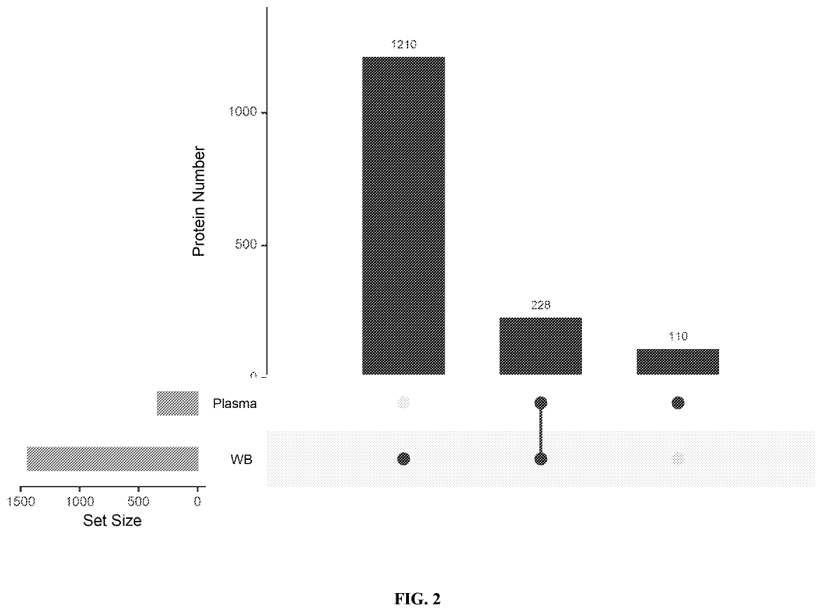

Using DDA on a nanoLC-MS H-FX orbitrap system, a label free quantitation of three replicates to characterize the proteins detected and their quantitative abundance for both whole blood (i.e. DBS) and dried plasma spots processed by VAMS device. As shown in FIG. 2, 1,438 proteins with a minimum of two peptides per protein were detected in WB two or more DBS replicates (of which 1210 proteins were unique to WB). Applying paired plasma to the VAMS yielded 338 proteins in two or more replicates (of which 110 proteins were unique to plasma). 228 proteins were common to both WB and plasma from VAMS devices which included most of the acute phase reactants typically seen in undepleted plasma LC MS analyses (data not shown).

Notably, the quantitative reproducibility (% CV) for the analysis of WB proteins from DBS was highly acceptable to established guidelines (Carr et al., Mol Cell Proteomics. 2014; 13 (3): 907-17) and superior to analysis of plasma proteins applied to VAMS (13.5% (Interquartile range (IQR) 8.8-19.5) vs 23.8% (IQR 14.2-35.8)). While the analysis of plasma using VAMS itself was promising and enabled similar depth of the plasma proteome compared to conventional LC-MS analysis of undepleted plasma, analysis of WB as DBS provided access to most of these plasma proteins in addition to hundreds of other cellular proteins that are not commonly detected in single-shot LC-MS analysis of undepleted plasma. This included 54 proteins annotated in ProteinAtlas (Uhlen et al., Sci Signal. 2019; 12 (609)) as cluster of differentiation markers (CD antigens) and many others with important established roles in various pathologies (e.g. SOD1, HTT, MTOR, IDH2). Of the 1438 proteins detected in WB in at least 2 of 3 DDA experiments with 2 or more peptides, 1180 are reported to have been detected previously in plasma/serum according to the Plasma Proteome Database (Nanjappa et al., Nucleic Acids Res. 2014; 42 (Database issue): D959-65.), with 318 additional proteins not described (data not shown).

As one example, demonstrating the potential of WB DBS for biomarker discovery, PDZK1 interacting protein 1 (MAP17), the product of PDZK1IP1, was identified and had not previously been reported in the Plasma Proteome Database, nor detected in blood according to ProteinAtlas (Uhlen et al., Science. 2015; 347 (6220): 1260419). Investigation in ProteinAtlas reveals MAP17 to be enriched in kidney tubules and subcellular antibody staining indicates locations of cytosol and nuclear speckles. MAP17 has been reported as a necessary activator of renal Na⁺/Glucose co-transport (Coady et al., J Am Soc Nephrol. 2017; 28 (1): 85-93). MAP17 is overexpressed in a variety of human carcinomas and tissue staining of MAP17 has been reported to predict response to neoadjuvant chemoradiotherapy in rectal cancer (Rivero et al., Oncotarget. 2018; 9 (68): 32958-71) and response to cisplatin, carboplatin and EGFR inhibitors in lung adenocarcinomas (Ferrer I et al., J Exp Clin Cancer Res. 2018; 37 (1): 195). The detection of MAP17 in WB using DBS microsampling underscores the general utility of our new approach, here providing an opportunity to establish the clinical utility of this protein as a secreted cancer biomarker.

It was noted that washing of WB on VAMS devices does not eliminate highly abundant erythrocyte proteins, but it was effective in removing sufficient quantities to facilitate deeper proteomic profiling compared to conventional plasma analyses using LC-MS. Therefore, analysis of proteins from WB using VAMS devices holds promise for increased discovery of proteomic biomarkers using a streamlined sample collection, preparation and LC-MS acquisition workflow.

In summary, FIG. 2 demonstrates that the microsampling devices provided herein can be used to isolate proteins from WB and plasma.

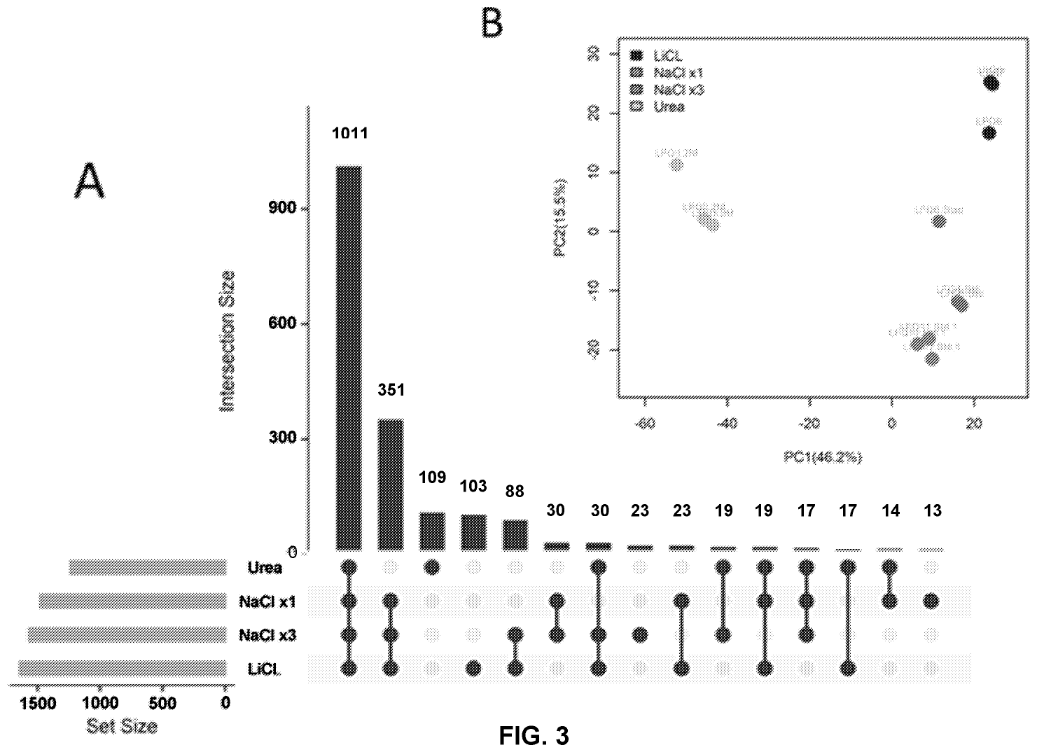

Differential protein extraction dependent on washing conditions. The effect of varying DBS washing conditions was tested, using NaCl as the reference reagent. As shown in FIG. 3, different proteins subsets could be detected dependent on the washing conditions used. While 1,011 proteins were recovered in all washing conditions, ANOVA analysis followed by post-hoc Tukey test demonstrated that 2M urea was effective at stripping additional proteins from the DBS (351 fewer proteins compared with other conditions) (data not shown). Interestingly, 109 proteins were detected uniquely following urea washing. Gene ontology enrichment showed that RNA binding proteins were selectively captured with urea washing (37% compared with 16% in whole dataset) (data not shown). The tips were washed with NaCl once or three-times, and it was found that more thorough washing enabled detection of an additional 91 proteins. Substituting NaCl with LiCl washing of DBS recovered the most proteins (1,642). This result led to use of LiCl for processing VAMS blood samples. These results highlight the potential to distribute protein subsets from complex mixtures of WB. This simple and streamlined protocol allows deep proteomic analysis of WB from DBS. Nakajima et al. (Nakajima et al., J Proteome Res. 2020; 19 (7): 2821-7.) achieved similar proteomic depth from DBS using DIA with in-house chromatogram libraries, but a key drawback of their approach is the use of multiple rounds of ultracentrifugation which requires specialized equipment and considerable preparation time.

In summary, FIG. 3A demonstrates that washing microsampling devices with LiCl prior to digestion recovers the most proteins and FIG. 3B demonstrates that urea washes recover a different subset of proteins compared to PBS or NaCl washes.

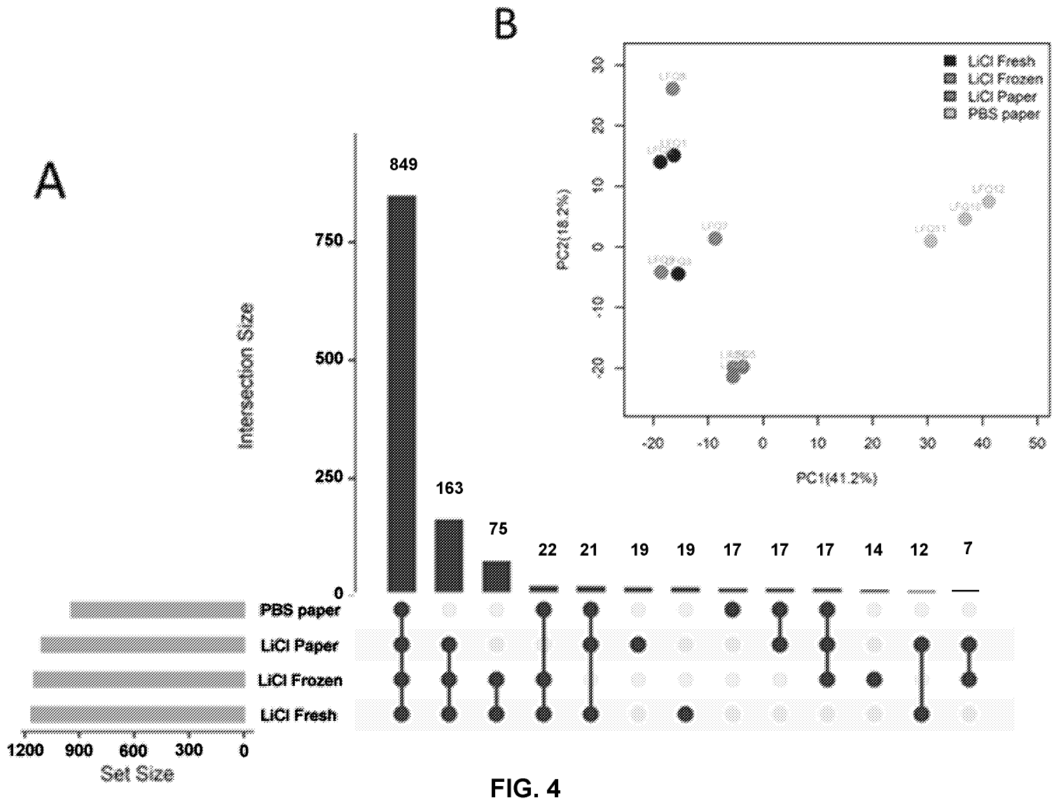

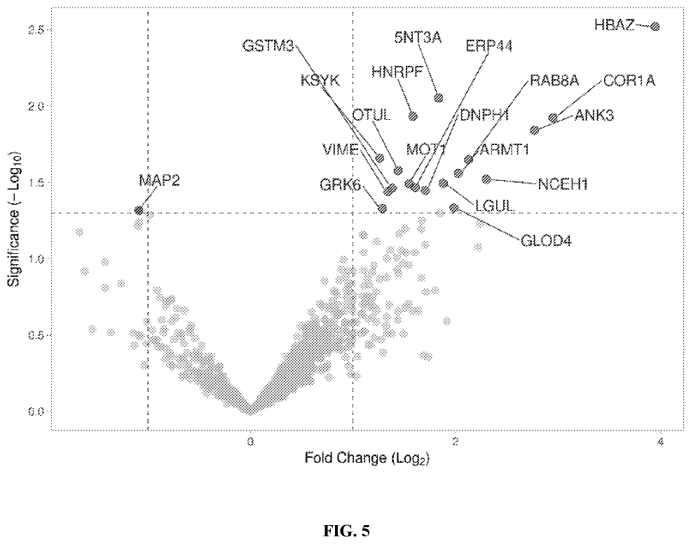

VAMS DBS Compared with Paper DBS. An advantage of VAMS blood collection compared with conventional paper DBS is the highly controlled volumetric blood collection that is achieved, facilitating precise user sampling while avoiding concerns regarding hematocrit levels that can complicate analyte quantitation; an issue well-known in clinical chemistry assays (Lehmann et al., Crit Rev Clin Lab Sci. 2017; 54 (3): 173-84; Zakaria et al., Ejifcc. 2016; 27 (4): 288-317). Conventional DBS using paper and PBS or LiCl washes was compared with VAMS DBS and LiCl washing. Further, it was determined whether freshly applied blood or blood stored frozen had any significant effect on protein detection after VAMs assay. As shown in FIG. 4, LC-MS analysis of proteins recovered from VAMS identified approximately 200 more proteins than those detected from PBS washed paper DBS and this was a significant finding (Tukey post-hoc q<0.01) (data not shown). Interestingly, many of the lost proteins were recovered by replacing PBS washing with LiCl washing of paper DBS. Only very minor, non-significant differences were observed between fresh and frozen blood applied to VAMS, although quantitative reproducibility was improved with fresh application of blood (14.9% CV vs 17.9% CV). Despite the caveats of inconsistent blood volume control for in-field use of paper DBS microsampling, it was noted that excellent recovery and reproducibility could be achieved when blood was applied in-laboratory settings using a pipette and our optimized workflow using LiCl washing (7.5% CV IQR 4.6-12.4). This suggests that washing and analysis pipeline disclosed herein is uniformly suited to either of these DBS matrices, although it is expected that the elimination of a hemocrit effect in microsampling will lead to more consistency with VAMS tips compared to filter paper in real-world settings.

As DIA-MS is rapidly evolving as a method of choice for proteomic data acquisition (Nakajima et al., J Proteome Res. 2020; 19 (7): 2821-7.; O'Rourke et al., J Proteomics. 2021; 231:103998), DIA-MS was used in the above mentioned fresh blood and frozen blood on VAMS tips washed with LiCl. Use of Spectronaut search software with a library constructed from DDA runs of representative samples allowed detection of 1892 proteins at 1% FDR (data not shown). The DIA-MS approach enabled detection of more than 650 additional proteins compared to the DDA analysis of these samples.

In summary, FIG. 4A demonstrates that microsampling devices washed with LiCl prior to digestion recover more proteins than washing traditional DBS filter paper with LiCl and FIG. 4B demonstrates that microsampling devices washed with LiCl recover a different subset of proteins relative to traditional DBS filter paper washed with LiCl.

Application of VAMS DBS with Long-term Frozen Whole Blood Cell Pellets from cancer Patients. VAMS may be useful for analysis of long-term frozen blood cell pellets. As a proof of concept, frozen blood cell pellets from five cancer patients and five matched controls (collected from age and sex matched healthy people), which had been stored at −20° C. and −80° C. for many years, were obtained. The cell pellets contained minimal residual plasma and could be slowly applied to VAMS device tips. The VAMS tips were washed with LiCl according to the method provided herein. The number of proteins detected from DDA of individual samples ranged from 381-1052, with a mean of 703 proteins detected across the ten samples. Following filtering and data imputation, 867 proteins in all 10 samples were quantified (data not shown). There was a reduced number of proteins detected in plasma compared to WB, which was due to the absence of most plasma proteins (data not shown). As the specimens were prepared and acquired in a single batch, the sample-sample variance was due to endogenous biological and storage factors rather than the sample preparation methodology. Based on a two-fold protein abundance threshold, 18 proteins were detected at higher abundance (upregulated) in cancer patient samples (upper right quadrant of FIG. 5) compared to controls, while 1 protein (MAP2) was lower (downregulated; upper left quadrant of FIG. 5). It was not possible to identify the cellular origin of these differentially abundant proteins which could arise from leukocytes, somatic or tumor cells, however, the majority of these proteins have been previously implicated in cancer prognosis or progression studies. Three of the proteins identified in higher abundance in the cancer samples, Ankyrin 3 (ANK3), Vimentin (VIME) and Monocarboxylate transporter 1 (MOT1) have been identified as markers of poor prognosis in cancer”

(Dauphin et al., Lung Cancer. 2013; 81 (1): 117-22; Koukourakis et al., Cancer Biology & Therapy. 2007; 6 (9): 1472-5; Liu Y et al. Onco Targets Ther. 2016; 9:7397-407).

In summary, FIG. 5 demonstrates that the microsampling devices provided herein are able to recover proteins in frozen, long-term stored cell pellets relative to fresh WBC/plasma samples. This is ideal for longitudinal studies that collect and store samples over long periods of time. It is also ideal for minimizing repeated sample collection.

Conclusion. The compatibility of single shot LC-MS for deep proteomic analysis of WB, by applying it to VAMS DBS or paper DBS and processing it with lithium salt washing, has been demonstrated. Using the methods provided herein, up to 1,600 proteins were detected and quantified from single-shot analysis of dried whole blood relative to undepleted plasma. Some proteins not previously reported in blood, including MAP17, were detected using this approach.

Use of VAMS for microsampling offers the advantage over paper of a suitable collection device that avoids hematocrit issues and acts as a useful matrix for sample preparation. This is an ideal situation for longitudinal blood sampling as required for precision medicine applications in oncology. The established protocol is simple to implement and is compatible with single-shot DDA or DIA LC-MS runs to gain access to hundreds of potential biomarker candidates. Subsequent stages for biomarker studies require quantitative validation in larger cohorts using stable isotope labelled reference standards with targeted mass spectrometry acquisition methods (Carr et al., Mol Cell Proteomics. 2014; 13 (3): 907-17). Future research will integrate use of VAMS DBS for sample collection and processing with SRM to enhance biomarker limits of quantitation.

### Example II: Methods for Fractionation and Sequential Sample Preparation Using Absorptive Devices

The methods described herein enable cells and/or membranes suspended in fluid to be absorbed into a single or multiple pieces of 3-D porous material, fractionated by centrifugation, and then dried. Drying causes the cell membranes to become trapped in the 3-D porous material. The material can then be washed to remove unwanted sample components, such as non-membrane associated proteins, salts, etc.

### Example II, Part A

Each piece of 3-D porous material is dried and then sequentially extracted, enabling multiple assays from each piece of 3-D porous material.

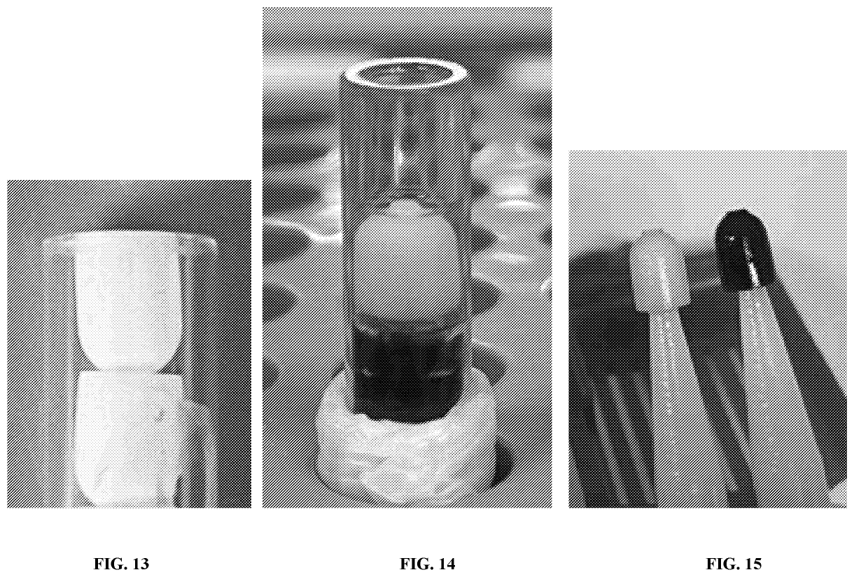

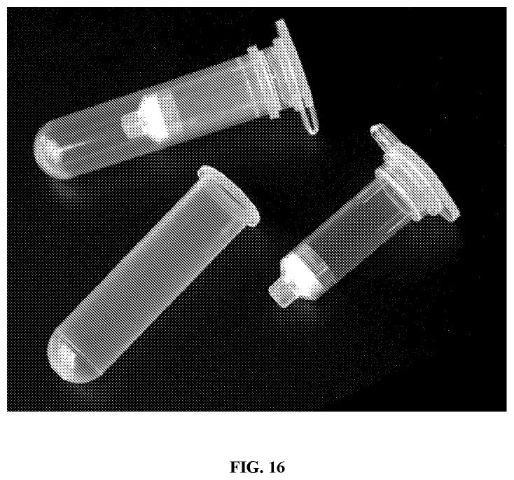

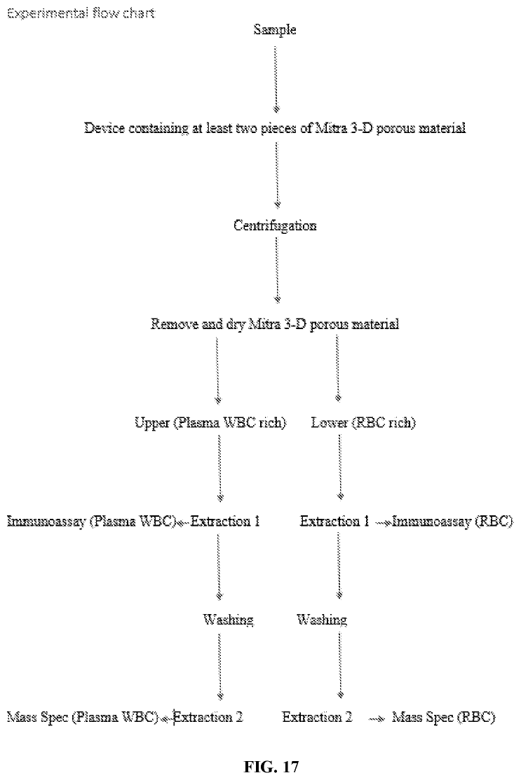

Mitra® 3-D porous material (30 μL volume) from Neoteryx was removed from the standard plastic handles and placed inside a plastic tube as shown in FIG. 13. An experimental flow chart is shown in FIG. 17.

Extraction 1: Each piece of Mitra® 3-D porous material was extracted overnight in 90 μL of phosphate buffered saline (PBS). The extraction solution was removed from the Mitra® 3-D porous material by centrifuging for 3 minutes at 3000 g in an empty spin column tube shown in FIG. 16. The piece of Mitra® 3-D porous material is retained in the top portion of the tube and the extraction solution is recovered in the bottom tube.

Washing: After extraction 1, each piece of Mitra® 3-D porous material was suspended in 1000 μL of wash solution in 1.5 mL Eppendorf tubes and left to incubate at room temperature for 24 hrs. whilst gently shaking (100 rpm). Wash solution was 0.1M Tris-HCl containing 0.5M LiCl. Following the incubation, each piece of Mitra® 3-D porous material was washed three times in fresh extraction solution. This was performed by pulse centrifugation of the tip in the wash solution (3× pulses 0-10,000 g) followed by centrifuging the wash solution out of the tips using a custom column insert device and resuspending them in fresh wash solution (3000 g, 3 min, full brake). After the final wash, the dry tip was placed into a fresh Eppendorf tube for storage until extraction 2 was performed.

Immunoassay of extraction 1: The immunoassays used were the MILLIPLEX MAP Human High Sensitivity T Cell Panel and MILLIPLEX MAP Human Chemokine Panel. The assays were performed according to manufacturer's instructions using an automated magnetic wash station (Bio-Plex Pro II, Bio-Rad) for the washing steps. The assays were run on the Luminex® 2001M system (Bio-Rad) and fluorescence values were collected. The calibration curve for each cytokine was analysed with 5 parametric logistic curve regression using Bio-Plex manager software (ver. 5.0, Bio-Rad). Standard values were considered acceptable if the points fell within 80-120% of the expected values.

Extraction 2: After extraction 1 and washing, as described above, each piece of Mitra® 3-D porous material was incubated with 90 μL digestion solution (0.1M triethylammonium bicarbonate, 1% (w/v) sodium deoxycholate (SDC), 10 mM TCEP, 40 mM chloroacetamide). The samples were then heated at 95° C. for 10 min. 1 μg trypsin was added and incubated at 37° C. overnight. Following incubation, the tips were removed from the solution and the remaining liquid was centrifuged out of the tips and combined. Formic acid was added to quench the reaction and precipitate the SDC. Peptides were desalted using Stage-tips.

Mass spectrometry acquisition: Peptides were loaded in 0.1% formic acid onto a 75 μm×15 cm 1.9 μm C18 (ReproSil-Pur C18-AQ) nanoLC column and eluted over 90 min gradient to a maximum of 30% solvent B (80% ACN, 0.1% FA).

Peptides were detected using a HF-X orbitrap mass spectrometer (Thermo, CA, USA) operating in DDA-mode with the following instrument settings: MS1-AGC 3e6, Resolution 60K, Scan range 300-1650 m/z. MS2-AGC 1e5, Resolution 15K, loop count 15.

Protein identification: Raw files were searched with MaxQuant V1.6.5 against the UniProt human proteome database (UP000005640, 20,350 reviewed protein entries) using the following settings: 1% FDR, reverse decoy mode, min peptides 2, FTMS mass tolerance 20 ppm, missed cleavages set to max 2, variable oxidation of methionine and fixed carbamidomethyl modification of Cys, LFQ enabled with default settings.

Quantification and Statistical analyses: Perseus was used to remove reverse hits and peptide matches to single peptides (two peptides was the minimum required for protein quantitation). Data was log 2 transformed and median normalization applied. Missing quantitation values were replaced by imputation with Perseus using default settings-values sampled from a Gaussian distribution down shifted by 1.8 standard deviations, with a width of 0.3.

Immunoassay analysis of extraction 1: The Multiplex immunoassay results on the chemokine kit are shown in Table 3 below. The upper plasma/WBC fraction is labelled A Plasma WBC. The lower RBC rich fraction is labelled A RBC. As expected for fractionated blood there are concentration differences between the upper and lower fraction for most chemokines.

The Multiplex immunoassay results for on the T cell kit are shown in Table 4 below. The upper plasma/WBC fraction is labelled A Plasma WBC. The lower RBC rich fraction is labelled A RBC. As expected for fractionated blood there are concentration differences between the upper and lower fraction for most cytokines. OOR<indicates that the concentration of that cytokine was below the reliable limit for quantification.

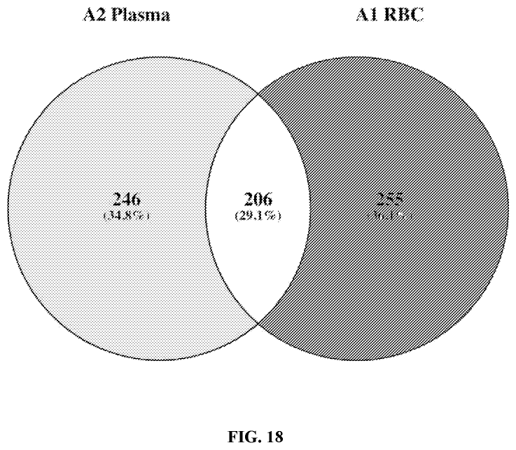

Mass Spectrometry analysis of extraction 2. The MS analysis of extraction 2 identified a total of 913 proteins across the extractions of both (upper plasma/WBC and lower RBC) pieces of Mitra® 3-D porous material. The top, plasma and WBC rich fraction produced 452 protein identifications and the bottom RBC rich fraction produced 461 protein identifications. The Venn diagram in FIG. 18 shows a Venn diagram depicting the number of identified proteins unique to the plasma fraction (246 proteins) and RBC rich fraction (255 proteins), and the number of proteins identified in both fractions (206 proteins).

Protein ranking by intensity-abundance. Protein names-Plasma and WBC rich fraction. The 25 most abundant proteins identified by MS analysis are shown in Table 5, with RBC specific proteins in bold.

Protein names-RBC rich fraction. The 25 most abundant proteins identified by MS analysis are shown in Table 6, with RBC specific proteins in bold.

Carbonic Anhydrase (CA1 and CA2) are in the top 25 most abundant proteins in the RBC fraction. These enzymes are known to be present in RBCs at high levels. They have not been listed as RBC proteins, because they have expression at high levels in other tissues. In the plasma and WBC fraction, CA1 was ranked 163 in abundance and CA2 ranked 360. The large difference in abundance of RBC proteins between the two fractions illustrates the separation and enrichment of RBCs in the lower fraction that has occurred during the centrifugation.

TABLE 3
Multiplex Immunoassay results for Example 1 on the chemokine kit.

| | Conc. (pg/ml) | | | | | | | | | | | |
|---|---|---|---|---|---|---|---|---|---|---|---|---|
| | ENA-78 | | Gro- | | | | | | MIP- | MIP- | MIP- | |
| Sample | (LIX) | Eotaxin | alpha/KC | IL-8 | IP-10 | ITAC | MCP-1 | MIG | 1alpha | 1beta | 3alpha | RANTES |
| RBC | 173.37 | 12.28 | 25.16 | 11.23 | 10.31 | 23.59 | 603.04 | 8.26 | 4.44 | 119.31 | 6.22 | 264.51 |
| Plasma | 695.07 | 12.04 | 17.99 | 3.61 | 12.79 | 24.79 | 103.6 | 11.53 | 10.04 | 156.7 | *2.95 | 357.41 |
| WBC | | | | | | | | | | | | |

TABLE 4
Multiplex Immunoassay results for Example 1 on the T cell kit.

| | Conc. (pg/ml) | | | | | | | | | |
|---|---|---|---|---|---|---|---|---|---|---|
| Sample | ITAC | GM-CSF | Fractalkine | IL-10 | IL-13 | IL-17A | IL-4 | IL-23 | IL-5 | IL-6 |
| RBC | 1.98 | 1.23 | 32.49 | OOR< | OOR< | OOR< | 3.82 | OOR< | OOR< | 1.35 |
| Plasma | OOR< | 3.37 | 45.82 | 4.28 | 10.17 | 1.44 | 31.36 | 49.18 | 0.9 | 6.93 |
| WBC | | | | | | | | | | |

TABLE 5
The 25 most abundant proteins identified by MS analysis in Plasma and WBC rich
fractions.

| Rank | Protein |
|---|---|
| 1 | Filamin-A |
| 2 | Hemoglobin subunit beta; LVV-hemorphin-7; Spinorphin |
| 3 | Vitronectin; Vitronectin V65 subunit; Vitronectin V10 subunit; Somatomedin-B |
| 4 | Histidine-rich glycoprotein |
| 5 | Apolipoprotein A-I; Proapolipoprotein A-I; Truncated apolipoprotein A-I |
| 6 | Serum albumin |
| 7 | Apolipoprotein B-100; Apolipoprotein B-48 |
| 8 | Talin-1 |
| 9 | Myosin-9 |
| 10 | Apolipoprotein E |
| 11 | Band 3 anion transport protein |
| 12 | Kininogen-1; Kininogen-1 heavy chain; T-kinin; Bradykinin; Lysyl-bradykinin; |
| | Kininogen-1 light chain; Low molecular weight growth-promoting factor |
| 13 | Hemoglobin subunit alpha |
| 14 | Integrin alpha-IIb; Integrin alpha-IIb heavy chain; Integrin alpha-IIb light chain, form |
| | 1; Integrin alpha-IIb light chain, form 2 |
| 15 | Histone H2A type 1-J; Histone H2A type 1-H; Histone H2A.J; Histone H2A type 1- |
| | D; Histone H2A type 1 |
| 16 | Inter-alpha-trypsin inhibitor heavy chain H4; 70 kDa inter-alpha-trypsin inhibitor |
| | heavy chain H4; 35 kDa inter-alpha-trypsin inhibitor heavy chain H4 |
| 17 | Actin, cytoplasmic 1; Actin, cytoplasmic 1, N-terminally processed; Actin, |
| | cytoplasmic 2; Actin, cytoplasmic 2, N-terminally processed |
| 18 | Tubulin alpha-1B chain |
| 19 | Tubulin beta-1 chain |
| 20 | Histone H2B type 1-L; Histone H2B type 1-M; Histone H2B type 1-N; Histone H2B |
| | type 1-H; Histone H2B type 2-F; Histone H2B type 1-C/E/F/G/I; Histone H2B type 1- |
| | D; Histone H2B type F-S; Histone H2B type 1-K; Histone H2B type 2-E; Histone |
| | H2B type 1-B; Histone H2B type 1-O; Histone H2B type 1-J; Histone H2B type 3-B; |
| | Histone H2B type 1-A |
| 21 | Lactotransferrin; Lactoferricin-H; Kaliocin-1; Lactoferroxin-A; Lactoferroxin-B; |
| | Lactoferroxin-C |
| 22 | Alpha-2-macroglobulin |
| 23 | Histone H4 |
| 24 | Integrin beta-3 |
| 25 | Spectrin alpha chain, erythrocytic 1 |

TABLE 6
The 25 most abundant proteins identified by MS analysis in RBC rich fractions.

| Rank | Protein |
|---|---|
| 1 | Band 3 anion transport protein |
| 2 | Hemoglobin subunit beta; LVV-hemorphin-7; Spinorphin |
| 3 | Hemoglobin subunit alpha |
| 4 | Spectrin alpha chain, erythrocytic 1 |
| 5 | Spectrin beta chain, erythrocytic |
| 6 | Ankyrin-1 |
| 7 | Solute carrier family 2, facilitated glucose transporter member 1 |
| 8 | Erythrocyte band 7 integral membrane protein |
| 9 | Blood group Rh(D) polypeptide; Blood group Rh(CE) polypeptide |
| 10 | Erythrocyte membrane protein band 4.2 |
| 11 | Protein 4.1 |
| 12 | Serum albumin |
| 13 | Carbonic anhydrase 2 |
| 14 | Actin, cytoplasmic 1; Actin, cytoplasmic 1, N-terminally processed; Actin, |
| | cytoplasmic 2; Actin, cytoplasmic 2, N-terminally processed |
| 15 | Apolipoprotein B-100; Apolipoprotein B-48 |
| 16 | Adenylate kinase isoenzyme 1 |
| 17 | Hemoglobin subunit delta |
| 18 | Cullin-associated NEDD8-dissociated protein 1 |
| 19 | Histone H2A type 1-J; Histone H2A type 1-H; Histone H2A.J; Histone H2A type 1- |
| | D; Histone H2A type 1 |
| 20 | Serine/threonine-protein phosphatase 2A 65 kDa regulatory subunit A alpha isoform |
| 21 | Histidine-rich glycoprotein |
| 22 | 55 kDa erythrocyte membrane protein |
| 23 | Lactotransferrin; Lactoferricin-H; Kaliocin-1; Lactoferroxin-A; Lactoferroxin-B; |
| | Lactoferroxin-C |
| 24 | Carbonic anhydrase 1 |
| 25 | Exportin-1 |

### Example II, Part B

The setup, in terms of the Mitra® 3-D porous material (30 μL volume), for this experiment was similar to Example II, Part A. The volume of blood used was similar, however, the device was configured with increased dead volume under the bottom Mitra®. This enabled a greater proportion of the WBC component to be removed from the top Mitra® during centrifugation.

The two pieces of Mitra® 3-D porous material were removed after centrifugation and the drying and sequential extraction and analyses were the same as Example II, Part A.

Immunoassay analysis of extraction 1. The Multiplex immunoassay results for the chemokine kit are shown in Table 7 below. The upper plasma/WBC fraction is labelled B Plasma WBC. The lower RBC rich fraction is labelled B RBC. As expected for fractionated blood there are concentration differences between the upper and lower fraction for most chemokines.

The Multiplex immunoassay results on the T cell kit are shown in Table 8 below. The upper plasma/WBC fraction is labelled B Plasma WBC. The lower RBC rich fraction is labelled B RBC. As expected for fractionated blood there are concentration differences between the upper and lower fraction for most cytokines. OOR (out of range) indicates that the concentration of that cytokine was below the reliable limit for quantification.

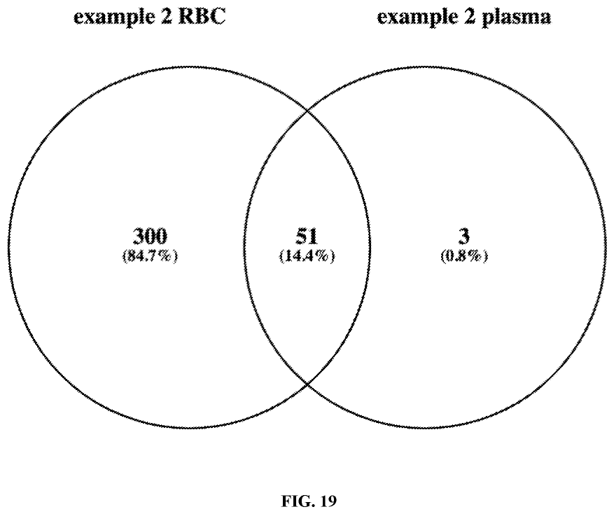

The MS analysis of extraction 2 identified a total of 414 proteins across the extractions of both (upper plasma and lower RBC) pieces of Mitra® 3-D porous material. The top plasma fraction produced 54 protein identifications and the bottom RBC rich fraction produced 351 protein identifications. The Venn diagram in FIG. 19 shows the number of identified proteins unique to the plasma fraction (3 proteins) and the RBC rich fraction (300 proteins), and the number of proteins identified in both fractions (51 proteins).

TABLE 7
Multiplex Immunoassay results for Example 2 on the chemokine kit.

| | Conc. (pg/ml) | | | | | | | | | | | |
|---|---|---|---|---|---|---|---|---|---|---|---|---|
| | ENA-78 | | Gro- | | | | | | MIP- | MIP- | MIP- | |
| Sample | (LIX) | Eotaxin | alpha/KC | IL-8 | IP-10 | ITAC | MCP-1 | MIG | 1alpha | 1beta | 3alpha | RANTES |
| RBC | 946.39 | 6.91 | 102.13 | 19.23 | 12.37 | 34.53 | 808.03 | 11.66 | 7.6 | 165.44 | 6.56 | 412.36 |
| Plasma | 428.55 | 8.5 | 16.14 | 4.44 | 8.12 | 27.35 | 104.56 | 10.33 | 7.67 | 128.09 | *3.54 | 271.39 |
| WBC | | | | | | | | | | | | |

TABLE 8
Multiplex Immunoassay results for Example 2 on the T cell kit.

| | Conc. (pg/ml) | | | | | | | | | |
|---|---|---|---|---|---|---|---|---|---|---|
| Sample | ITAC | GM-CSF | Fractalkine | IL-10 | IL-13 | IL-17A | IL-4 | IL-23 | IL-5 | IL-6 |
| RBC | 2.55 | OOR< | 77.97 | OOR< | OOR< | OOR< | 5.87 | OOR< | OOR< | 1.89 |
| Plasma | OOR< | 3.01 | 32.49 | 4.07 | 8.96 | 1.33 | 23.77 | 33.52 | 0.78 | 5.93 |
| WBC | | | | | | | | | | |

Example II, Part B demonstrates that if the cells are substantially removed from the Mitra® during the fractionation, there are very few proteins remaining after extraction 1 and washing.

### Example II, Part C

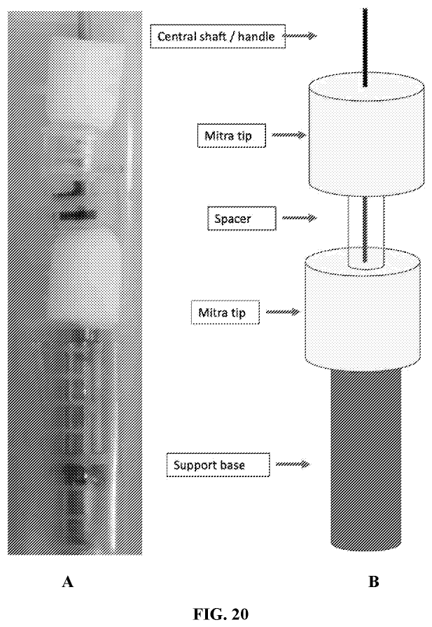

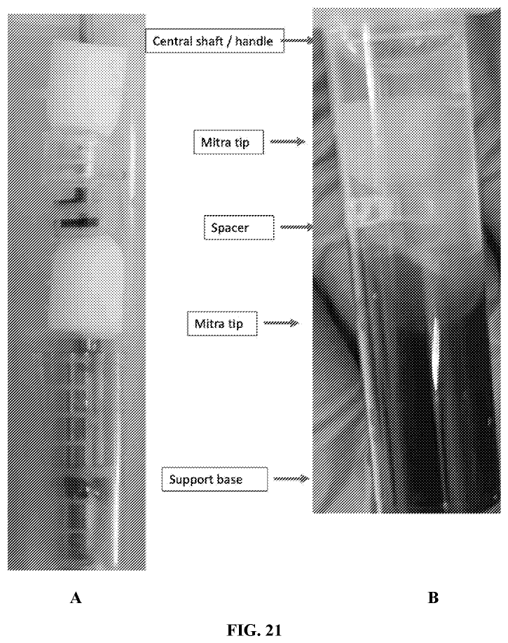

The setup, in terms of the Mitra® 3-D porous material (30 μL volume), is shown in FIGS. 20 and 21. The devices were placed in 2 mL Eppendorf tubes, which were filled with EDTA blood (approximately 1.7 mL)

The two pieces of Mitra® 3-D porous material were removed after centrifugation and the drying and sequential extraction and analyses were the same as Example II, Part A.

Mitra® 3-D porous material (30 μL volume) from Neoteryx was removed from the standard plastic handles and placed inside a plastic tube as shown in FIG. 20.

Immunoassay analysis of extraction 1. The Multiplex immunoassay results on the chemokine kit are shown in Table 9 below. The Multiplex immunoassay results on the T cell kit are shown in Table 10 below. As expected for fractionated blood there are concentration differences between the upper and lower fraction for most chemokines.

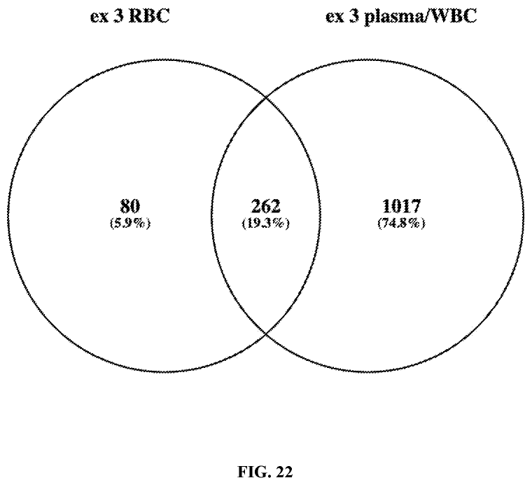

The MS analysis of Example II, Part C (extraction 2) identified a total of 1621 proteins across the extractions of both (upper plasma and lower RBC) pieces of Mitra® 3-D porous material. The top plasma fraction produced 1279 protein identifications and the bottom RBC rich fraction produced 342 protein identifications. The Venn diagram in FIG. 22 shows the number of identified proteins unique to the plasma fraction (1017 proteins) and the RBC rich fraction (80 proteins), and the number of proteins identified in both fractions (262 proteins).

Protein names. The 25 most abundant proteins identified by MS analysis in Plasma and WBC rich fractions are shown in Table 13, with RBC specific proteins labelled in red.

Carbonic Anhydrase (CA1 and CA2) are in the top 25 most abundant proteins in the RBC fraction. These enzymes are known to be present in RBCs at high levels. They have not been listed as RBC proteins, because they have expression at high levels in other tissues. In the plasma and WBC fraction, CA1 was ranked 781 in abundance and CA2 was not identified. The large difference in abundance of RBC proteins between the two fractions illustrates the separation and enrichment of RBCs in the lower fraction that has occurred during the centrifugation.

Table 14 shows protein names in the RBC rich fraction with RBC proteins in bold.

### Example II, Part D

The setup, in terms of the Mitra® 3-D porous material (30 μL volume), is shown in FIGS. 20 and 21. The devices were placed in 2 mL Eppendorf tubes, which were filled with EDTA blood (approximately 1.7 mL)

The two pieces of Mitra® 3-D porous material were removed after centrifugation and the drying and sequential extraction and analyses were the same as Example II, Part A.

Mitra® 3-D porous material (30 μL volume) from Neoteryx was removed from the standard plastic handles and placed inside a plastic tube as shown in FIG. 20.

Immunoassay analysis of extraction 1. The Multiplex immunoassay results on the chemokine kit are shown in Table 11 below. The Multiplex immunoassay results on the T cell kit are shown in Table 12 below. As expected for fractionated blood there are concentration differences between the upper and lower fraction for most cytokines. OOR<indicates that the concentration of that cytokine was below the reliable limit for quantification.

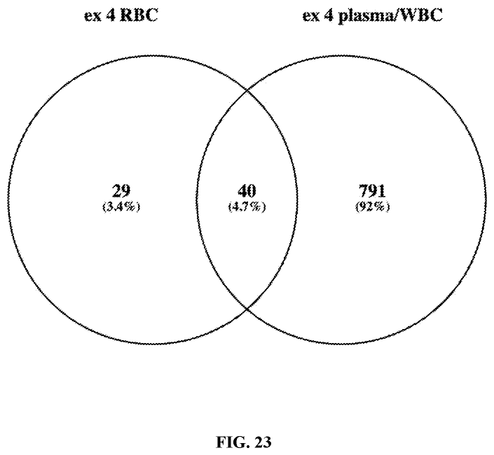

The MS analysis of Example II, Part D (extraction 2) identified a total of 900 proteins across the extractions of both (upper plasma and lower RBC) pieces of Mitra® 3-D porous material. The top plasma fraction produced 831 protein identifications and the bottom RBC rich fraction produced 69 protein identifications. The Venn diagram in FIG. 23 shows the number of identified proteins unique to the plasma/WBC fraction (791 proteins) and the RBC rich fraction (29 proteins), and the number of proteins identified in both fractions (40 proteins).

TABLE 9
Multiplex immunoassay results for Example II, Part C on the chemokine kit.

| | Conc. (pg/ml) | | | | | | | | | | | |
|---|---|---|---|---|---|---|---|---|---|---|---|---|
| | ENA-78 | | Gro- | | | | | | MIP- | MIP- | MIP- | |
| Sample | (LIX) | Eotaxin | alpha/KC | IL-8 | IP-10 | ITAC | MCP-1 | MIG | 1alpha | 1beta | 3alpha | RANTES |
| RBC | 2706.17 | 14.87 | 336.52 | 83.43 | 32.87 | 43.1 | 1089.16 | 27.71 | 13.7 | 259.62 | 8.03 | 181.9 |
| Plasma | 2485.58 | 29.72 | 40.19 | 4.39 | 30.16 | 28.48 | 117.79 | 13.83 | 13.29 | 191.71 | 4.47 | 181.07 |
| WBC | | | | | | | | | | | | |

TABLE 10
Multiplex immunoassay results for Example II, Part C on the T cell kit.

| | Conc. (pg/ml) | | | | | | | | | |
|---|---|---|---|---|---|---|---|---|---|---|
| Sample | ITAC | GM-CSF | Fractalkine | IL-10 | IL-13 | IL-17A | IL-4 | IL-23 | IL-5 | IL-6 |
| RBC | 2.19 | 1.23 | 216.32 | OOR< | 1.34 | OOR< | 9.48 | OOR< | OOR< | 2.48 |
| Plasma | OOR< | 3.37 | 83.1 | 4.38 | 8.49 | 1.77 | 25.86 | 47.45 | 0.99 | 6.57 |
| WBC | | | | | | | | | | |

TABLE 11
Multiplex immunoassay results for Example II, Part D on the chemokine kit.

| | Conc. (pg/ml) | | | | | | | | | | | |
|---|---|---|---|---|---|---|---|---|---|---|---|---|
| | ENA-78 | | Gro- | | | | | | MIP- | MIP- | MIP- | |
| Sample | (LIX) | Eotaxin | alpha/KC | IL-8 | IP-10 | ITAC | MCP-1 | MIG | 1alpha | 1beta | 3alpha | RANTES |
| RBC | 1354.7 | 11.33 | 231.92 | 59.11 | 27.3 | 47.24 | 1268.04 | 24.52 | 8.65 | 207.5 | 6.56 | 137.73 |
| Plasma | 2945.31 | 48.62 | 49.3 | 5.91 | 38.68 | 29.15 | 138.97 | 14.86 | 16.18 | 212.46 | *4.10 | 163.15 |
| WBC | | | | | | | | | | | | |

TABLE 12
Multiplex immunoassay results for Example II, Part D on the T cell kit.

| | Conc. (pg/ml) | | | | | | | | | |
|---|---|---|---|---|---|---|---|---|---|---|
| Sample | ITAC | GM-CSF | Fractalkine | IL-10 | IL-13 | IL-17A | IL-4 | IL-23 | IL-5 | IL-6 |
| RBC | 3.23 | OOR< | 177.46 | OOR< | 1.23 | OOR< | 6.13 | OOR< | OOR< | 1.18 |
| Plasma | OOR< | 2.83 | 93.45 | 3.81 | 7.86 | 1.77 | 26.91 | 26.46 | 0.92 | 6.83 |
| WBC | | | | | | | | | | |

TABLE 13
The 25 most abundant proteins identified by MS analysis in plasma and WBC rich
fractions.

| Rank | Protein |
|---|---|
| 1 | Filamin-A |
| 2 | Myosin-9 |
| 3 | Talin-1 |
| 4 | Apolipoprotein B-100; Apolipoprotein B-48 |
| 5 | Integrin alpha-IIb; Integrin alpha-IIb heavy chain; Integrin alpha-IIb light chain, |
| | form 1; Integrin alpha-IIb light chain, form 2 |
| 6 | Tubulin alpha-1B chain |
| 7 | Hemoglobin subunit beta; LVV-hemorphin-7; Spinorphin |
| 8 | Histone H2A type 1-J; Histone H2A type 1-H; Histone H2A.J; Histone H2A type 1- |
| | D; Histone H2A type 1 |
| 9 | Actin, cytoplasmic 1; Actin, cytoplasmic 1, N-terminally processed; Actin, |
| | cytoplasmic 2; Actin, cytoplasmic 2, N-terminally processed |
| 10 | Apolipoprotein A-I; Proapolipoprotein A-I; Truncated apolipoprotein A-I |
| 11 | Tubulin beta-1 chain |
| 12 | Vitronectin; Vitronectin V65 subunit; Vitronectin V10 subunit; Somatomedin-B |
| 13 | Serum albumin |
| 14 | Apolipoprotein E |
| 15 | Kininogen-1; Kininogen-1 heavy chain; T-kinin; Bradykinin; Lysyl-bradykinin; |
| | Kininogen-1 light chain; Low molecular weight growth-promoting factor |
| 16 | Histidine-rich glycoprotein |
| 17 | Hemoglobin subunit alpha |
| 18 | Histone H4 |
| 19 | Keratin, type II cytoskeletal 1 |
| 20 | Integrin beta-3 |
| 21 | Tubulin beta-4B chain; Tubulin beta-4A chain |
| 22 | Band 3 anion transport protein |
| 23 | Thrombospondin-1 |
| 24 | Histone H2B type 1-L; Histone H2B type 1-M; Histone H2B type 1-N; Histone |
| | H2B type 1-H; Histone H2B type 2-F; Histone H2B type 1-C/E/F/G/I; Histone H2B |
| | type 1-D; Histone H2B type F-S; Histone H2B type 1-K; Histone H2B type 2-E; |
| | Histone H2B type 1-B; Histone H2B type 1-O; Histone H2B type 1-J; Histone H2B |
| | type 3-B; Histone H2B type 1-A |
| 25 | Tubulin beta chain |

TABLE 14
The 25 most abundant proteins identified by MS analysis in RBC rich fractions.

| Rank | Protein |
|---|---|
| 1 | Hemoglobin subunit beta; LVV-hemorphin-7; Spinorphin |
| 2 | Band 3 anion transport protein |
| 3 | Hemoglobin subunit alpha |
| 4 | Erythrocyte band 7 integral membrane protein |
| 5 | Solute carrier family 2, facilitated glucose transporter member 1 |
| 6 | Spectrin alpha chain, erythrocytic 1 |
| 7 | Blood group Rh(D) polypeptide; Blood group Rh(CE) polypeptide |
| 8 | Spectrin beta chain, erythrocytic |
| 9 | Carbonic anhydrase 2 |
| 10 | Ankyrin-1 |
| 11 | Apolipoprotein B-100; Apolipoprotein B-48 |
| 12 | Erythrocyte membrane protein band 4.2 |
| 13 | Actin, cytoplasmic 1; Actin, cytoplasmic 1, N-terminally processed; Actin, |
| | cytoplasmic 2; Actin, cytoplasmic 2, N-terminally processed |
| 14 | Protein 4.1 |
| 15 | Histone H2A type 1-J; Histone H2A type 1-H; Histone H2A.J; Histone H2A type 1- |
| | D; Histone H2A type 1 |
| 16 | Hemoglobin subunit delta |
| 17 | Serum albumin |
| 18 | Myosin-9 |
| 19 | Lactotransferrin; Lactoferricin-H; Kaliocin-1; Lactoferroxin-A; Lactoferroxin- |
| | B; Lactoferroxin-C |
| 20 | Carbonic anhydrase 1 |
| 21 | Filamin-A |
| 22 | Histone H3.3; Histone H3.2; Histone H3.1t; Histone H3.1 |
| 23 | Integrin alpha-IIb; Integrin alpha-IIb heavy chain; Integrin alpha-IIb light chain, |
| | form 1; Integrin alpha-IIb light chain, form 2 |
| 24 | Vitronectin; Vitronectin V65 subunit; Vitronectin V10 subunit; Somatomedin-B |
| 25 | Histidine-rich glycoprotein |

### Example III: Label-Free Quantification (LFQ) Data

Basic Label-Free Quantification (LFQ) Workflow. LFQ workflow was performed as established by LFQ Analyst and DataSciencePlus.

Handling of Samples. Frozen whole blood cell pellets (plasma removed) from 5 healthy and 5 Stage III cancer donors, matched for both age and sex, were purchased from PrecisionMed Inc (Solana Beach, CA, USA). Samples were collected between 2014-2018. The supplier indicated that samples from healthy participants were stored at −20° C. for between 35.6-263.0 weeks and 43.0-165.4 weeks for cancer patients. Upon receipt in our laboratory, samples were stored at −80° C. for 101 weeks prior to the analysis reported herein. The samples were thawed on ice and applied to the VAMS tip until filled completely. The samples were then left to dry for 5 minutes at room temperature before being transferred to foil zip-lock bags with desiccant. These samples were all stored at 4° C. for a minimum of 24 hrs before extraction. Odd numbered samples (1, 3, 5, 7 and 9) are controls and even numbered samples (2, 4, 6, 8 and 10) are cancer samples.

Filtering. Data was filtered to remove reverse hits and contaminants (REV/CON proteins), which resulted in the removal of 36 proteins. Data was also filtered to remove single unique/razor peptide proteins, which resulted in the removal of 141 proteins.

Quantitation filtering. Data was filtered to remove sparse proteins, allowing quantitation of proteins with more than 3 quantitated values per group and also allowing proteins present with two samples in both groups.

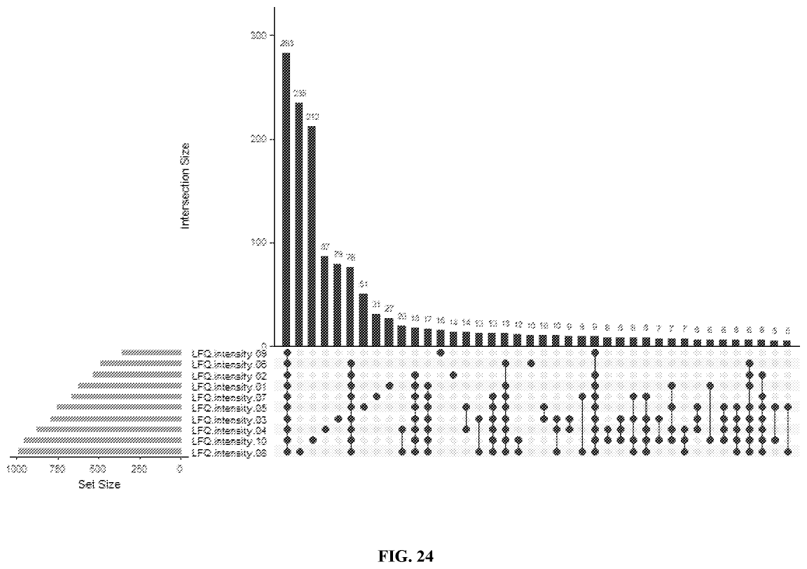

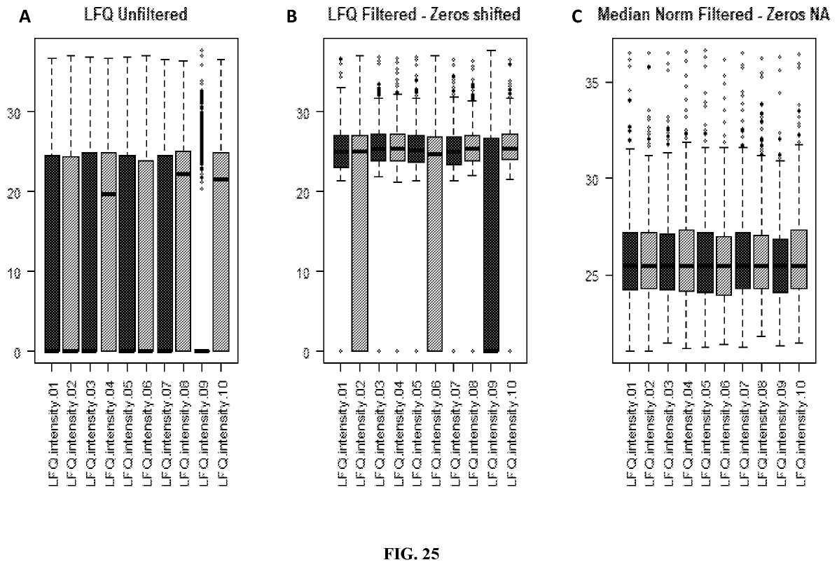

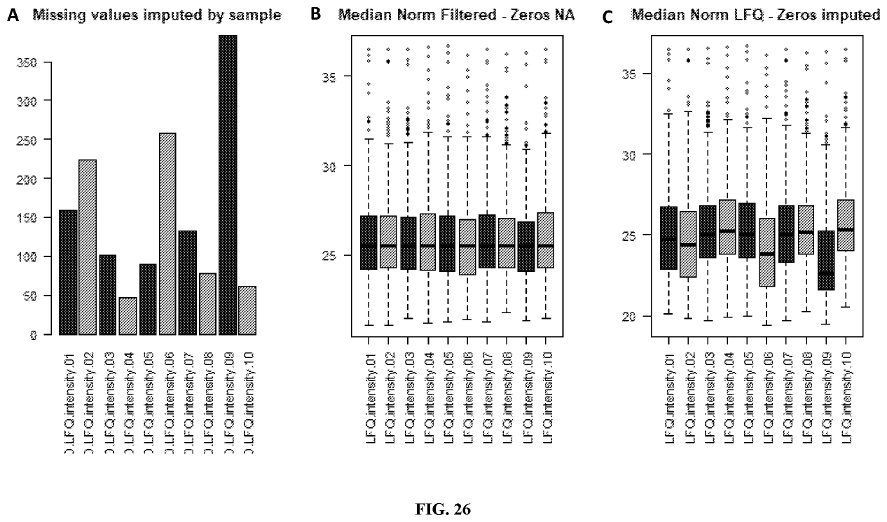

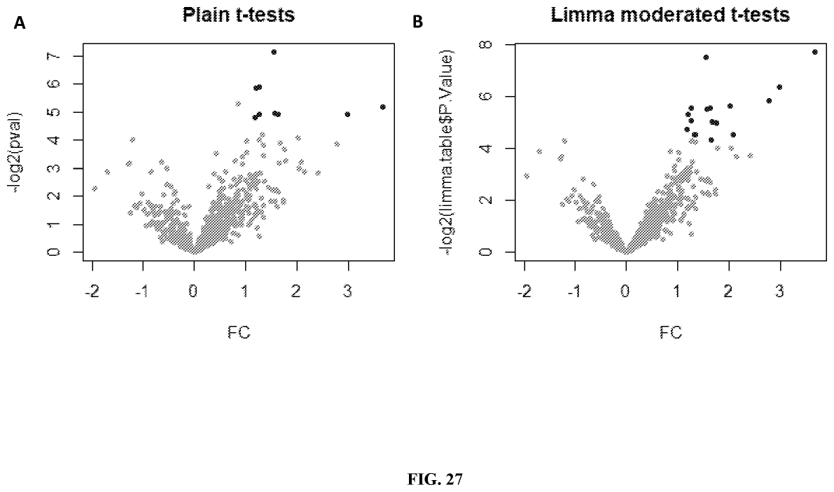

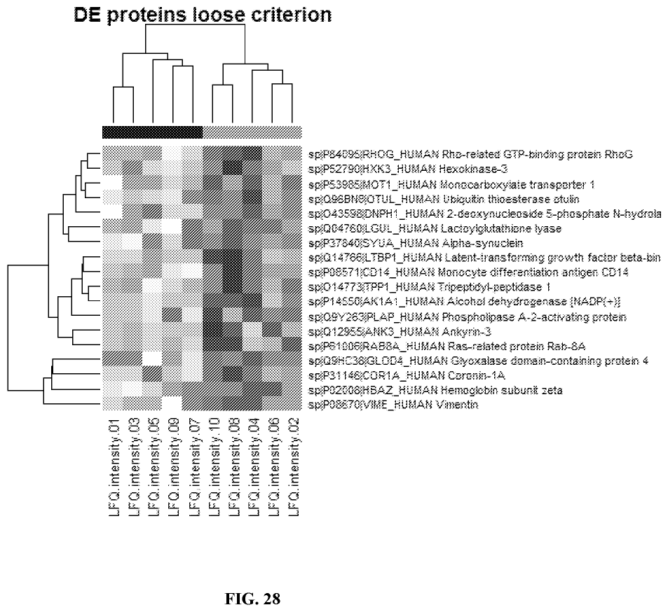

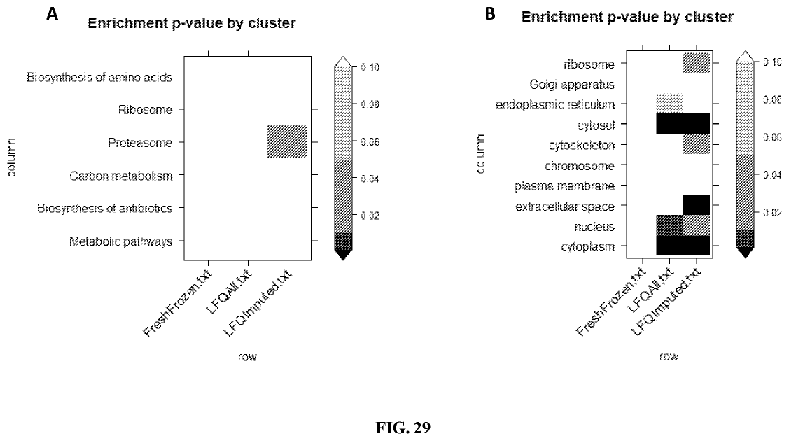

This resulted in the removal of 1026 proteins. Filtering can be found in FIG. 24.

Data imputation for missing values after filtering and median normalization. After removing proteins identified with few peptides, and proteins with sparse quantitation, the remaining protein quantitation still has missing values, which can be imputed using the default Perseus-style imputation (random numbers drawn from a normal distribution of 1.8 standard deviation down shift and with a width of 0.3 of each sample).

Median normalization was done before data imputation; otherwise the lower abundance samples will be up-shifted.

Differential expression using plain t-tests or moderated t-tests with the limma package. After data imputation, usual workflow of differential expression was carried out. Data between groups was compared with a two-sample t-test and/or the moderated t-tests from the limma package. A p-value of <0.05 was used to determine significance with either plain t-tests or moderated t-tests to find a number of proteins upregulated in cancer samples.

Types of proteins comparison. Functional comparison of all identified proteins (LFQAll), as well as the smaller set of proteins with full imputed data (LFQImputed) can be identified in fresh or frozen samples. However, there is not much difference, just more cytosolic proteins (percentage-wise), and slightly more nucleus proteins. Likewise, there were no changes when looking at KEGG pathways.

Results reliability: repeated imputation, bootstrapping, and label randomization. On a small dataset (n=5 cancer/healthy) results are easily obtained due to chance alone, or in error. The chance of such false positives decreases with larger experiment size, as it is harder to obtain chance positive results from larger experiments.

While there is no substitute for obtaining more samples, and randomization or bootstrapping on a small set do not guarantee results reproducibility on a biologically different set, the quality of the results can be checked in several ways.

### Example IV: Urea Wash

Broad aims. The purpose of the experiment described herein is to understand differences between methods, optimal preparations, and to set best procedures for such data/analyses for the future.

Differences in terms of identification. It is useful to look at differences in identifications, presumably especially for assessing different methodologies. They can be done manually using the online tool Venny for Venn diagrams. When using many sets at once, script online tool can be used to created UpSet plots.

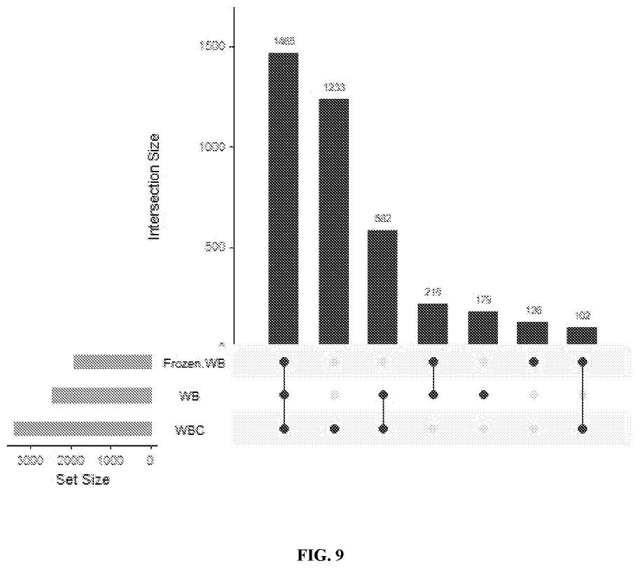

FIG. 9 shows an UpSet plot depicting that washing VAM devices 3 times with 1 mL of 0.1 M Tris-HCl containing 0.5 M NaCl results in the identification of 1465 proteins that are common to frozen whole blood (WB), WB and white blood cells (WBC), followed by 1233 proteins that are unique to WBC.

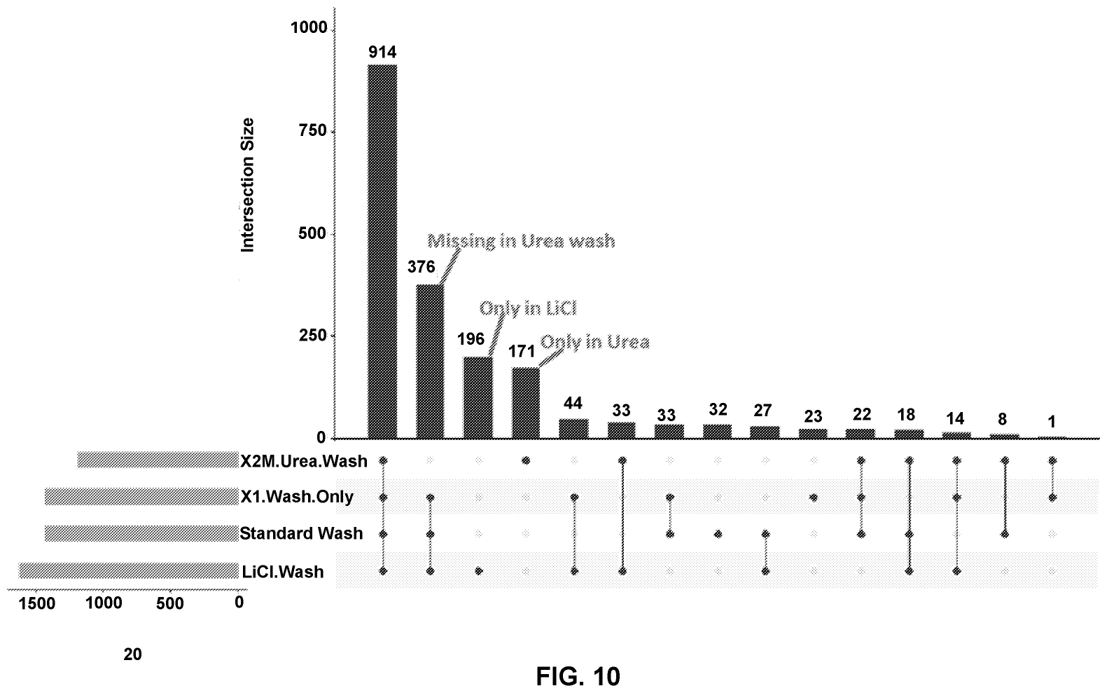

FIG. 10 shows an UpSet plot demonstrating that washing Neoteryx tips 2 times with 1 mL of 2M urea results in the identification of a different protein profile, with ˜550 less proteins identified relative to FIG. 9.

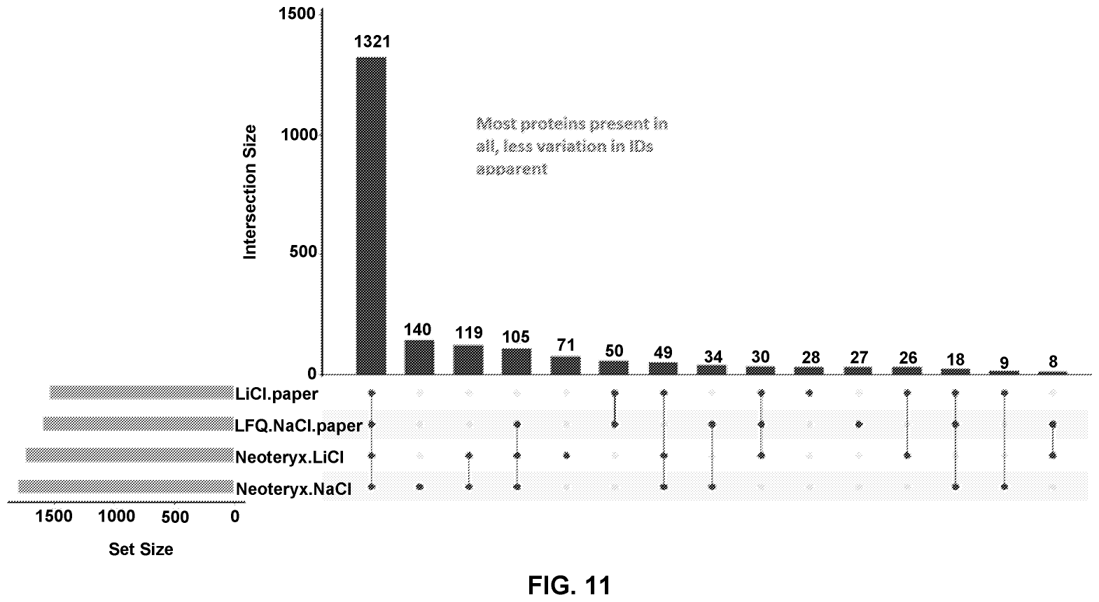

FIG. 11 shows an UpSet plot depicting that washing Neoteryx tips or filter paper with LiCl or NaCl, identifies 1321 proteins present in filter paper washed with LiCl, filter paper washed with NaCl, a Neoteryx tip washed with LiCl and a Neoteryx tip washed with NaCl.

Gene Ontology (GO) Annotation: GO annotation can be obtained live from Uniprot, and parsed via scripts (so eliminating the need to download the annotation sheets, and parsing them manually).

A number of categories of interest (as below) were selected, and the summaries for each of the sets below were obtained, including all proteins present in the experiment. This could be re-run with different subsets, and different GO categories, depending on the questions that need to be asked.

Urea wash results in the identification of more plasma membrane and ribosomal proteins based on GO annotations. Table 17 shows GO Annotations.

TABLE 17
GO Annotations

| GO categories selected | Protein Subcategories |
|---|---|
| Cytoplasm nucleus | “Frozen WB-5-Fresh, Frozen & Fresh on |
| | paper + LiCl wash” |
| Extracellular space | “WB-2-Standard Extraction” |
| plasma | |
| Membrane | “WB-3-1 Wash Only” |
| Cell surface | “WB-3-2M Urea Wash” |
| Chromosome | “WB-3-LiCl Wash” |
| Cytoskeleton | “WB-3-Standard Wash” |
| Cytosol | “WB-4-LFQ NaCl paper” |
| Endosome | “WB-4-LiCl paper” |
| Endoplasmic reticulum | “WB-4-Neoteryx LiCl” |
| Golgi apparatus | “WB-4-Neoteryx NaCl” |
| Mitochondrion protein | “WBC-1-Standard Extraction” |
| Complex ribosome | “WBC-2-Standard Extraction” |

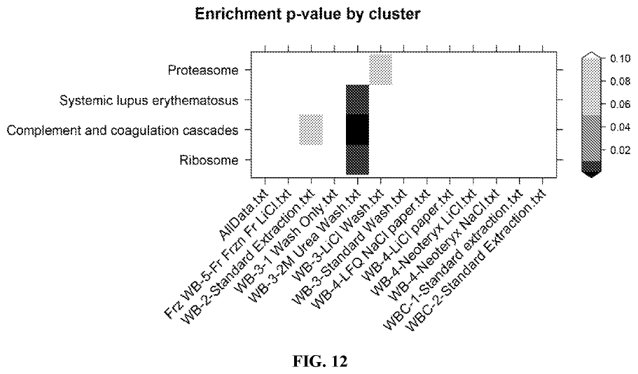

Kyoto Encyclopedia of Genes and Genomes (KEGG) Annotation: The same process can be repeated with KEGG pathway annotation. The urea wash results in the separation and identification of functional proteins including the ribosomes. FIG. 12 shows KEGG Annotation.

Abbreviations Used in the Detailed Description and in the Examples are as follows: Epithelial-neutrophil activating peptide/CXCL5 (ENA-78 (LIX)), Growth-regulated oncogene alpha (Gro-alpha/KC), Interleukin-8 (IL-8), Interferon gamma-induced protein 10 (IP-10), Interferon-inducible T Cell Alpha Chemoattractant (ITAC), Monocyte chemoattractant 1 (MCP-1), CXC motif ligand 9 (MIG), Macrophage inflammatory protein 1 alpha (MIP-1 alpha), Macrophage inflammatory protein 1 beta (MIP-1beta), Macrophage inflammatory protein 3 alpha (MIP-3alpha), Regulated upon Activation, Normal T Cell Expressed and Presumably Secreted (RANTES), Granulocyte-macrophage colony-stimulating factor (GM-CSF), Interleukin-10 (IL-10), Interleukin-13 (IL-13), Interleukin-17 alpha (IL-17A), Interleukin-4 (IL-4), Interleukin-23 (IL-23), Interleukin-5 (IL-5), Interleukin-6 (IL-6).

### Example V: Evaluation of Buffers and Peptide Cleanup

Purpose: evaluate impact of varying digestion buffers and/or peptide cleanup procedures, relative to the methods described in Example II, on peptide yields and/or protein identifications after mass spec analysis. It is noted that the method of Example II provides a resulting peptide yield recovery of about 0.8 μg/μL (a total of about 16 μg, which is near capacity of the Stage-tips; if the existing method is being limited by the capacity of the Stage-tips, the consequence will be missing some IDs in the mass spectrometry). The variations of digestion buffers and/or peptide cleanup procedures include: an additional centrifugation step after the trypsin digestion, use of solid phase extraction (SPE) instead of the Stage-tip for peptide desalting, and use of freshly prepared buffer (“FPB” buffer”; 1% SDC, 10 mM TCEP, 40 mM iodoacetamide (IAA), 100 mM triethylammonium bicarbonate buffer (TEAB) pH 8) instead of the digestion solution indicated in the above (Master mix buffer, Example II, Part A) for trypsin digestion.

A series of whole blood (WB) samples were prepared according to Example II (in 30 μL VAMS/Mitra® tips), and as described in Molloy et al., “Proteomic Analysis of Whole Blood Using Volumetric Absorptive Microsampling for Precision Medicine Biomarker Studies,” J Proteomics Res. 2022; 21 (4): 1196-1203; DOI: 10.1021/acs.jproteome.1c00971. Combinations of the following protocols were used to evaluate variations of digestion buffers and/or peptide cleanup procedures, relative to Example II.

Protocol as described in Example II:
1. Wash overnight with 1 mL of LiCl
2. Wash 2 times with 1 mL of LiCl by centrifuge pulsing
3. Add 100 μL of Master Mix buffer to each tip (1% sodium deoxycholate (SDC), 10 mM tris (2-carboxyethyl) phosphine (TCEP), 40 mM 2-Chloroacetamide (CLA), 100 mM trisaminomethane (tris), pH 8)
4. Heat at 95° C. for 10 minutes with gentle agitation
5. Add 1 μg (2 μL) of trypsin (0.5 μg/μL in Master Mix buffer)
6. Incubate overnight at 37° C. for 19 to 21 hours
7. Remove tips from tubes (slowly slide the tip along the inner side of the Eppendorf tube, allowing excess supernatant to drain back into the tube) and recover all supernatant including any solution accumulated in the probe mounting orifice.
8. Precipitate sodium deoxycholate (SDC) with 2 μL 100% formic acid
9. Centrifuge at 12,000 g for 5 minutes and transfer the supernatant to a clean tube
10. Cleanup via Stage-Tip using styrenedivinylbenzene reverse phase sulfonate (SDB-RPS) plugs (3 layers-basically 3 Stage-Tips stacked in a single device to increase capacity).
11. Centrifuge at 12,000 g for 5 minutes to prevent Stage-Tips from accumulating any solid SDC

Protocol utilizing FPB buffer:
1. Wash overnight with 1 mL of LiCl
2. Wash 2 times with 1 mL of LiCl by centrifuge pulsing
3. Add 100 μL of FPB buffer (1% SDC, 10 mM TCEP, 40 mM iodoacetamide (IAA), 100 mM triethylammonium bicarbonate buffer (TEAB) pH 8) to each tip
4. Heat at 95° C. for 10 minutes with gentle agitation
5. Add 1 μg (2 μL) of trypsin (0.5 μg/μL in FPB buffer)
6. Incubate overnight at 37° C. for 19 to 21 hours
7. Remove tips from tubes (slowly slide the tip along the inner side of the Eppendorf tube, allowing excess supernatant to drain back into the tube) and recover all supernatant including any solution accumulated in the probe mounting orifice
8. Precipitate sodium deoxycholate (SDC) with 2 μL 100% formic acid
9. Centrifuge at 12,000 g for 5 minutes and transfer the supernatant to a clean tube
10. Cleanup using “protocol utilizing Stage-Tips” or “protocol utilizing solid phase extraction (SPE)” described below.

Protocol utilizing Stage-Tips:
1. Prepare Stage-Tips using a needle and syringe by puncturing 3 layers of SDB-RPS disks with the a needle and packing them into a 200 μL pipette tip. Slight force from a retracted 10 ml syringe can be used to dislodge the disks from the needle (placed near the lower inner portion of the 200 μL pipette tip) into the tip.
2. Stage-Tip holders (lids needed during centrifugation) can be made by punching a hole through a lid of a 1.5 mL tube using a screwdriver.
3. Place the Stage-Tips into its holder mounted into an Eppendorf tube and add 100 μL of 100% methanol. Centrifuge at 1000×g for 1 minute
4. Add 100 μL of 0.2% trifluoroacetic acid (TFA) into each Stage-Tip to equilibrate. Centrifuge at 1000×g for 3 minutes
5. Add sample into each Stage-Tips. Centrifuge at 1000×g for 3 minutes
6. Wash the Stage-Tips using 100 μL 0.2% TFA and centrifuge at 1000×g for 3 minutes
7. Repeat step 6
8. Place the Stage-Tip into a new 1.5 mL tube and elute the sample using 100 μL 80% Acetonitrile (ACN), 5% Ammonium Hydroxide and centrifuge at 1000×g for 3 minutes
9. Dry samples in SpeedVac
10. Add 25 μL of 0.5% Formic acid (FA) to dried samples to reconstitute prior to Liquid chromatography-mass spectrometry (LC-MS)
11. Estimate sample concentrations using a Nanodrop
12. Once the concentration of protein is known, normalise each sample by taking an aliquot and diluting further in loading buffer to ensure equal loading of samples on the LC-MS. For a 75 μm column injections of 0.6-1 μg works well.

Protocol utilizing SPE:
1. Dilute samples with 700 μL of 0.5% Formic acid (FA)
2. Spin 12,000 g for 5 minutes
3. Activate column (Waters HLB 10 mg cc) with 500 μL of 90% Acetonitrile (ACN), 0.5% FA (HLB is a type of SPE, see, e.g., https://www.sigmaaldrich.com/AU/en/technical-documents/technical-article/analytical-chemistry/solid-phase-extraction/supel-select-hlb-spe)
4. Wash with 800 μL of 0.5% FA
5. Load sample
6. Wash with 1000 μL of 0.5% FA
7. Wash with 1000 μL of 0.5% FA
8. Elute with 300 μL of 70% ACN 0.5% FA
9. Elute with 150 μL of 90% ACN 0.5% FA
10. Dry down
11. Re-suspend in 25 μL 0.5% FA
12. Samples concentrations can be estimated using a Nanodrop

Samples prepared and evaluated:
Example V-1—Whole Blood Cells, Overnight Digestion, FPB buffer, and Stage-Tip
Example V-2—Whole Blood Cells, Overnight Digestion, FPB buffer, and solid phase extraction (SPE)
Example V-3—Whole Blood Cells, Overnight Digestion, Master Mix buffer, and SPE
Example V-4—Whole Blood Cells, Overnight Digestion, Master Mix buffer, and Stage-Tip

Results and Discussion:

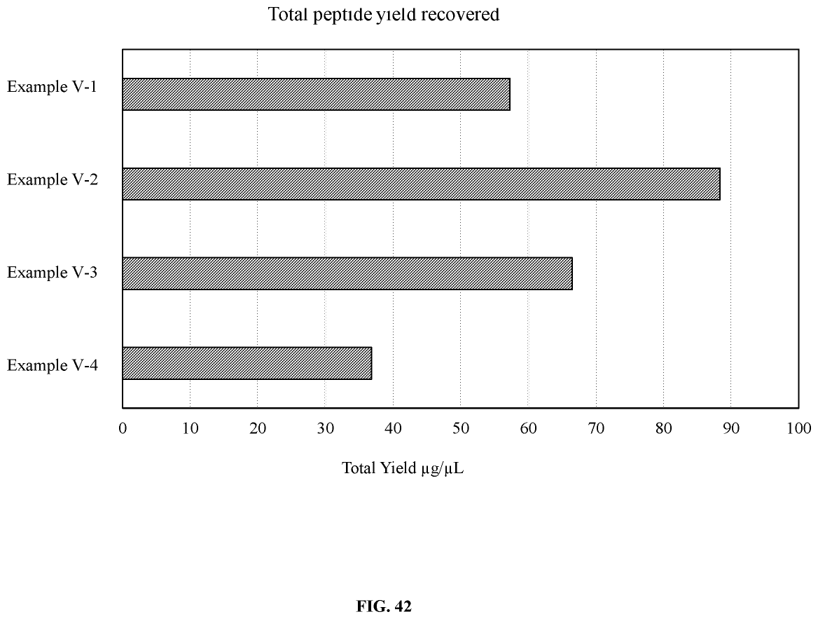

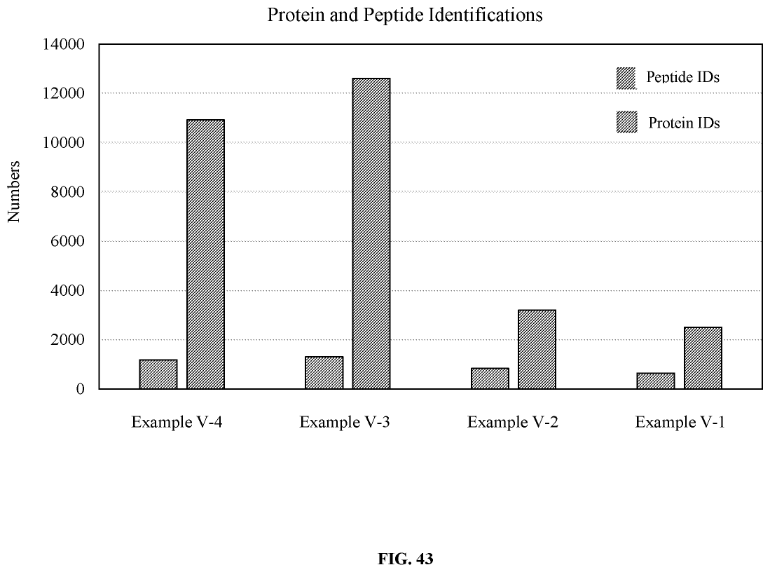

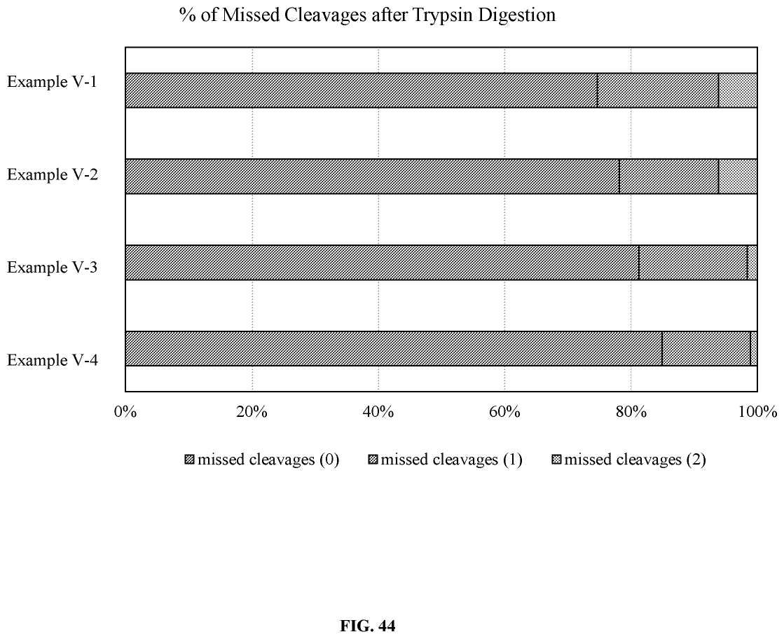

The resulting peptide yields recovered according to Examples V-1 to V-4 are shown in FIG. 42. For comparison, the resulting peptide yield recovered according to Example II is about 0.8 μg/μL (a total of about 16 μg, which is near capacity of the Stage-Tips). The number of protein and peptide IDs identified according to Examples V-1 to V-4 are shown in FIG. 43. As illustrated in FIG. 43, the use of the Master Mix buffer gave superior peptide IDs to the FPB buffer when utilizing either the Stage-Tip or SPE protocol (samples run on Mass Spec QEHFX4). Additionally, the use of the SPE protocol gave about 10% more protein IDs and about 15% more peptide IDs over using the Stage-Tips protocol. The percentage of missed cleavages following trypsin digestion according to Examples V-1 to V-4 are shown in FIG. 44. As illustrated in FIG. 44, samples with the FPB buffer had more missed cleavages than those with the Master Mix buffer, indicating an issue with digestion or cystine reduction/alkylation in the FPB buffer.

In the method of Example II, after the trypsin digestion the porous plastic material (Mitra®) was removed from the solution and the solution allowed to run into the tube-essentially gravity removal, resulting in a 50% recovery of solution-50 μL (out of 100 μL) in the tube. Using the Stage-Tip protocol of the instant method involves centrifuging and washing the tips to get the remaining trypsin digest buffer out. The centrifugal removal of digest from the tips resulted in nearly a doubling of liquid in the tube and a large increase in peptide yields (relative to about 16 μg according to Example II). Using the SPE protocol increased the total yield by around 30% (see Examples V-2 and V-3, compared to Example V-1).

Conclusion: Centrifugal removal of the peptide digest produces a higher yield of peptides compared to the Protocol as described in Example II. Solid phase extraction (SPE) produces a higher yield of peptides compared to Stage-Tip clean-up of the peptide digest.

### Example VI: Evaluation of Buffer Variations and Shorter Trypsin Digestion on PBMCs and Whole Blood

Purpose: evaluate impact on varying digestion buffers and/or shorter digestion time (2 hours vs. overnight (19-21 hours)) on peripheral blood mononuclear cells (PBMCs) and Whole Blood (WB), relative to the methods described in Example II, on peptide yields and/or protein identifications after mass spec analysis.

Samples prepared and evaluated:
Example VI-1—PBMCs in VAMS tips (30 μL), Overnight Digestion, FPB buffer Example VI-2—PBMCs in VAMS tips (30 μL), Overnight Digestion, FPB buffer
Example VI-3—PBMCs in VAMS tips (30 μL), 2-hour Digestion, FPB buffer
Example VI-4—PBMCs in VAMS tips (30 μL), 2-hour Digestion, FPB buffer
Example VI-5—WB in VAMS tips (30 μL), Overnight Digestion, FPB buffer
Example VI-6—WB in VAMS tips (30 μL), Overnight Digestion, FPB buffer
Example VI-7—WB in VAMS tips (30 μL), 2-hour Digestion, FPB buffer
Example VI-8—WB in VAMS tips (30 μL), 2-hour Digestion, FPB Buffer
Example VI-9—WB in VAMS tips (30 μL), Control-Overnight Digestion, Master Mix Buffer
Example VI-10—WB in VAMS tips (30 μL), Control-Overnight Digestion, Master Mix Buffer, No Spin

PBMC Preparation. PBMC tips were prepared using Vacutainer CPT™ Cell Preparation Tube with Sodium HeparinN as per the manufacturer's protocol (BD Vacutainer® CPT™, Cell Preparation Tube with Sodium HeparinN, Ref No. 362753; https://www.rch.org.au/uploadedFiles/Main/Content/Specimen_Collection/CellC%20Preparation %20Tube%20CPT%20BD%20Vacutainer.pdf). After isolation, the white blood cell (WBC) counts on the collected PBMC fraction was 21×10³ cells/μL. Extracted PBMCs were diluted 1:3 to reach a cell count of 7×10³ cells/μL.

VAMS 30 μL tips were dipped in PBMC preparation or whole blood and dried for 24-hour.

Overnight Digestion Protocol
1. Wash overnight with 1 mL of LiCl
2. Wash 2 times with 1 mL with of LiCl by centrifuge pulsing
3. Add 100 μL of Master Mix buffer to each tip (1% SDC, 10 mM TCEP, 40 mM CLA, 100 mM tris) or FPB Buffer (1% SDC, 10 mM TCEP, 40 mM IAA, 100 mM TEAB
4. Heat at 95° C. for 10 minutes with gentle agitation (tips should remain in position-invert any tips which are disoriented)
5. Add 1 μg (2 μL) of trypsin (0.5 μg/μL in master mix buffer)
6. Incubate overnight at 37° C. for 19 to 21 hours
7. Spin the tubes to remove liquid. (this step was omitted in Example VI-10 to see yield/results with no spin).
8. Wash with 100 μL of 0.5% Formic acid
9. Dilute with 700 μL of 0.5% Formic acid
10. Centrifuge at 12,000 g for 10 minutes and transfer the supernatant to conditioned SPE cartridge.
11. Cleanup using SPE protocol as indicated in Example V.

2-hour Digestion Protocol
1. Wash overnight with 1 mL of LiCl
2. Wash 2 times with 1 mL of LiCl by centrifuge pulsing
3. Add 50 μL of FPB Buffer (1% SDC, 10 mM TCEP, 40 mM IAA, 100 mM TEAB
4. Heat at 95° C. for 10 mins with gentle agitation (tips should remain in position-invert any tips which are disoriented)
5. Add 150 μL of Rapid Trypsin Digestion Buffer
6. Add 1 μg of trypsin/LysC mix
7. Incubate 70° C. for 2-hour on a thermomixer
8. Spin the tubes to remove liquid
9. Wash with 100 μL of 0.5% Formic acid
10. Dilute with 700 μL of 0.5% Formic acid
11. Centrifuge at 12,000 g for 10 minutes and transfer the supernatant to conditioned SPE cartridge.
12. Cleanup using SPE protocol as above for Example V.

### Results and Discussion:

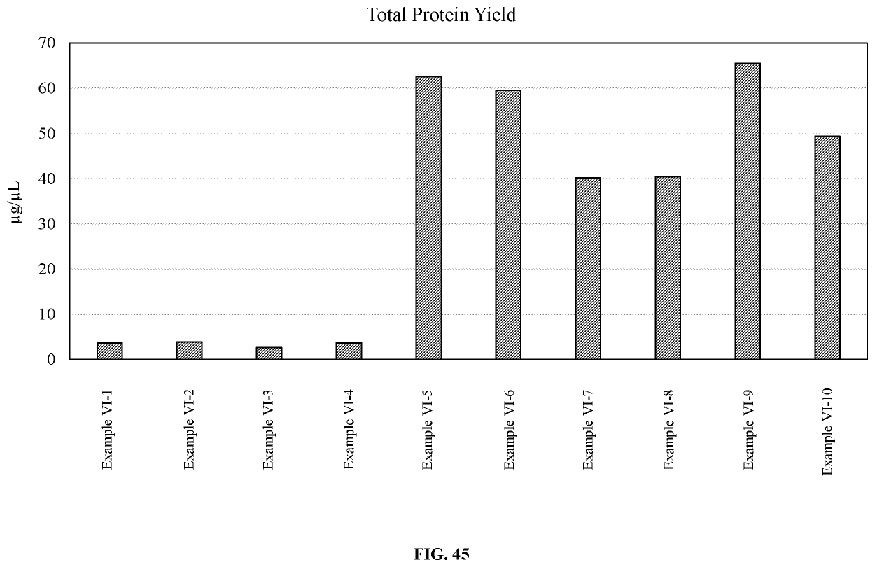

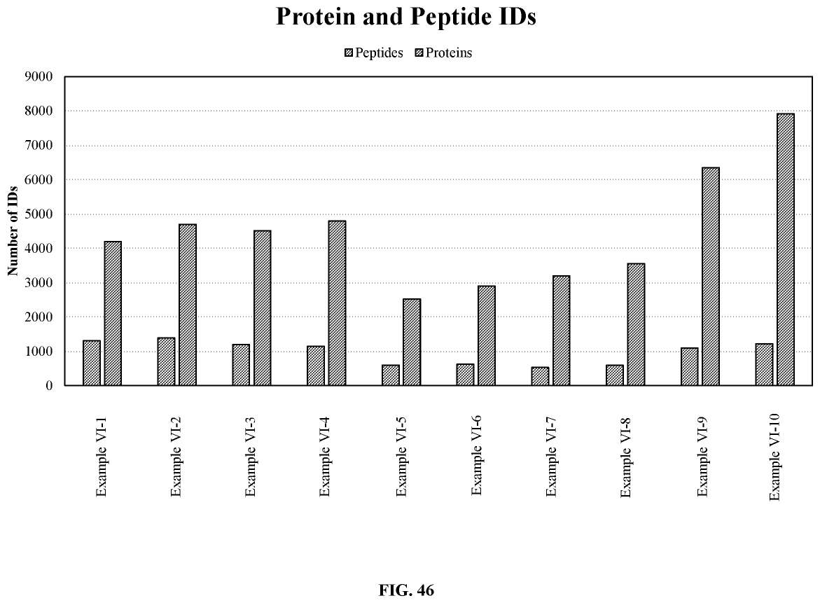

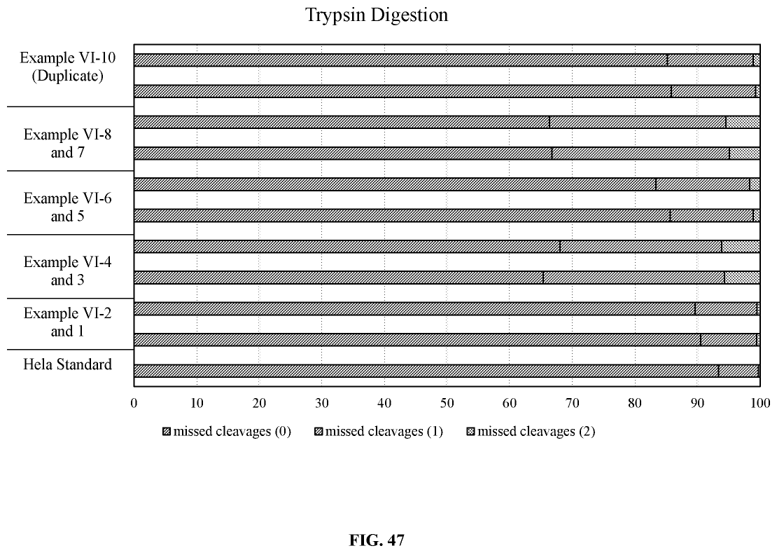

Total protein yield shown for all samples in FIG. 45. PBMC's were all below 5 μg of total protein. The Example VI-10, which does not include the spin step, had a lower yield than the sample with the spin step (Example VI-9). The 2-hour digestion samples (Examples VI-3, 4, 7 and 8) had a lower yield, relative to the overnight digestion samples, though mainly for the WB samples (Examples VI-7 and 8). FIG. 46 illustrates the number of protein and peptide IDs observed from the PBMC and WB samples. The PBMCs samples (Examples VI-1 to VI-4) showed a considerable number of IDs, even though the IDs for FPB buffer (Examples VI-1 to VI-8) were still lower than those with the Master Mix buffer (Examples VI-9, 10, and Hela standard (use of Hela cells in Master Mix buffer)). Overall, the rapid 2-hour digestion did identify more peptides, even though there was a larger number of missed cleavages (as illustrated in FIG. 47). The “no spin” sample (Example VI-10) showed the greatest number of IDs. Samples that included the spin step had a lot of higher charged/larger peptides.

The percentages of missed cleavages after trypsin digestion are illustrated in FIG. 47.

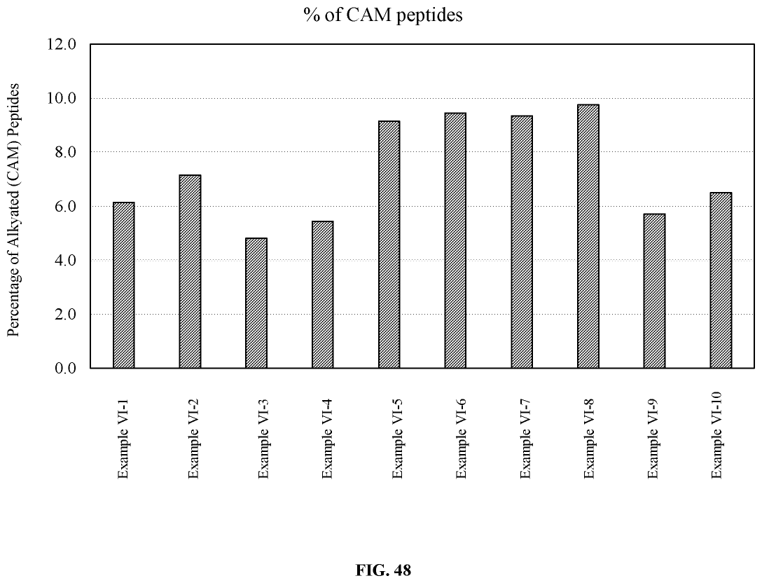

The number of alkylated peptides (containing carbamidomethyl modification (CAM)) in the sample was also determined (see FIG. 48). Samples with FPB buffer gave a higher percentage of CAM peptides.

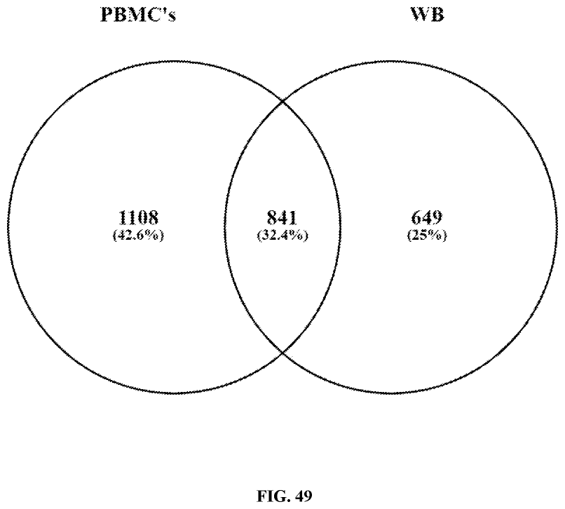

A Venn-diagram illustrating the overlap of protein IDs between PBMCs and WB is provided in FIG. 49.

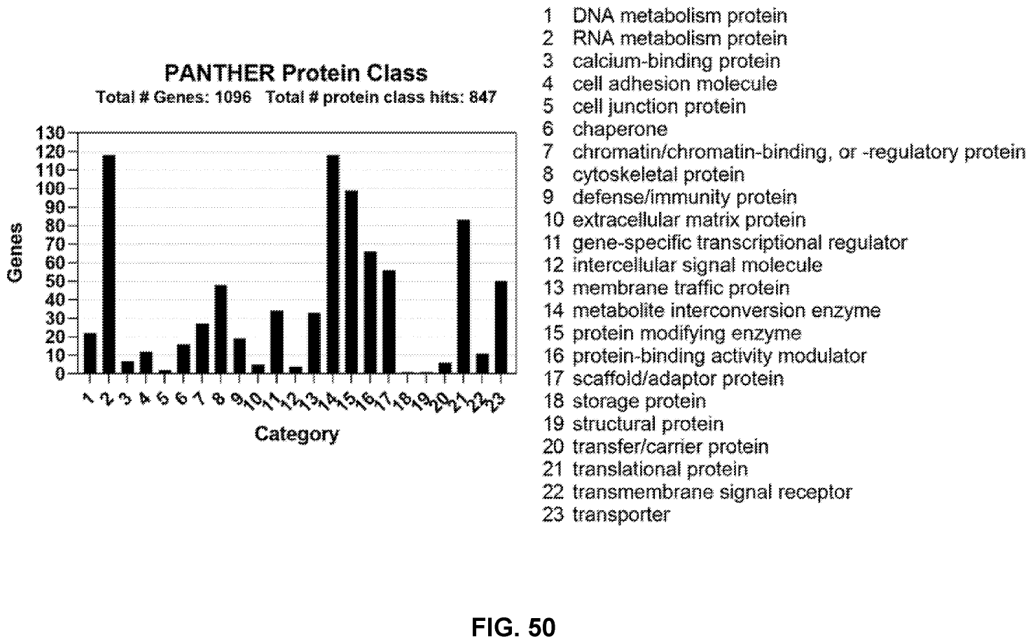

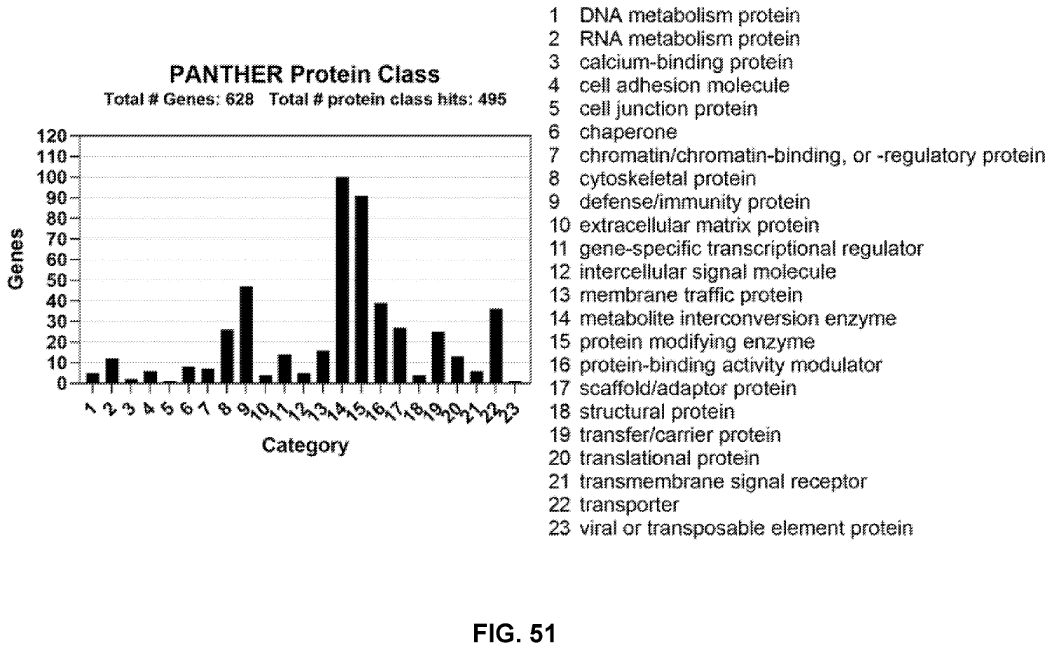

The protein classes of those proteins found exclusively in PBMCs are illustrated in FIG. 50, and the protein classes of those proteins found exclusively in WB are illustrated in FIG. 51. Focusing on the proteins exclusive to either PBMCs or WB, the PBMCs samples show a lot more RNA metabolism proteins and translational proteins and also some storage proteins that do not show up in WB samples. The WB samples contain a larger proportion of defence/immunity proteins.

Conclusion: Variations on the buffer contribute to a lower amount of protein and peptide IDs. Centrifuging the digested solution out of the tip results in a higher yield (see Example V), but results in lower protein and peptide IDs. Utilization of a 2-hour trypsin digestion resulted in a reasonable number of protein and peptide IDs, but also resulted in a higher proportion of missed cleavages.

### Example VII: Evaluation of Buffer Variations and Shorter Trypsin Digestion on PBMCs, WB, and WB in Streck Tubes

Purpose: investigate impact of various buffer variations and test a rapid trypsin/LysC 2-hour digestion using higher amount of trypsin (to improve missed cleavages). Evaluated using whole blood, whole blood in Streck tubes and PBMC tips. Streck Cell-free DNA BCT tubes contain stabilisers for cell-free DNA, which provided an opportunity to evaluate protein recovery from Streck tubes (which cannot be used for immunoassay). It is noted that extensive disease sample collections in many biobanks utilize stabilisation techniques like those used with Streck tubes. Accordingly, stabilized samples, including nucleic acid-containing stabilized samples or stabilized blood samples, such as Streck tube samples, may be suitable for proteomics analysis using the methods disclosed herein.

Tubes designed to stabilise blood, such as Streck tubes, contain imidazolidinyl urea, which release formaldehyde in solution after the blood sample has entered the tube. Formaldehyde has been used as a fixative and preservative for many decades. It is reactive toward both proteins and DNA, and forms inter-molecular cross-links between macromolecules as well as intra-molecular chemical modifications. The high reactivity of formaldehyde together with its high permeability into cells and tissues has led to its use in numerous applications in biology, biotechnology, and medicine. Stabilised blood samples often produce incorrect results when assayed using immuno-assays because the sample doesn't contain individual macromolecules after stabilisation. Crosslinks form between proteins and nucleic acids and between dissimilar molecules-protein to DNA etc (https://www.nature.com/articles/s41467-020-16935-w).

Samples prepared and evaluated (due to the results from Example VI with centrifuging the tips post digestion, each sample was conducted with ‘no spin’):
Example VII-1—PBMCs in VAMS tips (30 μL), Overnight digestion, FPB buffer
Example VII-2—PBMCs in VAMS tips (30 μL), Overnight digestion, Master Mix buffer
Example VII-3—PBMCs in VAMS tips (30 μL), 2-hour digestion, Master Mix buffer
Example VII-4—WB in VAMS tips (30 μL), Overnight digestion, FPB buffer
Example VII-5—WB in VAMS tips (30 μL), Overnight digestion, Master Mix buffer
Example VII-6—WB in VAMS tips (30 μL), 2-hour digestion, Master Mix buffer
Example VII-7—WB in Streck (“WB-Streck”) tubes—in VAMS tips (30 μL), Overnight digestion, FPB buffer
Example VII-8—WB-Streck tubes—in VAMS tips (30 μL), Overnight digestion, Master Mix buffer
Example VII-9—WB-Streck tubes—in VAMS tips (30 μL), 2-hour digestion, Master Mix buffer

Overnight Digestion Protocol
1. Wash overnight with 1 mL of LiCl.
2. Wash 2 times with 1 mL of LiCl (centrifuge pulsing)
3. Add 100 μL of Master Mix buffer to each tip (1% SDC, 10 mM TCEP, 40 mM CLA, 100 mM tris) or FPB Buffer (1% SDC, 10 mM TCEP, 40 mM CLA, 100 mM Tris
4. Heat at 95° C. for 10 mins with gentle agitation (tips should remain in position-invert any tips which are disoriented)
5. Add 1 μg (2 μL) of trypsin (0.5 μg/μL in master mix buffer)
6. Incubate overnight at 37° C. for 19 to 21 hours
7. Draw the tip up the side of the tube
8. Dilute with 800 μL of 0.5% Formic acid (SDC will precipitate out)
9. Centrifuge at 12,000 g for 10 mins and transfer the supernatant to conditioned SPE cartridge.
10. Cleanup using SPE protocol as above for Example V.

2-hour Digestion Protocol
1. Wash overnight with LiCl (1 mL)
2. Wash 2 times 1 mL with LiCl (centrifuge pulsing)
3. Add 100 μL of Master Mix buffer to each tip (1% SDC, 10 mM TCEP, 40 mM CLA, 100 mM tris)
4. Heat at 95° C. for 10 mins with gentle agitation (tips should remain in position-invert any tips which are disoriented)
5. Add 200 μL of Rapid Trypsin Digestion Buffer
6. Add 3 μg of trypsin/LysC mix
7. Incubate 70° C. for 2-hour on a thermomixer
8. Draw the tip up the side of the tube
9. Dilute with 800 μL of 0.5% Formic acid (SDC will precipitate out)
10. Centrifuge at 12,000 g for 10 mins and transfer the supernatant to conditioned SPE cartridge.
11. Cleanup using SPE protocol as above for Example V.

### Results and Discussion:

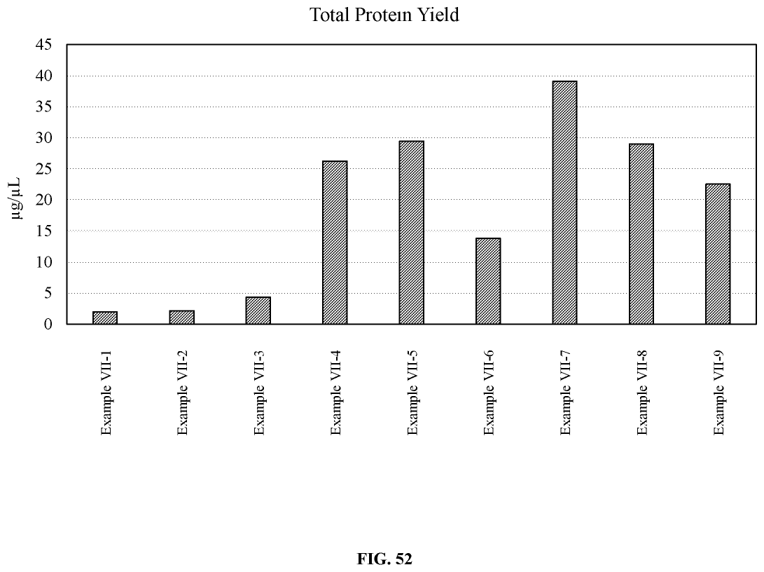

Total protein yield for samples Examples VII-1 to VII-9 are shown in FIG. 52. PBMC samples (Examples VII-1 to VII-3) were all below 5 μg of total protein. The samples with the 2-hour digestion showed a lower yield for the WB samples, yet higher for the PBMCs. Both WB rapid trypsin digestion samples (Examples VII-6 and VII-9) resulted in a lower yield, relative to overnight digestion samples (Examples VII-5 and VII-8), respectively. PBMC rapid trypsin digestion sample provided higher yield than the overnight digestion (Example VII-3 compared to VII-2, respectively).

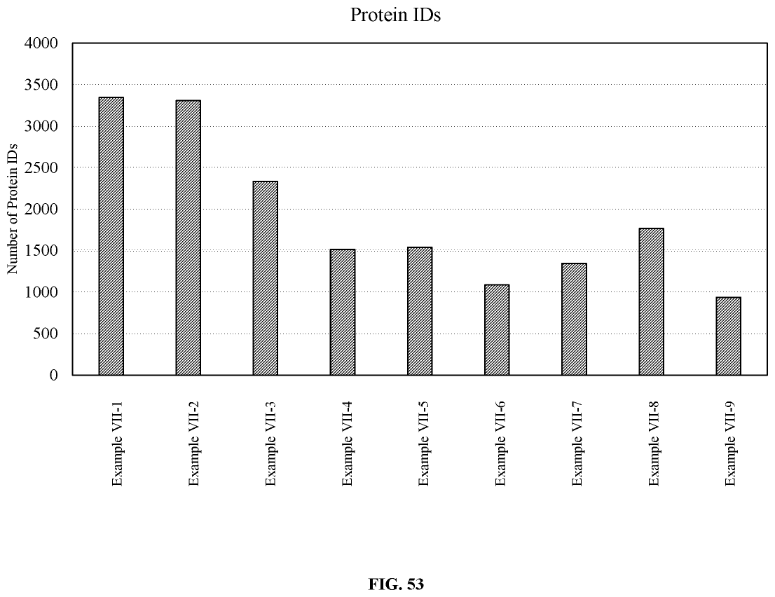

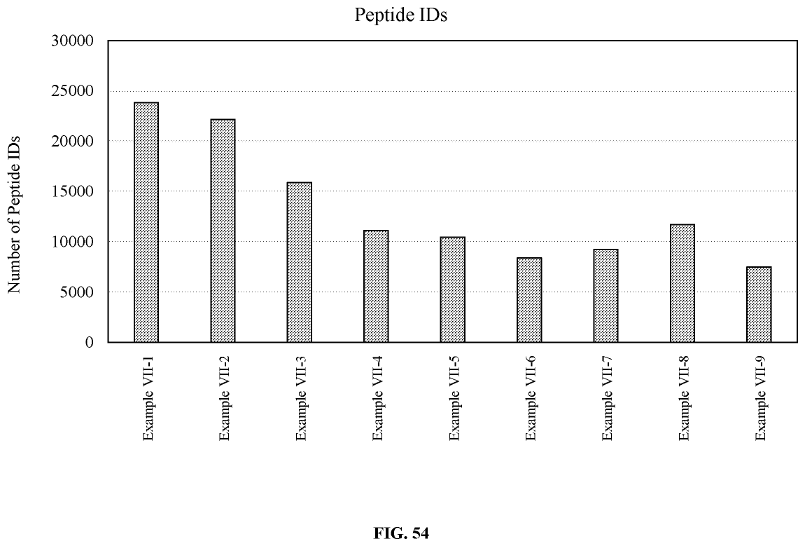

The number of proteins IDs and peptide IDs were measured, as shown in FIG. 53 and FIG. 54, respectively. As illustrated in FIG. 53, protein IDs for PBMCs were over 3000. No issues were observed with the use of the FPB buffer. The number of peptide IDs and protein IDs observed and recovered from the WB-Streck tubes (Examples VII-7 to VII-9) was on par with those recovered and observed from the WB samples (Examples VII-4 to VII-6), respectively. The relative number of Peptide IDs across the samples was similar to and reflected in the protein ID results.

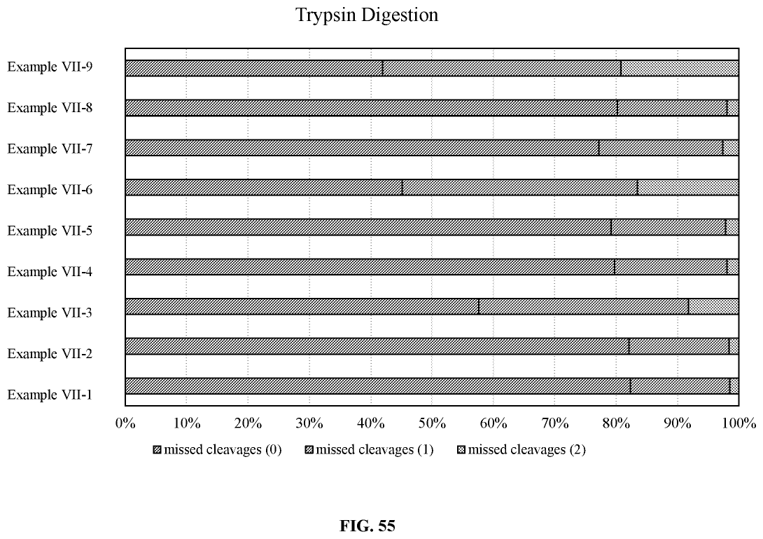

The percentage of missed cleavages following trypsin digestion is illustrated in FIG. 55. Here, the rapid digestion samples (e.g., Examples VII-3 and VII-6) resulted in an even higher number of missed cleavages, compared to those in Example VI. Of note, in Example VI, 50 μL was added and diluted with 150 μL before addition of trypsin, while in Example VII, 100 μL of buffer was added and diluted with 200 μL prior to trypsin addition.

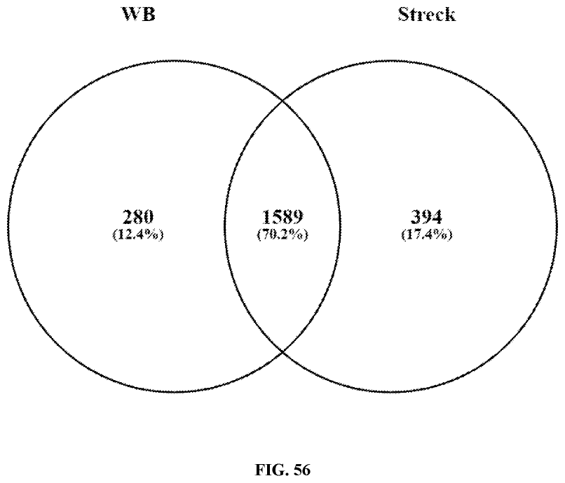

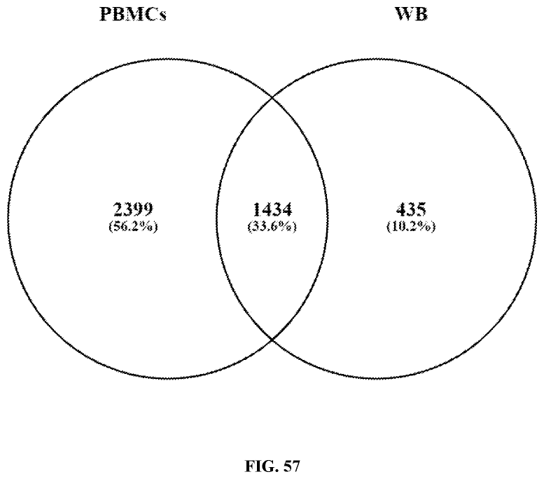

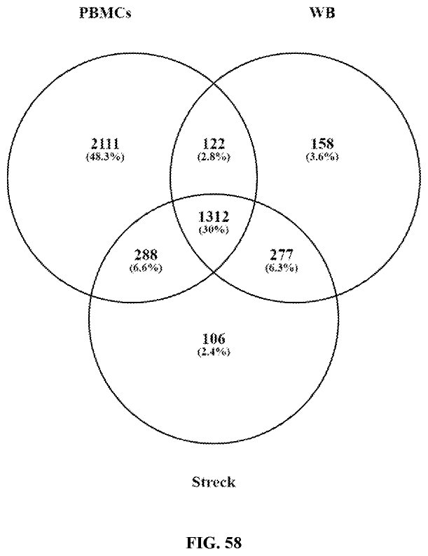

A Venn-diagram illustrating the overlap of protein IDs between WB and WB-Streck tube samples is provided in FIG. 56. WB and WB-Streck tubes had an excellent overlap, indicating good recovery of the samples. A Venn-diagram illustrating the overlap of protein IDs between PBMCs and WB samples is provided in FIG. 57. WB and PBMCs showed a decent overlap, with a large number of extra IDs coming from PBMCs. A Venn-diagram illustrating the overlap of protein IDs between PBMCs, WB and WB-Streck tube samples is provided in FIG. 58.

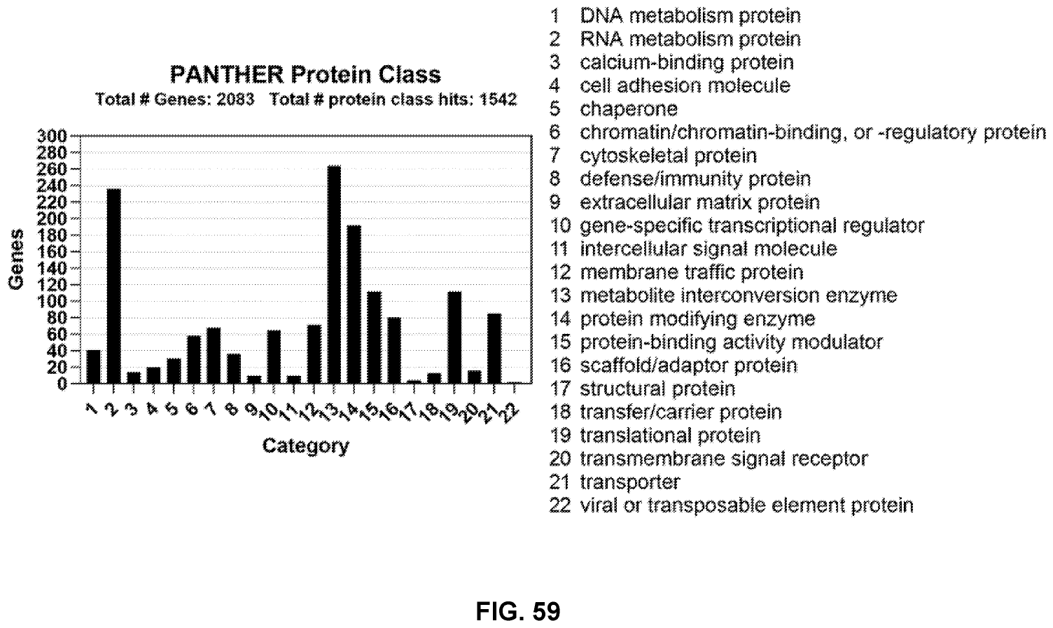

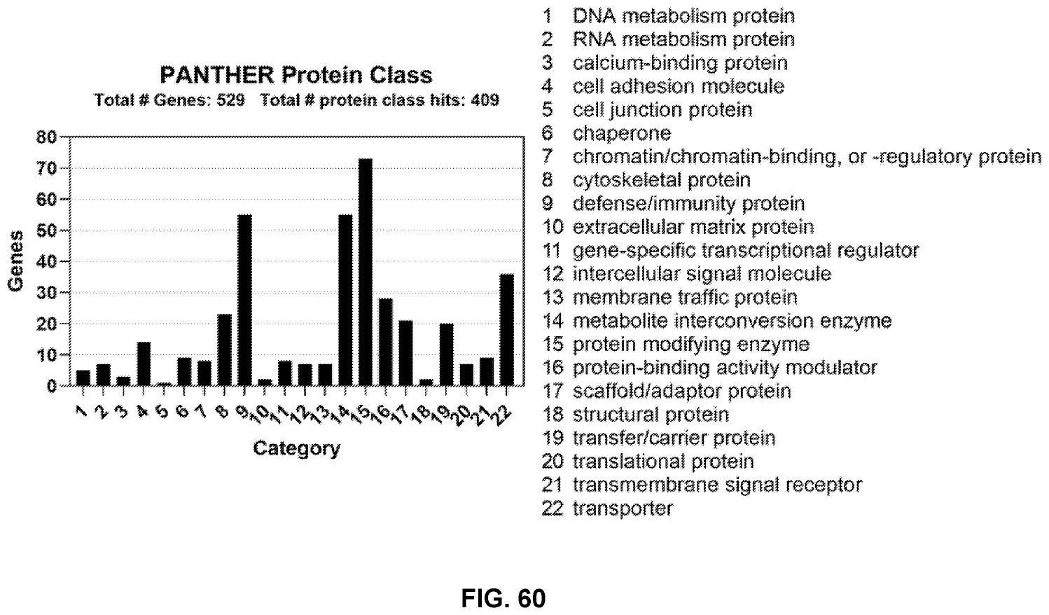

FIG. 59 illustrates protein classes of those proteins found exclusively in PBMCs, and FIG. 60 illustrates protein classes of those proteins found exclusively in WB and WB-Streck tube samples. Focusing on the proteins exclusive to the PBMCs, WB or WB-Streck tube samples, the PBMC samples show a lot more RNA metabolism proteins and translational proteins and also some storage proteins that do not show up in WB samples. WB samples contains a larger proportion of defence/immunity proteins.

Conclusion: Use of the methods disclosed herein resulted in a high number of protein and peptide IDs from PBMC, WB and WB-Streck tube samples. Use of the buffer conditions provided in Example II produced similar results. Use of the 2-hour trypsin digestion resulted in a lower number of peptide and protein IDs, and showed a larger proportion of missed cleavages.

### Example VIII: Optimisation of Different Wash Conditions Using Buffy Coat, and Trypsin Digestion Optimisation Using Whole Blood

Purpose: evaluate impact of different initial washing mechanisms for tips dipped in buffy coat. Buffy coat is the fraction of an anticoagulated blood sample that contains most of the white blood cells and platelets. This experiment also evaluated use of LiCl washing and a urea/thiourea wash. Using whole blood tips, this experiment also investigated the use of iodoacetamide compared with chloroacetamide for alkylation of the proteins after reduction. For the 2-hour trypsin digestion, the volume of lysis buffer added prior to trypsin digestion was also evaluated.
Example VIII-1—Buffy Coat, LiCL
Example VIII-2—Buffy Coat, Urea/Thiourea
Example VIII-3—WB, Urea/Thiourea
Example VIII-4—WB, Control
Example VIII-5—WB, IAA
Example VIII-6—WB, 50 μL RedAlk, Overnight Digestion
Example VIII-7—WB, 50 μL RedAlk, 2-hour Digestion

Buffy Coat Tips Preparation. Whole blood was centrifuged at 1500 g for 10 mins. Plasma was removed and buffy coat layer extracted to new tube. Tube was centrifuged again, and second separation transferred to new tube. 30 μL VAMS tips dipped into buffy coat extract and dried overnight.

### Overnight Digestion Protocol

1. Wash overnight with LiCl (1 mL) or with 7M Urea, 2M Thiourea (1 mL)
2. Wash 2 times with 1 mL with LiCl (centrifuge pulsing) or 2 times with 1 mL of 7M Urea, 2M Thiourea (centrifuge pulsing)
3. Add 100 μL Lysis Buffer (1% SDC, 10 mM TCEP, 40 mM CLA, 100 mM Tris, pH 8) (for sample Example VIII-5-40 mM IAA was used and 50 μL of Lysis buffer was added to Example VIII-6)
4. Heat at 95° C. for 10 minutes with gentle agitation (tips should remain in position-invert any tips which are disoriented)
5. Add 1 μg (2 μL) of trypsin (0.5 μg/μL in master mix buffer)
6. Incubate overnight at 37° C. for 19 to 21 hours
7. Draw the tip up the side of the tube
8. Dilute with 800 μL of 0.5% Formic acid (SDC will precipitate out)
9. Centrifuge at 12,000 g for 10 minutes and transfer the supernatant to conditioned SPE cartridge.
10. Cleanup using SPE protocol as above for Example V.

2-hour Digestion Protocol Used for
1. Wash overnight with 1 mL of LiCl
2. Wash 2 times with 1 mL with LiCl by centrifuge pulsing
3. Add 50 μL of Master Mix buffer to each tip (1% SDC, 10 mM TCEP, 40 mM CLA, 100 mM tris)
4. Heat at 95° C. for 10 minutes with gentle agitation (tips should remain in position-invert any tips which are disoriented)
5. Add 150 μL of Rapid Trypsin Digestion Buffer
6. Add 2 μg of trypsin/LysC mix
7. Incubate 70° C. for 2-hour on a thermomixer
8. Draw the tip up the side of the tube
9. Dilute with 800 μL of 0.5% Formic acid (SDC will precipitate out)
10. Centrifuge at 12,000 g for 10 minutes and transfer the supernatant to conditioned SPE cartridge.
11. Cleanup using SPE protocol as above for Example V.

### Results and Discussion:

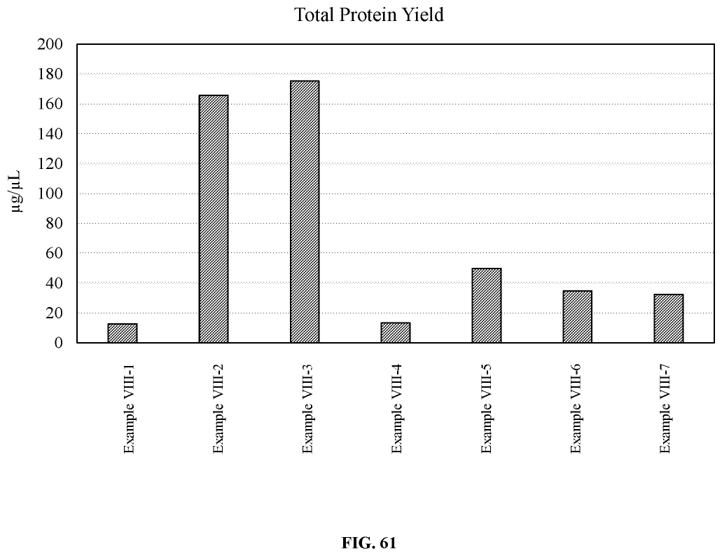

Total protein yield for samples Examples VIII-1 to VIII-7 are shown in FIG. 61. The Urea/Thio Urea washed samples had a large increase in total yield (μg/μL).

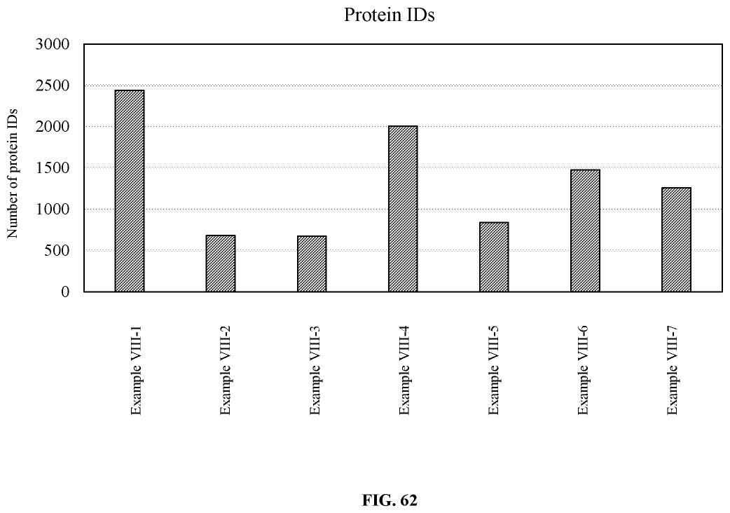

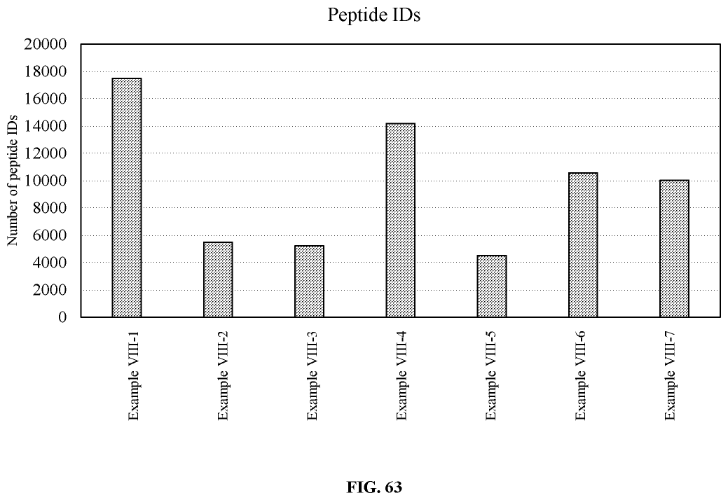

The number of proteins IDs and peptide IDs were measured, as shown in FIG. 62 and FIG. 63, respectively. The number of protein IDs and peptide IDs for the Urea/Thiourea wash samples (Examples VIII-2 and VIII-3) are greatly reduced. Such results could be due to poor digestion and/or the presence of more high abundant proteins, such as Serum albumin and Complement C3 (see section below). The alkylation using iodoacetamide (IAA) produced poor number of protein and peptide IDs. The lower volume of red/alkylation buffer produced results on par with and close to the control. The 2-hour digestion identified roughly the same number of proteins and peptides as the overnight digest with the same volume of buffer, even though the digestion efficiency was still only about 50%.

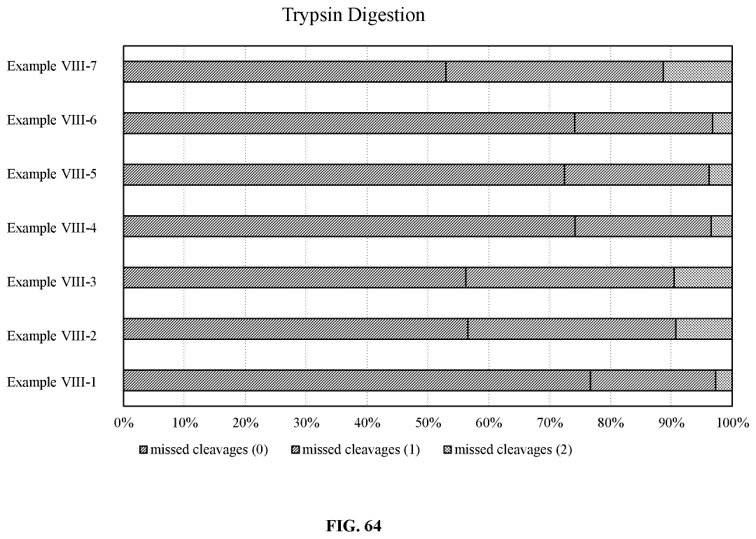

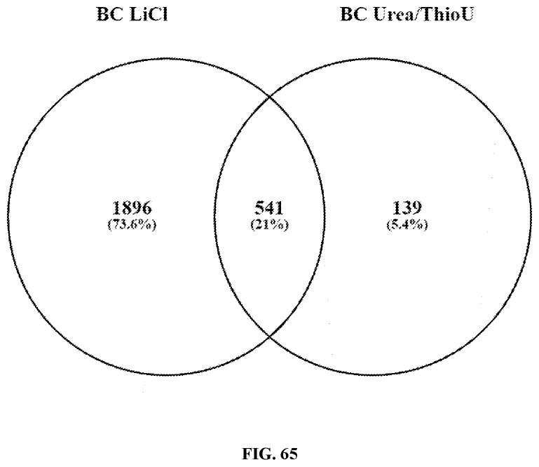

The percentage of missed cleavages following trypsin digestion is illustrated in FIG. 64. Here, trypsin digestion was poor for those samples washed with Urea/ThioUrea. This could be due to residual high concentrations of Urea in the tip as concentrations >1M urea can have negative effect on trypsin digestion. The 2 hour digest resulted in lower digestion efficiency even though the IDs are comparable.

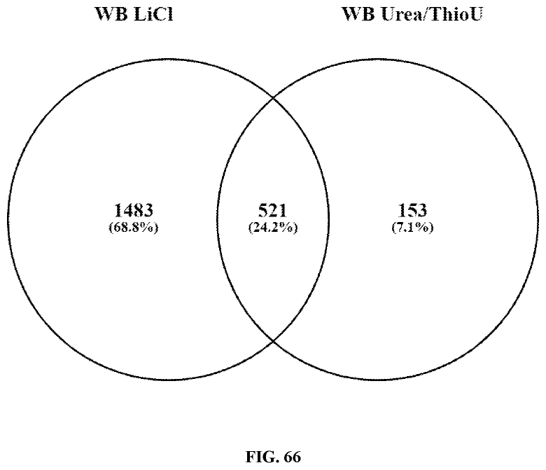

A Venn-diagram illustrating the overlap of protein IDs between Buffy Coat samples washed with LiCl and Urea/Thio Urea is provided in FIG. 65. A Venn-diagram illustrating the overlap of protein IDs between WB samples washed with LiCl and Urea/Thio Urea is provided in FIG. 66. The results show that a majority of proteins identified in the Urea/ThioUrea wash samples overlap with those identified in the LiCl wash samples for both the buffy coat and the whole blood tip samples.

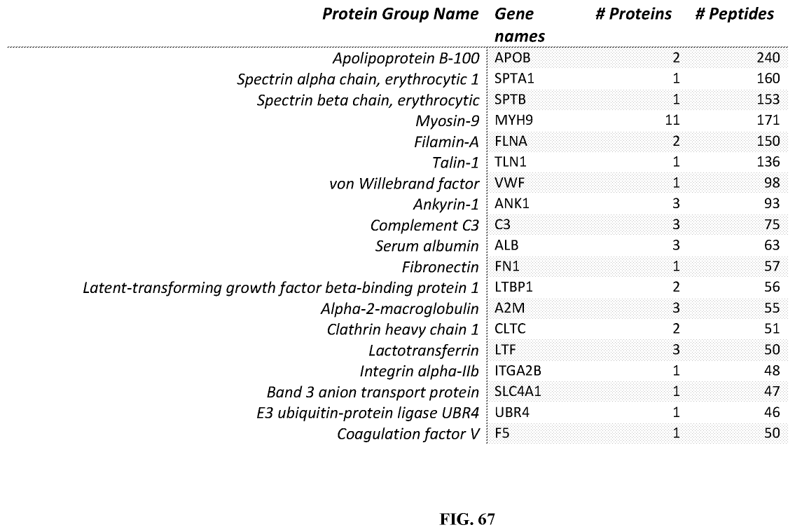

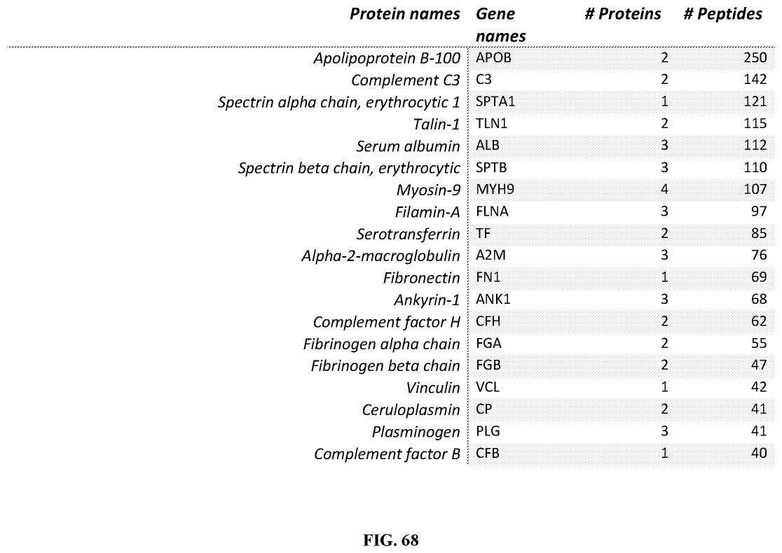

FIG. 67 details the top protein IDs observed from the Buffy Coat with LiCl wash samples, and FIG. 68 details the top protein IDs observed from the Buffy Coat with Urea/ThioUrea wash samples. The Buffy Coat with LiCl wash samples reduced the number of peptides for high abundant proteins Serum albumin and Complement C3 by about a factor of two.

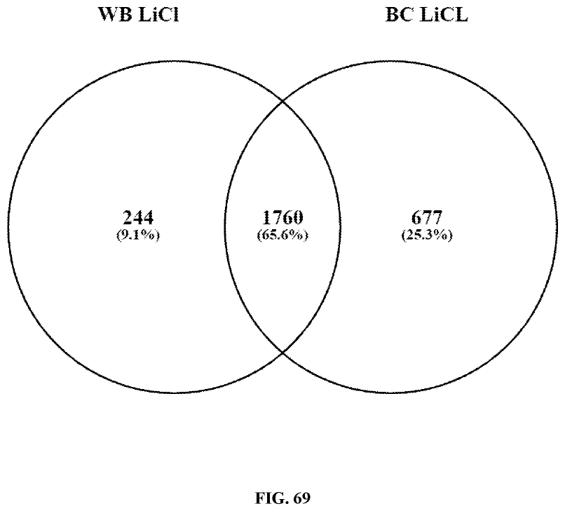

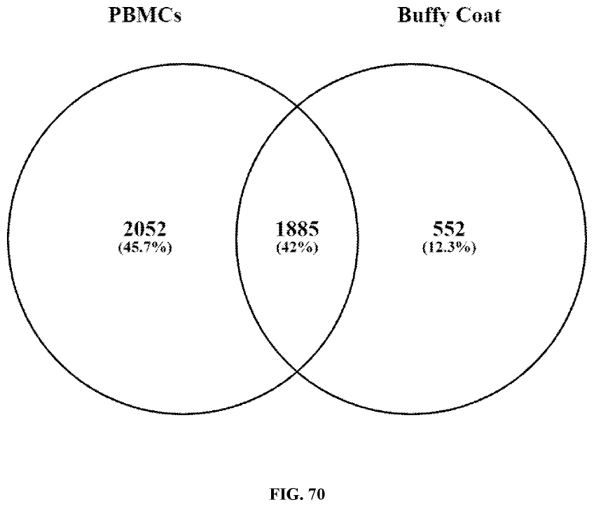

A Venn-diagram illustrating the overlap of protein IDs between Buffy Coat samples and WB samples each washed with LiCl is provided in FIG. 69. A considerable overlap between the WB and Buffy Coat tips was observed, with a greater number of IDs provided from the Buffy Coat tip samples. A Venn-diagram illustrating the overlap of protein IDs between PBMC samples (Example VII) and Buffy Coat samples is provided in FIG. 70.

Conclusion: Utilizing the methods disclosed herein, the Urea/Thiourea washed samples had a large increase in total yield, however the number of protein and peptide IDs are greatly reduced. In addition, the methods disclosed herein resulted in a greater presence of high abundant proteins, such as Serum albumin and Complement C3, and there was a great deal of overlap between the Urea/Thiourea wash and LiCl wash samples. The digestion for the Urea/Thiourea was samples was poor. Alkylation using iodoacetamide (IAA) produced a poor number of protein and peptide IDs. The lower volume of red/alkylation buffer produced results on par with and close to the control. The 2-hour digestion identified roughly the same number of proteins and peptides as the overnight digest with the same volume of buffer, even though the digestion efficiency was still only about 50%. Buffy coat tip samples produced a high number of protein and peptide IDs, getting close to what was identified from PBMC samples, even though the preparation is a lot simpler.

### Example IX: Further Optimisation of Different Wash Conditions Using Buffy Coat, and Assessment of Freezing Blood Samples Prior to Fractionation Using VAMS Tips

Aim: To look at different initial washing mechanisms for tips dipped in buffy coat. Buffy coat is the fraction of an anticoagulated blood sample that contains most of the white blood cells and platelets. In this experiment we do a LiCl wash first, followed by variations on a urea/thiourea wash, one containing a detergent to help keep proteins soluble; one containing a reducing agent to help denature proteins, and the urea/thiourea alone. We also looked at the effects of freezing blood prior to sampling with VAMS tips.

### Methods

Buffy Coat Tips Preparation. Whole blood was centrifuged at 1500 g for 10 mins. Plasma was removed and buffy coat layer extracted to new tube. Tube was centrifuged again, and second separation transferred to new tube. 30 μL VAMS tips dipped into buffy coat extract and dried overnight.

Frozen Blood Tips Preparation. Blood was collected in both Streck and EDTA tubes. Whole blood was centrifuged at 1500 g for 10 mins and plasma was removed. The remaining RBC pellet was frozen for a minimum of 24 hours, then diluted 1:2 in PBS and then 30 μL tips were dipped into the thawed diluted RBC blood. The thawed RBC (total volume 1400 μL) was spun at 16000 g for 20 mins to pellet the red blood cells membranes, 700 μL of the supernatant was removed and used to fill a 30 μL tip. 700 μL of PBS was then added to dilute the remaining supernatant further and the sample was spun again at 16000 g for 10 mins. The supernatant was completely removed and the pellet was resuspended in 700 μL PBS and 30 μL tips were dipped. The tips were allowed to air dry, then were stored with desiccant for 24 hrs.

### Protocol Used for Overnight Digestion

Once dried, the tips were washed overnight with LiCl (1 mL) then 2× 30 min washes with either (a) 1 mL 7M Urea, 2M Thiourea; (b) 1 mL 2M Urea, 2M Thiourea, 1% sodium deoxycholate; (c) 1 mL 7M Urea, 2M Thiourea, 1% sodium deoxycholate (SDC), tributylphosphine (TBP). 3× centrifuge pulses (10,000 g) where performed at the start of each urea wash incubation. The tips were washed in 1 mL of water after the urea/thiourea washes after which 100 μL Lysis Buffer (1% SDC, 10 mM TCEP, 40 mM CLA, 100 mM Tris, pH 8). Tips were heated at 95° C. for 10 mins with gentle agitation. 1 ug (2 μL) of trypsin (0.5 μg/μL in 100 mM Tris) was then added and the solution was incubated overnight at 37° C. (19-21 hours). After incubation the tip was removed, and the remaining digest was diluted with 800 μL of 0.5% Formic acid. This solution was then centrifuged at 12,000 g for 10 mins and the supernatant was transferred to a conditioned SPE cartridge for clean-up. Peptides were then analysed using mass spectrometry.

### Results and Discussion

### Total Yield

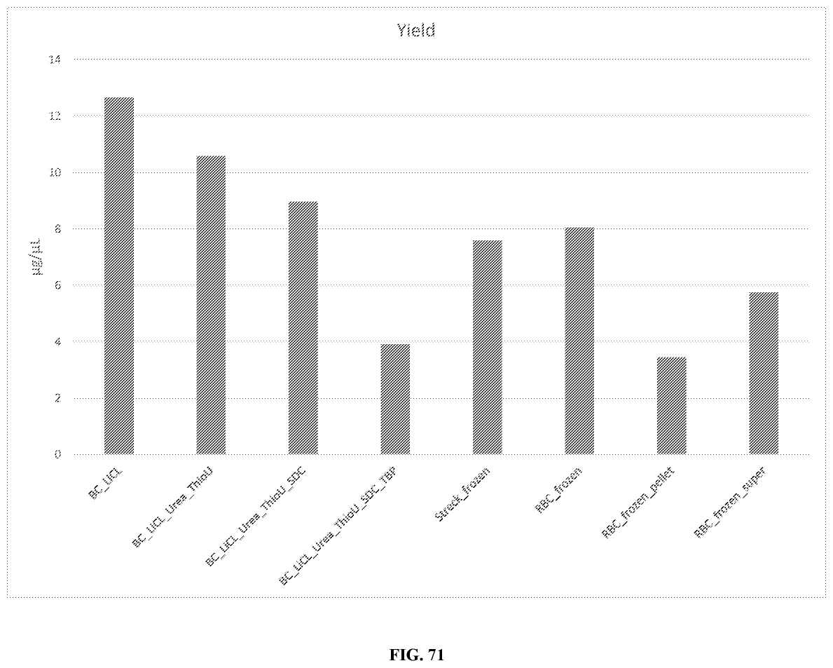

The total protein yield for the buffy coat extractions, decrease with increasing washes, which is to be expected as each wash you will remove more protein. Streck tubes and WB averaged around 30-35 μg of total protein. Total protein yield for samples are shown in FIG. 71.

### Number of Protein and Peptide IDs

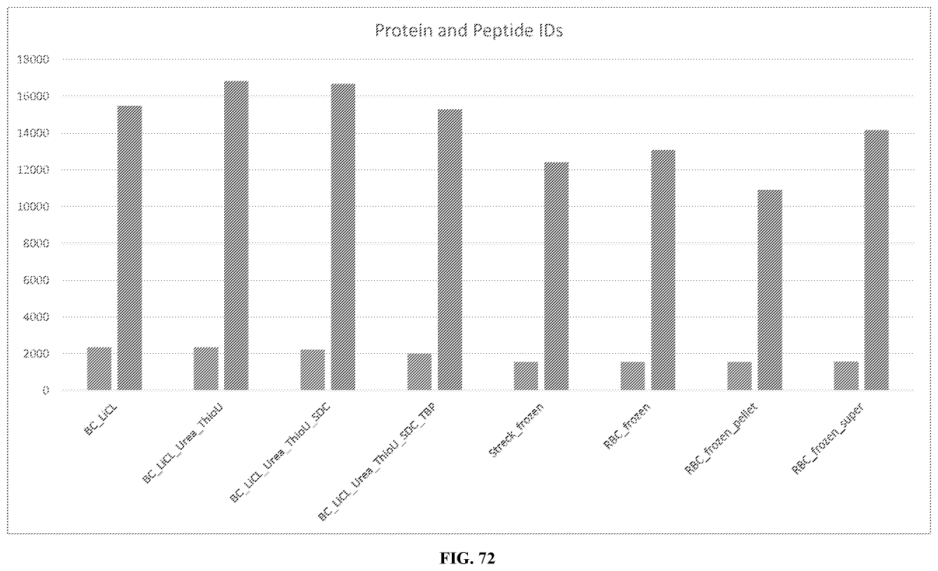

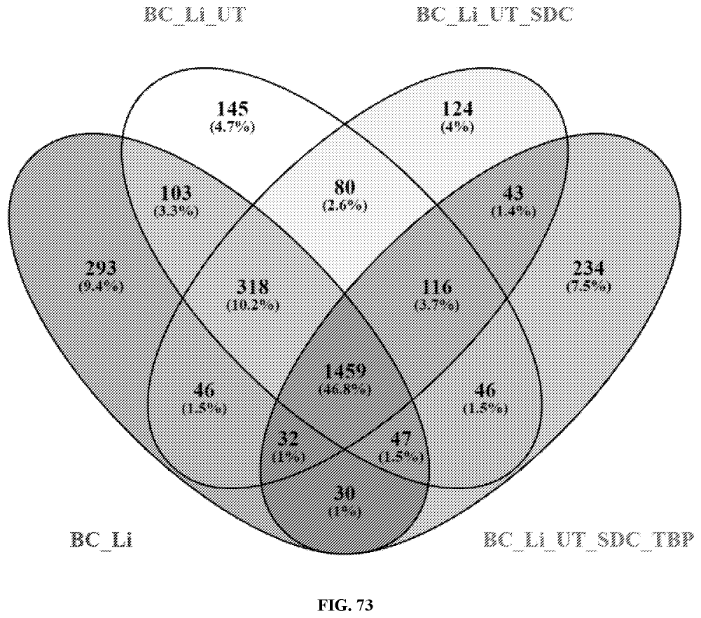

The number of proteins IDs only decreased slightly with each wash and would not be considered significant. The frozen samples also produced a similar number of IDs as we have seen previously for fresh samples. Indicating their successful utility for processing and analysis using these methods. The number of proteins IDs and peptide IDs were measured, as shown in FIG. 72.

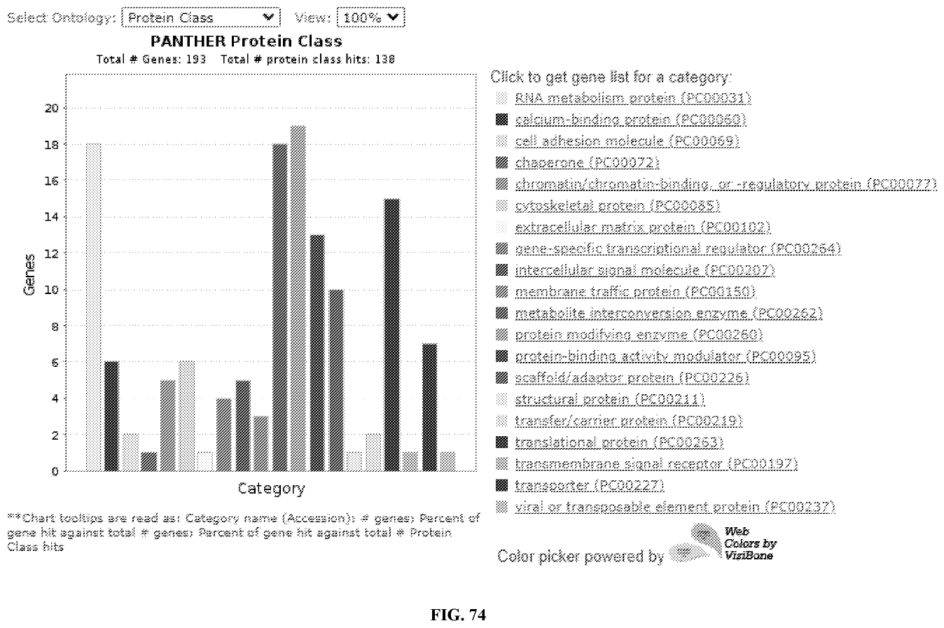

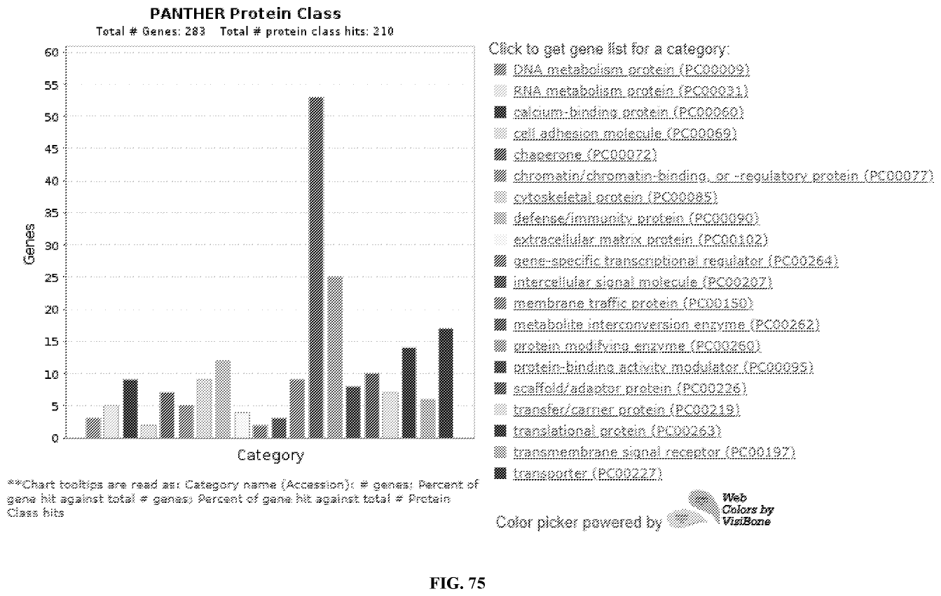

Results show that majority of proteins found in the Urea/Thiourea wash overlap with those in the LiCl wash for both the buffy coat and the whole blood tips. A higher number of unique proteins appear to be coming from the wash containing TBP. FIG. 73. illustrates the overlap of Protein ID's for Buffy Coat (BC) tips washed with LiCl; Urea/Thiourea (UT); Urea/Thiourea/SDC; Urea/Thiourea/SDC/TBP. FIG. 74 and FIG. 75 show the proteins expressed exclusively in TBP and LiCl, respectively.

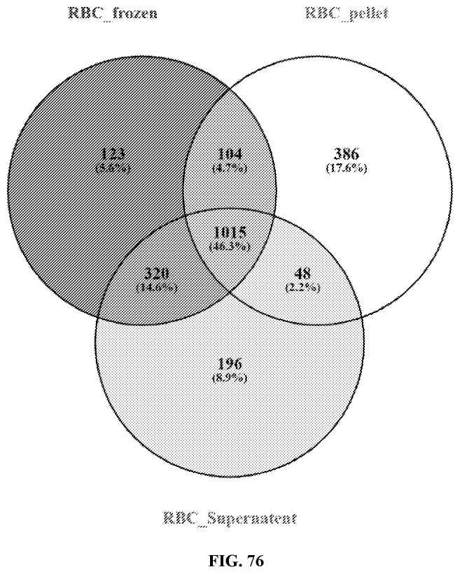

A comparison of the protein IDs for the 3 RBC fractions (RBCs, RBC pellet, and RBC supernatant), revealed the RBC pellet produced the greatest number of different IDs. FIG. 76 shows overlap of Protein IDs for RBC, RBC pellet and supernatant.

The three DIA files for the frozen RBC fractions were searched using DIA-NN. Overall there were 3588 proteins in the DIA-NN matrix. When comparing the RBC frozen pellet to the RBC frozen sample proteins 637 were down-regulated (log 2 fold change<−1.5 pval 0.05) and 343 were up-regulated (log 2 fold change >1.5 pval 0.05). A list of protein classes (PantherDB) for the up and down regulated groups are shown below.

TABLE 18
A list of protein classes (PantherDB) for the up and down
regulated groups.

| | | Number of | Number of |
|---|---|---|---|
| | | proteins up- | proteins down- |
| Comparison | Denominator | regulated | regulated |
| RBC_frozen_pellet | RBC_frozen | 343 | 637 |
| RBC_frozen_supernatant | RBC_frozen | 36 | 114 |
| RBC_frozen_pellet | RBC_frozen_ | 402 | 624 |
| | supernatant | | |

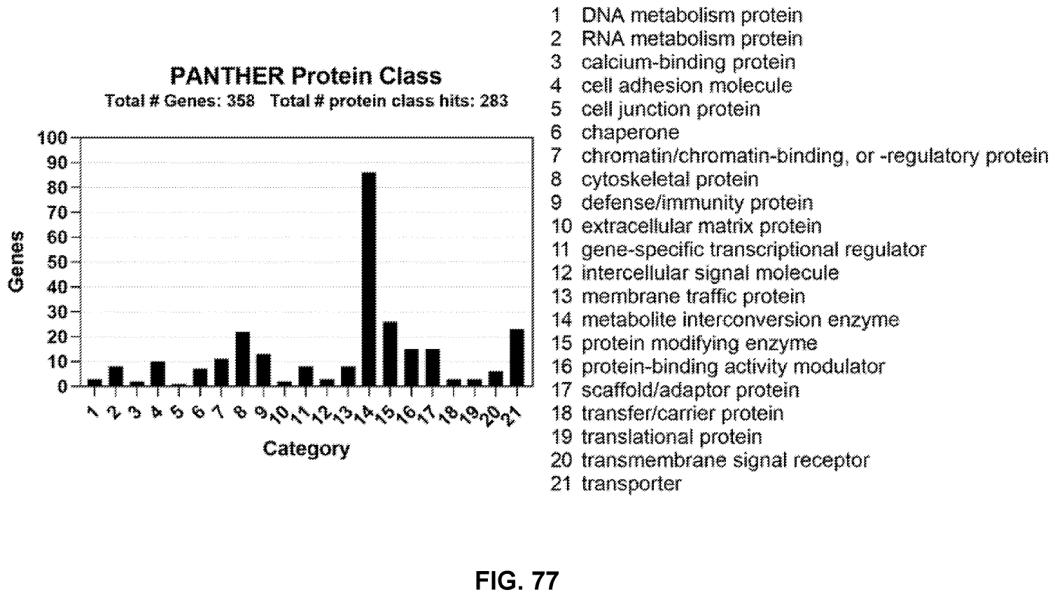

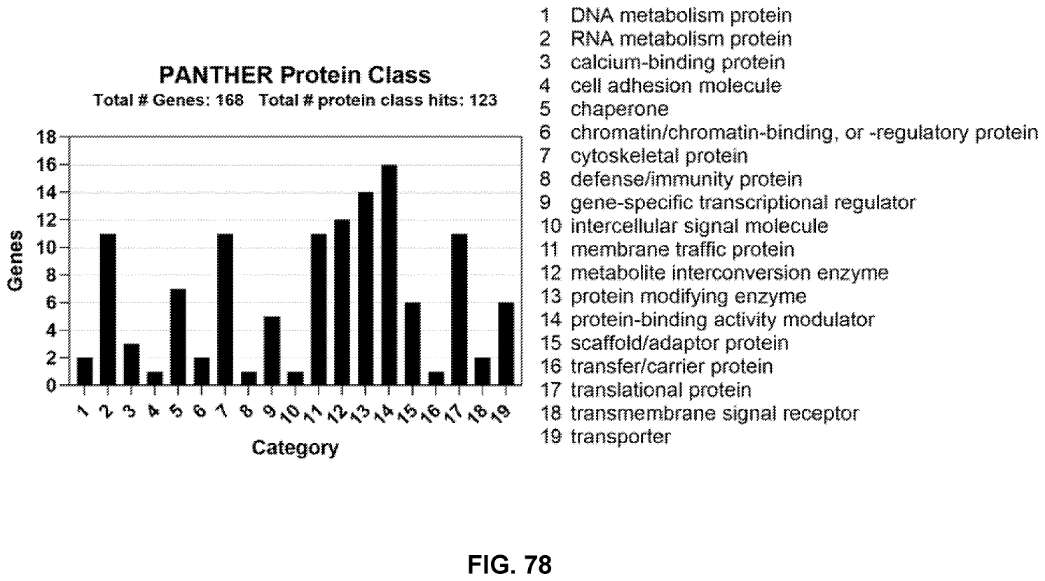

Proteins found exclusively in the RBC pellet seemed to be cell junction proteins, extracellular matrix proteins and a large number of metabolite interconversion enzymes. FIG. 77 and FIG. 78 shows Proteins found exclusively in RBC pellet and RBC supernatant, respectively.

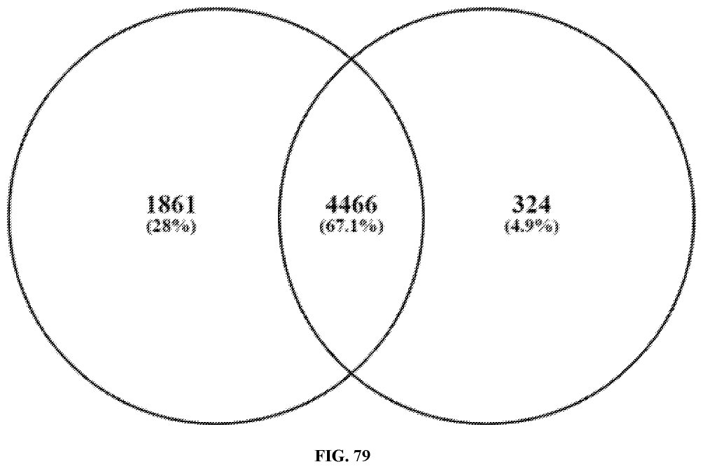

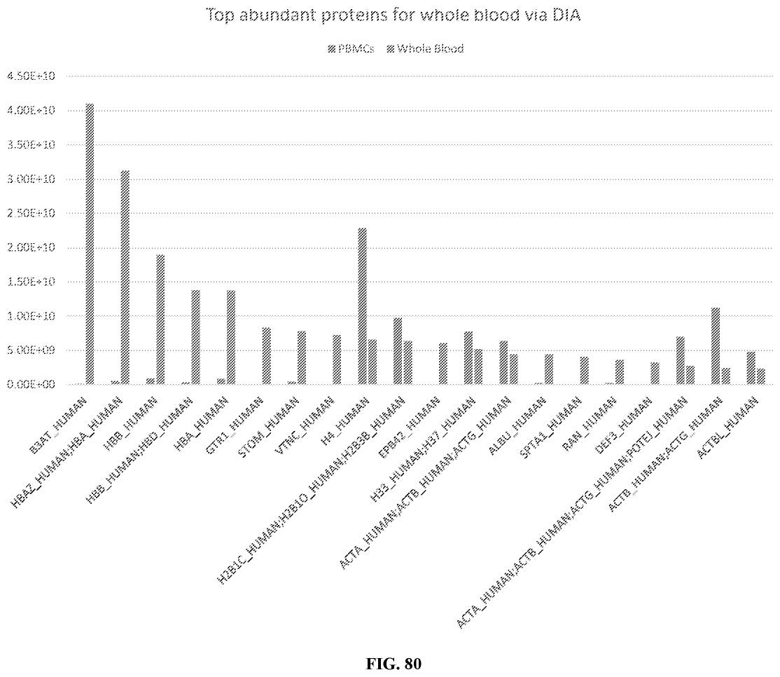

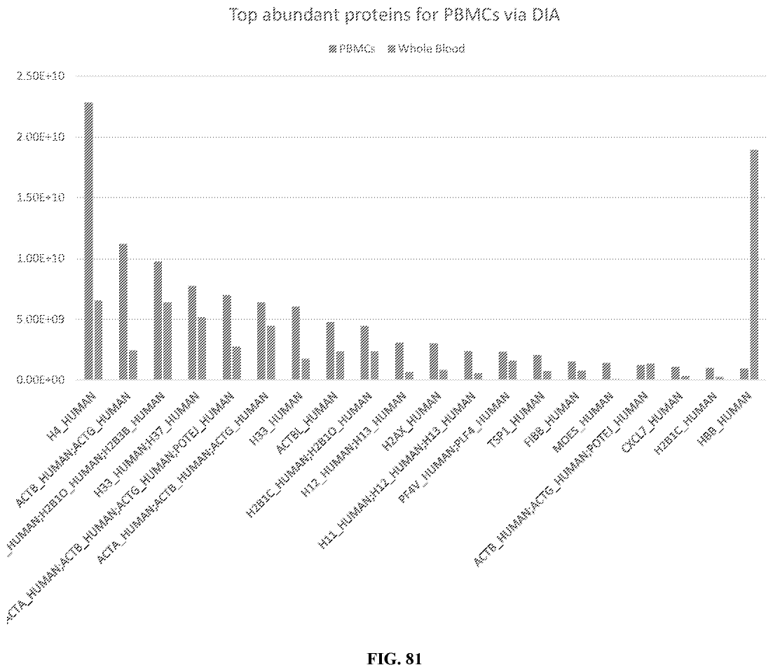

FIG. 79 is a Venn diagram showing the overlap of proteins for PBMCs and whole blood. FIG. 80 and FIG. 81 show the top abundant proteins in whole blood and PBMCs, respectively. The top 8 abundant proteins in whole blood are present in the PBMCs at very low levels. When you look at the reverse, similar levels of the top proteins in PBMCs are found in whole blood. Haemoglobin (HBB) is the 20^{th} most abundant peptide in PBMCs but the abundance is a fraction of that for the whole blood (3^{rd} most abundant).

Conclusion: Washing with the urea/thiourea on its own and with SDC produced similar profiles to that of the LiCl wash, but with a greatly reduced yield. The yield of the frozen blood was reduced (almost half that of the fresh). The most unique proteins came from the RBC pellet.

### Example X: Evaluation of Buffer Variations on WB, and Analysis of PBMCs Digested within VAMS Tips Vs Solution

Purpose: To look at different initial washing mechanisms for tips dipped in whole blood. In previous experiments we tested various washing with urea/thiourea with and without detergents and reducing agents on tips dipped in buffy coat. In this experiment we expand further using whole blood and washing with urea/thiourea/detergent (SDC)/reducing agent (TBP) and include an alkylating agent (CLA). We also look at the difference of analysing PBMCs, in tips or just in solution.

### Methods:

PBMC Preparation. PBMC tips were prepared using Vacutainer CPT Cell Preparation Tube with Sodium Heparin as per the manufacturer's instructions. Extracted PBMCs were diluted 1:3 to reach a cell count of 7×10\hat{ }3 cells/μL and were then frozen at −80° C. Frozen PBMCs were then thawed. A 30 μL aliquot was taken to digest in solution, and another aliquot was applied to a VAMS 30 μL tip and dried overnight.

Protocol Used for Overnight Digestion. Once dried, the tips were washed overnight with LiCl (1 mL) then either (a) 2× LiCl washes (with centrifugal pulses (10,000 g) or (b) 2× 30 min washes with either (b1) 1 mL of LiCl; (b2) 1 mL 7M Urea, 2M Thiourea, 1% sodium deoxycholate (SDC), tributylphosphine (TBP), 100 mM Tris; (b3) 1 mL 7M Urea, 2M Thiourea, 1% sodium deoxycholate (SDC), tributylphosphone (TBP), choloractamide (CLA). 100 mM Tris. 3× centrifuge pulses (10,000 g) were performed at the start of each urea wash incubation. The tips were washed in 1 mL of water after the urea/thiourea washes. PBMCs in solution were not washed and digestion continued as for the rest of the methods. 100 μL Lysis Buffer (1% SDC, 10 mM TCEP, 40 mM CLA, 100 mM Tris, pH 8) was added to all samples. Tips were heated at 95° C. for 10 mins with gentle agitation. 1 μg (2 μL) of trypsin (0.5 μg/μL in 100 mM Tris) was then added and the solution was incubated overnight at 37° C. (19-21 hours). After incubation the tip was removed, and the remaining digest was diluted with 800 μL of 0.5% Formic acid. This solution was then centrifuged at 12,000 g for 10 mins and the supernatant was transferred to a conditioned SPE cartridge for clean-up. Peptides were then analysed using mass spectrometry.

### Results and Discussion:

### Total Yield

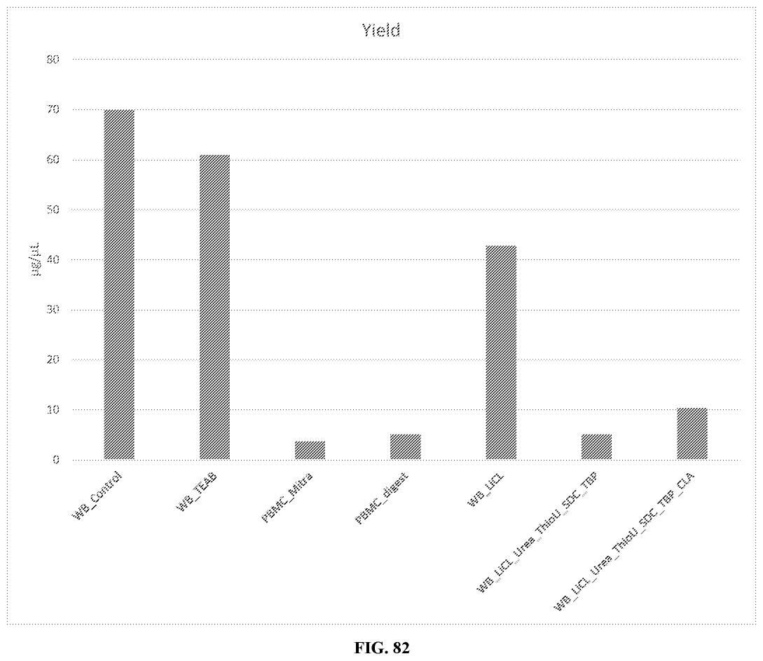

The total protein yield for the whole blood extractions decrease with increasing washes, although with the alkylating agent CLA it increased slightly. It was also interesting to note that visually when preparing the samples during the wash steps, the CLA containing washes continued to extract colour out of the tip in each consecutive wash, which does not occur with the LiCl washes and only happened on the first wash with TBP only. The yield for the washed PBMC in the Mitra® tip is only marginally lower than the PBMC not in the tip. TEAB buffer produced a slightly lower yield than Tris, but not significant. Total protein yield for samples is shown in FIG. 82.

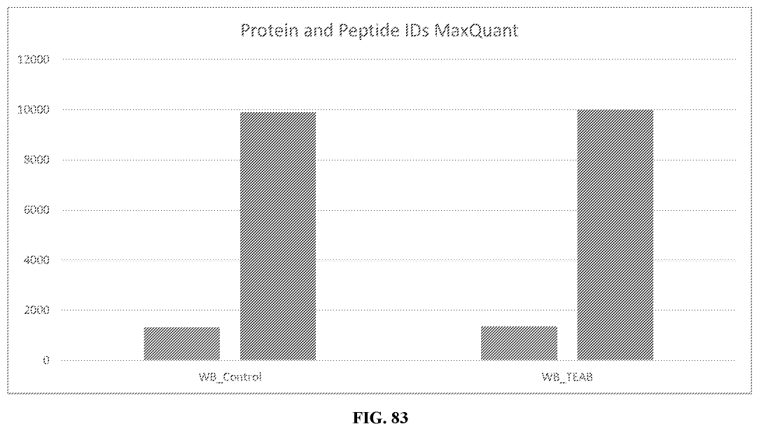

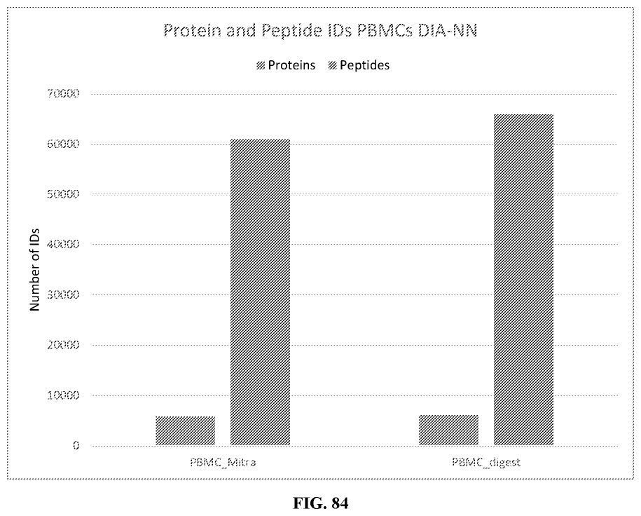

The TEAB buffer and Tris buffer performed very similarly in regards to protein and peptide IDs as well as trypsin digest efficiency. FIG. 83 shows protein and peptide IDs for the substitution of TEAB and Tris.

### PBMCs in Solution Vs PBMCs in VAMS

A comparison of PBMCs in solution with PBMCs collected into VAMS was performed. Overall, there were 7006 proteins in the DIA-NN matrix with more IDs from the cells in solution compared to the Mitra® sample. 622 proteins were down-regulated (log 2 fold change<−1.5 pval 0.05) and 136 were up-regulated (log 2 fold change>1.5 pval 0.05). A list of protein classes (PantherDB) for the up and down regulated groups are shown below. Thus, the additional step of adding the cells to VAMS and processing through multiple washes and extractions, we were able to collect a new subset of proteins otherwise lost with standard methods. FIG. 84 shows protein and peptide IDs from DIA-NN for PBMCs digested directly in solution or digested in-tip following sequential extraction.

TABLE 19
Top 10 proteins down-regulated from PBMCs in VAMS.

| | Mapped | | | Fold |
|---|---|---|---|---|
| Gene ID | ID | Gene Name | Protein Class | Change |
| HUMAN\|HGNC = 543\| | P08758 | Annexin | calcium-binding | −3.9 |
| UniProtKB = P08758 | | A5; ANXA5; ortholog | protein(PC00060) | |
| HUMAN\|HGNC = 12013\| | P67936 | Tropomyosin alpha-4 | actin binding motor | −3.9 |
| UniProtKB = P67936 | | chain; TPM4; ortholog | protein(PC00040) | |
| HUMAN\|HGNC = 12010\| | P09493 | Tropomyosin alpha-1 | actin binding motor | −4.1 |
| UniProtKB = P09493 | | chain; TPM1; ortholog | protein(PC00040) | |
| HUMAN\|HGNC = 12009\| | P60174 | Triosephosphate | isomerase(PC00135) | −3.9 |
| UniProtKB = P60174 | | isomerase; TPI1; | | |
| | | ortholog | | |
| HUMAN\|HGNC = 7097\| | P14174 | Macrophage migration | decarboxylase(PC00089) | −3.7 |
| UniProtKB = P14174 | | inhibitory | | |
| | | factor; MIF; ortholog | | |
| HUMAN\|HGNC = 3711\| | P62942 | Peptidyl-prolyl cis- | chaperone(PC00072) | −3.8 |
| UniProtKB = P62942 | | trans isomerase | | |
| | | FKBPIA; FKBP1A; | | |
| | | ortholog | | |
| HUMAN\|HGNC-8630\| | P30086 | Phosphatidylethanolamine- | protease | −3.7 |
| UniProtKB = P30086 | | binding protein | inhibitor(PC00191) | |
| | | 1; PEBP1; ortholog | | |
| HUMAN\|HGNC = 10499\| | P06702 | Protein S100- | calmodulin- | −4.1 |
| UniProtKB = P06702 | | A9; S100A9; ortholog | related(PC00061) | |
| HUMAN\|HGNC = 5526\| | P01859 | Immunoglobulin | immunoglobulin | −3.4 |
| UniProtKB = P01859 | | heavy constant gamma | receptor | |
| | | 2; IGHG2; ortholog | superfamily(PC00124) | |
| HUMAN\|HGNC = 4638\| | P09211 | Glutathione S- | transferase(PC00220) | −3.7 |
| UniProtKB = P09211 | | transferase | | |
| | | P; GSTP1; ortholog | | |

TABLE 20
Top 10 proteins up-regulated from PBMCs in VAMS.

| | Mapped | | | Fold |
|---|---|---|---|---|
| Gene ID | ID | Gene Name | Protein Class | Change |
| HUMAN\|HGNC = 957\| | P80723 | Brain acid soluble protein | | 3.0 |
| UniProtKB = P80723 | | 1; BASP1; ortholog | | |
| HUMAN\|HGNC = 29810\| | Q99549 | M-phase phosphoprotein | scaffold/adaptor | 2.8 |
| UniProtKB = Q99549 | | 8; MPHOSPH8; ortholog | protein(PC00226) | |
| HUMAN\|HGNC = 6444\| | P04259 | Keratin, type II cytoskeletal | | 2.7 |
| UniProtKB = P04259 | | 6B; KRT6B; ortholog | | |
| HUMAN\|HGNC = 8920\| | O75151 | Lysine-specific | histone modifying | 2.9 |
| UniProtKB = O75151 | | demethylase | enzyme(PC00261) | |
| | | PHF2; PHF2; ortholog | | |
| HUMAN\|HGNC = 6207\| | P14923 | Junction | | 2.8 |
| UniProtKB = P14923 | | plakoglobin; JUP; ortholog | | |
| HUMAN\|HGNC = 11005\| | P11166 | Solute carrier family 2, | secondary carrier | 3.4 |
| UniProtKB = P11166 | | facilitated glucose | transporter | |
| | | transporter member | (PC00258) | |
| | | 1; SLC2A1; ortholog | | |
| HUMAN\|HGNC = 6881\| | P45983 | Mitogen-activated protein | non-receptor | 2.8 |
| UniProtKB = P45983 | | kinase 8; MAPK8; ortholog | serine/threonine | |
| | | | protein | |
| | | | kinase(PC00167) | |
| HUMAN\|HGNC = 11027 | P02730 | Band 3 anion transport | secondary carrier | 3.6 |
| \|UniProtKB = P02730 | | protein; SLC4A1; ortholog | transporter | |
| | | | (PC00258) | |
| HUMAN\|HGNC = 26633 | Q7Z7L1 | Schlafen family member | endoribonuclease | 2.8 |
| \|UniProtKB = Q7Z7L1 | | 11; SLFN11; ortholog | (PC00094) | |
| HUMAN\|HGNC = 957\| | P80723 | Brain acid soluble protein | | 3.0 |
| UniProtKB = P80723 | | 1; BASP1; ortholog | | |

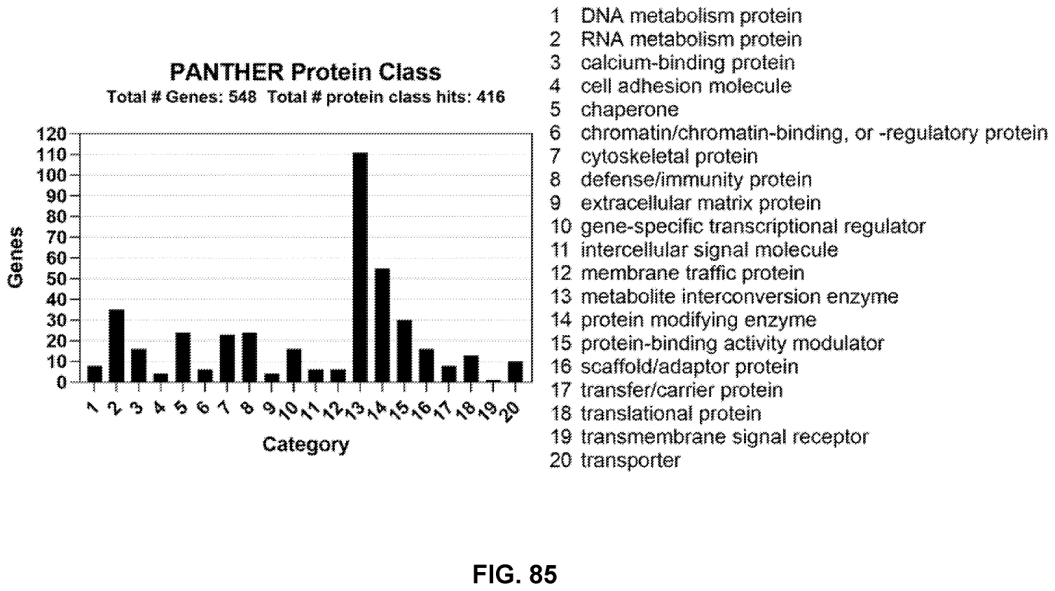

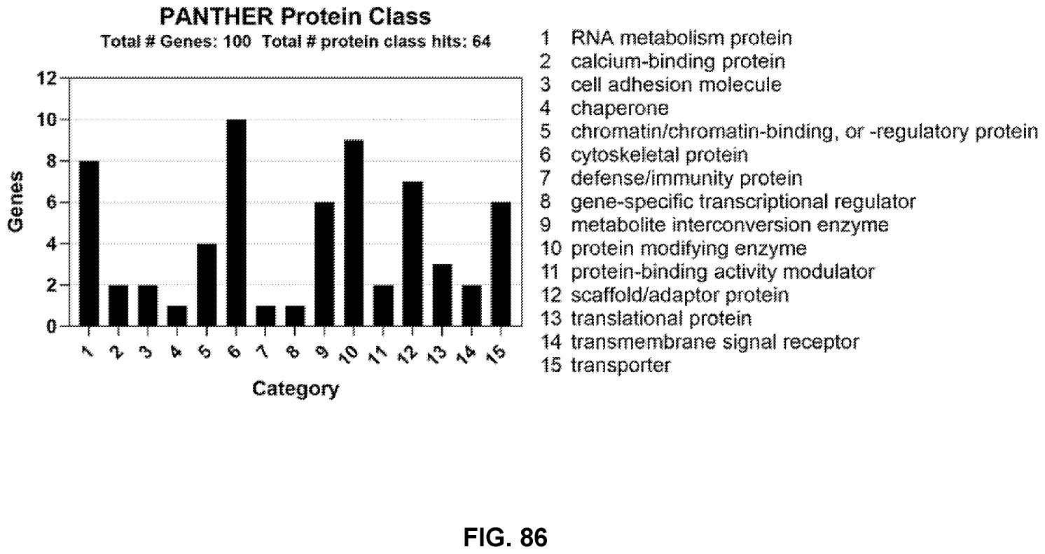

FIG. 85 shows a summary of proteins down-regulated from PBMCs in VAMS. FIG. 86 Shows a summary of proteins up-regulated from PBMCs in VAMS.

Washing of Tips with Urea/Thio-Urea Combinations

The slight variation on washing with LiCl (centrifuge pulses vs 2×30 min incubations) did appear to give higher number of protein and peptide IDs. The WB sample washing with the addition of alkylating agent CLA (SB044) gave an increase in peptides also.

The six DIA files for the different washing conditions were searched using DIA-NN. Overall, there were 5091 proteins in the DIA-NN matrix. The below table shows the number of up and down regulated proteins, when comparing to the control (LiCl).

TABLE 21
Number of up-regulated proteins and down-regulated proteins from
various conditions compared to the standard method (LiCl).

| | | Number of proteins | Number of proteins |
|---|---|---|---|
| Comparison | Denominator | up-regulated | down-regulated |
| LiCl(30) | LiCl | 108 | 81 |
| TBP | LiCl | 321 | 392 |
| CLA | LiCl | 272 | 244 |

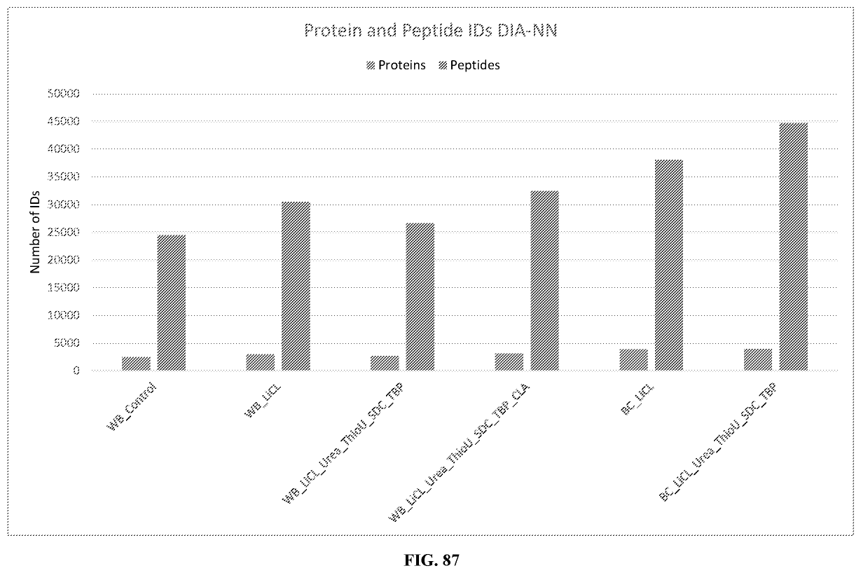

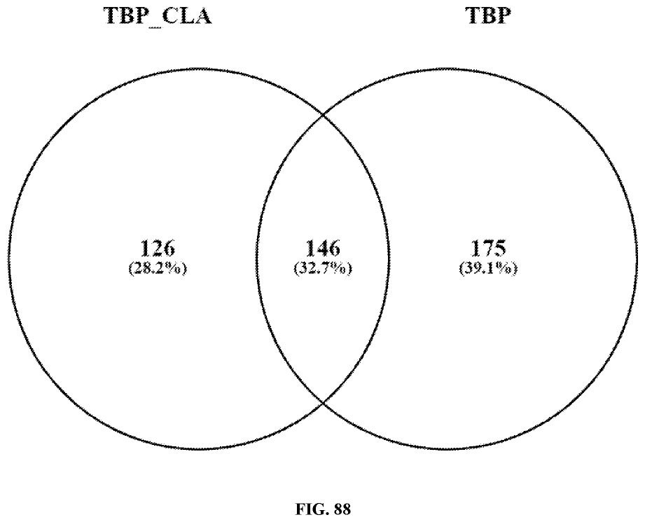

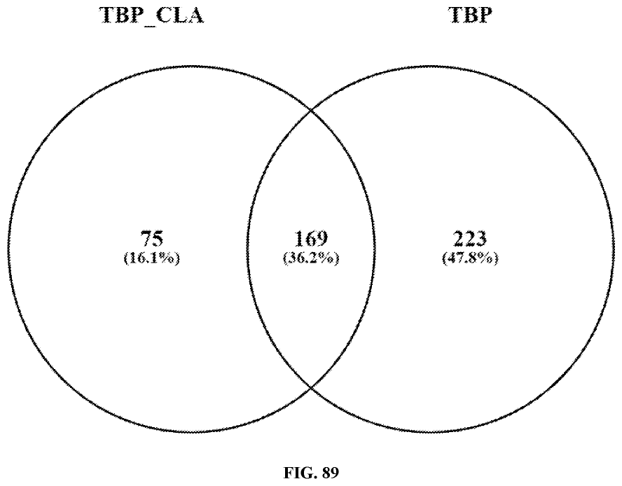

FIG. 87 shows protein and peptide IDs from DIA-NN for samples subjected to different washing conditions. FIG. 88 and FIG. 89 are Venn diagrams displaying the overlap of upregulated and down regulated proteins, respectively, in TBP and CLA.

### Example XI: Evaluation of Samples Obtained from Healthy and Diseased Subjects

Aim: To analyse a series of frozen samples from cancer patients and matching healthy controls.

### Methods

Sample preparation. Frozen whole blood cell pellets were thawed and diluted 1:2 in PBS. Diluted samples were applied to 30 μL VAMS tips which were left to dry for 24 hrs.

Protocol used for overnight digestion. Once dried, the tips were washed overnight with LiCl (1 mL) then 2× LiCl washes with centrifuge pulses (10,000 g). 100 μL Lysis Buffer (1% SDC, 10 mM TCEP, 40 mM CLA, 100 mM TEAB, pH 8) was added to all samples. Tips were heated at 95° C. for 10 mins with gentle agitation. 1 μg (2 μL) of trypsin (0.5 μg/μL in 100 mM TEAB) was then added and the solution was incubated overnight at 37° C. (19-21 hours). After incubation the tip was removed, and the remaining digest was diluted with 800 μL of 0.5% Formic acid. This solution was then centrifuged at 12,000 g for 10 mins and the supernatant was transferred to a conditioned SPE cartridge for clean-up. Peptides were then analysed using mass spectrometry.

### Results

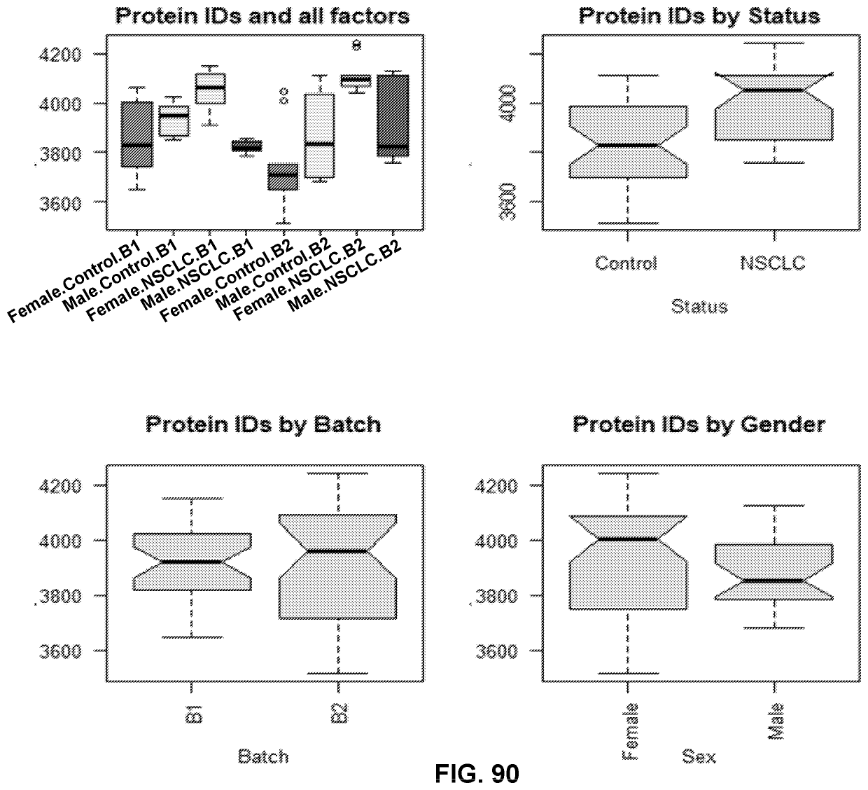

Overall, there were slightly more quantitative values in the cancer samples, especially the female ones. There is no strong apparent correlation of protein quantitation number with age or sample age. FIG. 90 shows protein quantitation views for the various samples.

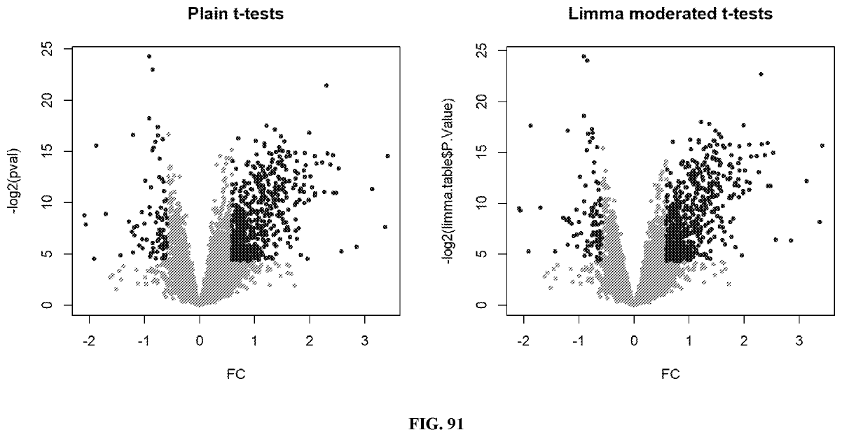

Using standard criteria (FDR adjusted p-values <0.05) there are a large number of differentially expressed proteins. FIG. 91 shows volcano plot depicting differential expression.

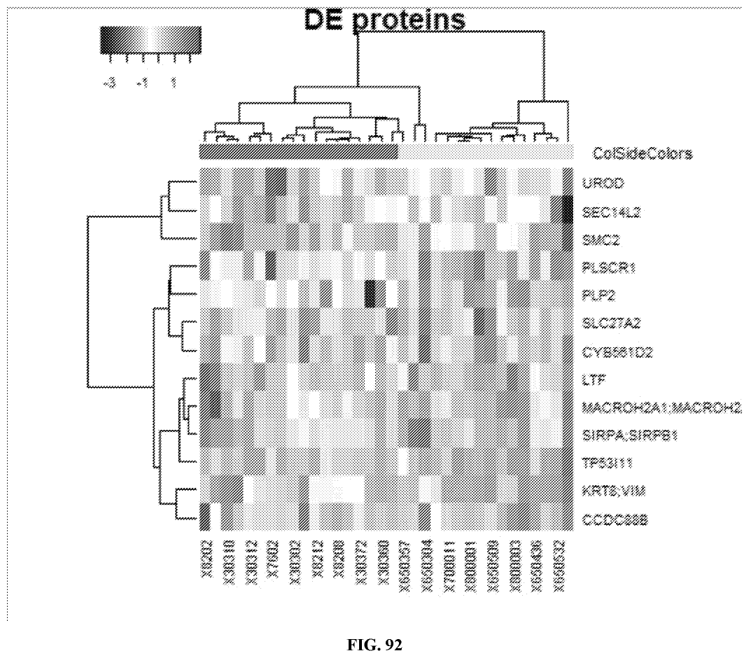

It was also found that there were a subset of female cancer samples that are very well distinguished from the rest. 508 differentially expressed proteins were identified, and applying filters to the data revealed a set of 13 differentially expressed proteins that were highly predictive of disease status. FIG. 92 shows a heatmap of selected 13 differentially expressed proteins that clustered well with each participant group.

### Example XII: Evaluation of Intra- and Inter-Assay Reproducibility of Methods

Aim: To assess reproducibility of the sample preparation, extraction, and digest methods across replicate DBS samples using mass spectrometry (intra-tip reproducibility). Intra-assay reproducibility was assessed by analysing a single sample 3 consecutive times.

### Methods

### Sample Preparation

A total of ten VAMS 30 μL tips were dipped into whole blood and dried for a minimum of 24 hrs. The tips were prepped in 2 batches, 1 week apart.

### Protocol Used for Overnight Digestion

Once dried, the tips were washed overnight with LiCl (1 mL) then 2× LiCl washes with centrifuge pulses (10,000 g). 100 μL Lysis Buffer (1% SDC, 10 mM TCEP, 40 mM CLA, 100 mM TEAB, pH 8) was added to all samples. Tips were heated at 95° C. for 10 mins with gentle agitation. 1 μg (2 μL) of trypsin (0.5 μg/μL in 100 mM TEAB) was then added and the solution was incubated overnight at 37° C. (19-21 hours). After incubation the tip was removed, and the remaining digest was diluted with 800 μL of 0.5% Formic acid. This solution was then centrifuged at 12,000 g for 10 mins and the supernatant was transferred to a conditioned SPE cartridge for clean-up. Peptides were then analysed using mass spectrometry.

### Results

### Total Yield

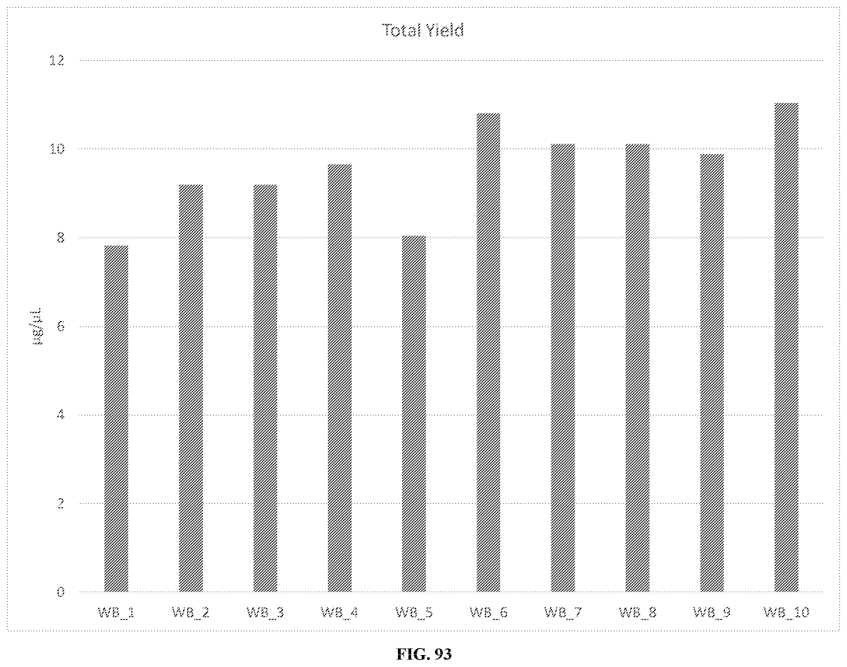

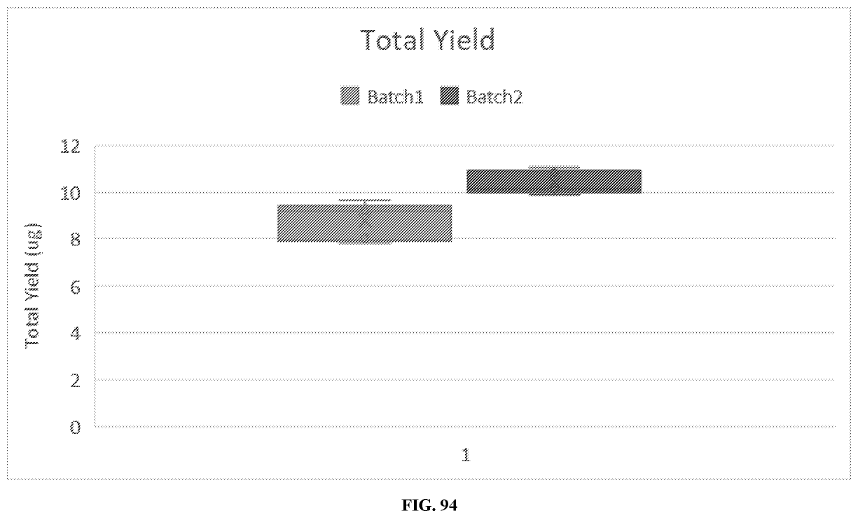

Overall, the reproducibility in yield across the tips is very good. FIG. 93 shows protein yield for individual tip replicates, FIG. 94 shows total yield between batches.

The results of the yield from the two batches is statistically significant (t-test analysis). This indicates that the length of time for drying does impact the protein yield recovered.

### DIA-NN Analysis

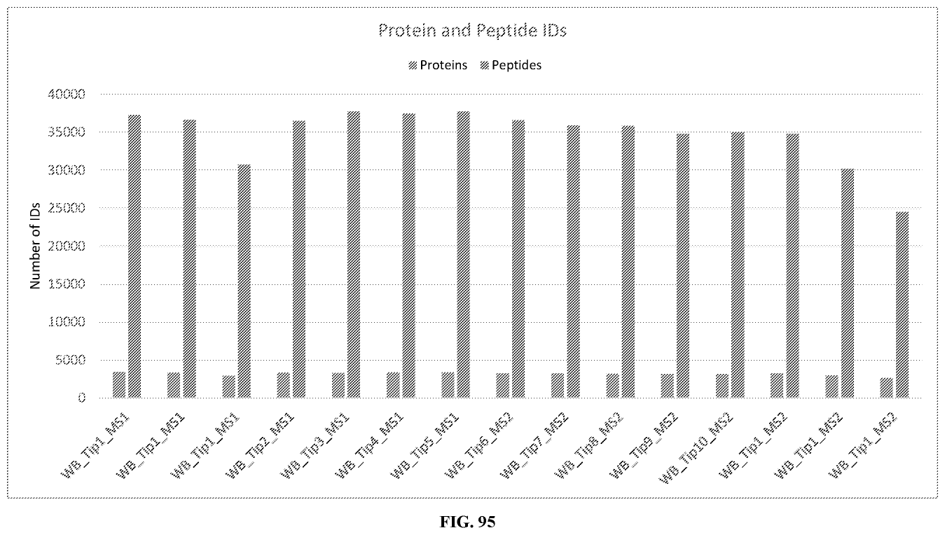

All 15 DIA files (from both batches) were searched using DIA-NN. Overall, the DIA-NN matrix contained a total of 4481 proteins and 49634 peptides. They all showed a very similar number of protein and peptide IDs, average protein IDs 3206 and peptide IDs 34784. The replicate sample injections of WB_Tip1 shows a drop in peptide IDs in the third injection. This could be due to injection issues, as all injections come from the same well. In the case of the third injection on MS, the drop is considered significant and could potentially be removed as an outlier. FIG. 95 shows total protein and peptide IDs from DIA-NN.

### Digestion Efficacy

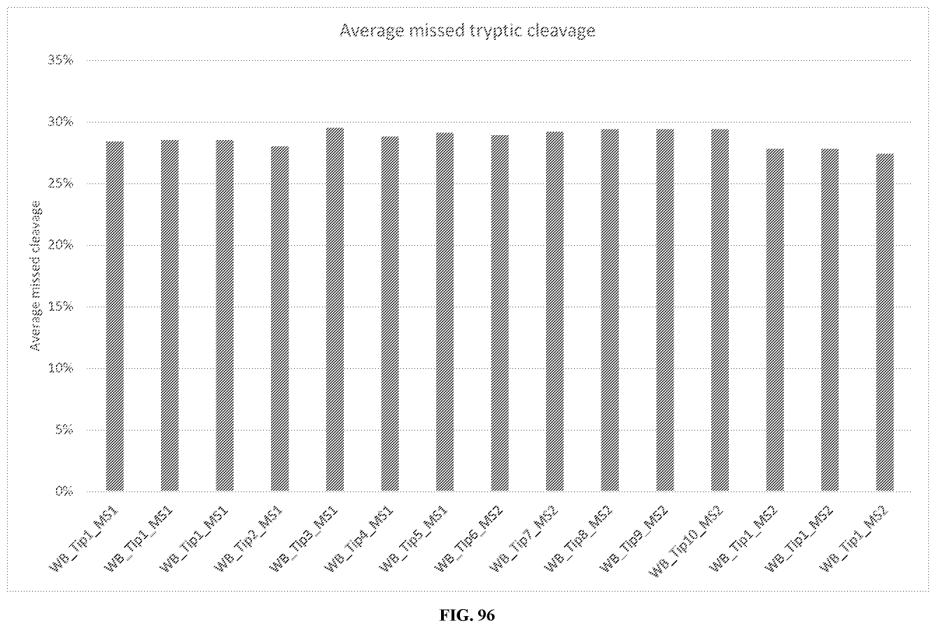

Digestion efficiency was consistent across the batches with an average missed cleavage value of 29%. FIG. 96 shows average missed cleavages for individual tip replicates.

### Intra- and Inter-Tip Variation

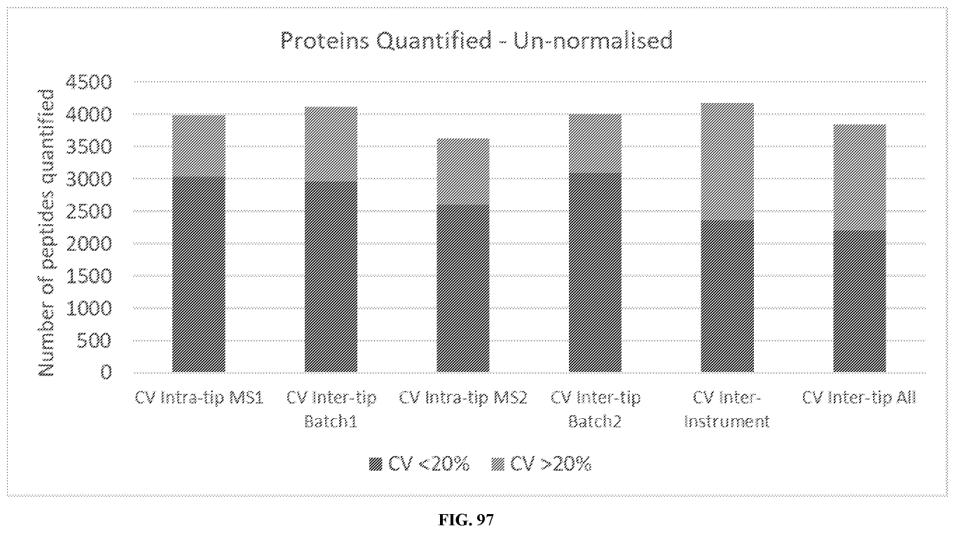

Co-efficient of variation (CV) was calculated from raw peptide and protein intensities (DIA-NN output). Across a single instrument Intra-tip CVs (n=5) were similar for both Batch 1 and Batch 2, quantifying 2962 and 3088 proteins respectively at 20% CV. For the total set of 10 tips, across two mass specs a total of 2200 proteins and 8884 peptides were quantified with a CV <20%. The biggest variation came from the mass spectrometer, rather than the sample preparation & VAMS devices. FIG. 97 shows the number of proteins with % CV of <20% (bottom) and % CV of >20% (top) from analytical replicates using the same tip (intra-tip MS1), tips from each batch (inter-tip Batch 1 or 2), tips from Batch 1 re-analysed when Batch 2 were analysed (intra-tip MS2), all tips on each instrument (inter-instrument) and finally, all tips together (inter-tip all).

Intra- and inter-tip protein median CVs are below 20% which is the industry standard. Comparing data across two mass specs without normalisation gave median % CVs below 30%.

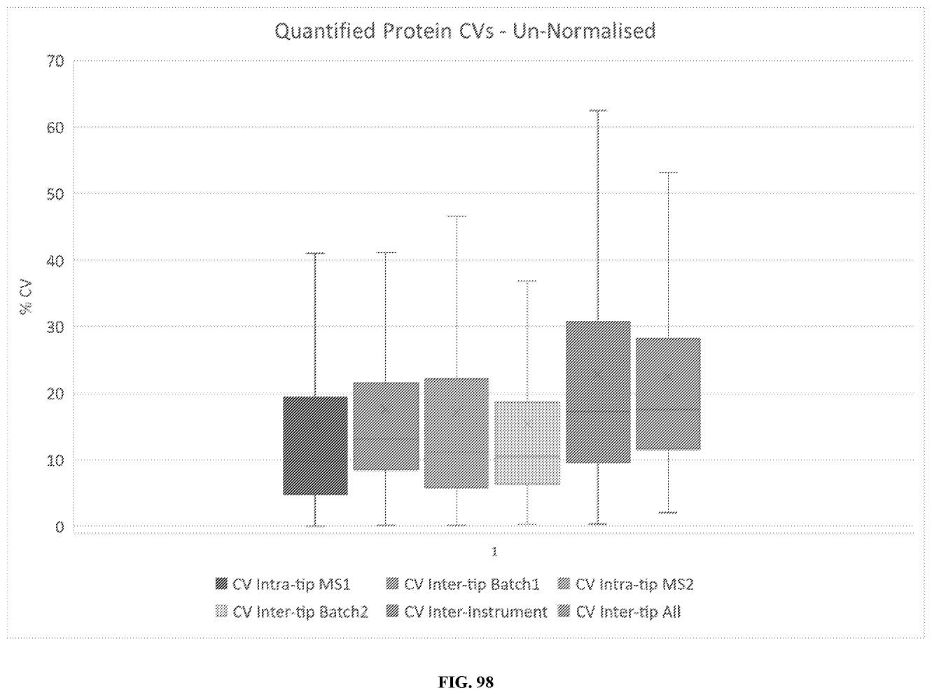

FIG. 98 shows a box and whiskers plot of % CV from analytical replicates using the same tip (intra-tip MS1), tips from each batch (inter-tip Batch 1 or 2), tips from Batch 1 re-analysed when Batch 2 were analysed (intra-tip MS2), all tips on each instrument (inter-instrument) and finally, all tips together (inter-tip all).

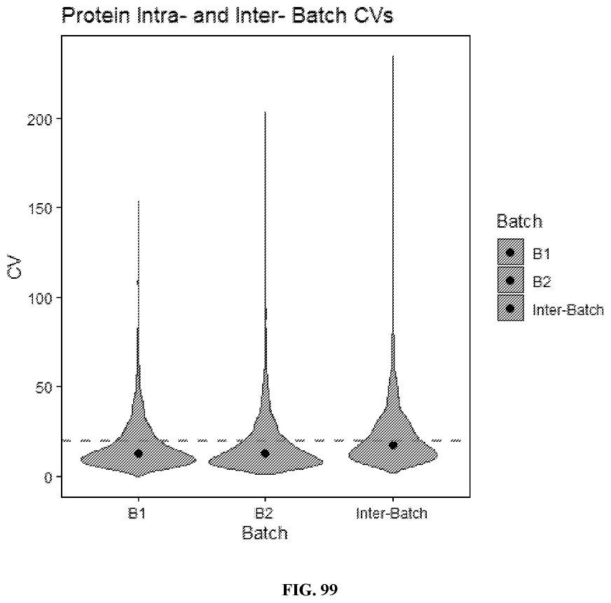

Violin plot shows the distribution of % CVs within each batch (intra) and across the two batches (inter). FIG. 99 shows violin plot of % CV for each batch (B1 and B2) and both batches together (intra-batch) left to right. Dotted line marks 20% CV. Dot marks the median % CV.

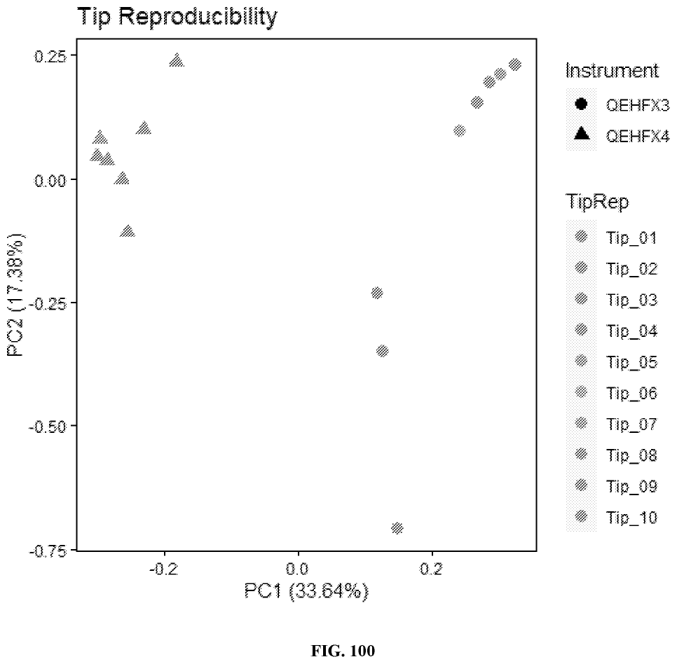

With PCA analysis, data clusters by mass spectrometer rather than by preparation batch or tip variation. This further indicates that the instrument contributes the most to data variability. FIG. 100 shows PCA plot of tip and batch reproducibility.

### Comparison to Literature

Comparing these % CVs to those of those of the literature revealed that our data is in line with other methods (Strategies to enable large-scale proteomics for reproducible research, R. Poulos et. al. Nature Communications volume 11, Article number: 3793 (2020)).

### Conclusions

Most of the observed variation in the runs came from the mass spectrometer itself (intra-tip) and inter-instrument, with only a small variation added when compared to the sample preparation/VAMS (intra-tip). Overall median % CVs for the individual batches run on one instrument. The whole set across two instruments % CVs are still below 30% without normalisation which is acceptable.

### Example XIII. Assessment of Alternative Drying/Storage Methods

Aim: To assess the effect of drying at room temperature on protein yield and extraction.

### Methods: Sample Preparation

Six VAMS 30 μL tips were dipped into whole blood. Half of the tips were left to dry with desiccant at room temperature for a week, whilst the other half were left for 5 mins, then transferred to Eppendorf tubes and frozen at −80° C. for a week. Over that time, samples were removed from the freezer, left to come to room temperature then refrozen, for a total of three times.

Protocol used for overnight digestion. Following the one-week period of drying and/or storage, the dried tips and frozen tips were washed overnight with LiCl (1 mL) then 2× LiCl washes with centrifuge pulses (10,000 g). 100 μL Lysis Buffer (1% SDC, 10 mM TCEP, 40 mM CLA, 100 mM TEAB, pH 8) was added to all samples. Tips were heated at 95° C. for 10 mins with gentle agitation. 1 μg (2 L) of trypsin (0.5 μg/μL in 100 mM TEAB) was then added and the solution was incubated overnight at 37° C. (19-21 hours). After incubation the tip was removed, and the remaining digest was diluted with 800 μL of 0.5% Formic acid. This solution was then centrifuged at 12,000 g for 10 mins and the supernatant was transferred to a conditioned SPE cartridge for clean-up. Peptides were then analysed using mass spectrometry.

### Results

### Total Yield

Without wishing to be bound by theory, the results of total yield support the hypothesis, that samples which are not left to dry, will not form a protein gel in the tip and hence more protein should be removed via the washing steps, resulting in a lower overall total yield. It was also visually apparent (via lack of colour in the tips post-washing) that more haemoglobin had washed out.

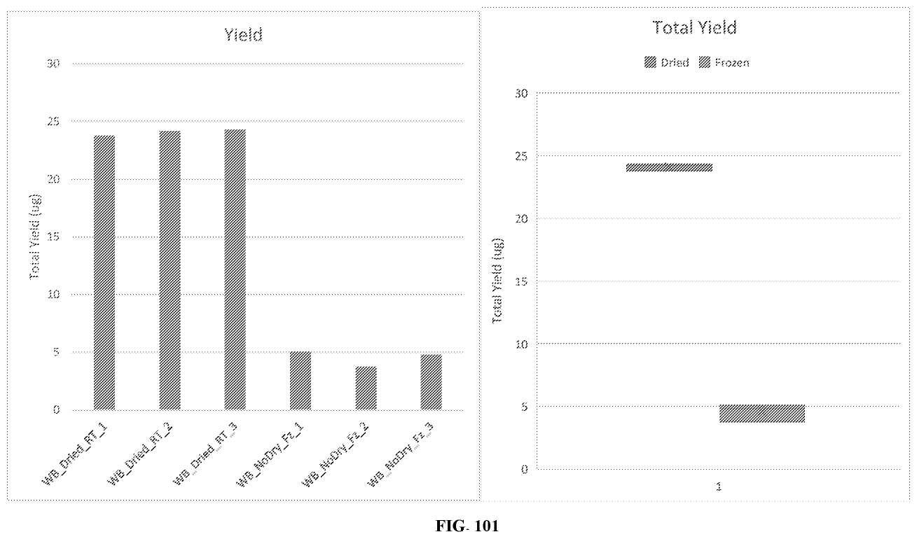

FIG. 101 shows total yield from DBS allowed to dry at room temperature for a week (dried) and from DBS prevented from drying by freezing and cycling through 3× freeze thaws.

### DIA-NN Analysis

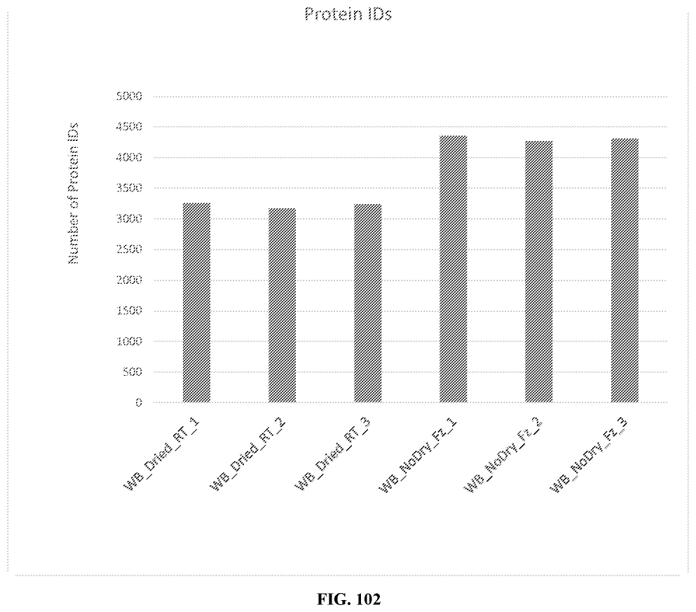

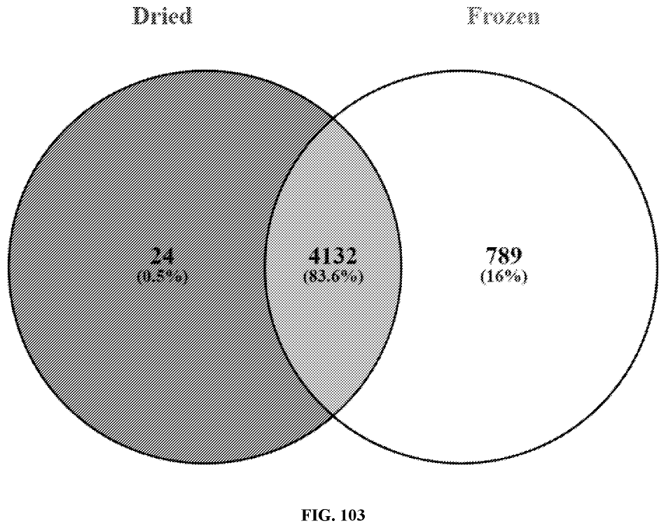

The dried sample gave an average of 3200 proteins compared with 4300 in the frozen. FIG. 102 shows protein IDs from DIA-NN for dried vs frozen DBS. FIG. 103 is a Venn diagram showing the overlap of protein IDs from dried vs frozen VAMS.

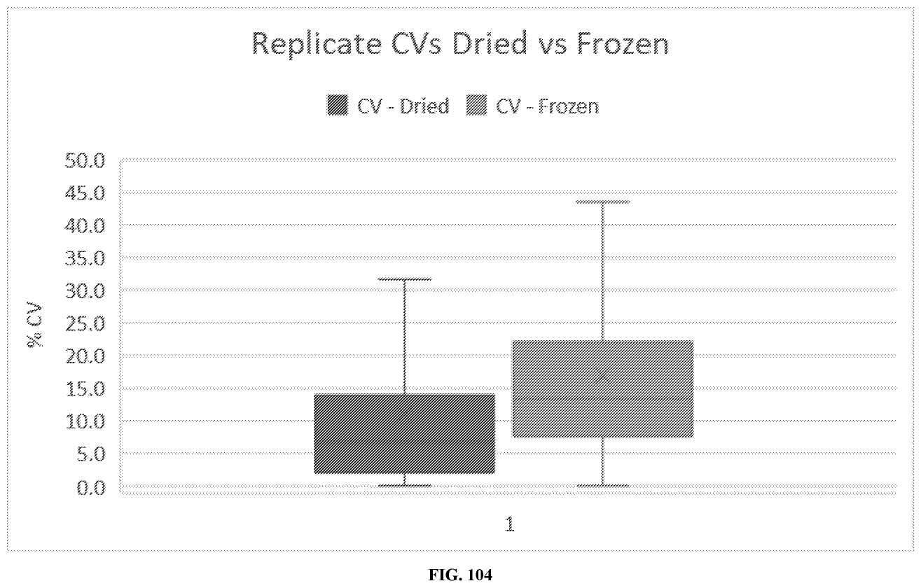

From analysis of the % CVs, it appears that the dried tips were more reproducible than the frozen tips. FIG. 104 shows % CVs of replicates in dried DBS vs frozen DBS.

The top most abundant proteins were heavily reduced in the frozen sample compared to the dried, especially in the case of haemoglobin alpha and beta subunits.

Analysis of the most commonly found blood proteins, the frozen sample actually contains higher amount in every instance with the exception of albumin and transthyretin.

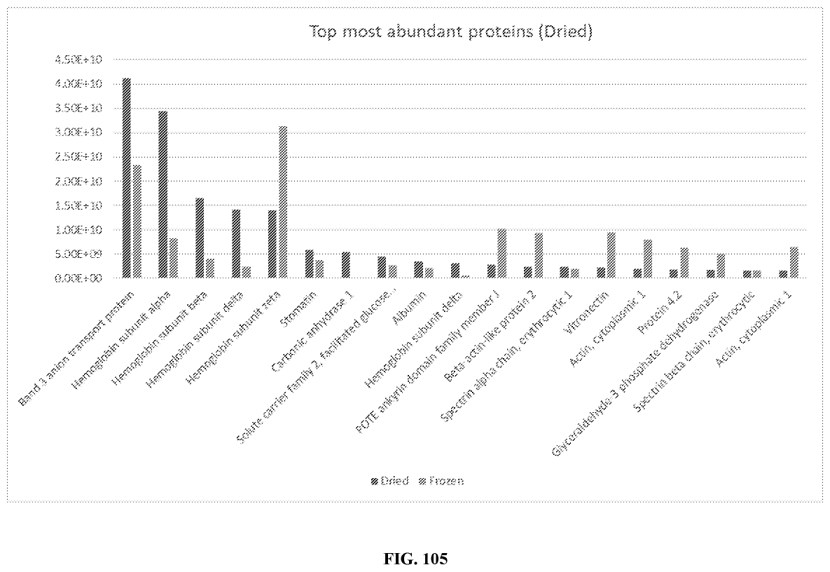

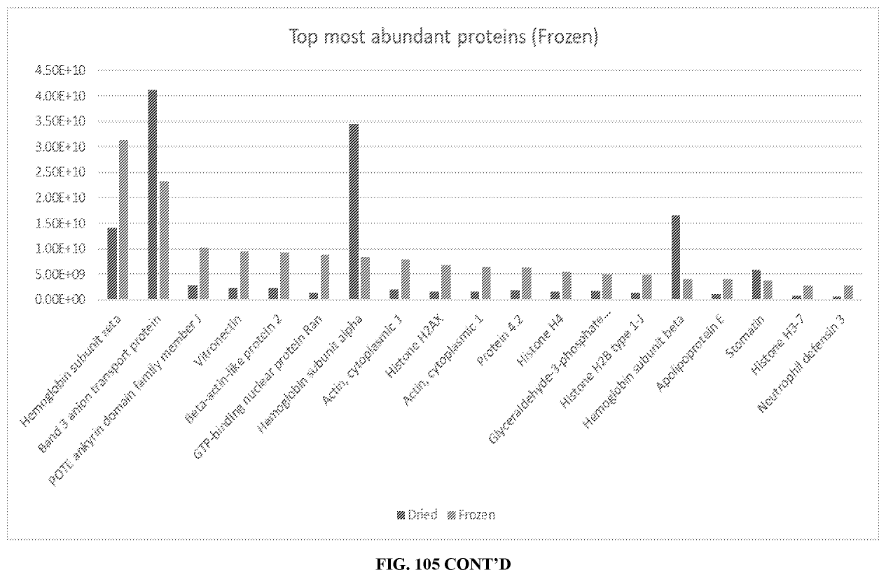

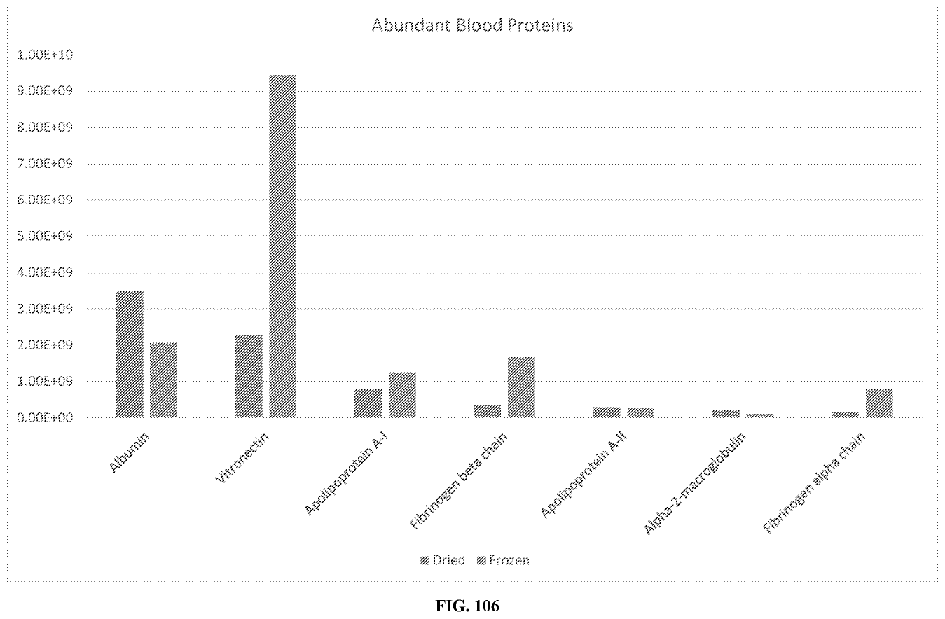

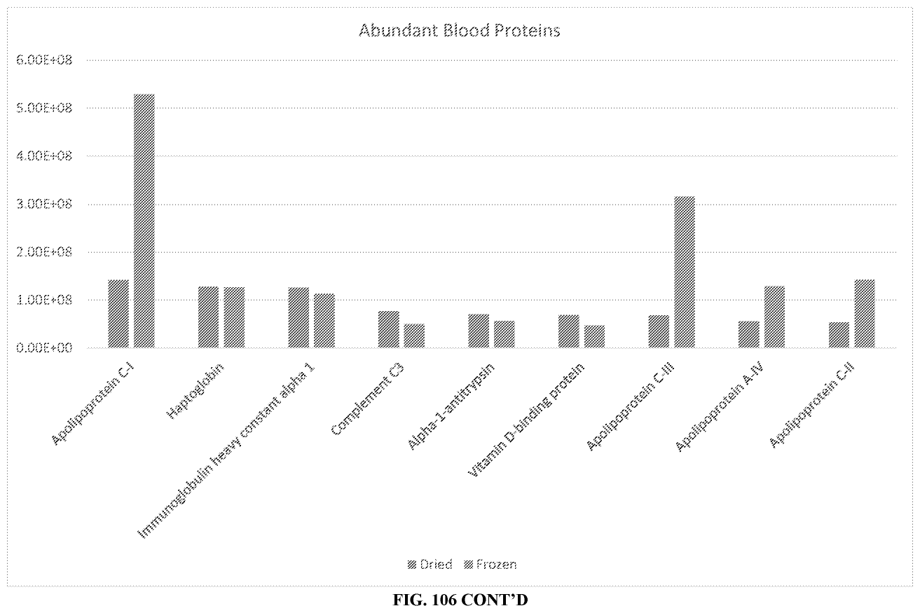

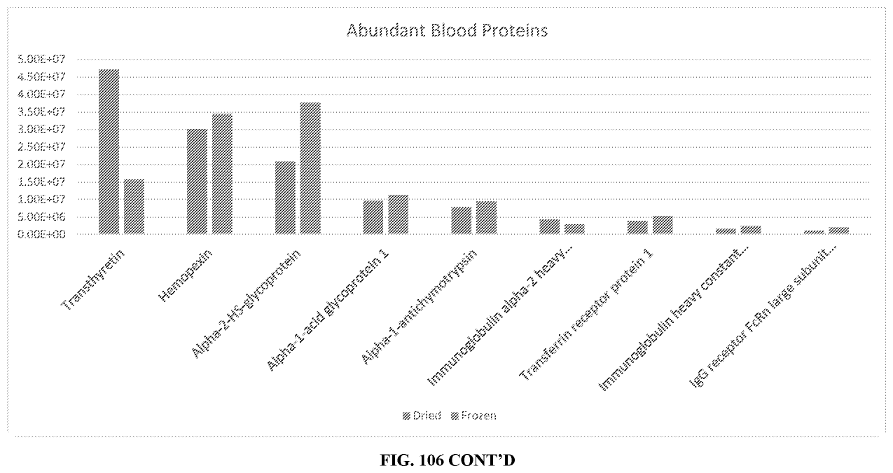

FIG. 105 shows the most abundant proteins in dried vs frozen samples. FIG. 106 shows the most abundant blood proteins in dried vs frozen samples.

In addition, the frozen sample had a reduced dynamic range compared to the dried (64% decrease), which contributes to why we see more proteins overall in the frozen sample.

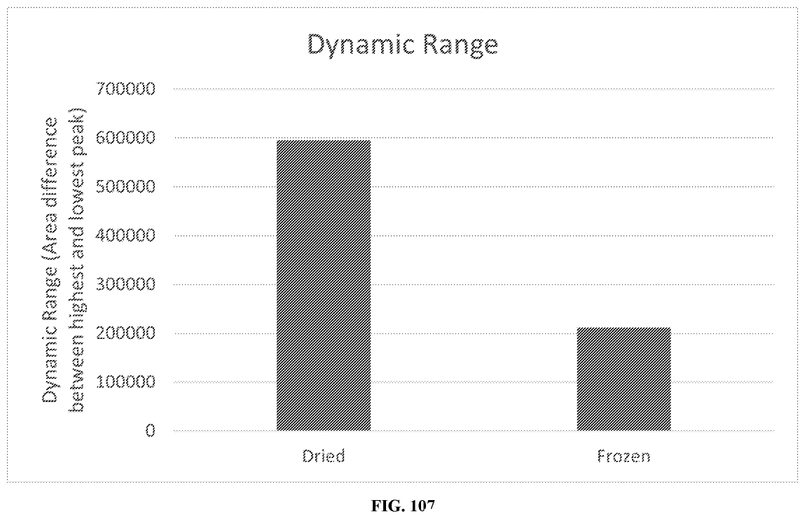

FIG. 107 shows the Dynamic range defined as the area difference between the highest and lowest peak from both DBS dried at room temperature and DBS frozen.

Conclusions: The results of this analysis demonstrate that differing storage methods can produce very different protein profiles. Based on visual observations, freezing the tip without allowing it to dry and performing freeze-thaw cycles resulted in more haemoglobin being washed out. This is also confirmed in the down-regulated proteins with haemoglobin resulting in a fold change of 2.9. The frozen sample also resulted in a higher number of protein and peptide IDs, most likely due to the more effective removal of high abundance proteins. The removal of high abundance proteins, however, does not appear to be the most common high abundant proteins in blood, except for albumin and transthyretin. Vitronectin is substantially higher in the frozen sample for example.

### Example XIV. Assessment of Alternative Storage Methods

To assess the effect of different extraction methods on DBS that have been stored at various temperatures. Samples stored at 37° C. were included to assess if an increased temperature accelerates the effect observed with room temperature stored samples. The requirement for cells to be intact was also observed by lysing cells using a sonic probe prior to loading, and the requirement for cells to be present was observed by assessing plasma in VAMS at various temperatures was also assessed.

### Methods

### Sample Preparation

Initially, six VAMS 30 μL tips were dipped into whole blood. 2 of the tips were left to dry with desiccant at room temperature for 5 days, 2 more tips were left to dry at 37° C. for 5 days, and the final 2 tips were left to dry for 5 mins then transferred to Eppendorf tubes and frozen at −80° C. for 5 days. Over that time, samples were removed from the freezer, left to come to room temperature then refrozen for a total of three times.

A portion of the liquid whole blood was also lysed using sonic probe (3× on ice). This was then loaded into 2 VAMS 30 μL tips. Tips were transferred to Eppendorf tubes and frozen at −80° C. for 5 days. Over that time, samples were removed from the freezer, left to come to room temperature then refrozen for a total of three times.

Finally, 4 VAMS 30 μL tips were loaded with plasma. 2 of the tips were left to dry with desiccant at room temperature for 5 days, and the final 2 tips were left to dry for 5 mins then transferred to Eppendorf tubes and frozen at −80° C. for 5 days. Over that time, samples were removed from the freezer, left to come to room temperature then refrozen for a total of three times.

### Protocol Used for Overnight Digestion

Following the period of drying and/or storage, the dried tips and frozen tips were washed overnight with either (a) LiCl (1 mL) then 2× LiCl washes with centrifuge pulses (10,000 g) or (b) 1 mL DUTRA (1% SDC, 7M Urea, 2M Thiourea, TBP, CLA, 100 mM Tris). 3× centrifuge pulses (10,000 g) were performed at the start of each urea wash incubation. The DUTRA samples were washed in 1 mL of water at the end of the washing. 100 μL Lysis Buffer (1% SDC, 10 mM TCEP, 40 mM CLA, 100 mM TEAB, pH 8) was added to all samples. Tips were heated at 95° C. for 10 mins with gentle agitation. 1 μg (2 μL) of trypsin (0.5 μg/μL in 100 mM TEAB) was then added and the solution was incubated overnight at 37° C. (19-21 hours). After incubation the tip was removed, and the remaining digest was diluted with 800 μL of 0.5% Formic acid. This solution was then centrifuged at 12,000 g for 10 mins and the supernatant was transferred to a conditioned SPE cartridge for clean-up. Peptides were then analysed using mass spectrometry.

### Results

### Total Yield

Total yield follows the same trends observed previously with a lower total yield for the frozen samples. Following the same trend, increasing the temperature to 37° C. also increased the total yield. Drying the whole blood sample in the tips increased the yield 40% over freezing and drying at 37° C. increased the yield by 70% over room temperature.

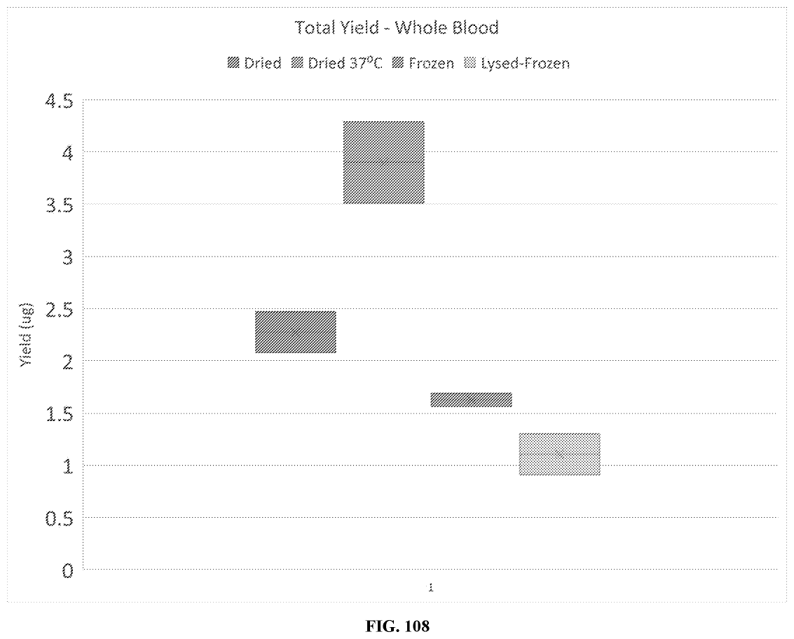

FIG. 108 shows total protein yield for whole blood DBS after storage at room temperature (dried), 37° C. (dried 37° C.), frozen at −80° C. (frozen) or lysed and then frozen at −80° C. (lysed-frozen).

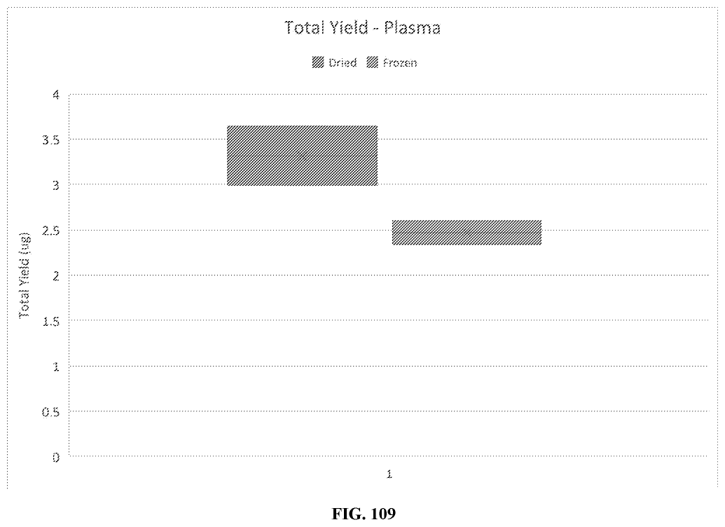

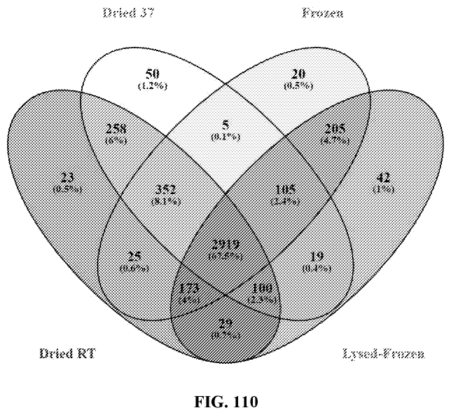

Drying the plasma sample in the tip at room temperature also increased the yield over freezing. Average yield was 34% higher in the room temperature samples. FIG. 109 shows total protein yield for plasma DBS after storage at room temperature (dried) or frozen at −80° C. (frozen).

### Comparison 1: Whole Blood Dried Vs Frozen

Both the dried sample (room temperature and 37° C.) show good overlap, and likewise the two frozen sample types (frozen and lysed-frozen). There were only 331 proteins unique to the dried samples and 267 unique to the frozen samples. FIG. 110 is a Venn diagram showing overlap of dried vs frozen whole blood DBS that were extracted with DUTRA wash.

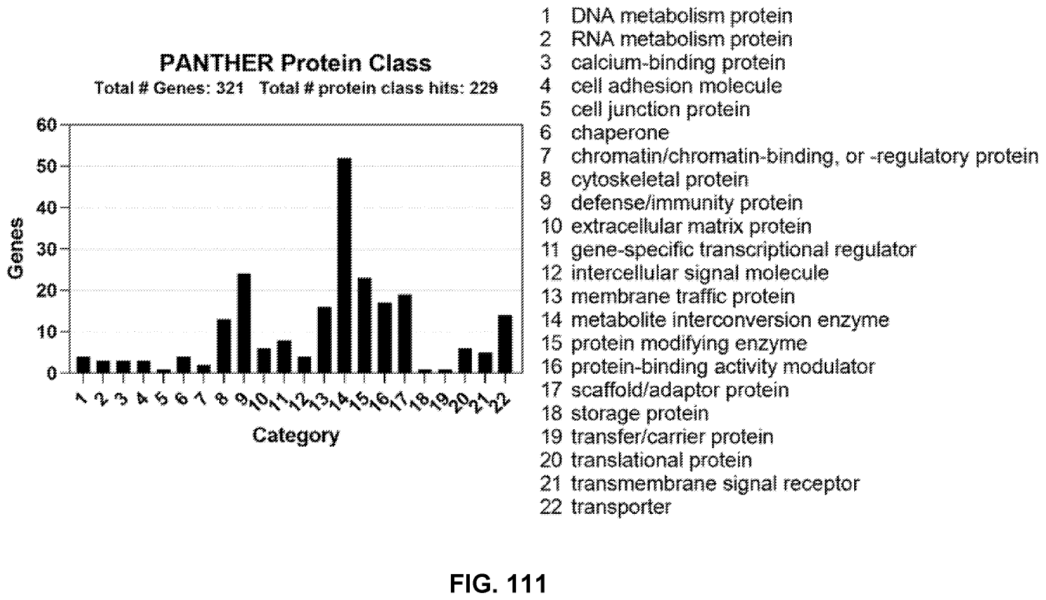

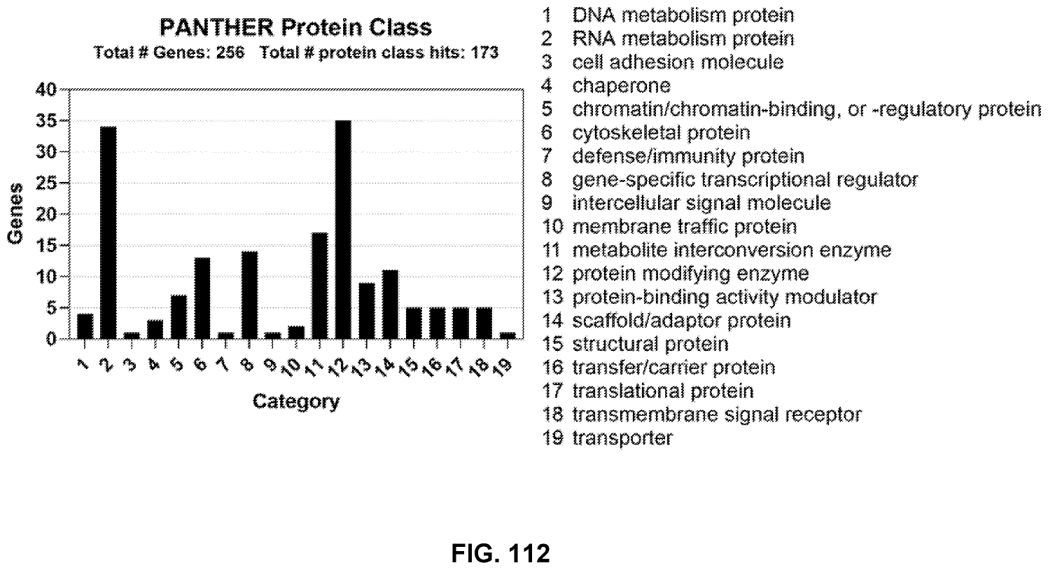

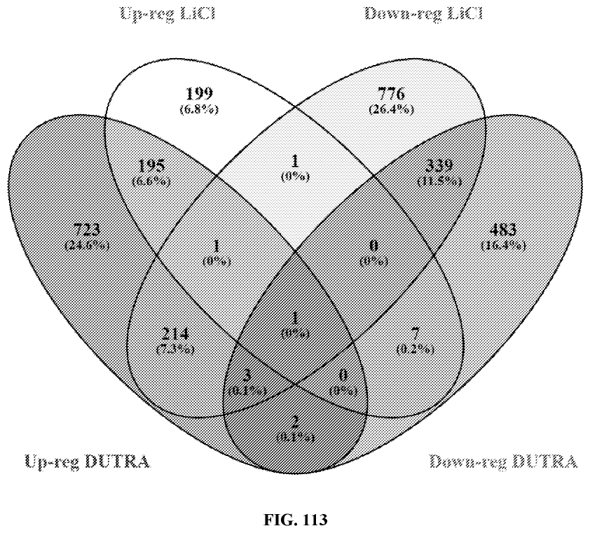

FIG. 111 shows proteins found exclusively in dried samples. FIG. 112 shows proteins found exclusively in frozen samples.

TABLE 22
Top up-regulated proteins in dried vs frozen DBS.

| | | | | Fold |
|---|---|---|---|---|
| ID | Gene Name | Gene ID | Protein Class | Change |
| P02763 | Alpha-2-macroglobulin | A2M | | 6.691414 |
| A0A075B6P5 | Alpha-1-acid glycoprotein | ORM1 | immunoglobulin(PC00123) | 6.138842 |
| | 1 | | | |
| P01023 | Immunoglobulin heavy | IGHG1 | protease inhibitor(PC00191) | 5.623319 |
| | constant gamma 1 | | | |
| Q8WVK2 | Immunoglobulin kappa | IGKV3D- | | 5.59707 |
| | variable 3D-11 | 11 | | |
| P61769 | Carbonic anhydrase 2 | CA2 | major histocompatibility complex | 5.432695 |
| | | | protein(PC00149) | |
| P02787 | Beta-2-microglobulin | B2M | transfer/carrier protein(PC00219) | 5.319128 |
| Q9NRL3 | Zinc-alpha-2-glycoprotein | AZGP1 | | 5.278844 |
| P01857 | Clathrin heavy chain 2 | CLTCL1 | immunoglobulin receptor | 5.216666 |
| | | | superfamily(PC00124) | |
| P00915 | | | dehydratase(PC00091) | 4.970068 |
| P01780 | Carbonic anhydrase 1 | CA1 | immunoglobulin(PC00123) | 4.89077 |
| A0A0A0MRZ8 | Striatin-4 | STRN4 | immunoglobulin(PC00123) | 4.746659 |
| P02724 | Erbin | ERBIN | | 4.741236 |
| Q6PJ69 | Serotransferrin | TF | ubiquitin-protein | 4.668996 |
| | | | ligase(PC00234) | |
| P00918 | Immunoglobulin kappa | IGKV2- | dehydratase(PC00091) | 4.658175 |
| | variable 2-28 | 28 | | |
| P53675 | Immunoglobulin heavy | IGHV3-7 | vesicle coat protein(PC00235) | 4.645924 |
| | variable 3-7 | | | |
| Q96RT1 | U4/U6.U5 small nuclear | SNRNP27 | scaffold/adaptor | 4.635263 |
| | ribonucleoprotein 27 kDa | | protein(PC00226) | |
| | protein | | | |
| P25311 | Tripartite motif-containing | TRIM65 | major histocompatibility complex | 4.633012 |
| | protein 65 | | protein(PC00149) | |

TABLE 23
Top down-regulated proteins in dried vs frozen DBS.

| | | | | Fold |
|---|---|---|---|---|
| ID | Gene Name | Gene ID | Protein Class | Change |
| Q9NXR1 | Non-homologous end-joining | NHEJ1 | | −6.36187 |
| | factor 1 | | | |
| P08651 | ATP-dependent RNA helicase | DDX3Y | DNA-binding transcription | −6.32709 |
| | | | factor(PC00218) | |
| P62633 | Phosphatidate phosphatase | LPIN2 | RNA metabolism | −5.30568 |
| | | | protein(PC00031) | |
| Q6NXT1 | Nuclear distribution protein nudE | NDE1 | | −5.0606 |
| | homolog 1 | | | |
| O15523 | ATP-dependent RNA helicase | DDX3Y | RNA helicase(PC00032) | −5.01427 |
| Q8IZQ5 | Cellular nucleic acid-binding | CNBP | | −4.90178 |
| | protein | | | |
| Q96QR8 | Serine/threonine-protein kinase | RIOK3 | DNA-binding transcription | −4.85995 |
| | | | factor(PC00218) | |
| Q9H9Q4 | Eukaryotic translation initiation | EIF2S3 | | −4.85454 |
| | factor 2 subunit 3 | | | |
| Q15046 | Phosphatidylinositol 4-phosphate | PIP5K1C | aminoacyl-tRNA | −4.72202 |
| | 5-kinase type-1 gamma | | synthetase(PC00047) | |
| O60331 | Nuclear factor 1 C-type | NFIC | kinase(PC00137) | −4.68794 |
| Q96AZ6 | ATP-dependent RNA helicase | DDX3X | exoribonuclease(PC00099) | −4.64226 |
| P04049 | Transcriptional activator protein | PURB | non-receptor serine/threonine | −4.40123 |
| | Pur-beta | | protein kinase(PC00167) | |
| Q92539 | Lysine-tRNA ligase | KARS1 | | −4.31267 |
| O14730 | ATP-dependent 6- | PFKM | non-receptor serine/threonine | −4.31186 |
| | phosphofructokinase, muscle | | protein kinase(PC00167) | |
| | type | | | |
| P41091 | N-alpha-acetyltransferase 16, | NAA16 | translation initiation | −4.28583 |
| | NatA auxiliary subunit | | factor(PC00224) | |
| P08237 | Selenoprotein H | SELENOH | carbohydrate kinase(PC00065) | −3.77024 |
| O00571 | Ankyrin repeat domain- | ANKRD54 | RNA helicase(PC00032) | −3.65705 |
| | containing protein 54 | | | |
| Q6N069 | RAF proto-oncogene | RAF1 | acetyltransferase(PC00038) | −3.03406 |
| | serine/threonine-protein kinase | | | |

### Comparison 2: DUTRA Vs LiCl Washing

Some overlap is seen between the two different washes in both up and down regulated proteins. There is an overlap of 214 proteins that are down-regulated in LiCl and up-regulated in DUTRA. FIG. 113 is a Venn diagram showing overlap of up- and down-regulated proteins in LiCl extraction and DUTRA extraction.

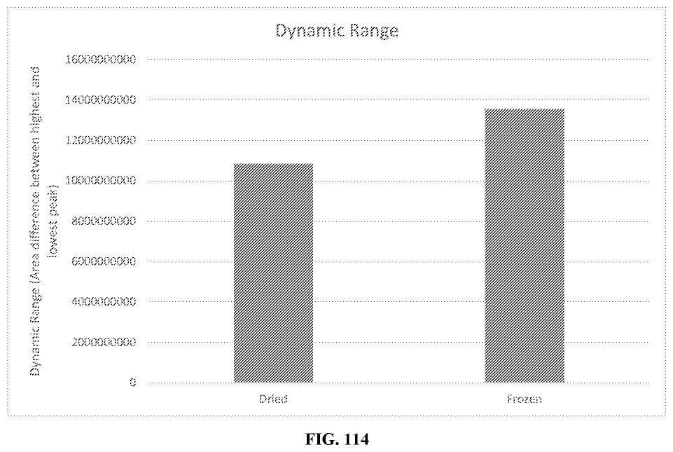

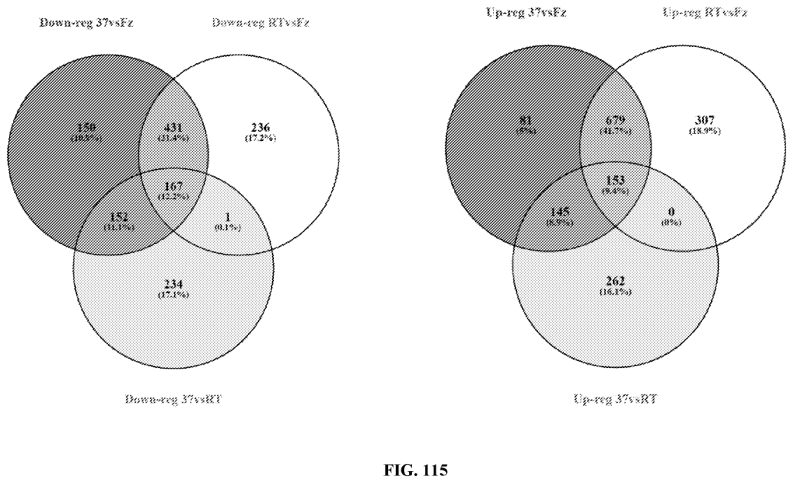

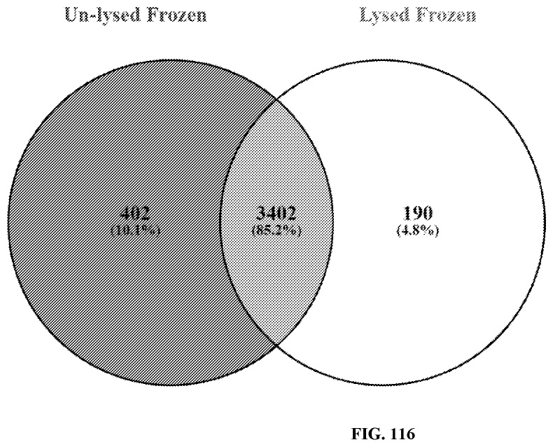

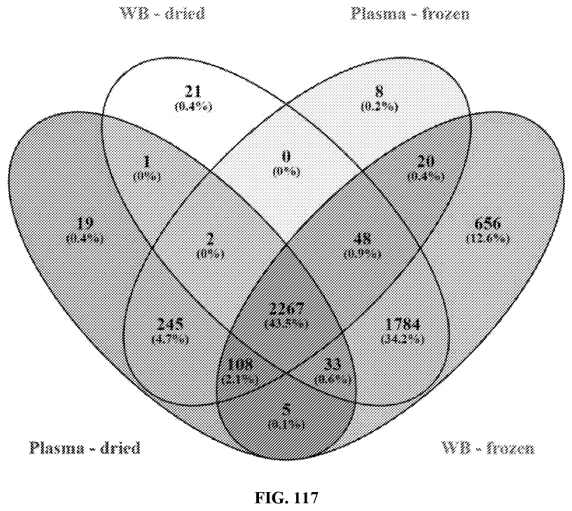

Compared to the LiCl extraction, there was not such a dramatic difference in dynamic range between dried and frozen for the DUTRA washed samples, but the dried has a slightly lower dynamic range. This is the opposite of what we saw with the LiCl washed samples. FIG. 114 shows the dynamic range defined as the area difference between the highest and lowest peak from both DBS dried at room temperature and DBS frozen followed by DUTRA extraction.

### Comparison 3: Whole Blood Dried at 37° C. Vs Room Temperature Vs Frozen

Overall, there were 610 proteins up-regulated in the 37° C. whole blood samples, and 603 proteins that were down-regulated when compared directly to the room temperature samples. There was a good overlap between the 37° C. and room temperature samples. The frozen samples were the most distinct in both up- and down-regulated proteins.

FIG. 115 shows overlap of down-regulated and up-regulated protein IDs from VAMS stored at room temperature (RT), 37° C. (37) or frozen at −80° C. (Fz).

### Comparison 4: Lysed Whole Blood Vs Un-Lysed

Whole blood that was lysed prior to application to the DBS tips, had 212 fewer IDs. Overall, the lysed sample produced the lowest yield and the lower protein and peptide numbers.

FIG. 116 shows overlap of proteins identified in whole blood (un-lysed) tips (un-lysed frozen) and whole blood lysed prior to loading into tip (lysed frozen). Both sets of tips were stored frozen at −80° C.

### Comparison 5: Plasma Dried Vs Frozen

There was a substantial amount of similarity between the dried and frozen plasma in terms of protein IDs and the majority of the plasma proteins were found in the whole blood samples. The whole blood frozen sample was the most unique of the set with 656 unique protein IDs.

FIG. 117 shows overlap of protein IDs from whole blood and plasma VAMS that have been dried at room temperature or else frozen immediately.

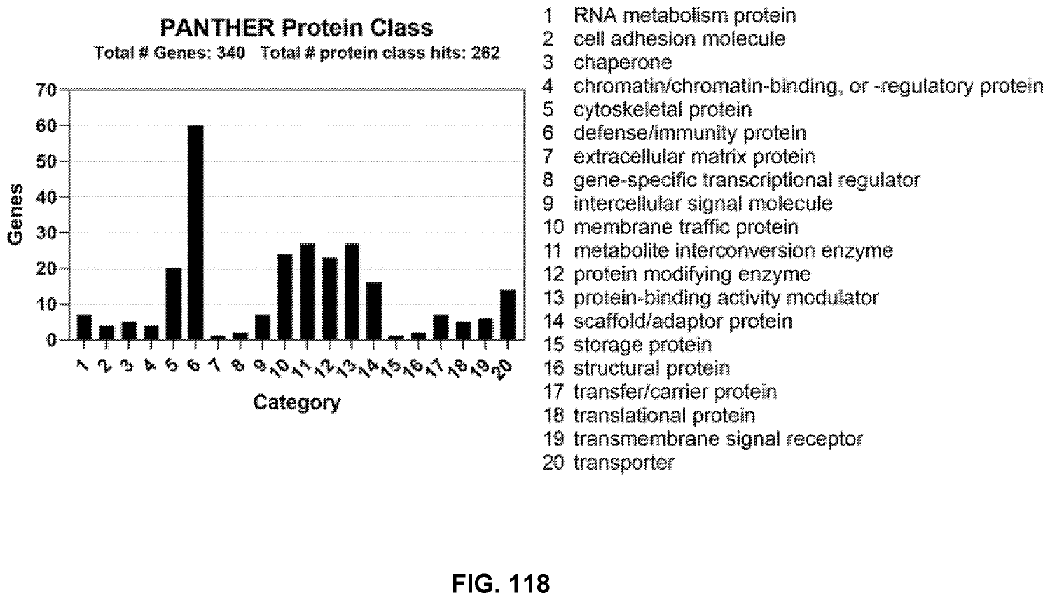

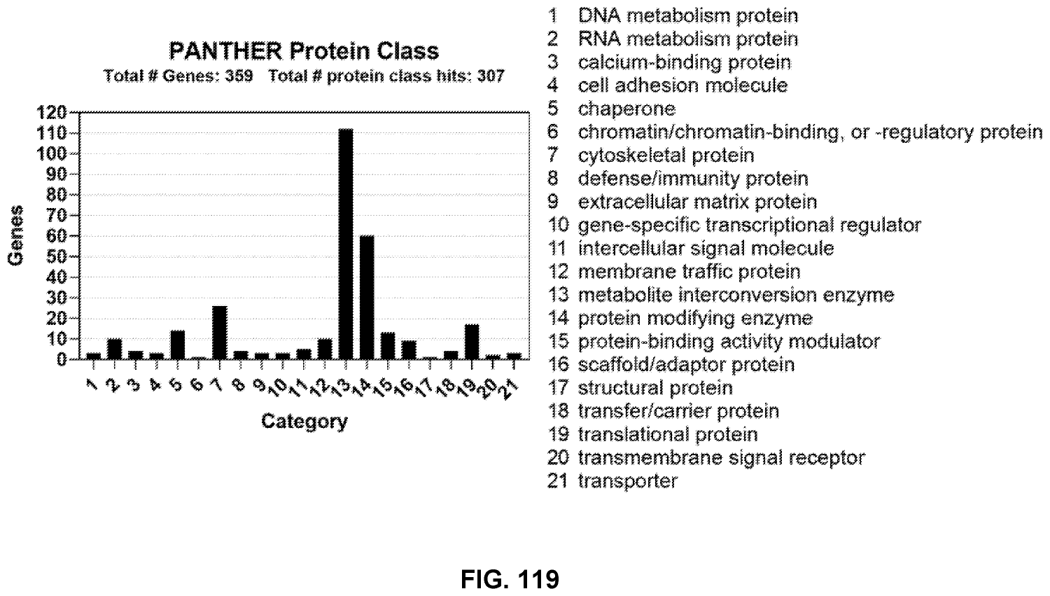

In plasma VAMS that had been dried at room temperature, 378 proteins were up-regulated comparted to the frozen equivalent. There were also 368 proteins down-regulated. The protein class of each group were vastly different to each other. FIG. 118 and FIG. 119 shows protein classes up-regulated and down-regulated, respectively in plasma VAMS dried at room temperature vs frozen at −80° C.

### Conclusions:

### Comparison 1: Whole Blood Dried Vs Frozen

The frozen samples (lysed and un-lysed) were most similar to each other, as the dried samples were also most similar to each other. Storing the samples at −80° C. significantly reduced total protein yield and produced a substantially different protein profile compared to room temperature or 37° C. storage.

### Comparison 2: DUTRA Vs LiCl Washing

DUTRA washing resulted in fewer unique differences in proteins between fresh vs frozen compared to LiCl wash. For fold change comparison using the DUTRA washing, there were a similar number of up and down regulated proteins overall when comparing dried vs frozen as found in the LiCl wash. The dynamic range did not change as drastically using the DUTRA washing vs LiCl and showed the opposite trend of less dynamic range in the dried tip vs the frozen tip.

### Comparison 3: Whole Blood Dried 37° C. Vs Room Temperature

Drying at 37° C. did significantly increase yield over the room temperature tips. Drying at 37° C. also uniquely produced quite a lot of up-regulated cytoskeletal proteins compared to the other samples. There was a lot of overlap in up and down regulated proteins between drying at 37° C. and room temperature compared to frozen.

### Comparison 4: Lysed Whole Blood Vs Un-Lysed

Similar to the 37° C. sample, there was a lot of cytoskeletal proteins up-regulated in the lysed sample compared to the frozen sample. Down-regulated proteins were primarily RNA metabolism proteins and scaffold proteins.

### Comparison 5: Plasma Dried Vs Frozen

The majority of the plasma proteins were also identified in the whole blood. There was not as many unique proteins identified in the comparison between dried and frozen plasma (compared to whole blood).

### Example XV. Assessment of Different Extraction Solutions Including DNA Extraction

Aim: to assess the effect of various extraction solutions on overall protein yield and IDs and to demonstrate an example of sequential extraction wherein the first extract is processed for DNA analyse, and the subsequent extract is processed for protein MS analysis. Finally, to present an example of pre-loading VAMS tips to effect protein extraction.

### Methods

### Sample Preparation

Eleven VAMS 30 μL tips were dipped into whole blood. A portion were left to dry with desiccant at room temperature overnight. One tip was left to dry with desiccant at 37° C. overnight. One tip was incubated at 99° C. (lid open) for 10 mins, then placed back on the spindle and left to dry with desiccant at room temperature overnight.

For tip pre-loading, one tip was dipped in a solution of benzonase (1 U/μL). The tip was left to dry for 10 minutes the store in the fridge overnight. It was then loaded with whole blood, and left to dry with desiccant at room temperature overnight. Another tip was loaded with whole blood that has been treated with benzonase (1 U/μl) for 10 minutes prior to loading the tip. One tip was loaded with fingertip whole blood and left to dry with desiccant at room temperature overnight. Finally, one tip was loaded with plasma which was double spun to ensure a higher purity and then left to dry with desiccant at room temperature overnight.

### DNA Extraction

DNA was extracted from one of the whole blood tips according to the following methods. A 30 μL VAMS tip containing whole blood was dispensed into a 1.5 mL microfuge tube. 200 μL PBS was applied to the tip and vortexed for 10 seconds. 200 μL proteinase K and 300 μL lysis buffer B was then added and vortexed for 10 seconds. The solution was then incubated at 56° C. for 20 minutes. 250 μL ethanol was added and vortexed to mix, after which the DBS tup was collected and processed as below for mass spectrometry. The remaining solution was processed according to manufacturer's instructions for DNA preparation (QIAamp blood kit).

### Protocol Used for Overnight Digestion

Once dried, the tips were washed with one of the following methods:
a) Wash overnight with 500 mM LiCl (1 mL), 100 mM Tris, then 2×LiCl wash (with centrifuge pulses (10,000 g)
b) Wash overnight in 2% CHAPS, 500 mM LiCl, 100 mM Tris Overnight, then 2× wash with same buffer with centrifuge pulses (10,000 g). Tips were then washed three times in 1 mL of water at the end of the washing.
c) Wash overnight in 10 mM Citric Acid, 500 mM LiCl overnight, then 2× wash with same buffer with centrifuge pulses (10,000 g). Tips were then washed three times in 1 mL of water at the end of the washing.
d) Wash with 25% trifluoroacetic acid (TFA) for 10 mins, then overnight in 100 mM Tris, followed by 2× wash in 100 mM Tris with centrifuge pulses (10,000 g).
e) Wash overnight in Methanol (MeOH) 40% v/v, followed by 2× wash with same buffer with centrifuge pulses (10,000 g).
f) Add 1 U of Benzonase in 100 μL to tip leave 10 mins then make up to 1 mL with 500 mM LiCl, 100 mM Tris, and leave overnight, followed by 2×LiCl wash (with centrifuge pulses (10,000 g).
g) Wash overnight with 500 mM LiCl (1 mL), 100 mM Tris, then 2×LiCl wash with sonication (3× sonic probe, each wash).

All tips were processed the same after this point. 100 μL Lysis Buffer (1% SDC, 10 mM TCEP, 40 mM CLA, 100 mM TEAB, pH 8) was added to all samples. Tips were heated at 95° C. for 10 mins with gentle agitation. 1 μg (2 μL) of trypsin (0.5 μg/μL in 100 mM TEAB) was then added and the solution was incubated overnight at 37° C. (19-21 hours). After incubation the tip was removed, and the remaining digest was diluted with 800 μL of 0.5% Formic acid. This solution was then centrifuged at 12,000 g for 10 mins and the supernatant was transferred to a conditioned SPE cartridge for clean-up. Peptides were then analysed using mass spectrometry.

### Results

### Total Yield

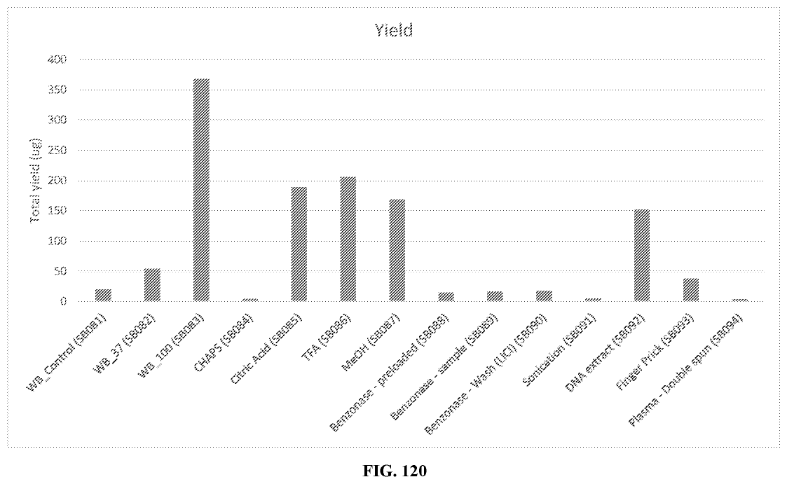

Total yield for the heating experiments follows the same trends as seen previously, with a 170% increase in yield drying at 37° C. and a further 580% increase at 99° C. For the various washing methods those that gave the lowest yield were the CHAPS and the Sonicated LiCl tip. The two acid treated tips gave a similar result in yield, followed closely by the MeOH and the DNA extracted tip. The finger prick blood sample gave a higher yield than the venous control which is most likely due to clotting factors. The three tips with benzonase treatment did not change the yield over the control. The double spun plasma gave similar yield to the plasma from prior experiments. FIG. 120 shows total protein yield from all samples.

A portion of tips were visually different following extraction. Specifically, the tip heated to 99° C. went a dark black colour and remaining this colour even after washing. The 2 acid treated tips turned a similar colour and stayed dark even post washing. The MeOH tip was somewhere in the middle, but both the CHAPS and sonicated LiCl were almost white again after washing, even more so than the control.

These colour differences are reflected in the total protein yield, with the coloured tips resulting in high protein yields. Further, the tips that remaining dark after washing generally showed lower IDs and the mass spec trace indicated a majority of high abundance peaks. The mass spectra of the CHAPS containing sample looked good and so did the IDs. CHAPS is typically avoided in mass spectrometry work due to mass spec artefacts, however, using it with these methods produced high quality results.

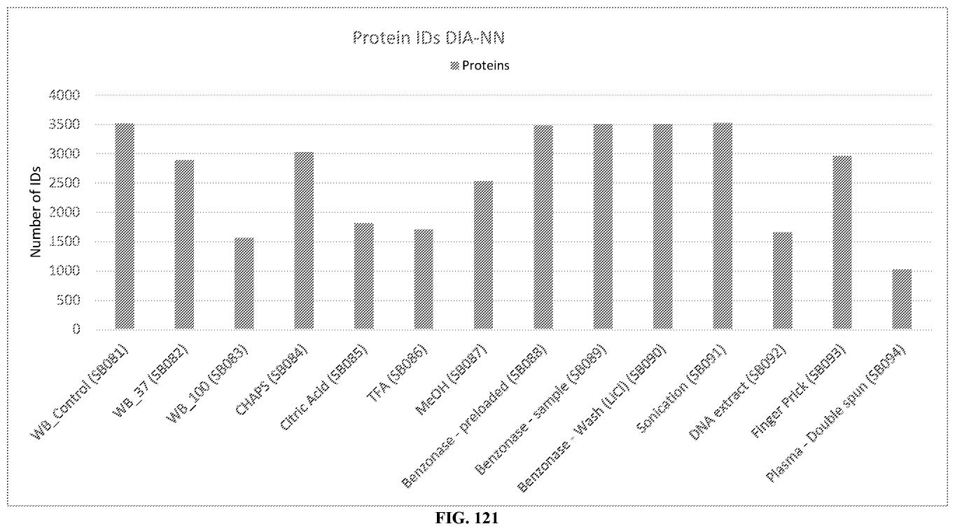

FIG. 121 shows the number of protein IDs from a variety of processing methods.

### Comparison 1: Temperature Comparison

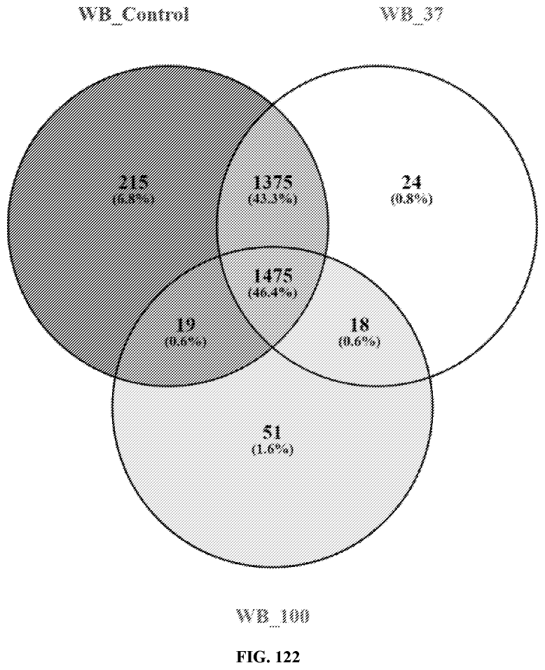

Overlap of proteins from drying the whole blood tips at room temperature, 37° C. or 99° C. The room temperature drying produced the most unique proteins. Drying at 99° C. produced half as many IDs as the room temperature and 37° C. tips, this is mainly due to a large amount of high abundance proteins in the samples dried at elevated temperatures. This is further evidence that drying and storage temperature can be used to produce different protein profiles. FIG. 122 is a Venn diagram showing overlap of protein IDs from VAMS dried and stored at a variety of temperatures, room temperature (control), 37° C. (37), or 99° C. (100).

TABLE 24
Significantly up-regulated proteins (37° C. vs room temperature).

| Row_ID | Description | Fold Change | Pval |
|---|---|---|---|
| P02008; P69905 | Hemoglobin subunit zeta | 6.759333 | 0.005982 |
| Q8NCM8 | Cytoplasmic dynein 2 heavy chain 1 | 3.58024 | 0.002471 |
| P31942 | Heterogeneous nuclear ribonucleoprotein H3 | 3.274654 | 0.003251 |
| A0A075B619; P04211 | Immunoglobulin lambda variable 7-46 | 2.872386 | 0.008913 |
| P06744 | Glucose-6-phosphate isomerase | 2.744619 | 0.001324 |
| P15121 | Aldo-keto reductase family 1 member B1 | 2.603082 | 0.002801 |
| P05160 | Coagulation factor XIII B chain | 2.593073 | 0.004649 |
| P04040 | Catalase | 2.563792 | 0.001405 |
| P02042; P02100; P68871; | Hemoglobin subunit delta | 2.360063 | 0.001496 |
| P69891; P69892 | | | |
| P21291 | Cysteine and glycine-rich protein 1 | 2.317422 | 0.001273 |
| O75636 | Ficolin-3 | 2.156194 | 0.001625 |
| Q99733 | Nucleosome assembly protein 1-like 4 | 2.121347 | 0.003492 |
| P22352 | Glutathione peroxidase 3 | 2.055728 | 0.001581 |
| O43665 | Regulator of G-protein signaling 10 | 1.920067 | 0.002787 |
| P31153 | S-adenosylmethionine synthase isoform type-2 | 1.8857 | 0.000976 |
| P23588 | Eukaryotic translation initiation factor 4B | 1.881737 | 0.002614 |
| Q8WZA0 | Protein LZIC | 1.822642 | 0.005906 |
| P54725; P54727 | UV excision repair protein RAD23 homolog A | 1.820616 | 0.002998 |
| Q9H299 | SH3 domain-binding glutamic acid-rich-like | 1.804471 | 0.004144 |
| | protein 3 | | |
| Q96FJ2 | Dynein light chain 2, cytoplasmic | 1.729673 | 0.000725 |

TABLE 25
Significantly up-regulated proteins (99° C. vs room temperature).

| Row_ID | Description | Fold Change | Pval |
|---|---|---|---|
| P02008; P69905 | Hemoglobin subunit zeta | 7.896837 | 0.005982 |
| A0A075B619; P04211 | Immunoglobulin lambda variable 7-46 | 7.101591 | 0.008913 |
| Q9NZD4 | Alpha-hemoglobin stabilizing protein | 6.717235 | 0.01132 |
| P02750 | Leucine-rich alpha-2-glycoprotein | 6.257299 | 0.007985 |
| A0A087WW87; P01614 | Immunoglobulin kappa variable 2-40 | 6.106067 | 0.0069 |
| P02763 | Alpha-1-acid glycoprotein 1 | 5.962386 | 0.008112 |
| P02753 | Retinol-binding protein 4 | 5.729886 | 0.006814 |
| Q9UNZ2 | NSFL1 cofactor p47 | 5.700197 | 0.009068 |
| P51884 | Lumican | 5.287029 | 0.007488 |
| P80748 | Immunoglobulin lambda variable 3-21 | 5.282133 | 0.00689 |
| P01011 | Alpha-1-antichymotrypsin | 5.225791 | 0.005608 |
| A0A075B6K5; P80748 | Immunoglobulin lambda variable 3-9 | 5.094083 | 0.006156 |
| P01857; P01859; P01860; P01861 | Immunoglobulin heavy constant gamma 1 | 4.904275 | 0.002734 |
| P01709 | Immunoglobulin lambda variable 2-8 | 4.887718 | 0.004837 |
| P02747 | Complement C1q subcomponent subunit C | 4.868544 | 0.004974 |
| Q8WZA0 | Protein LZIC | 4.865817 | 0.005906 |
| P01857; P01860 | Immunoglobulin heavy constant gamma 1 | 4.864903 | 0.003474 |
| P01009 | Alpha-1-antitrypsin | 4.860954 | 0.003779 |
| P36955 | Pigment epithelium-derived factor | 4.809029 | 0.005676 |
| A0A0B4J1V0 | Immunoglobulin heavy variable 3-15 | 4.790385 | 0.006389 |

TABLE 26
Significantly down-regulated proteins (37° C. vs room temperature).

| Row_ID | Description | Fold Change | Pval |
|---|---|---|---|
| P04264; P35908 | Keratin, type II cytoskeletal 1 | −1.82009 | 0.003648 |
| P13647 | Keratin, type II cytoskeletal 5 | −1.86856 | 0.0045 |
| P12955 | Xaa-Pro dipeptidase | −1.88508 | 0.00182 |
| P08519 | Apolipoprotein(a) | −1.89476 | 0.001208 |
| P13645 | Keratin, type I cytoskeletal 10 | −1.92021 | 0.006195 |
| P0C0S8; P20671; Q16777; | Histone H2A type 1 | −2.01078 | 0.003336 |
| Q6FI13; Q96KK5; Q99878; Q9BTM1 | | | |
| P61626 | Lysozyme C | 2.05232 | 0.000698 |
| P14780 | Matrix metalloproteinase-9 | −2.09006 | 0.00127 |
| P02533; P08779; P13645 | Keratin, type I cytoskeletal 14 | −2.09548 | 0.008528 |
| Q13231 | Chitotriosidase-1 | −2.12886 | 0.001663 |
| P35908 | Keratin, type II cytoskeletal 2 epidermal | −2.1452 | 0.005629 |
| P80188 | Neutrophil gelatinase-associated lipocalin | −2.16083 | 0.002899 |
| P05771; P17252 | Protein kinase C beta type | −2.17241 | 0.001671 |
| P05164 | Myeloperoxidase | −2.18229 | 0.000921 |
| Q9BY43 | Charged multivesicular body protein 4a | −2.1831 | 0.001586 |
| P02538; P04259; P13647; P19013; | Keratin, type II cytoskeletal 6A | −2.33744 | 0.011583 |
| P35908; P48668; Q7Z794; Q86Y46 | | | |
| P04264; P35908; Q7Z794 | Keratin, type II cytoskeletal 1 | −2.40909 | 0.006206 |
| P22894 | Neutrophil collagenase | −2.63731 | 0.001758 |
| P54108 | Cysteine-rich secretory protein 3 | −2.86914 | 0.002302 |
| P08185 | Corticosteroid-binding globulin | −3.61191 | 0.005106 |

TABLE 27
Significantly up-regulated proteins (99° C. vs room temperature).

| | | Fold | |
|---|---|---|---|
| Row_ID | Description | Change | Pval |
| Q9NRQ2 | Phospholipid scramblase 4 | −5.42519 | 0.006424 |
| Q68D91 | Acyl-coenzyme A thioesterase MBLAC2 | −5.43993 | 0.008185 |
| P49753; Q86TX2 | Acyl-coenzyme A thioesterase 2, | −5.45402 | 0.008791 |
| | mitochondrial | | |
| Q9C0E2 | Exportin-4 | −5.4761 | 0.007347 |
| Q8N699 | Myc target protein 1 | −5.55201 | 0.009168 |
| P62805 | Histone H4 | −5.57469 | 0.004059 |
| Q96DZ9 | CKLF-like MARVEL transmembrane | −5.59798 | 0.00725 |
| | domain-containing protein 5 | | |
| P16671 | Platelet glycoprotein 4 | −5.61175 | 0.006186 |
| Q00765 | Receptor expression-enhancing protein 5 | −5.68142 | 0.006958 |
| P16615 | Sarcoplasmic/endoplasmic reticulum | −5.6868 | 0.007312 |
| | calcium ATPase 2 | | |
| P02538; P04259; P13647; P35908; | Keratin, type II cytoskeletal 6A | −5.71126 | 0.006167 |
| P48668; Q86Y46; Q8N1N4 | | | |
| P18577; Q02161 | Blood group Rh(CE) polypeptide | −5.76321 | 0.005588 |
| P35908 | Keratin, type II cytoskeletal 2 epidermal | −6.32808 | 0.005629 |
| P08237; P17858 | ATP-dependent 6-phosphofructokinase, | −6.35566 | 0.009205 |
| | muscle type | | |
| P04264; P35908; Q7Z794 | Keratin, type II cytoskeletal 1 | −6.37325 | 0.006206 |
| P13645 | Keratin, type I cytoskeletal 10 | −6.59518 | 0.006195 |
| O95197 | Reticulon-3 | −6.64304 | 0.008875 |
| P02533; P08779; P13645 | Keratin, type I cytoskeletal 14 | −7.59495 | 0.008528 |
| P02538; P04259; P13647; P19013; | Keratin, type II cytoskeletal 6A | −8.1282 | 0.011583 |
| P35908; P48668; Q7Z794; Q86Y46 | | | |
| P49755 | Transmembrane emp24 domain- | −8.21364 | 0.017028 |
| | containing protein 10 | | |

### Comparison 2: Finger Prick Vs Venous Blood

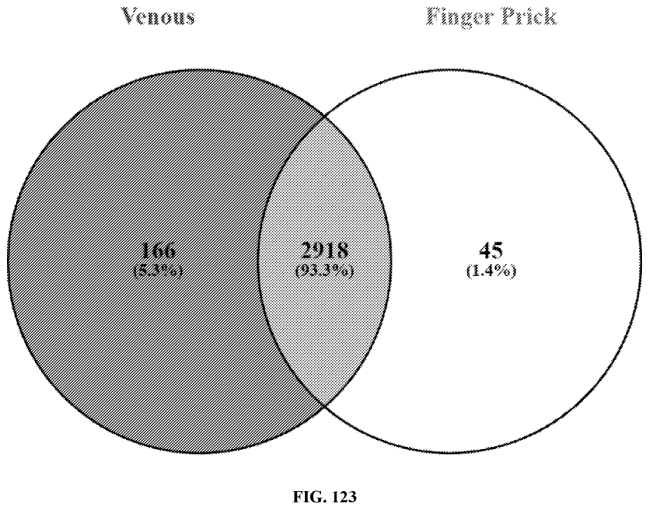

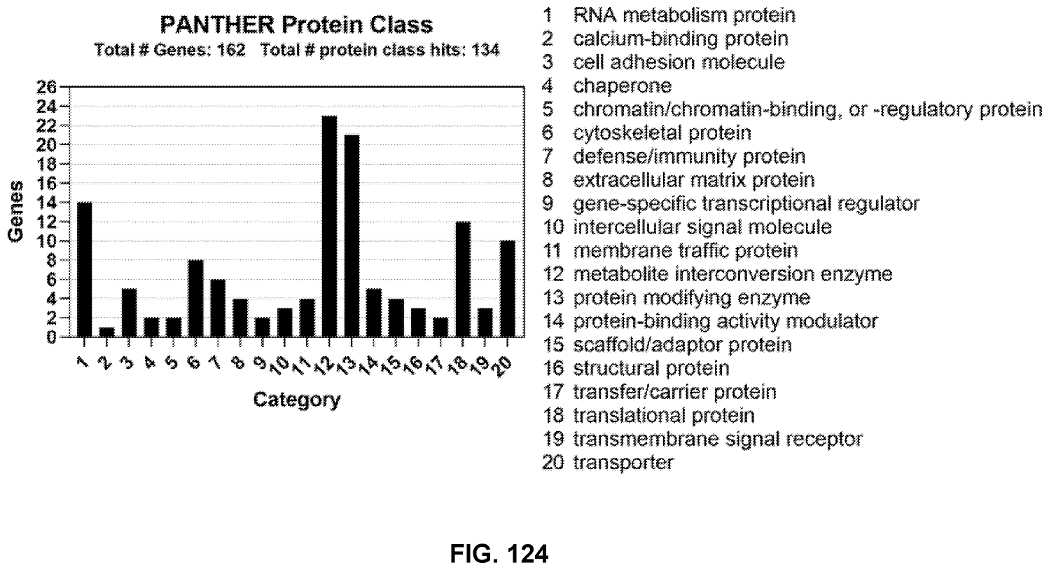

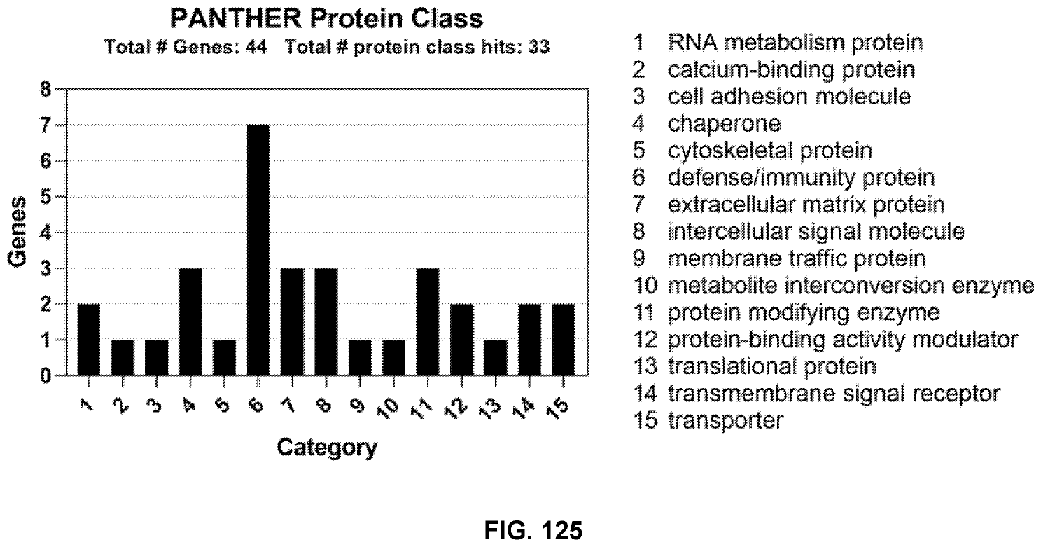

There was a large amount of overlap between the finger prick and venous blood samples. The venous blood gave slightly more IDs overall and hence had a few more uniquely identified proteins. Thus both samples can be successfully collected and processed according to these methods. FIG. 123 is a Venn diagram showing overlap in protein IDs from venous blood vs finger prick blood. FIG. 124 shows protein classes unique to venous blood samples. FIG. 125 shows protein classes unique to fingerprick blood samples.

### Comparison 3: DNA Extract Vs Whole Blood

The initial extraction from the DBS extract sample produced 0.5 ng/μL DNA, a quantity that will likely be increased with further method optimisation. When subsequently processed for protein extraction, the DNA extract sample had fewer protein IDs compared to whole blood processed using the standard method but still had over 1500 IDs. This method is proof that both DNA and protein can be extracted in series from a single VAMS tip.

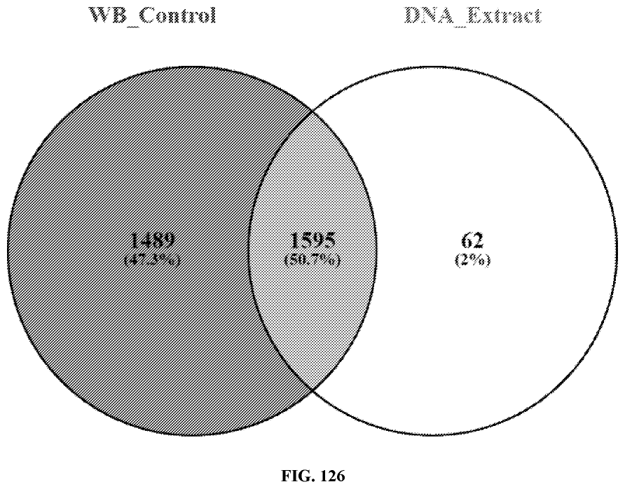

FIG. 126 is a Venn diagram showing overlap of protein IDs from samples processed using the standard methods and samples initially extracted for DNA then extracted for proteins.

### Comparison 4: Test of Various Washing Buffers

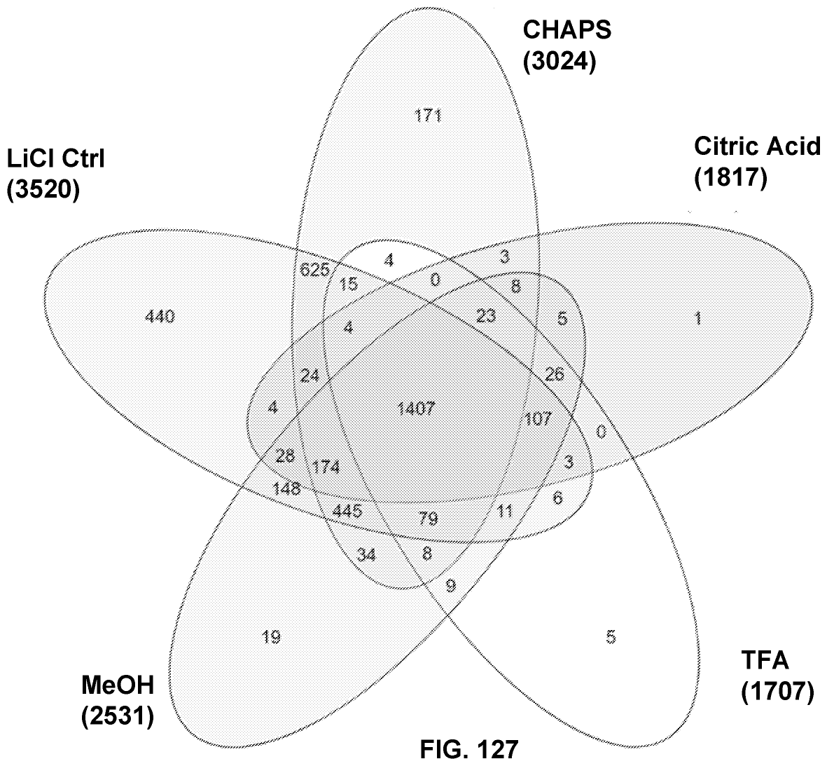

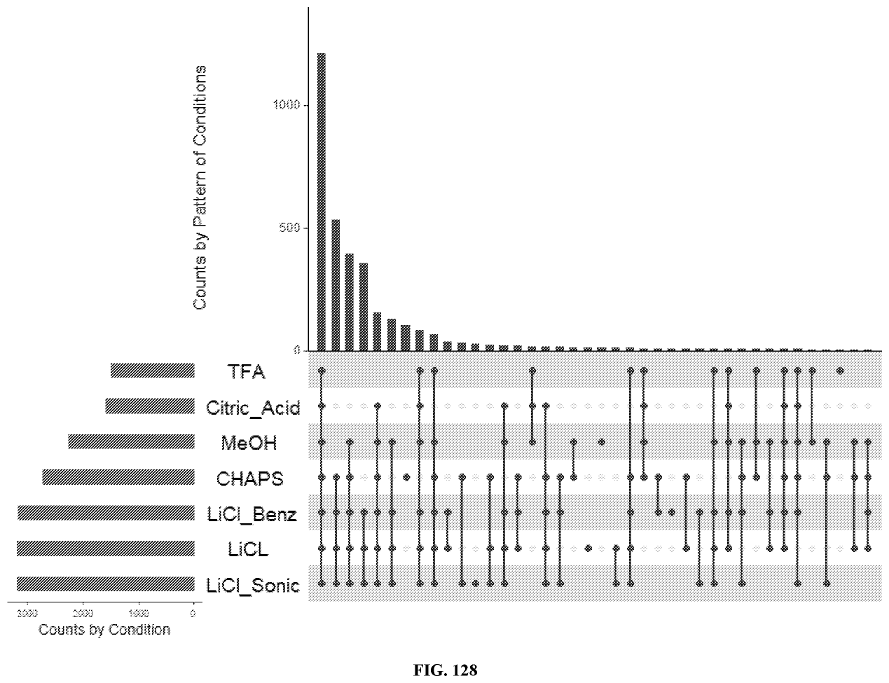

Both the LiCl and CHAPS produced the most unique protein profiles, followed by MeOH. FIG. 127 is a Venn diagram showing overlap of protein IDs after using various washing buffers for extraction. FIG. 128 is an upset Upset plot of protein ID overlap from various washing and extraction methods including sonication and benzonase during extraction.

Each washing/extraction methods produced different protein profiles. But the profile of heating to 99° C. had similarities to the acid and solvent preps, whilst heating to 37° C. had similarities with the salt preps. CHAPS was the only washing solvent to produce its own completely distinct profile when compared to salts, acids, and organic solvents.

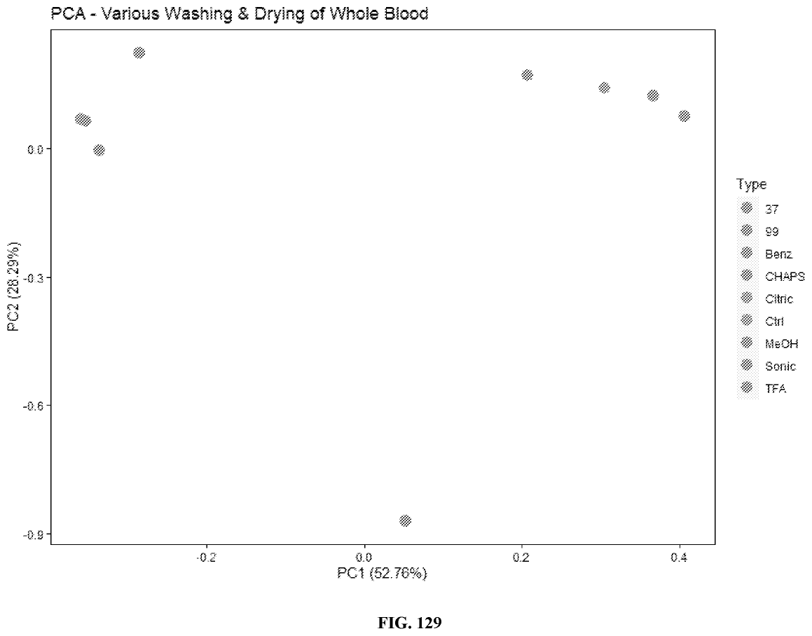

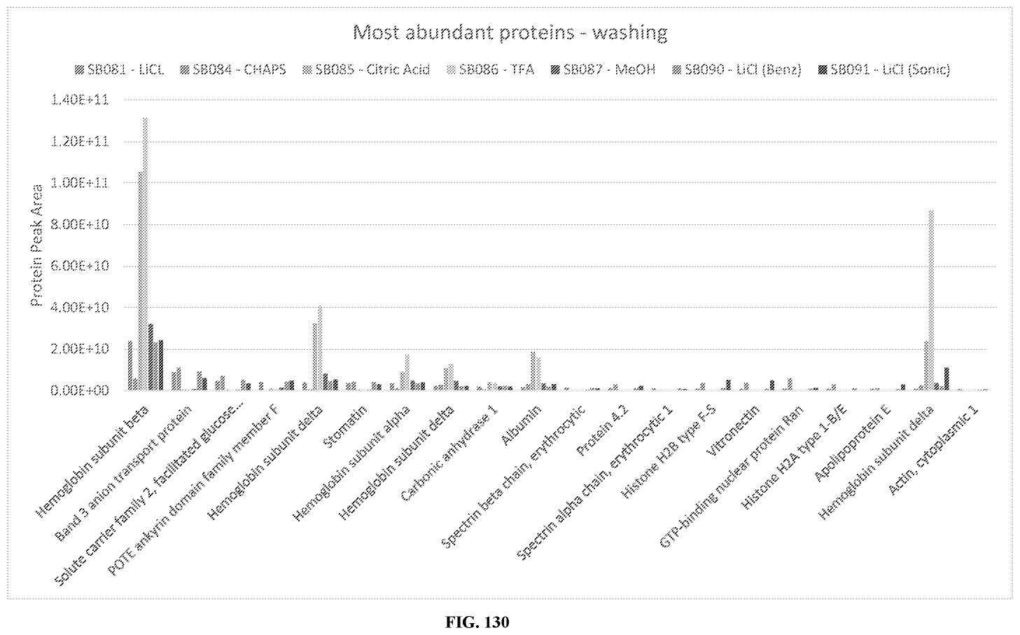

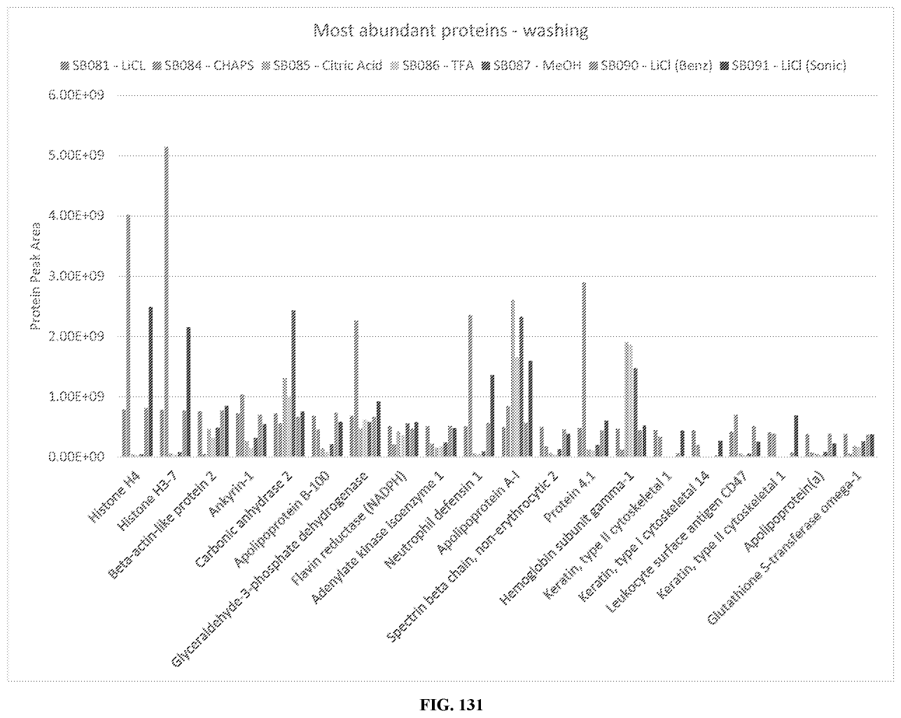

FIG. 129 shows a PCA plot comparing each of the washing and drying methods of whole blood VAMS. FIG. 130 shows a summary of the protein peak areas for the most abundant proteins using various washing methods (1-20 most abundant proteins) and FIG. 131 shows a summary of the protein peak areas for the most abundant proteins using various washing methods (21-30 most abundant proteins).

### Comparison 5: Use of Benzonase

Addition of benzonase at any step did not significantly change the profile of the extraction. Overall it seemed to perform better when added in at the wash step, but this difference is minor.

Although pre-loading the tips with benzonase didn't have a significant effect on the protein profile, it provides evidence that these tips can be pre-loaded with molecules and does not interfere with the subsequent collection of blood into the tip.

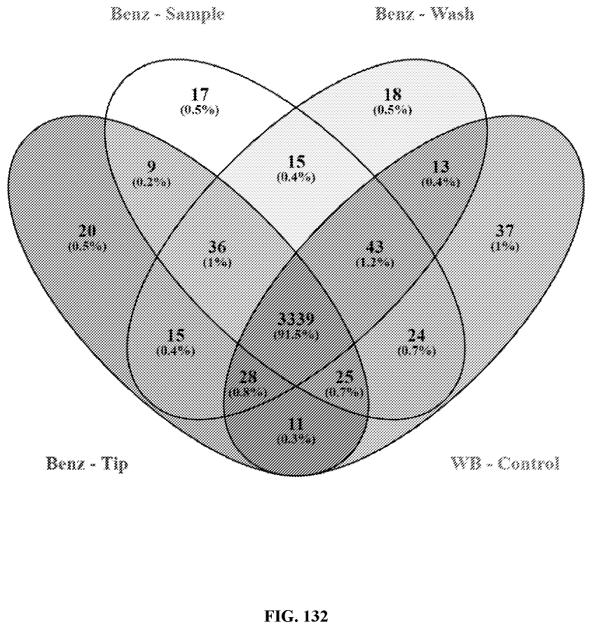

FIG. 132 is a Venn diagram showing overlap in protein IDs for samples treated with benzonase at different stages of the extraction compared to an untreated control.

### Conclusions

### Comparison 1: Temperature Comparison

Heating to 99° C. had a detrimental effect on protein IDs, as it made it harder to wash away high abundant proteins such as haemoglobin. Drying at 37° C. didn't give much difference in overall IDs, however high abundant proteins such as haemoglobin were upregulated with the elevated temperature indicating it does suffer from some of the issues with the higher 99° C. temperature.

### Comparison 2: Finger Prick Vs Venous Blood

Venous blood did give slightly more unique IDs over the fingertip blood, but overall they were fairly comparable set and demonstrate usable samples for type of analysis.

### Comparison 3: DNA Extract Vs Whole Blood

DNA and protein was successful extracted in series from a single VAMS tip. Further optimisation is likely to achieve better yield of both.

### Comparison 4: Test of Various Washing Buffers

Of the different washing solutions LiCl and CHAPS produce the most unique proteins, though they were all somewhat different to each other.

### Comparison 5: Use of Benzonase

Use of benzonase in general gave a similar result to the LiCl control but did appear to marginally increase protein ID numbers, however this wasn't significant. The pre-loading of benzonase in the tips proved that VAMS can be pre-loaded with a molecule and still successfully collect a blood sample.

### Example XVI. Assessment of Various Sample Types Including Spiked Blood Samples

Aims: To investigate the sensitivity of the mass spectrometer in detecting mouse brain cancer cells in human whole blood. To investigate the lysis performance of tips. To investigate the sonication performance on protein quantification.

### Methods

### Sample Preparation

Mouse brain cancer cells (MBCC) and yeast cells were sonicated (4× 100 AMP on ice). Sonicated MBCC were loaded on 1 VAMS 30 μL tip and stored at room temperature. Another 30 μL aliquot of the sonicated MBCC were frozen at −80° C. Sample was thawed prior to digestion. Sonicated yeast cells were loaded onto 1 VAMS 30 μL tip and stored at room temperature. Another 30 μL aliquot of the sonicated yeast cells were frozen at −80° C. Sample was thawed prior to digestion. Finally, a series of 7 dilutions of sonicated MBCC in whole blood were prepared according to Table 10. 1 VAMS 30 μL tip was prepared from each dilution and stored at room temperature.

TABLE 28
Summary of serial dilutions of MBCC in whole blood.

| Sample | volume of | volume of | Cells in DBS tip | Cells/ |
|---|---|---|---|---|
| ID | WB in ul | cells in ul | (30 ul) total | ul |
| 50 ul | 50.00 | 50.00 | 127125 | 2543 |
| 25 ul | 75.00 | 25.00 | 63563 | 1271 |
| 12 ul | 87.50 | 12.50 | 31781 | 636 |
| 6 ul | 93.75 | 6.25 | 15891 | 318 |
| 3 ul | 96.88 | 3.13 | 7945 | 159 |
| 2 ul | 98.44 | 1.56 | 3973 | 79 |
| 1 ul | 99.22 | 0.78 | 1986 | 40 |

### Protocol Used for Overnight Digestion

Once dried, the tips were washed overnight with LiCl (1 mL) then 2× LiCl washes with centrifuge pulses (10,000 g). All samples (tips and liquid samples) were then processed according to the following methods. 100 μL Lysis Buffer (1% SDC, 10 mM TCEP, 40 mM CLA, 100 mM TEAB, pH 8) was added to all samples. Tips were heated at 95° C. for 10 mins with gentle agitation. 1 μg (2 μL) of trypsin (0.5 μg/μL in 100 mM TEAB) was then added and the solution was incubated overnight at 37° C. (19-21 hours). After incubation the tip was removed, and the remaining digest was diluted with 800 μL of 0.5% Formic acid. This solution was then centrifuged at 12,000 g for 10 mins and the supernatant was transferred to a conditioned SPE cartridge for clean-up. Peptides were then analysed using mass spectrometry.

### Results

### Total Yield

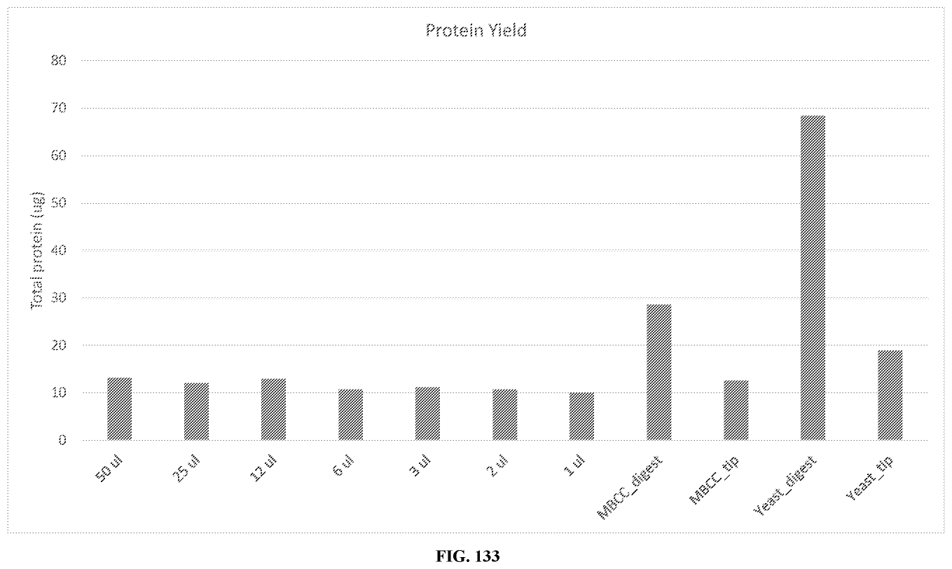

The total yield across serial dilutions was consistent. The MBCC and yeast cells had higher yield from the in-tip samples and higher still in the liquid samples. FIG. 133 shows total protein yield from all digested samples.

### DIA-NN Analysis for Tip Vs Liquid Sample

There were fairly similar protein and peptide identifications (IDs) between digests and tips, however there were slightly more protein and peptide IDs for both MBCC (419 more IDs) and yeast (94 more IDs).

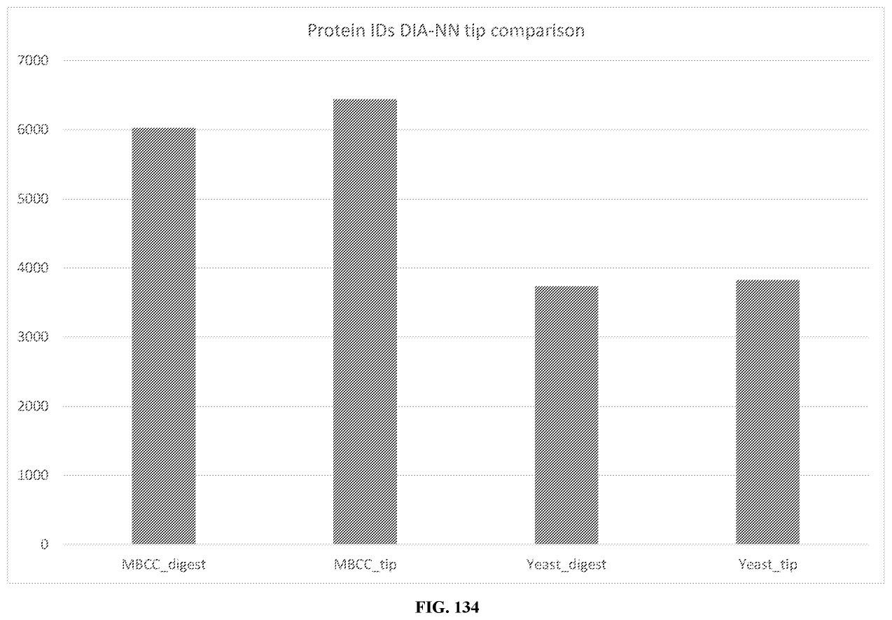

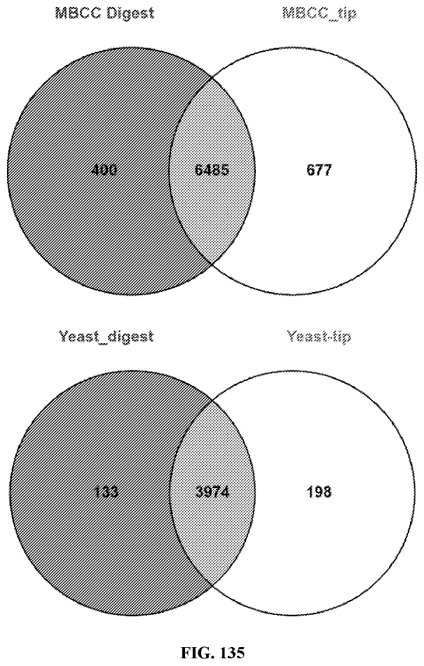

FIG. 134. Shows protein IDs from liquid samples (digest) or in-tip processed samples (tip) for MBCC and yeast cells.

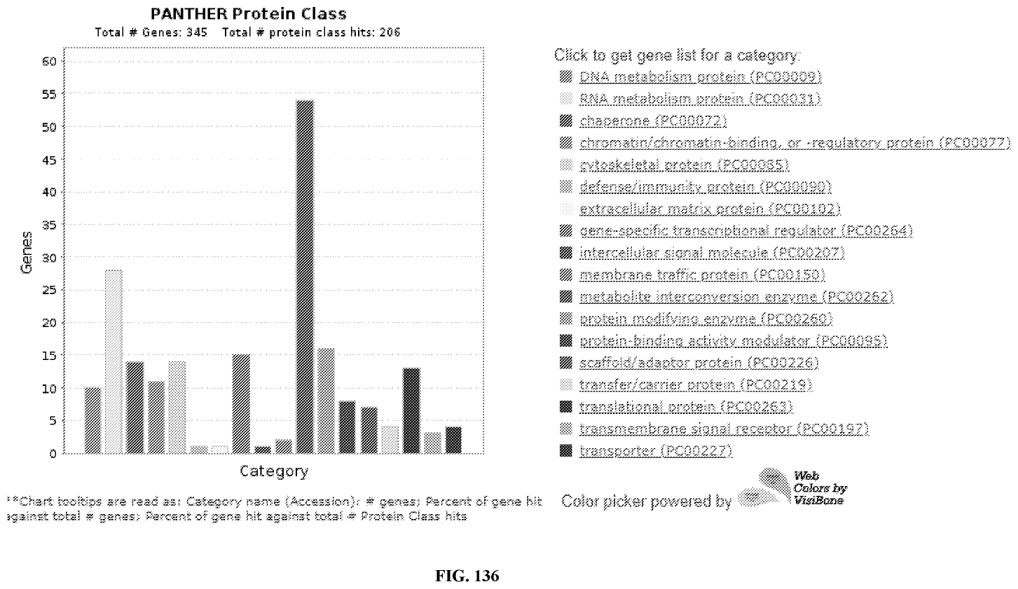

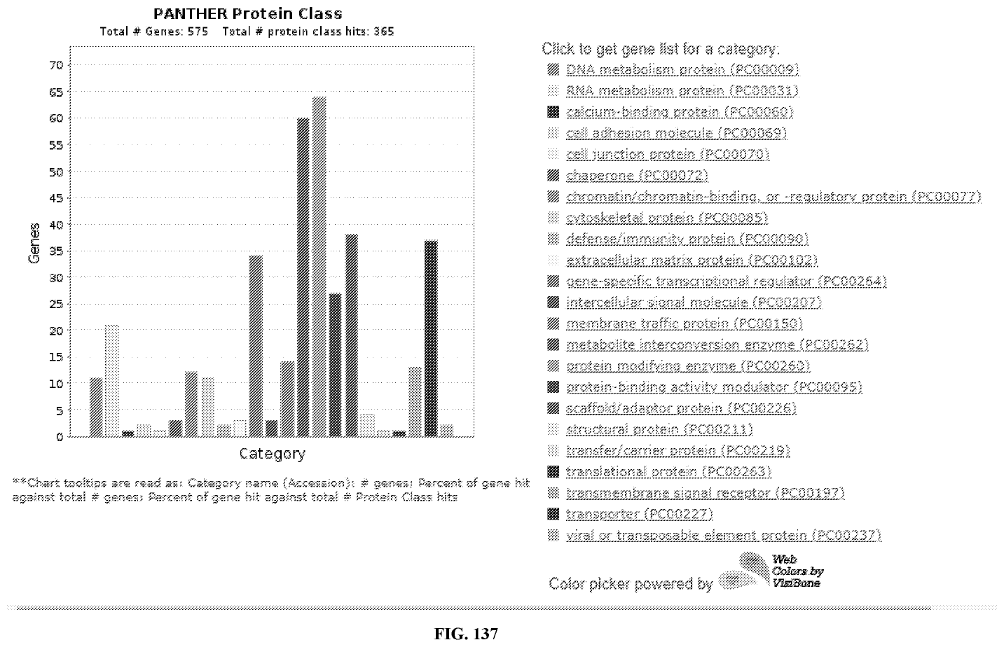

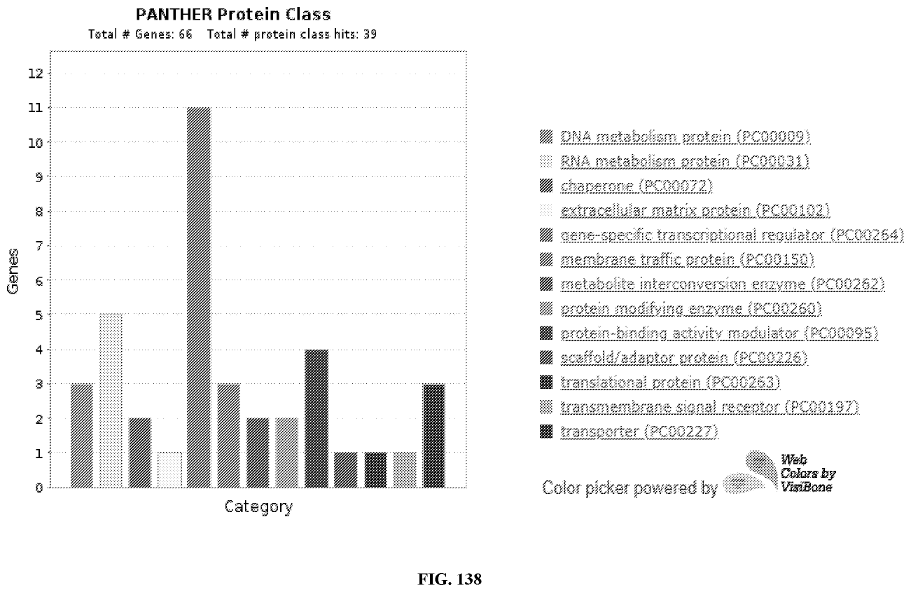

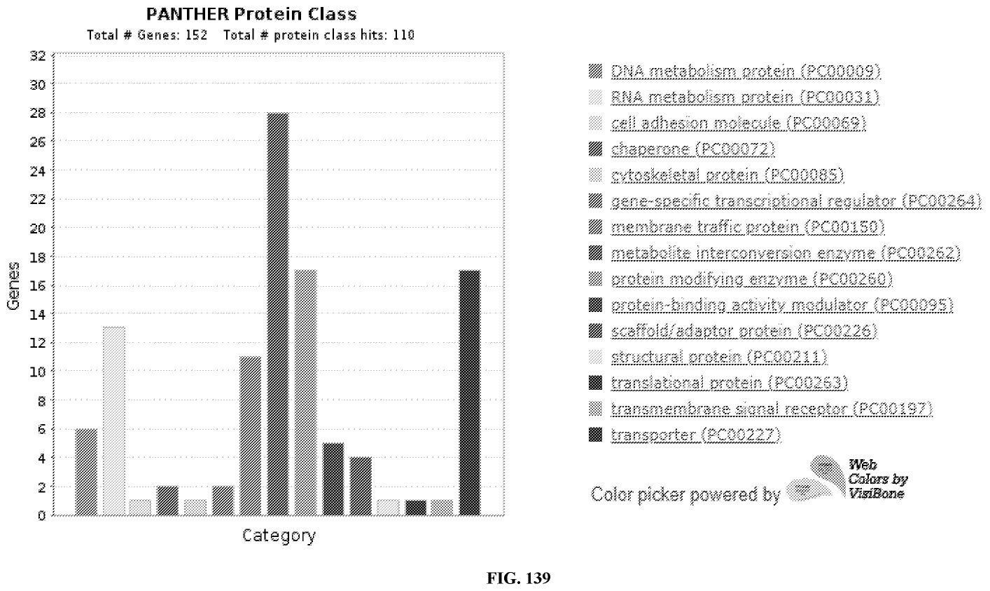

Analysis of the overlap revealed that the protein IDs for each sample type (liquid vs DBS) was ˜10% unique proteins for the MBCC and ˜5% unique for the yeast cells. This provides evidence that the sample collection and digestion methods can produce a set of unique IDs that would otherwise be lost with conventional methods. FIG. 135 shows the overlap of protein IDs between liquid (digest) and in-tip samples for MBCC and yeast cells. FIG. 136 and FIG. 137 show proteins found exclusively in MBCC liquid, and MBCC in-tip samples, respectively. FIG. 138 and FIG. 139 shows proteins found exclusively in yeast liquid samples, and yeast in-tip samples.

### Serial Dilution of MBCC in Whole Blood

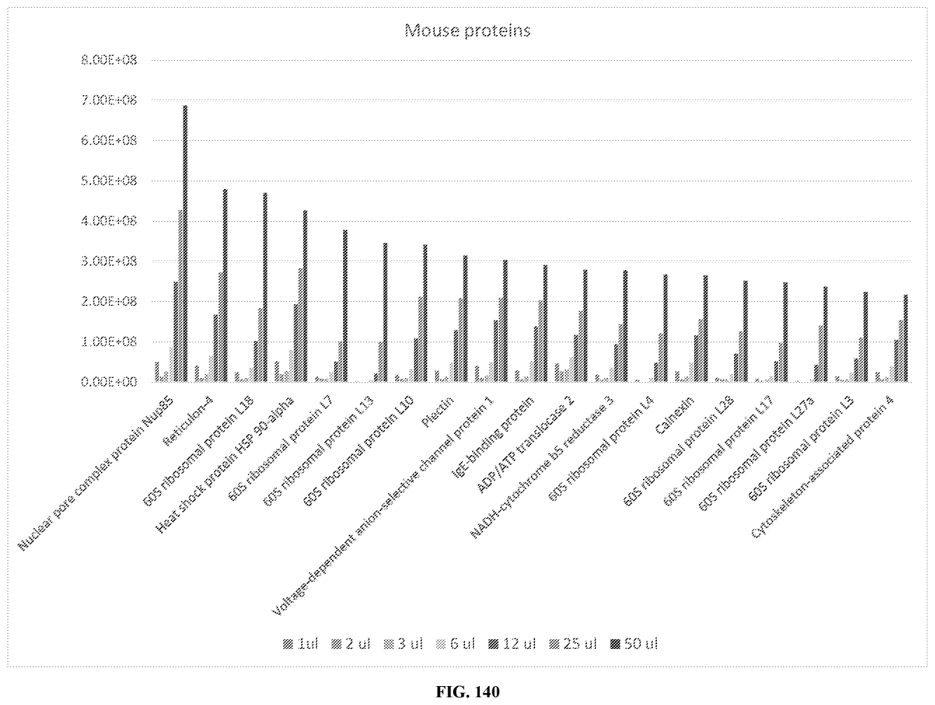

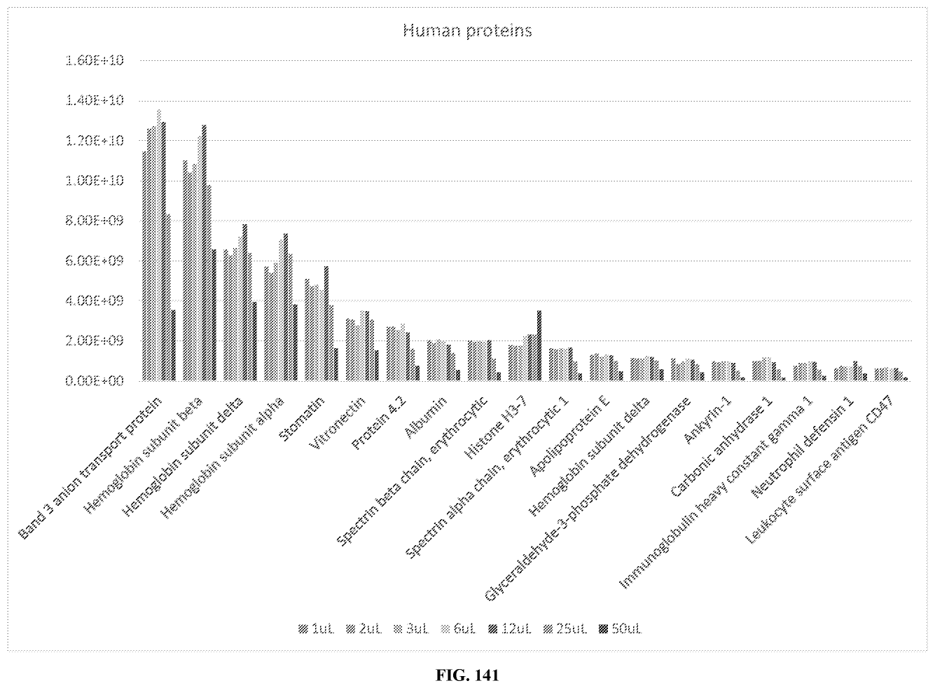

In all MBCC dilutions, mouse proteins were identified. There is a serial dilution trend in both mouse and human proteins. This trend begins to plateau at the lower dilutions (1-6 μL). FIG. 140 shows abundant mouse proteins across serially diluted MBCC in whole blood. FIG. 141 shows abundant human proteins across serially diluted MBCC in whole blood.

### Conclusions

Total protein yield concentration was greater in liquid samples compared to tips, however the DIA-NN analysis revealed slightly greater protein IDs in both MBCC and yeast. Additionally, there were more unique proteins identified in the tip samples with overall improved coverage over the protein classes. MBCC spiked into whole blood and collected in-tip was detectable and discrimination between mouse proteins and human proteins was possible. The level of detection for MBCC in whole blood was determined to be between 6-12 μL spike, which equates to between 318-636 cells/μL or 15,891-31,781 cells/30 μL VAMS.

### Example XVII. Assessment of Various Extraction Solutions, Wash Buffers, Digestions, Porous Materials and Pre-Treatment

Aims: To assess the viability of extracting protein from a tip that has been previously extracted for DNA or immunoassay. To look at various types of washing for whole blood tips including various combinations and the use of alternative enzymes to trypsin (Asp-N and Glu-C). To test the feasibility of phosphorylated peptide enrichment on peptides extracted from whole blood. Testing smaller volume extraction (10 μL), use of alternative porous materials, and testing protein quantification in saliva. Finally, to test the effect of pre-loading samples with various solutions.

### Methods

### Sample Preparation

Eight VAMS 30 μL and one VAMS 10 μL were loaded with whole blood and left to dry for at least 1 week. One VAMS 30 μL was loaded with saliva and left to dry with desiccant overnight. To test alternative porous materials, two small 2 mm×2 mm squares of PVDF membrane was cut and wet with methanol. 30 μL of blood was applied to the top of each. One was placed in an Eppendorf tube and store at −80° C. overnight and the other was left to dry with desiccant overnight. Double spun plasma was loaded into two VAMS 30 μL. One was placed in an Eppendorf tube and store at −80° C. overnight and the other was left to dry with desiccant overnight.

Two VAMS 30 μL were pre-loaded with BSA by incubating the empty tips in 10% BSA for 2 hours then washed (without drying) in either 500 mM LiCl & 100 mM Tris. Tips were then left to dry overnight at room temperature with desiccant. Plasma was subsequently loaded into the tips and one was left to dry with desiccant overnight while the other was placed in an Eppendorf tube and stored at −80° C. overnight.

Two VAMS 30 μL were pre-loaded with SDS by incubating the empty tips in 2% SDS for 2 hours and left to dry overnight at room temperature with desiccant. Plasma was subsequently loaded into the tips and one was left to dry with desiccant overnight while the other was placed in an Eppendorf tube and stored at −80° C. overnight.

### Protocol Used for Overnight Digestion

Once dried, the tips were washed with one of the following methods:
a) Wash overnight with 500 mM LiCl (1 mL), 100 mM Tris, then 2×LiCl wash (with centrifuge pulses (10,000 g)
b) PBS and protease inhibitor (PI) extraction overnight for DNA extraction and immunoassay, then overnight was with 500 mM LiCl (1 mL) and 100 mM Tris, then 2×LiCl wash (with centrifuge pulses (10,000 g)
c) 1 U benzonase in 100 μL added and incubated for 10 mins, then washed overnight in 2% CHAPS, 500 mM LiCl and 100 mM Tris then 2× wash with the same buffer using sonication (3× sonic probe, each wash). Tips were washed three times in 1 mL of water at the end of washing.
d) Wash overnight with 10 mM citric acid, 500 mM LiCl, 10 mM TBP, 40 mM CLA, then wash 2× in the same buffer with centrifuge pulses (10,000 g). Tips were washed three times in 1 mL of water at the end of washing.
e) Wash overnight with 500 mM LiCl (1 mL) and 100 mM Tris then wash with 2M urea and 2% CHAPS (2×) with centrifuge pulse (10,000 g). Tips were washed three times in 1 mL of water at the end of washing.
f) Wash overnight with 500 mM LiCl (1 mL) and 100 mM Tris, then wash with 500 mM LiCl, 100 mM dithiothreitol (DTT) for 30 mins, followed by a wash with 500 mM LiCl, 100 mM Tris, and 40 mM chloroacetamide (CLA) for 30 mins with centrifuge pulses (10,000 g).

100 μL Lysis Buffer (1% SDC, 10 mM TCEP, 40 mM CLA, 100 mM TEAB, pH 8) was added to all samples except those to be digested with Asp-N or Glu-C, to these only 50 L buffer was added. All tips were heated at 95° C. for 10 mins with gentle agitation. 1 μg (2 μL) of trypsin (0.5 μg/μL in 100 mM TEAB) was then added to all samples except where alternate proteases were used. For these, the tips were resuspended to a final volume of 500 μL in 100 mM TEAB and 1 μg of Asp-N or 1 μg of Glu-C. All tips were then incubated overnight at 37° C. (19-21 hours). After incubation the tip was removed, and the remaining digest was diluted with 800 μL of 0.5% Formic acid for the alternative proteases, 505 μL of 1.5% formic acid was added. This solution was then centrifuged at 12,000 g for 10 mins and the supernatant was transferred to a conditioned SPE cartridge for clean-up. Peptides were then analysed using mass spectrometry.

### Phosphorylated Peptide Enrichment

For enrichment of phosphorylated peptides, digested peptides from prior experiments were pooled (all processed with LiCl washing and trypsin digestion), totaling 175 μg of digested peptides. Samples were enriched for phosphorylated peptides using Zr-IMAC-HP beads (35 μL) following the manufacturer's instructions. Peptides were then analysed using mass spectrometry.

### Data Analysis

Whole blood samples were all combined in one DIA-NN search, all plasma samples were combined in one search, and the Glu-C, Asp-N, saliva, and phosphorylation samples were search independently. Phosphorylation samples were also searched using MS Fragger and MaxQant.

### Results

### Total Yield

The yields of the PBS extracts (for DNA and immunoassay analysis) are very similar to the LiCl control. Citric acid and the combination wash include CHAPS are both lower than observed in Example XV indicating more has been washed out in the initial washes/extracts. The yield for the 10 μL tip was approximately a third of that of the 30 μL tip. The PVDF membrane had a high yield, and visually did not appear that much had been washed out.

For the BSA pre-loaded tips, the dried sample had double the yield than frozen, but were all significantly lower than the controls. The SDS pre-loaded tips had a higher yield than the control plasma samples indicating that more potentially study to the tip.

### Protein IDs

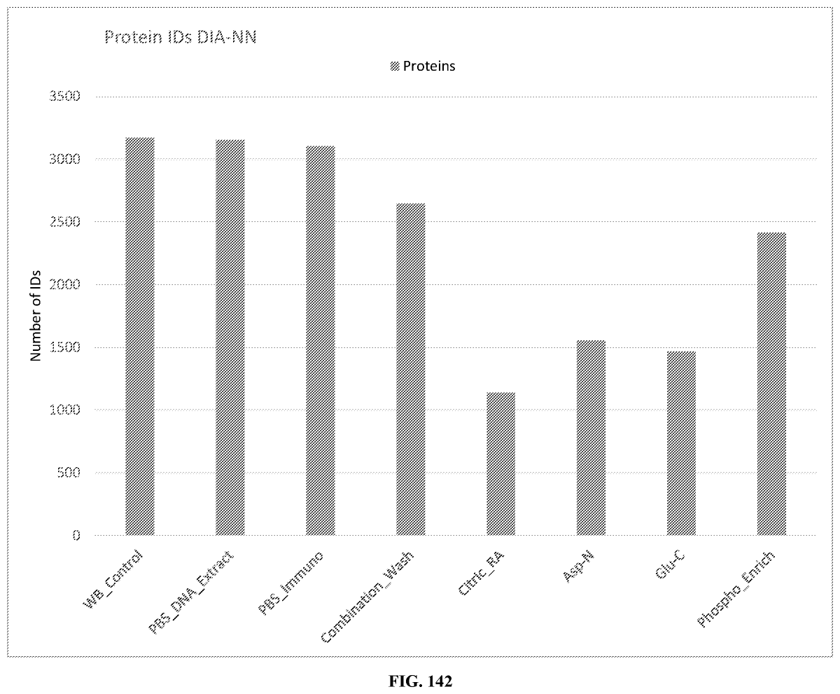

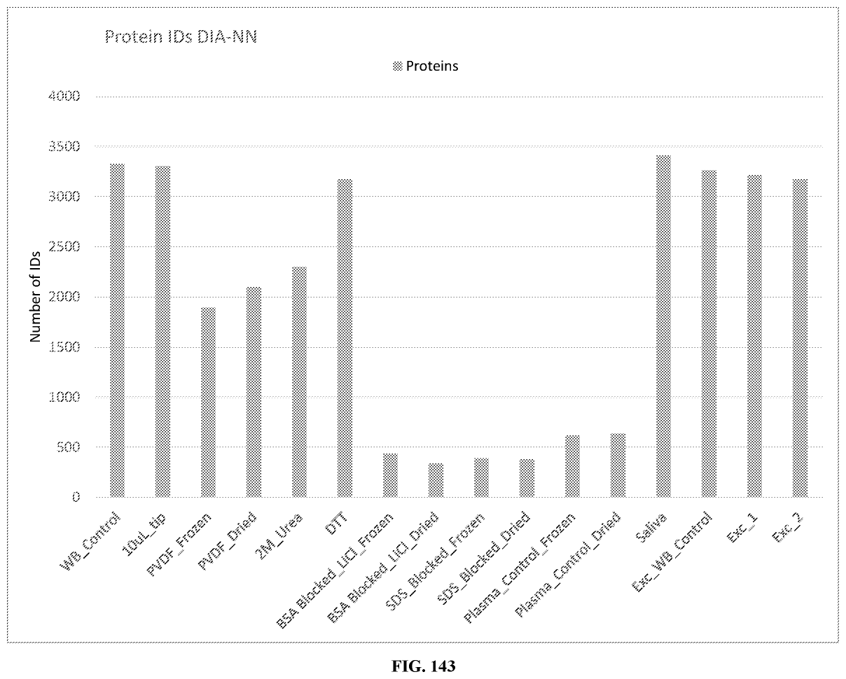

In the samples with high protein yields, typically lower proteins were identified due to higher levels of high abundance proteins. This specifically affected, the citric acid wash (FIG. 142), and the PDVF samples (FIG. 143). An unexpectantly high protein ID amount was found in the phosphorylated peptide enriched sample (FIG. 142) and in the saliva sample (FIG. 143). Although the trypsin samples produced more protein IDs, the Asp-N and Glu-C both produced a respectable number of IDs (3100 for trypsin vs 1500 for Asp-N and Glu-C). The combination of proteases may produce are larger total ID number still.

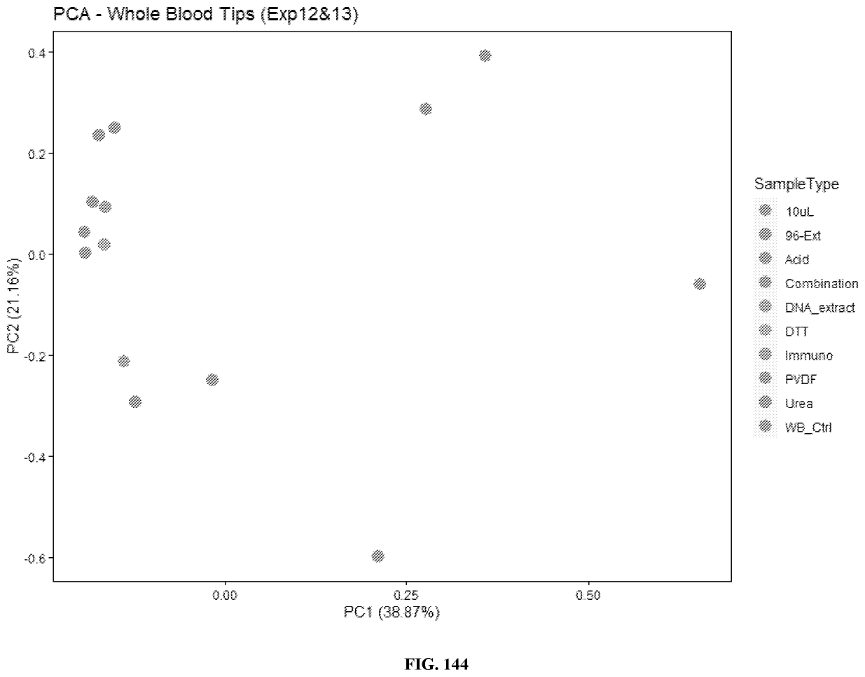

Principal component analysis (PCA) revealed that the standard LiCl method, PBS extraction for sequential analysis DNA/immunoassay before mass spectrometry analysis, and 10 μL VAMS all clustered with similar profiles. However, the results of the PVDF membrane were distinct as well as the urea-CHAPS wash (FIG. 144—urea) and the citric acid wash. This further supports that these methods are applicable to other porous materials and wash solutions and that the methods can be used to produce a particular desired profile.

### Comparison 1: Sequential Extraction for DNA/Immunoassay Analysis Followed by Mass Spectrometry Analysis

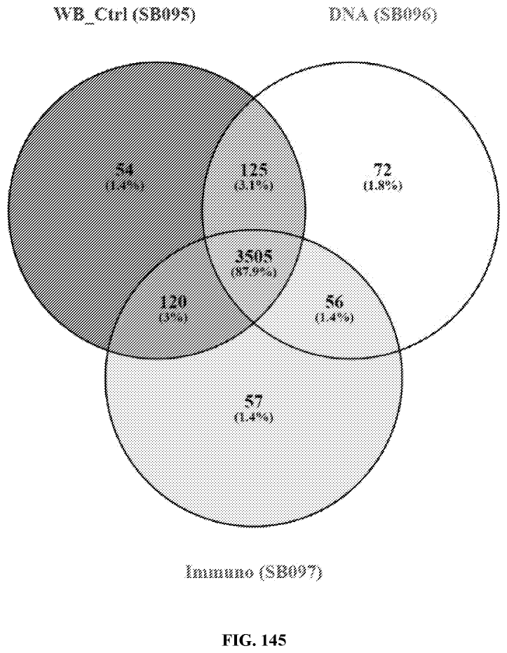

There was significant overlap between the control and the PBS extracts (DNA and immunoassay extracts) in protein IDs, demonstrating that sequential extraction can be performed to maximise the number and type of analyses from a single sample, without compromising quality of the samples for mass spectrometry analysis (FIG. 145).

### Comparison 2: Utilising Different Wash Buffer and Wash Buffer Combinations

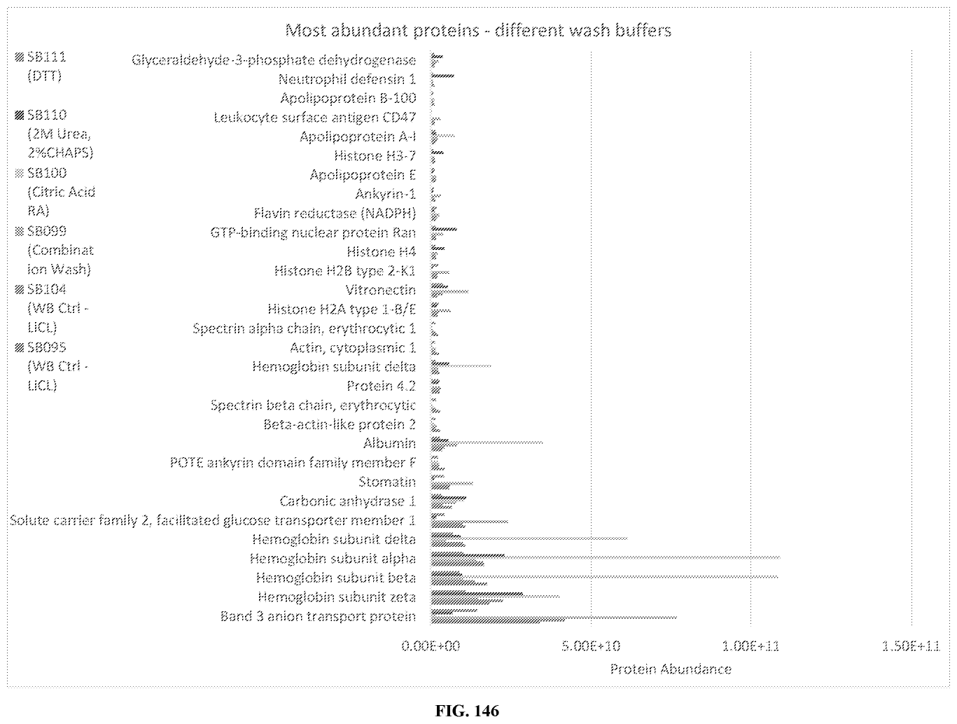

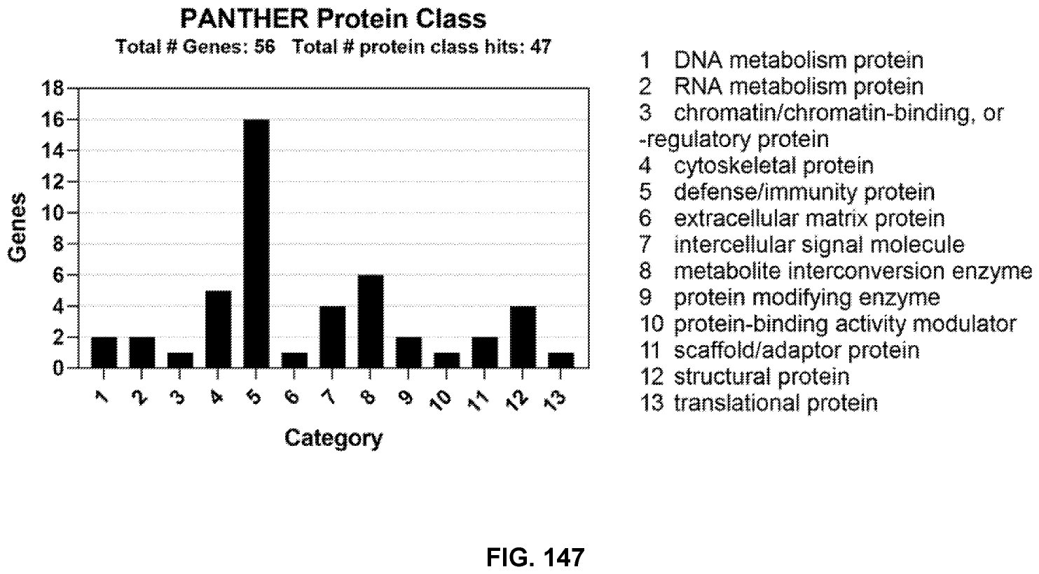

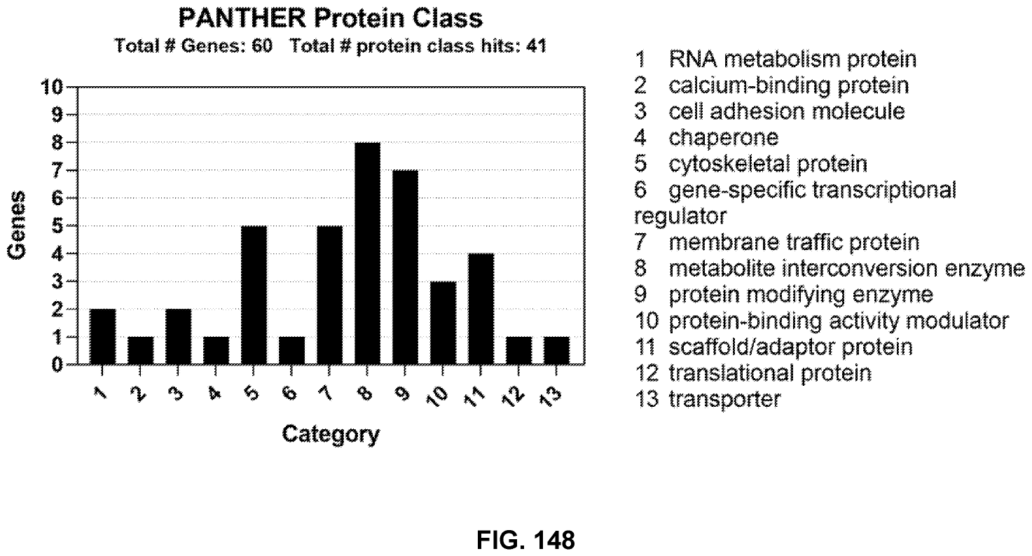

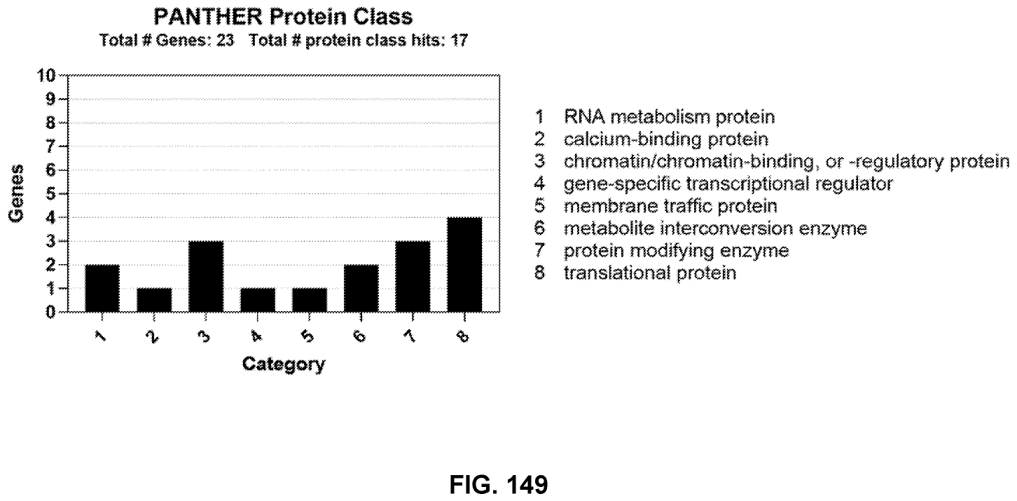

Each of the different wash buffers gave slightly different profiles. The citric acid wash had large amounts of haemoglobin and albumin, which gave reduced proteins IDs. The 2M Urea & 2% CHAPS wash enriched for a unique set of proteins with a high proportion of defence/immunity proteins and metabolite interconversion enzymes (FIG. 146 & FIG. 147). Using DDT as a reducing agent also produced metabolite interconversion proteins, but also protein modifying enzymes, cytoskeletal proteins and membrane traffic proteins (FIG. 146 & FIG. 148). The combination wash alternatively produced primarily translational proteins, chromatin/chromatin-binding or regulatory proteins, and protein modifying enzymes (FIG. 146 & FIG. 149).

### Combination 3: Use of Different Proteases

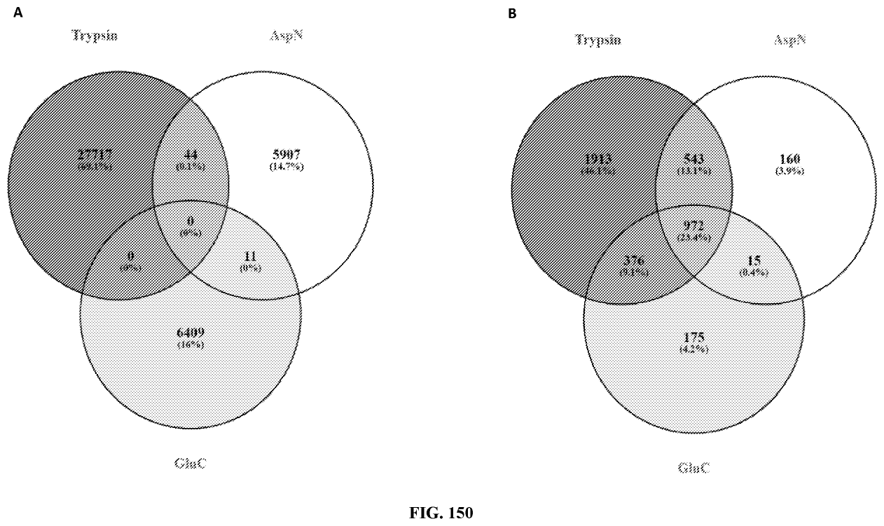

There was minimal overlap in the peptides produced by the different proteases, which is to be expected, but substantial overlap of proteins. Despite the overlap, there were still 350 proteins identified when Glu-C and Asp-N were used that were not seen with trypsin (FIG. 150).

### Comparison 4:10 μL VAMS vs 30 μL VAMS

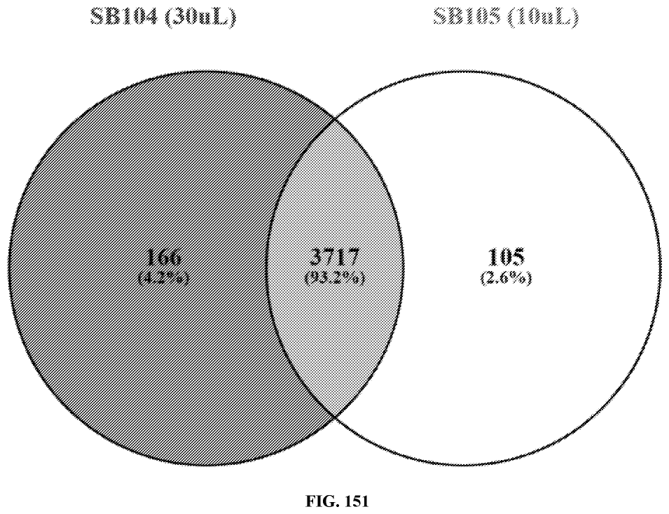

The 10 μL VAMS and 30 μL had substantial overlap of proteins as was anticipated. The 10 μL VAMS had lower levels of haemoglobin than the 30 μL VAMS, likely due to the greater wash buffer volume to VAMS capacity ratio as both tips were washed in 1 mL volumes (1:100 vs 1:33 for 10 μL and 30 μL VAMS respectively). FIG. 151 shows overlap of proteins extracted from 30 μL VAMS and 10 μL VAMS using the same protocol.

### Comparison 5: Utilisation of Alternative Porous Material

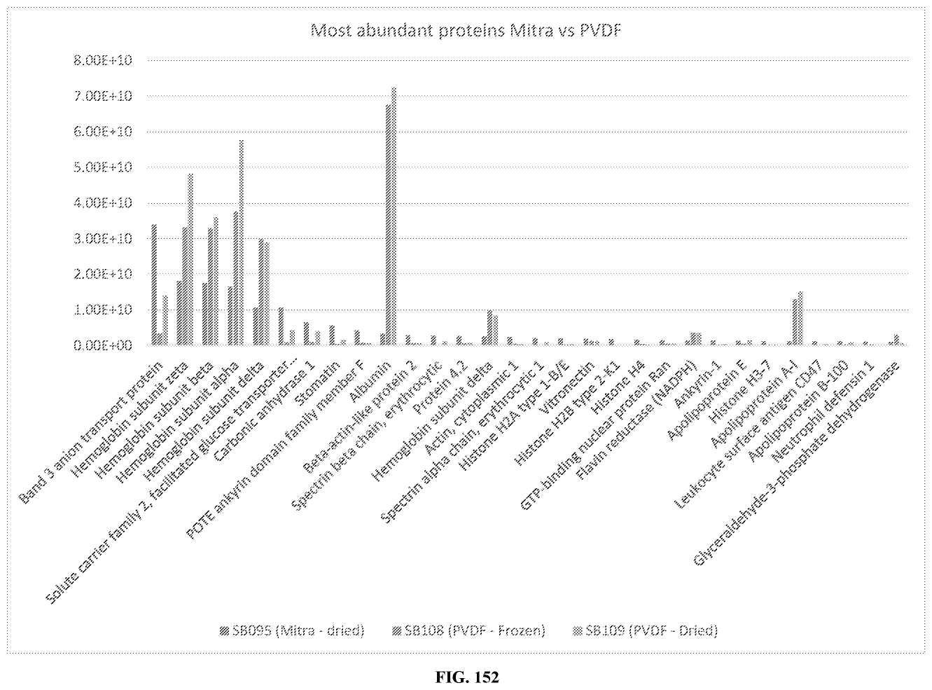

Whole blood in VAMS produced 1011 more proteins IDs compared to PVDF membrane. This is hypothesised to be result of high abundance proteins not being as adequately removed. Regardless, use of the alternative porous material still successfully extracted proteins and produced an altered profile to traditional VAMS. There was little difference between drying and freezing the PVDF sample prior to extraction. FIG. 152 shows Relative abundance of top ranked abundant proteins for whole blood samples collected either in 30 μL VAMS (Mitra®) or onto PVDF membrane.

### Comparison 6: Pre-Loading Tips

Pre-loading with BSA and SDS caused absorption of plasma into the VAMS to be slower and reduced the overall protein ID number to be reduced by almost 40%. Overall, freezing the sample compared to drying the sample had little to no effect.

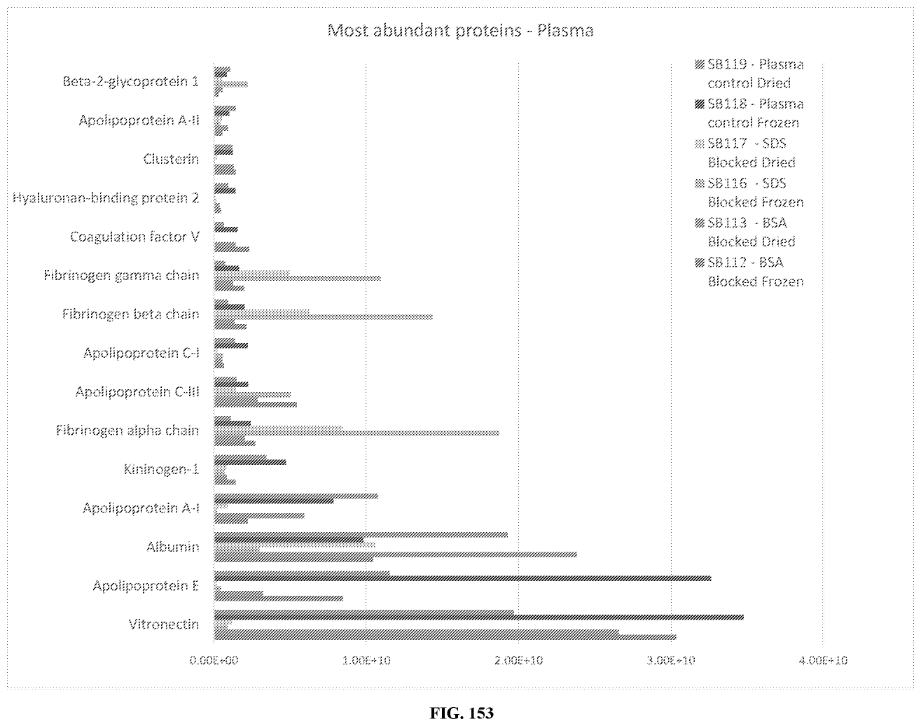

The SDS pre-loaded tips reduced a lot of the high abundance proteins (i.e. vitronectin and apolipoproteins) but the intensity was increased for the fibrinogens (FIG. 153). There were 89 significantly up-regulated proteins in the BSA pre-loaded tips compared to the control with a higher abundance of metabolite interconversion enzymes and cytoskeletal proteins. The 136 significantly down-regulated proteins were comprised of protein modifying enzymes and scaffold/adaptor proteins. For the SDS pre-loaded samples, there were 77 significantly up-regulated proteins (primarily protein-binding activity modulator) and 322 significantly down-regulated proteins (defence/immunity proteins and protein modifying enzymes) compared to the control.

### Comparison 7: Phosphorylation Enrichment of Dried Blood Spot Samples

The enriched phosphorylated peptide fraction was run in both DDA and DIA modes on a 90 minute gradient. DDA was analysed with Max Quant and Fragpipe. Fragpipe results were also used to form a spectral reference library for DIA analysis using DIANN.

In total, 4887 phosphorylated peptides were identified across all three platforms, with 1883 common to all three. Across each of the platforms the phosphorylated peptides accounted for ˜50% of the total spectra.

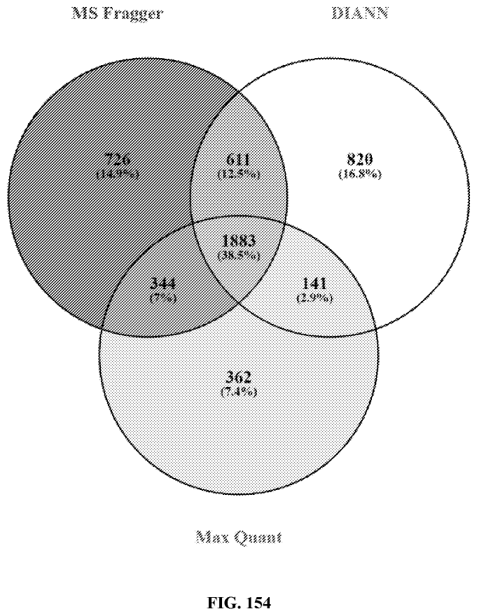

FIG. 154 shows overlap of phosphorylated peptides identified across three analytical platforms, Fragger, DIANN, and Max Quant.

Overall, there was successful enrichment and analysis of phosphorylated peptides from these samples. No literature exists on phosphorylation studies for dried blood spots, but these results demonstrate a greater level of detection that for that reported for liquid blood samples.

### Comparison 8: Saliva in VAMS

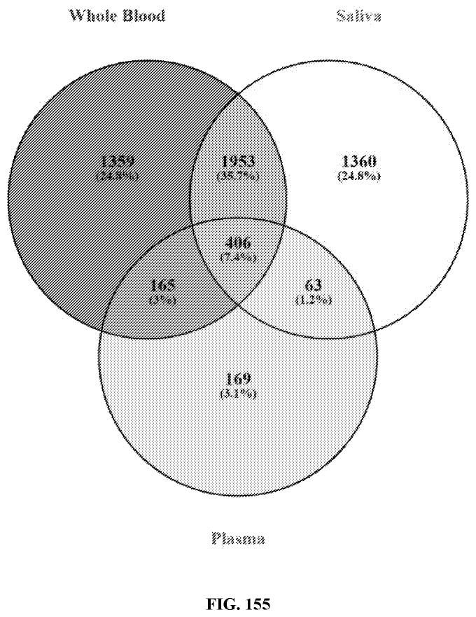

Proteins were successfully detected from saliva collected into VAMS. DIANN quantified 3415 proteins from saliva. Prior literature searches reveal the average detected from liquid saliva is 2000, thus our methods are producing greater numbers. There is substantial overlap in protein IDs between whole blood, plasma, and saliva (FIG. 155).

### Conclusions

### Comparison 1: Sequential Extraction for DNA/Immunoassay Analysis Followed by Mass Spectrometry Analysis

An initial PBS extraction enables analysis of DNA and immunoassays prior to the standard mass spectrometry extraction and digestion. The mass spectrometry results from each protocol were very similar indicating no detrimental effects. This provides evidence that multiple extractions can be performed from a single sample.

### Comparison 2: Utilising Different Wash Buffer and Wash Buffer Combinations

All of the wash buffers produced different profiles as observed previously. The use of 2M urea and CHAPS in combination as well as the combination of benzonase, LiCl, CHAPS and sonication produced unique protein profiles.

### Combination 3: Use of Different Proteases

Both alternative tested proteases (Asp-N and Glu-C) successfully digested unique peptides and increased the total number of identified unique proteins when combined with the trypsin results.

### Comparison 4:10 μL VAMS Vs 30 μL VAMS

The extraction from the 10 μL VAMS was equally as successful as the 30 μL VAMS.

### Comparison 5: Utilisation of Alternative Porous Material

The PVDF membrane produced a different profile compared to what is typically seen using the VAMS. However, the haemoglobin was still present in the samples which resulted in in lower protein IDs overall.

### Comparison 6: Pre-Loading Tips

The tips pre-loaded with BSA and SDS resulted in fewer protein IDs compared to the control plasma, with more down-regulated proteins overall. A different profile was observed in the pre-loaded samples, particularly in those pre-loaded with SDS.

### Comparison 7: Phosphorylation Enrichment of Dried Blood Spot Samples

The phosphorylation enrichment was successful, and a good number of phosphorylated peptide IDs were found.

### Comparison 8: Saliva in VAMS

This achieved an excellent result compared with the literature on liquid samples. DIA quantitated 3415 proteins from the saliva sample.

### Example XVIII. Assessment of Various Extraction Solutions, Wash Buffers, Digestions, Porous Materials

Aim: To assess the viability of extracting multiple samples from a tip for sequential mass spectrometry analysis. To look at various types of washing for whole blood tips including various salts using a range of concentration and the use of alternative enzymes to trypsin (Glu-C). Finally, testing the use of alternative porous materials.

### Methods

### Sample Preparation

Ten VAMS 30 μL were loaded with whole blood and nine were left to dry with desiccant overnight, the other was placed in an Eppendorf tube and stored at −80° C. overnight. To test alternative porous materials, 30 μL of blood was applied to two pieces of filter paper. One was placed in an Eppendorf tube and store at −80° C. overnight and the other was left to dry with desiccant overnight.

### Protocol Used for Overnight Digestion

a) Once dried, the tips were washed with one of the following methods:
b) Wash overnight with 250 mM LiCl+100 mM Tris (1 mL), then 2×LiCl wash (with
c) Wash overnight with 500 mM LiCl+100 mM Tris (1 mL), then 2×LiCl wash (with
d) Wash overnight with 1M LiCl+100 mM Tris (1 mL), then 2×LiCl wash (with centrifuge pulses (10,000 g)
e) Wash overnight with 500 mM CaCl₂)+100 mM Tris (1 mL), then 2×LiCl wash (with centrifuge pulses (10,000 g)
f) Wash overnight with 500 mM LiCl+100 mM Tris (1 mL), then wash with 500 mM LiCl+100 mM Tris (1 mL) with centrifuge pulses (10,000 g). This extract was collected for immunoassay analysis (wash A). Then a final wash of 7M Urea, 2M Thiourea, 1% sodium deoxycholate (SDC)+tributylphosphine (TBP)+choloractamide (CLA)+100 mM Tris (1 mL) with centrifuge pulses (10,000 g), this fraction was collected for digestion and mass spectrometry analysis (wash B). Wash A and B were acetone precipitated with 10 mL of acetone for 2 hours. The pellets were resuspended in lysis buffer (1 mL for wash A and 100 μL for wash B) 100 μL was taken from each for tryptic digestion as below. The remaining tip was washed three times in 1 mL of water at the end of the third wash.

100 μL Lysis Buffer (1% SDC, 10 mM TCEP, 40 mM CLA, 100 mM TEAB, pH 8) was added to all samples except the filter paper samples, to these 300 μL lysis buffer was added. All samples were heated at 95° C. for 10 mins with gentle agitation. 1 μg (2 μL) of trypsin (0.5 μg/μL in lysis buffer) was then added to all samples. After incubation the samples were removed, and the remaining digest was diluted with 800 μL of 0.5% Formic acid. The tips to be digested further, were removed from the solution and diluted with 700 μL of 0.5% Formic acid. A further 100 μL of 100 mM TEAB was then added to the tips and 1 μg trypsin or 1 μg Glu-C was added. These were incubated for a further 2 hours and the digested material was then pooled with the original 800 μL of collected digest. All samples were centrifuged at 12,000 g for 10 mins and the supernatant was transferred to a conditioned SPE cartridge for clean-up. Peptides were then analysed using mass spectrometry.

### Data Analysis

Whole blood samples were all combined in one DIA-NN search, with the exception of the Glu-C samples that were searched independently.

### Results

### Total Yield and Protein IDs

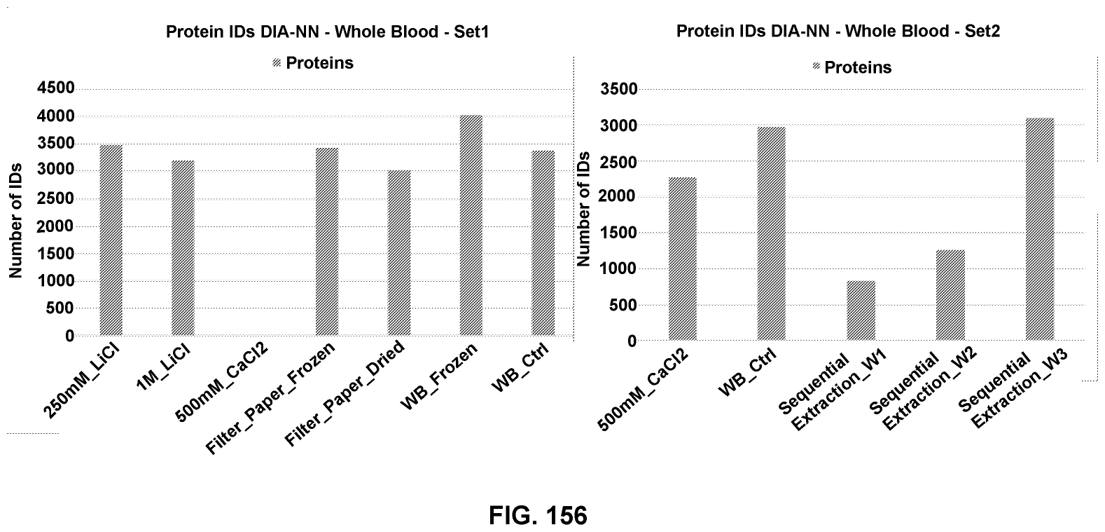

The CaCl₂) washed sample had a much higher yield that any of the other conditions, indicating that a lot of higher abundance proteins (ie haemoglobin) weren't as effectively removed. A minimum of 3000 IDs were found in each of the samples, with the exception of CaCl₂) which only had ˜2250 IDs and the sequentially extracted samples (FIG. 156). The frozen filter paper had slightly higher IDs compared to the dried sample, which is the same trend observed from the frozen vs dried VAMS. The filter paper had ˜500 fewer IDs than the VAMS overall, but still produced a good number.

### Comparison 1: Sequential Extraction for Multiple Mass Spec Analyses

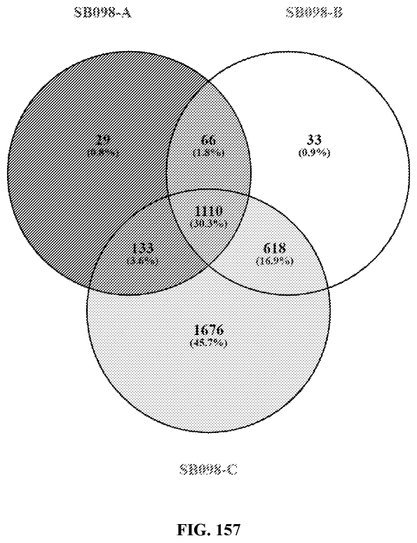

Each subsequent extraction produced more IDs than the prior extraction with a total of 3665 unique proteins identified (FIG. 156). Of the unique proteins identified, 30% was found in all samples, and 96.5% was found in the final extraction (FIG. 157). The first two contributed very few unique proteins, though 36% and 50% of the total were also identified in the first and second extraction respectively. This indicates that sequential extraction can be performed and does not have a significant effect on the final in-tip digest and analysis. These results also highlight the value of washing the tips before performing the digest, as a simple liquid extraction is not nearly as effective at maximising protein IDs.

### Comparison 2: Concentrations of LiCl

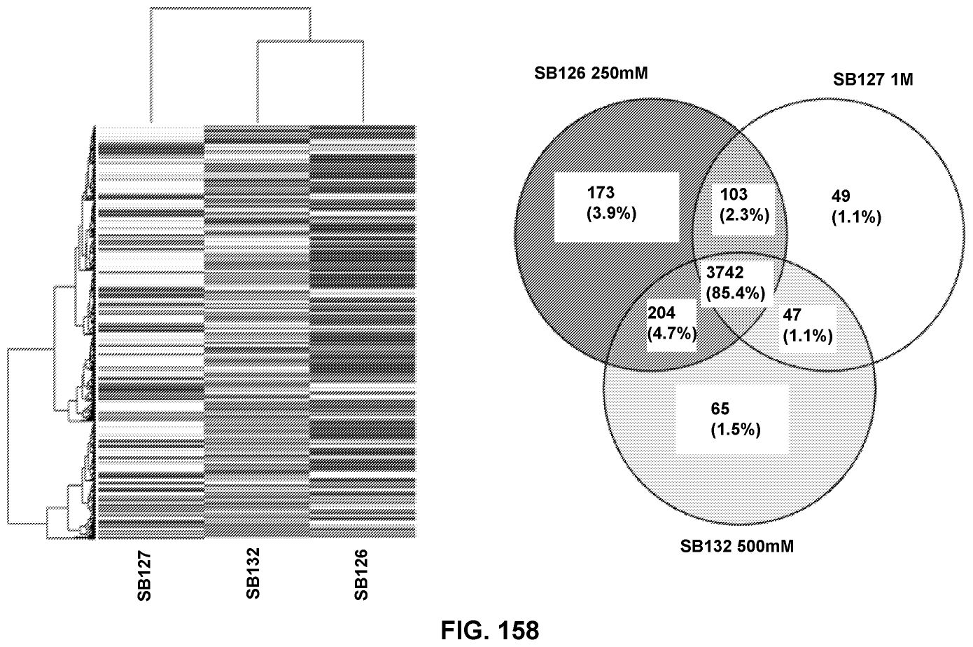

Washing with 250 mM, 500 mM, or 1M LiCl gave distinct protein profiles (FIG. 158). Washing with 250 mM LiCl gave the largest number of unique proteins as well as the largest total number of IDs, whilst the 1M comparison gave the least of both. The proteins unique to 250 mM LiCl were primarily metabolite interconversion enzymes and RNA metabolism proteins.

### Comparison 3: Use of CaCl₂ Instead of LiCl

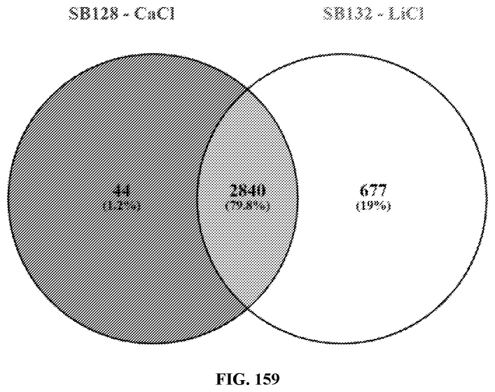

More proteins were identified in the samples washed with LiCl compared to CaCl₂, with 20% more IDs (FIG. 159). This is likely to due poor washing, and abundant proteins being left behind, analysis revealed increased amounts of hemoglobin (subunit delta, beta, alpha) compared to the LiCl samples.

Washing with CaCl₂) did result in identification of 44 unique proteins. These were comprised of protein modifying enzymes, chromatin/chromatin-binding or regulatory protein, and defence/immunity proteins.

### Comparison 4: Utilisation of Alternative Porous Material

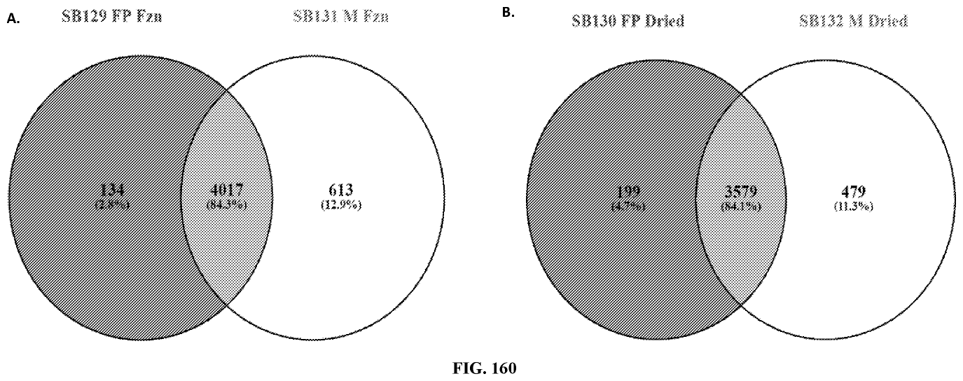

The same trend of higher protein IDs from samples stored in the freezer compared to dried was observed both for the filter paper samples and for the VAMS. Overall, there were many similarities observed between the use of filter paper and VAMS, with ˜84% overlap between sample types, indicating that these methods are compatible with either. Overall, more unique ID's were identified from VAMS (FIG. 160).

The proteins unique to VAMS compared to filter paper were largely comprised of membrane interconversion enzymes, structural proteins and translational proteins, whilst the proteins unique to filter paper were comprised of metabolite interconversion enzymes and intercellular signal molecules.

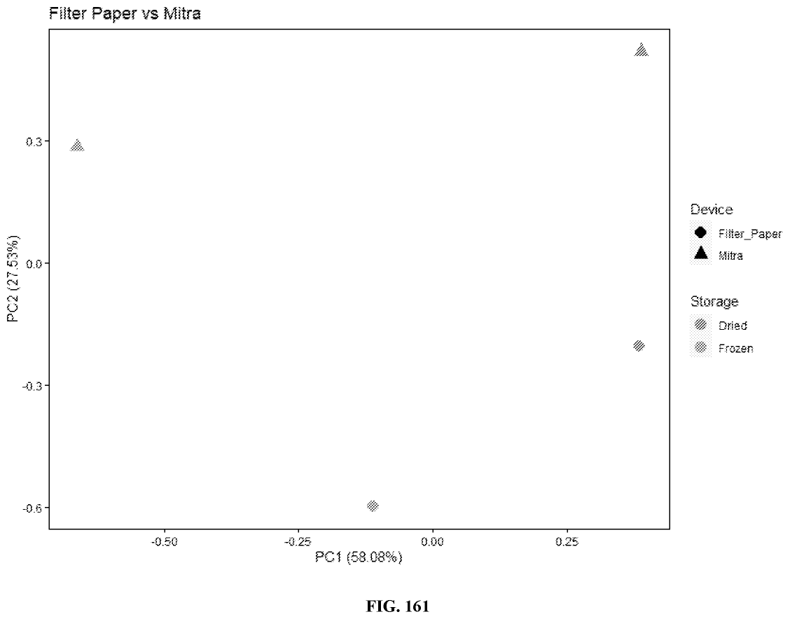

Based on the PCA there is less variation between filter paper and VAMS than there is between dried vs freezing samples immediately after collection (FIG. 161). Similarly, heatmap hierarchical clustering clusters the frozen and dried samples together, as opposed to the VAMS and the filter paper.

### Comparison 5: Use of Different Proteases

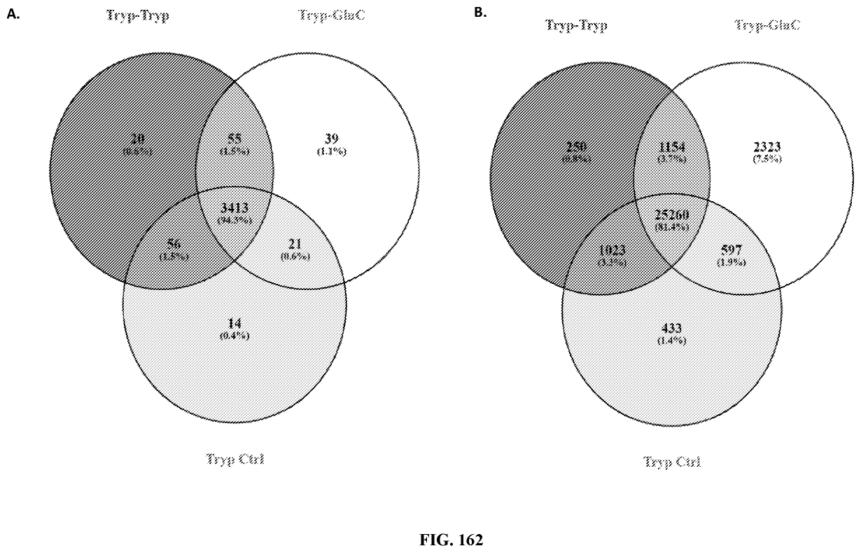

The data was searched using DIA-NN in two ways, the first using Glu-C and Trypsin specifying cuts at K, R, D, E and the second just using trypsin. The Trypsin-only search many more peptides and proteins overall, but the Glu-C search did give unique IDs for the Glu-C sample. Results from both searches are shown in FIG. 162. The inclusion of Glu-C resulted in many more peptide IDs overall, though not significantly protein IDs.

The proteins unique to the Glu-C digested sample include protein modifying enzymes, protein-binding activity modulator, and defence/immunity proteins.

### Conclusions

### Comparison 1: Sequential Extraction for Multiple Mass Spec Analyses

Multiple extraction and mass spectrometry analysis can be performed on a single tip, each giving slightly different profiles, though the final in-tip digestion gave the largest number of unique proteins, indicating that protein immobilisation of the tip is highly effective.

### Comparison 2: Concentrations of LiCl

The 250 mM LiCl performed the best in producing the most protein IDs, but all concentrations gave their own unique profile.

### Comparison 3: Use of CaCl₂) Instead of LiCl

The CaCl₂) washed samples had higher amounts of hemoglobin compared to the LiCl control. Considering that lower concentrations of LiCl were found to perform better (comparison 2), it may be that lower concentration of CaCl₂) could also produce improved results.

### Comparison 4: Utilisation of Alternative Porous Material

The filter paper performed similarly to the VAMS overall and had the same trend for frozen vs dried samples. These methods are compatible with multiple porous materials.

### Comparison 5: Use of Different Proteases

Digesting a second time with trypsin did not appear to have an effect on the overall protein IDs. Digesting with Glu-C produced more unique peptides, but only a few more proteins IDs.

### Example XIX. Assessment of Dilution of Whole Blood or pH Adjustment

Aims: To investigate the effect of dilution of whole blood and plasma or adjustment of pH in samples prior to loading VAMS. To test another porous material that has been treated to enable longer shelf-life, and to test lower concentrations of wash/extraction solutions.

### Methods

### Sample Preparation

Four VAMS 30 μL were loaded with whole blood and left to dry with desiccant for at least 24 hours. One VAMS 30 μL was loaded with plasma and left to dry with desiccant for at least 24 hours. Additionally, whole blood and plasma were diluted in either 1:2 or 1:4 in PBS prior to loading VAMS and were left to dry with desiccant for at least 24 hours.

For pH adjustment of samples, either 1) 1M TEAB (10 μL) or 2) 1M citric acid (10 μL) was added to 90 μL whole blood or plasma to a pH of 8 and 5.5-6 respectively. Samples were applied to VAMS and left to dry with desiccant for at least 24 hours.

Two new material Mitra® VAMS were also loaded with whole blood or plasma and were left to dry with desiccant for at least 24 hours.

### Protocol Used for Overnight Digestion

Once dried, the tips were washed with one of the following methods:
a) Wash overnight with 500 mM LiCl+100 mM Tris (1 mL), then 2×LiCl wash (with centrifuge pulses (10,000 g)
b) Wash overnight with 125 mM LiCl+100 mM Tris (1 mL), then 2×LiCl wash (with centrifuge pulses (10,000 g)
c) Wash overnight with 50 mM LiCl+100 mM Tris (1 mL), then 2×LiCl wash (with centrifuge pulses (10,000 g)
d) Wash overnight with 50 mM CaCl¬2+100 mM Tris (1 mL), then 2×LiCl wash (with centrifuge pulses (10,000 g)

100 μL Lysis Buffer (1% SDC, 10 mM TCEP, 40 mM CLA, 100 mM TEAB) was added to all samples. Tips were then heated at 95° C. for 10 mins with gentle agitation. 1 μg (2 μL) of trypsin (0.5 μg/μL in Lysis Buffer) was then added to all samples. Tips were incubated overnight at 37° C. (19-21 hours). After incubation the sample diluted with 800 μL of 0.5% Formic acid and the tip was removed. This solution was then centrifuged at 12,000 g for 10 mins and the supernatant was transferred to a conditioned SPE cartridge for clean-up. Peptides were then analysed using mass spectrometry.

### Data Analysis

Samples were searched in two sets, plasma or whole blood, using DIA-NN.

### Results and Discussion

### Total Yield

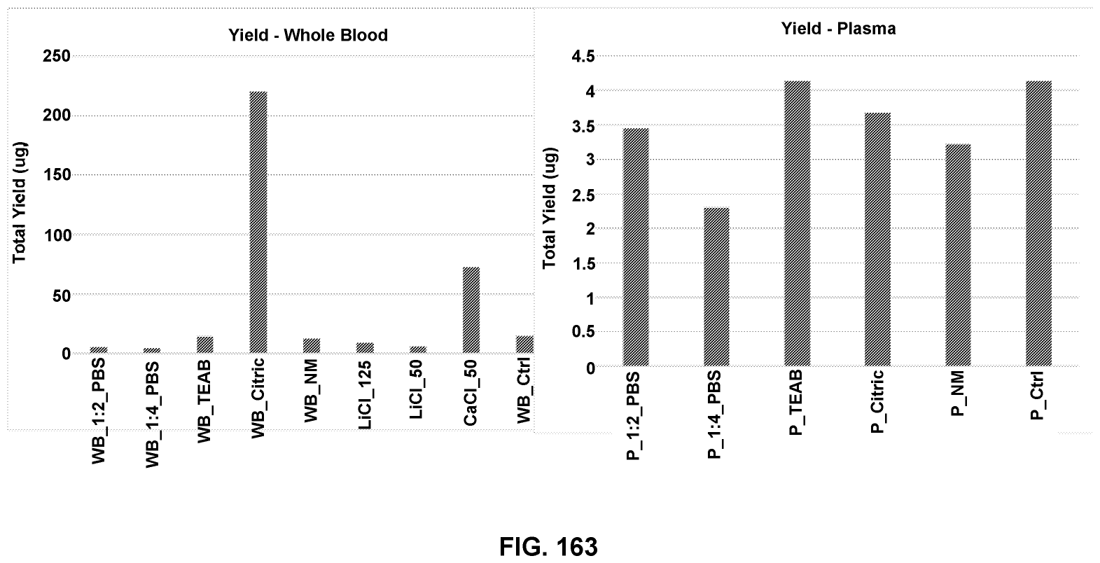

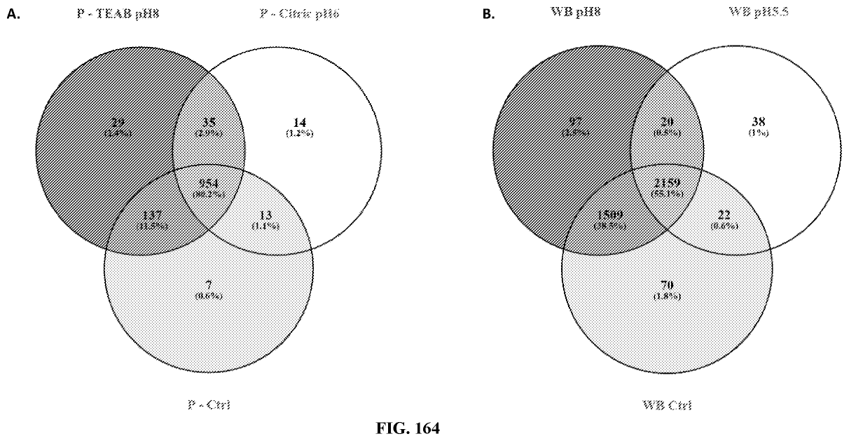

As seen previously, the citric acid treated samples produced the highest yield (FIG. 1). Interestingly, it did not produce the same effect in the plasma. CaCl2 also had a higher yield (FIG. 163). The total yield decreased with decreasing concentrations of LiCl, suggesting more proteins were washed away.

Protein IDs for whole blood samples were quite consistent across all samples, with the exception of the acidified blood and the CaCl2 wash which were lower, which is consistent with total yield results (high yield, low ID count). Protein IDs for remaining samples were all between 3000-3250 IDs. Protein IDs for the plasma samples were consistent across the whole set (700-1000 IDs). There were slightly more IDs observed in the 1:2 diluted sample, and the acidified samples had lower IDs as for the whole blood sample. The lower IDs observed for the acidified samples and CaCl2 samples are likely due to higher abundance proteins remaining in the tip thus reducing overall dynamic range of detection.

### Comparison 1: Dilution of Samples Prior to Loading

The plasma sample diluted 2× produced the most unique proteins (51 unique proteins) compared to the 4× dilution and the undiluted control. These proteins were comprised of translational proteins, RNA metabolism proteins, and uncategorised proteins. Conversely, the most unique proteins (125 unique proteins) were found in the undiluted control for whole blood compared to the 2× and 4× dilution.

### Comparison 2: Adjusting pH of Samples Prior to Loading VAMS

The TEAB pH 8 adjusted plasma gave slightly higher number of protein IDs compared to the untreated control and the Citric Acid pH 6 adjusted plasma (FIG. 164). The pH 8 samples had an increased proportion of translational proteins. In the whole blood samples, the pH 8 samples produced the most unique samples however only by a small margin (FIG. 164).

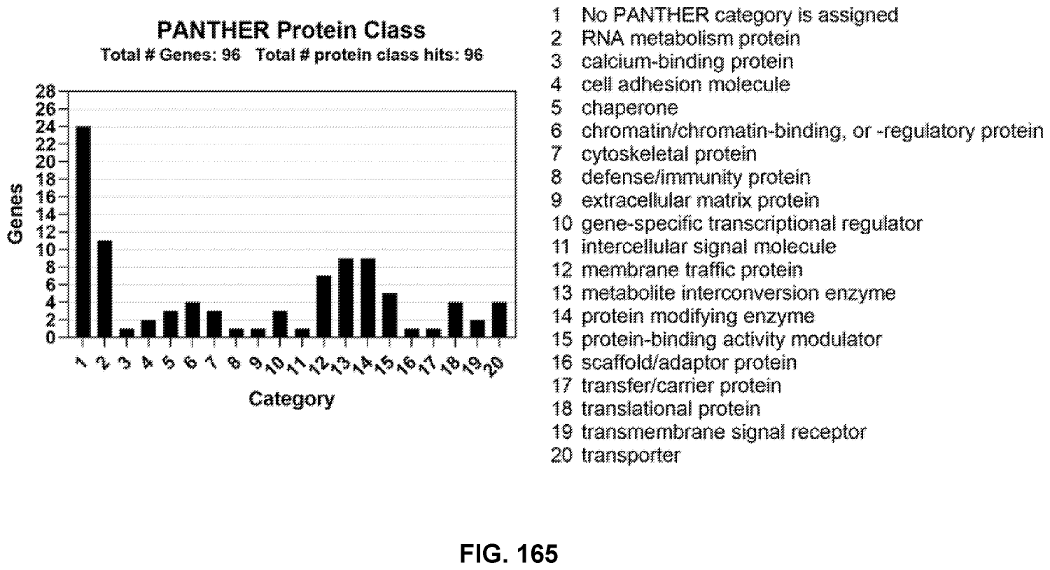

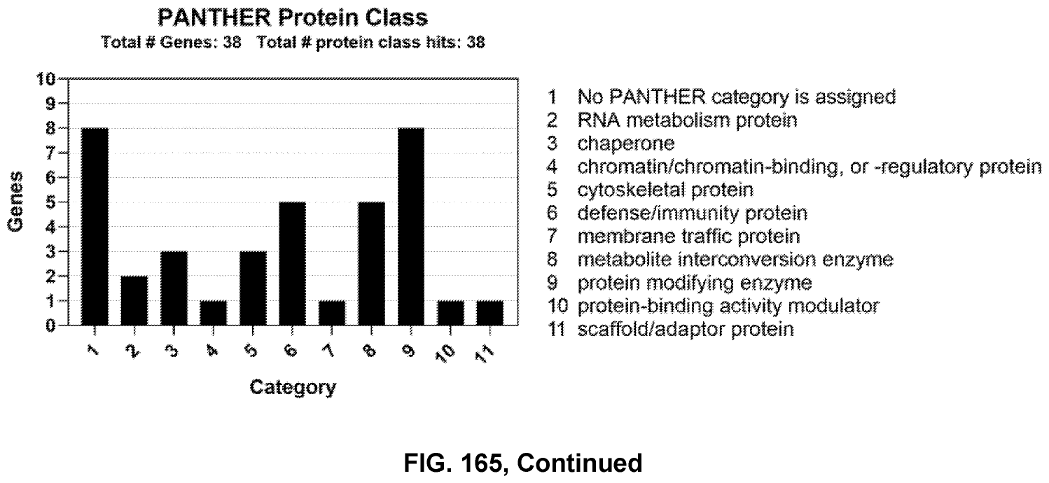

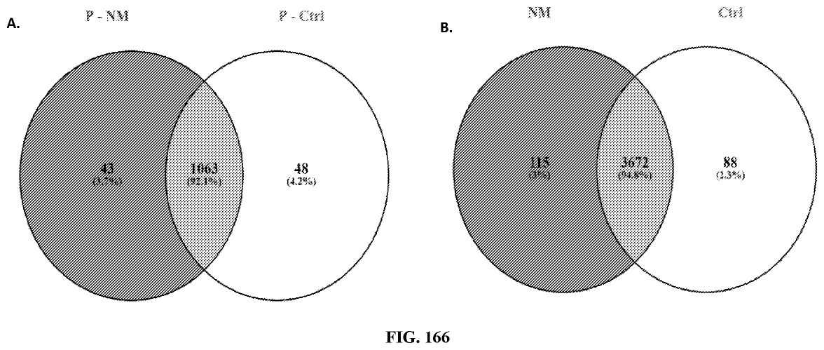

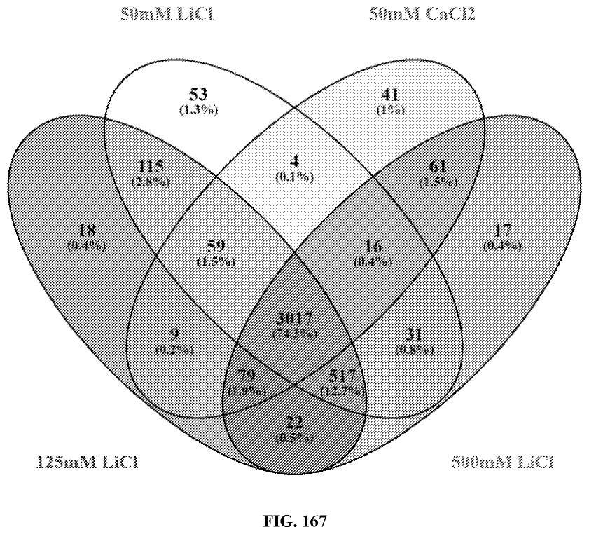

The protein classes of the unique proteins in the pH adjusted samples were different to each other, revealing a unique profile of unique proteins from each treatment (FIG. 165).

### Comparison 3: Testing Alternative Porous Material

There was good agreement between the IDs for the standard VAMS and the long shelf-life VAMS for both plasma and whole blood samples (FIG. 166). There was good agreement in the detected abundant proteins between the two devices for both plasma and whole blood samples.

### Comparison 4: Various Washing Concentrations

Although there were slightly more IDs in the 125 mM LiCl compared to the 500 mM LiCl control, the 50 mM LiCl gave the highest number of unique IDs (FIG. 167). Even though the CaCl2 had the lowest number of IDs overall, it did produce a set of unique proteins (FIG. 167). The protein profiles of the samples washed with 50 mM LiCl or 50 mM CaCl2 were similar, except for a higher number of metabolite interconversion enzymes with LiCl, and protein modifying enzymes with CaCl2.

The 50 mM LiCl washed sample gave the lowest peak area for most hemoglobin subunits and the tip after washing was visibly whiter. As expected, the CaCl2 washed sample had the highest levels of hemoglobin, explaining the reduced number of IDs.

### Conclusions

### Comparison 1: Dilution of Samples Prior to Loading

For plasma, the 2× dilution produced the highest number of unique IDs, whilst the undiluted whole blood control produced the highest number of unique IDs. However, the difference in IDs between dilutions were overall small.

### Comparison 2: Adjusting pH of Samples Prior to Loading VAMS

Some proteins bound more strongly in the presence of acid (notably hemoglobin and albumin) and other bound more strongly under alkaline conditions (notably vitronectin).

### Comparison 3: Testing Alternative Porous Material

There was little to no difference between the tested porous materials.

### Comparison 4: Various Washing Concentrations

The 50 mM LiCl did remove more hemoglobin from samples and produced more unique IDs, but not necessarily more IDs overall. CaCl2 produced a reduced number of total IDs due but also had a number of unique IDs.

### Example XX. Extraction and Analysis of Glycans

Aims: To investigate the effect of dilution of whole blood and plasma or adjustment of pH in samples prior to loading VAMS. To test another porous material that has been treated to enable longer shelf-life, and to test lower concentrations of wash/extraction solutions.

To assess the suitability of the tips and current methods for extraction of glycans to test the effect of washing tips exclusively with Tris or H2O.

### Methods

### Sample Preparation

Four VAMS 30 μL were loaded with whole blood and left to dry with desiccant for at least 24 hours. Four VAMS 30 μL were loaded with plasma and left to dry with desiccant for at least 24 hours.

### Glycan Digest

Once dried, the tips were washed with one of the following methods:

### No Washing was Performed for Glycomic Samples Prior to Analysis

Wash overnight with 500 mM LiCl+100 mM Tris (1 mL), then 2×LiCl wash (with centrifuge pulses (10,000 g)

100 μL Lysis Buffer (1% SDC, 10 mM TCEP, 40 mM CLA, 100 mM TEAB) was added to all samples. Tips were then heated at 95° C. for 10 mins with gentle agitation. After reduction and alkylation, 4 whole blood and 4 plasma tips were prepared for glycan analysis. Half of the samples were left with the tip suspended in the reduction/alkylation solution, and the 2 μL PNGase F added directly to this suspension. The tips were removed from the reduction/alkylation solution for the remaining half and the tips were washed 2× in 1 mL H2O. The reduction/alkylation solution was retained for trypsin digest and protein analysis. 2 μL PNGase F in 98 μL Milli-Q was then added for glycan cleavage. All samples were then incubated for 16 hours to release N-glycans. The resulting digest solution was then collected and remaining tips were washed twice with 100 μL Milli-Q H2O. Tips were then removed and washed separately with 50 μL Milli-Q twice. The first microtube was washed separately with 20 μL of Milli-Q. All washes were combined for subsequent treatment.

50 μL of ammonium acetate (pH 5) was added to the solution and incubated (1 hour at room temperature). Samples were then dried and storage at −20° C. overnight. The samples were then reduced in 70 μL of 1M NaBH¬4 in 50 mM KOH for 3 hours at 50° C. Following reduction, 350 μL Milli-Q H2O was added, immediately followed by 7 μL of glacial acetic acid. After gentle mixing, samples were cleaned using self-packed carbon tips (15 mm of carbon). Following cleaning and elution, eluent containing glycans were taken, dried down, then resuspended in 20 μL of Milli-Q H2O.

Purified glycans were injected onto a Hypercarb Porous Graphitized Carbon (PGC) column (3 μm, 1 mm×30 mm) using Agilent 1260 HPLC coupled to a ThermoFisher Velos Pro at a flow rate of 20 μL/min with the following gradient parameters: Buffer A: 10 mM ammonium bicarbonate, Buffer B: 70% acetonitrile with 10 mM ammonium bicarbonate, 0.3 min-0% B, 4 min-14% B, 40 min-40% B, 48 min-56% B, 50-54 min-100% B, 56 min-0% B. Glycans were detected in negative mode with a m/z acquisition window of 660-2000. Glycan composition was identified by accurate mass.

### Peptide Digest

The resulting washed tips were dried and were resuspended in 100 μL of 100 mM TEAB. 1 μg (1 μL) of trypsin (1 μg/μL in Lysis Buffer) was then added to all samples. Tips were incubated overnight at 37° C. (19-21 hours). After incubation the sample diluted with 800 μL of 0.5% Formic acid and the tip was removed. This solution was then centrifuged at 12,000 g for 10 mins and the supernatant was transferred to a conditioned SPE cartridge for clean-up. Peptides were then analysed using mass spectrometry.

### Data Analysis

Peptide samples were searched in two sets plasma or whole blood for glycan analysis using DIA-NN. Data analysis for glycoproteins in previously acquired samples was performed through Byonic using default instrument settings, with June 2022 human fasta database for proteins, and the standard Byonic 309 N-glycan and 9 most common O-glyancs included for potential Glycomics modifications.

### Results and Discussion

### Glycan Extraction and Results

The top 9 (by relative abundance) glycans observed between 660 and 2000 m/z are described in Table 30 and have been organised in order of abundance. Using one randomly selected sample, the top 9 most abundant glycans were identified and used to create a Skyline list for EIC analysis. Contaminating masses were also identified and included in this skyline list.

TABLE 30
Top glycans present in sample, ranked by abundance.

| | | Neutral | M/z |
|---|---|---|---|
| Rank | Glycan | mass | observed |
| 1 | 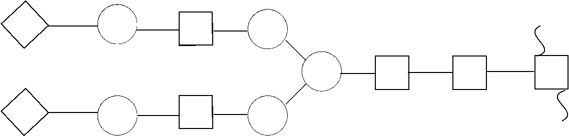 | 2221.78 | 1111.39²⁻ |
| 2 | 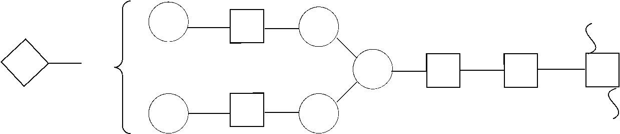 | 1930.68 | 965.84²⁻ |
| 3 | 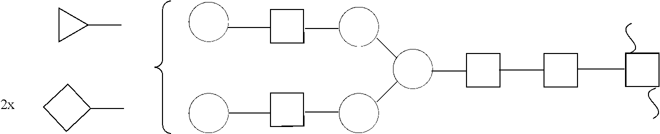 | 2367.84 | 1184.42²⁻ |
| 4 | 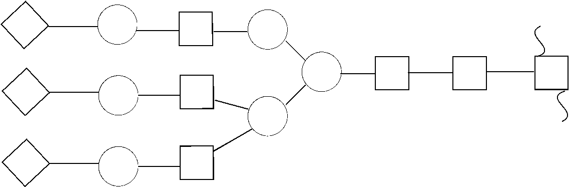 | 2878.04 | 1439.52²⁻ |
| 5 | 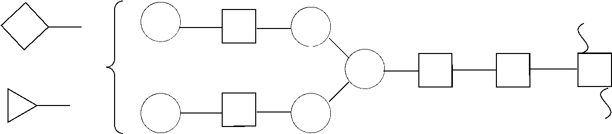 | 2076.74 | 1038.87²⁻ |
| 6 | 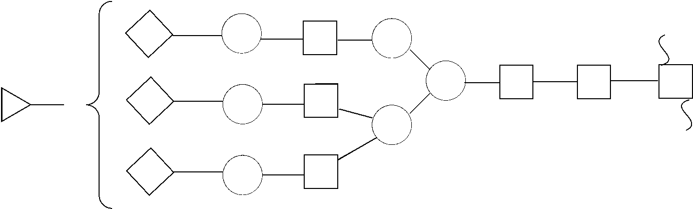 | 3024.10 | 1512.55²⁻ |
| 7 | 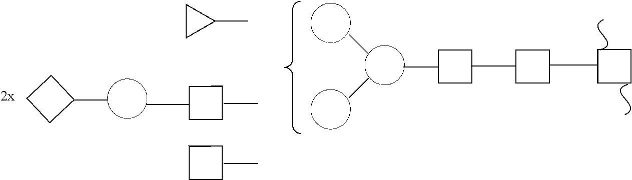 | 2570.1 | 1285.96²⁻ |
| 8 | 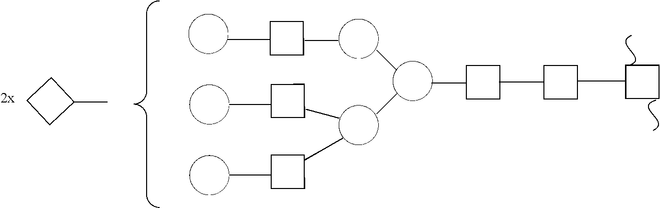 | 2586.9 | 1293.95²⁻ |
| 9 | 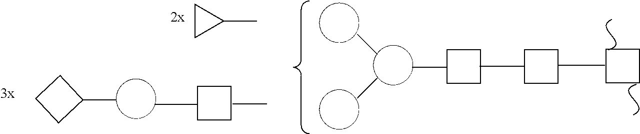 | 3170.12 | 1585.56²⁻ |

The relative abundance of all glycans is consistent between sample preps, and wet vs dry samples. This change has been quantified below in Table 31. All of the 9 most abundant glycans were detected in every sample, with the exception of the 1585.56²⁻ glycan which was only detected in 5 samples.

TABLE 31
Relative abundances of top 9 glycans, organised by abundance.

| Glycan mass | Relative Abundance | | | | | | | | |
|---|---|---|---|---|---|---|---|---|---|
| Sample | 1 | 2 | 3 | 4 | 5 | 6 | 7 | 8 | Average |
| 1111.39²⁻ | 0.517 | 0.573 | 0.583 | 0.524 | 0.532 | 0.563 | 0.564 | 0.571 | 0.553 |
| 965.84²⁻ | 0.192 | 0.187 | 0.126 | 0.250 | 0.222 | 0.218 | 0.149 | 0.229 | 0.197 |
| 1184.42²⁻ | 0.141 | 0.078 | 0.146 | 0.074 | 0.118 | 0.064 | 0.136 | 0.060 | 0.102 |
| 1439.52²⁻ | 0.061 | 0.096 | 0.079 | 0.087 | 0.042 | 0.081 | 0.063 | 0.078 | 0.073 |
| 1038.87²⁻ | 0.047 | 0.023 | 0.020 | 0.025 | 0.045 | 0.027 | 0.039 | 0.027 | 0.032 |
| 1512.55²⁻ | 0.017 | 0.028 | 0.036 | 0.025 | 0.015 | 0.029 | 0.030 | 0.022 | 0.025 |
| 1285.96²⁻ | 0.014 | 0.008 | 0.004 | 0.008 | 0.016 | 0.009 | 0.009 | 0.006 | 0.009 |
| 1293.95²⁻ | 0.010 | 0.007 | 0.003 | 0.007 | 0.012 | 0.010 | 0.006 | 0.007 | 0.008 |
| 1585.56²⁻ | 0.001 | 0.001 | 0.003 | 0.000 | 0.001 | 0.000 | 0.003 | 0.000 | 0.001 |

### Protein Total Yield and Protein IDs

The saved reduction/alkylation fraction collected prior to glycan analysis gave similar yield to the control and to the digested tip post-glycan extraction. This was consistent for both whole blood samples as well as plasma samples. Notably, the whole blood reduction/alkylation fraction contained a lower amount of IDs compared to controls, but still ˜2500 proteins were identified in these samples.

### Sequential Extraction of Proteins Following Glycan Extraction

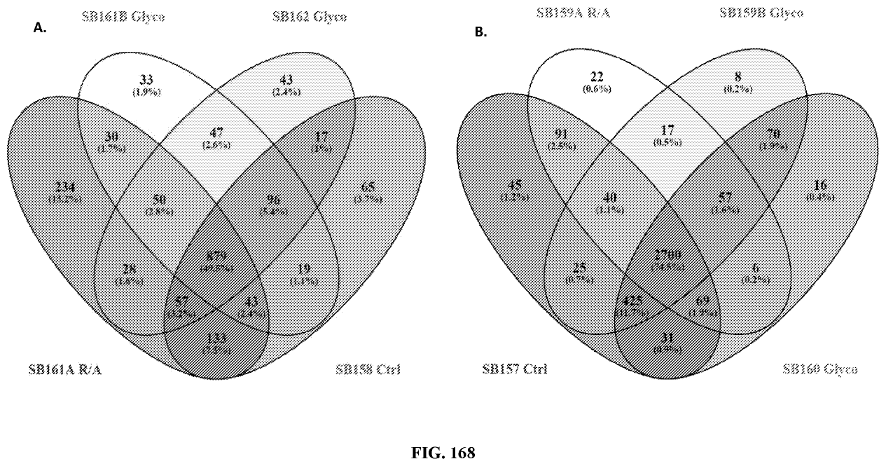

The reduction/alkylation extract contained the largest number of unique proteins, demonstrating that there was good protein extraction from that fraction (FIG. 168). Overall, the glycan extracted samples and controls had reasonable overlap. These data are demonstrative that sequential extraction can be performed to analyse glycans and proteins from a single sample. These results were consistent for both plasma samples and whole blood samples.

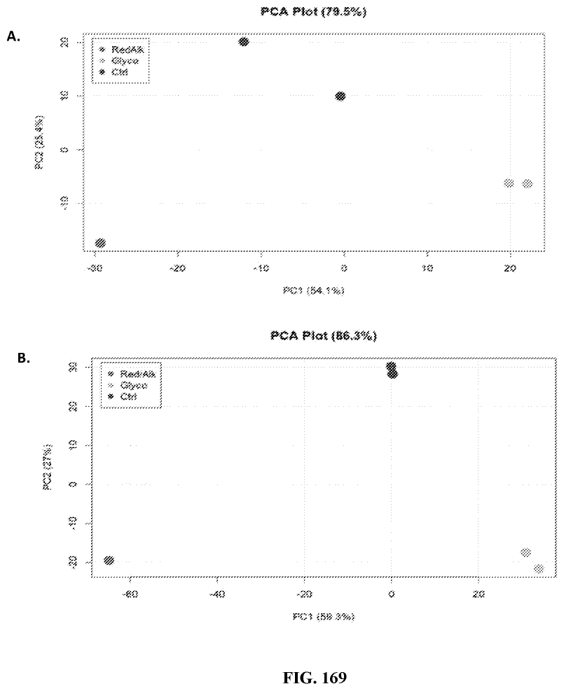

The two glycan extracted plasma samples clustered nicely together showing that even though the reduction/alkylation solution was removed prior to digestion, the results remained consistent (FIG. 169). The reduction/alkylation solution contained a higher proportion of most of the abundant proteins, with the exception of Vimentin). The abundances do not appear to change much in the glycan extracted samples.

Similar to the plasma results, the whole blood samples also had good correlation between the two glycan fractions and the controls (FIG. 169). The whole blood reduction/alkylation fraction was more contaminated with hemoglobin. The glycan cleaved samples have slightly lower abundances of other proteins, but overall follow the same trends.

Analysis of Glycoproteomics on Whole Blood Samples Previously Digested with Trypsin

Glycopeptides were detected using this data analysis approach, with a total number of unique glycopeptides numbering 589. For increased glycopeptide coverage, an enrichment strategy such as zic-HILIC is recommended.

### Conclusions

Glycan Extraction and Analysis from VAMS

Glycans were successfully extracted and detected from whole blood and plasma VAMS. The standard N-glycomics analysis pathway is compatible with the current VAMS proteomics methodologies and can be included after sample reduction and alkylation and before tryptic digestions. This workflow can further include a multi-glycomics workflow wherein different glycans can be sequentially released prior to tryptic digestion. Glycosaminoglycans (GAGs), Glycosphingolipids (GSLs) and N-glycans can all be released sequentially through enzymatic means while preserving the peptide backbone.

There were more glycans present in these samples at lower amounts, and these can be further determined with a more in-depth analysis. Total abundance of these glycans is not high and the majority of glycans were determined in the top 9 analysis performed.

Extraction of Proteins after Glycan Extraction and Analysis

Overall, the protein yield and extraction from VAMS previously prepared for glycan cleavage was successful. Removal of the reduction/alkylation solution prior to trypsin digestion didn't negatively affect the overall protein yield, but in fact additional analysis of the reduction/alkylation fraction produced extra information that may otherwise be lost.

Analysis of glycoproteomics on whole blood samples previously digested with trypsin

Glycopeptides are released during the standard method of VAMS extraction and trypsin digestion these can be detected by using specific data analysis methods.

### Example XXI. Assessment of Tris or H2O Wash

Aim: To test the effect of washing tips exclusively with Tris or H2O.

### Methods

### Sample Preparation

Four VAMS 30 μL were loaded with whole blood and left to dry with desiccant for at least 24 hours.

### Protocol Used for Overnight Digestion

Once dried, the tips were washed with one of the following methods:
a) Wash overnight with 500 mM LiCl+100 mM Tris (1 mL), then 2×LiCl wash (with centrifuge pulses (10,000 g)
b) Wash overnight with 100 mM Tris (1 mL), then 2×Tris wash (with centrifuge pulses (10,000 g)
c) Wash overnight with MQ H2O (1 mL), then 2×H2O wash (with centrifuge pulses (10,000 g)

100 μL Lysis Buffer (1% SDC, 10 mM TCEP, 40 mM CLA, 100 mM TEAB) was added to all samples. Tips were then heated at 95° C. for 10 mins with gentle agitation. 1 μg (1 μL) of trypsin (1 μg/μL in Lysis Buffer) was then added to all samples. Tips were incubated overnight at 37° C. (19-21 hours). After incubation the sample diluted with 800 μL of 0.5% Formic acid and the tip was removed. This solution was then centrifuged at 12,000 g for 10 mins and the supernatant was transferred to a conditioned SPE cartridge for clean-up. Peptides were then analysed using mass spectrometry.

Data analysis

Samples were searched in one set for whole blood samples using DIA-NN.

### Results and Discussion

### Total Yield and Protein IDs

The Tris washing gave the lowest overall yield, followed by the H2O, although the H2O was not significantly different from the LiCl. Protein IDs for the whole blood samples were consistent across the set, with the H2O extraction giving slightly lower results. Effect of washing VAMS with Tris or H2O

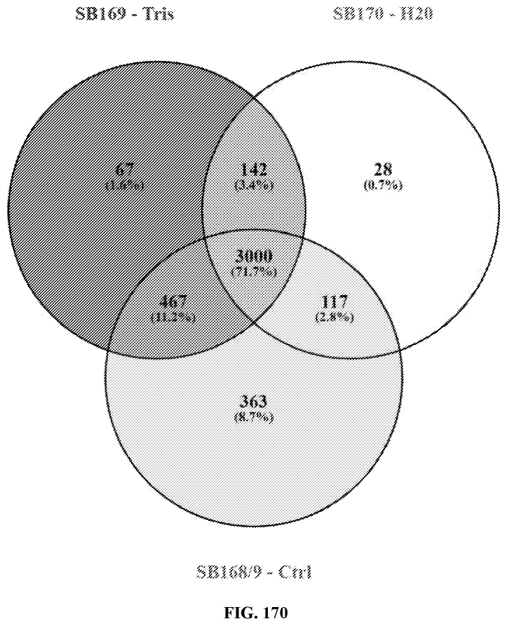

The LiCl wash solution (Ctrl) resulted in the highest number of unique protein IDs compared to Tris and H2O (FIG. 170). The unique proteins in the LiCl wash are comprised of chaperone proteins, metabolite interconversion enzymes, and protein-binding activity modulators. The H2O wash produced a high number of defence/immunity proteins, and the Tris wash produce a high diversity of proteins. Interestingly, the Tris wash removed large amounts of hemoglobin subunit delta compared to the other washes.

### Conclusions

Tris and H2O as wash solutions performed well, and both washed away a lot of high abundance proteins. But LiCl still produced the highest protein ID count overall.

### Example XXII. Assessment of Tissue Samples

Aim: To assess the suitability of the tip and these methods for tissue samples.

### Methods

### Sample Preparation

Two small pieces of tissue were excised from a frozen bovine liver into Eppendorf tube. Either (a) 500 μL of 5% sodium deoxycholate (SDC)+100 mM Tris or (b) 500 μL PBS was added to the samples. Tissue was then probe sonicated three times. Sample was applied to one 30 μL VAMS each which was then left to dry with desiccant for at least 24 hours. A 30 μL of the remaining sonicated samples was transferred to an Eppendorf and stored at −80° C. until digestion.

### Protocol Used for Overnight Digestion

Once dried, the tips were washed with one of the following methods:

Wash overnight with 500 mM LiCl+100 mM Tris (1 mL), then 2×LiCl wash (with centrifuge pulses (10,000 g)

100 μL Lysis Buffer (1% SDC, 10 mM TCEP, 40 mM CLA, 100 mM TEAB) was added to all samples. Samples were then heated at 95° C. for 10 mins with gentle agitation. 1 μg (1 μL) of trypsin (1 μg/μL in Lysis Buffer) was then added to all samples. Samples were incubated overnight at 37° C. (19-21 hours). After incubation the samples were diluted with 800 μL of 0.5% Formic acid and the tip was removed. This solution was then centrifuged at 12,000 g for 10 mins and the supernatant was transferred to a conditioned SPE cartridge for clean-up. Peptides were then analysed using mass spectrometry.

### Data Analysis

Samples were searched in one set for the bovine samples against bovine fasta using DIA-NN.

### Results and Discussion

### Total Yield

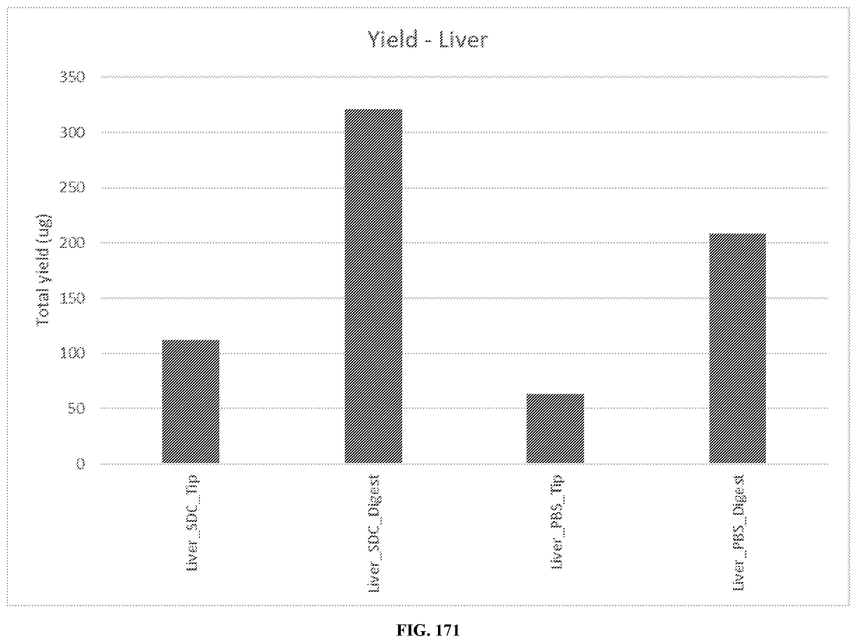

The yield for the liver loaded into the VAMS was approximately half of the in-tube digest (FIG. 171).

### Protein IDs

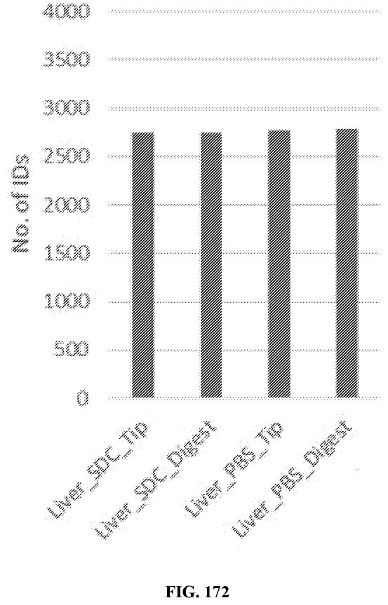

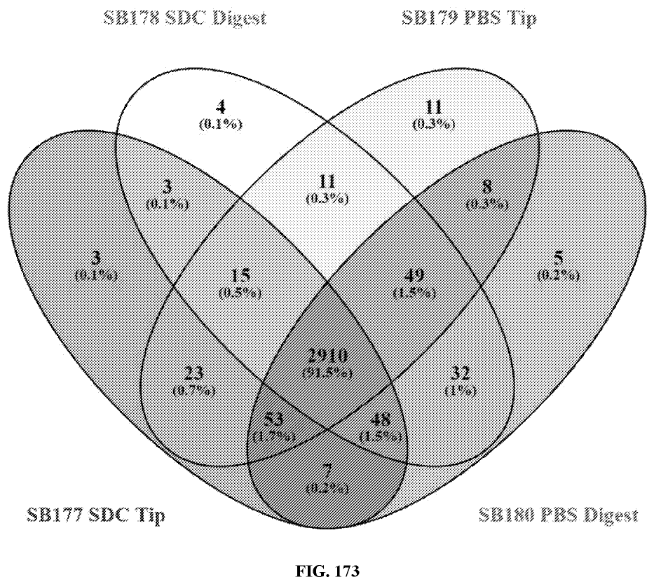

The number of protein IDs for the liver tissue samples were consistent between the in-tip and in-tube digestions (FIG. 172). There was also no difference between the SDC and PBS solubilization.

### Protein Overlap

The protein overlap of all of the liver samples was high, and both the in-tube digest and in-tip digest was highly consistent as well as the solubilisation with PBS or SDC (FIG. 173).

### Conclusions

There was not a significant advantage to loading a tissue lysate into a VAMS in terms of proteins identified, however, the advantage of improved storage conditions (can store at room temperature for an extended period of time) after loading into VAMS is advantageous. It is also advantageous that the in-tip digest was consistent with the regular liquid digest methods.

### Exemplary Embodiments

One or more than one (including for instance all) of the following exemplary Embodiments may comprise each of the other embodiments or parts thereof.

A1. In an Embodiment, a method of fractionating a sample, comprising:
a) introducing a sample into a porous material;
b) optionally, centrifuging the porous material containing the sample;
c) drying the sample in the porous material;
d) extracting a first set of proteins from the dried sample-containing porous material, comprising:
   1) incubating the dried sample-containing porous material in a first extraction solution; and
   2) separating the first extraction solution from the first incubated porous material;
e) detecting the first set of proteins in the separated extraction solution.

A2. In an Embodiment, a method of fractionating a sample, comprising:
a) introducing a sample into a porous material;
b) optionally, centrifuging the porous material containing the sample;
c) drying the sample in the porous material;
d) extracting a first set of proteins from the dried sample-containing porous material, comprising:
   1) incubating the dried sample-containing porous material in a first extraction solution;
   2) separating the first extraction solution from the first incubated porous material; and
   3) optionally, washing the separated first extracted porous material;
e) extracting a second set of proteins from the first extracted porous material, comprising:
   1) incubating the first extracted porous material in a second extraction solution;
   2) separating the second extraction solution from the second incubated porous material; and
f) detecting the first or second set of proteins from the separated first or second extraction solution, respectively.

A3. The method of Embodiment A2, wherein the method further comprises:
3. The method of claim 2, wherein the method further comprises:
   g) optionally, washing the separated second extracted porous material;
   h) extracting a third set of proteins from the second extracted porous material, comprising:
      1) incubating the second extracted porous material in a third extraction solution; and
      2) separating the third extraction solution from the third incubated porous material; and
   i) detecting the third set of proteins in the separated third extraction solution.

A4. The method of Embodiment A3, wherein the method further comprises sequentially extracting one or more further sets of proteins from the third extracted porous material comprising repeating the steps g)-i) with one or more further extraction solutions.

A5. In an Embodiment, a method of fractionating a sample, comprising:
a) introducing a sample into a porous material;
b) optionally, centrifuging the porous material containing the sample;
c) drying the sample in the porous material;
d) extracting a first set proteins from the dried sample-containing porous material, comprising:
   1) incubating the dried sample-containing porous material in a first extraction solution;
   2) separating the first extraction solution from the first incubated porous material; and
   3) optionally, washing the separated first extracted porous material;
e) digesting proteins remaining in the first extracted porous material, comprising:
   1) incubating the first extracted porous material in a digestion solution; and
   2) separating the digestion solution from the digestion incubated porous material; and
f) detecting one or more proteins in the separated digestion solution.

A6. The method of Embodiment A5, wherein prior to the digestion step the method further comprises:
A) extracting a second set of proteins from the first extracted porous material, comprising:
   1) incubating the first extracted porous material in a second extraction solution;
   2) separating the second extraction solution from the second incubated porous material; and
   g) optionally, washing the separated second extracted porous material; and
B) optionally, detecting the second set of proteins in the separated second extraction solution.

A7. The method of Embodiment A6, wherein prior to the digestion step the method further comprises sequentially extracting one or more further sets of proteins from the second extracted porous material comprising repeating the steps A)-B) with one or more further extraction solutions.

A8. The method of any one of Embodiments A1-A7, wherein the sample is or comprises a body fluid sample.

A9. The method of any one of Embodiments A1-A8, wherein the sample comprises cells or tissue.

A10. The method of any one of Embodiments A1-A9, wherein the sample comprises cells suspended in a liquid.

A11. The method of any one of Embodiments A1-A10, wherein the sample comprises cultured cells suspended in a culture media.

A12. The method of any one of Embodiments A1-A11, wherein the sample is or comprises blood, blood fractions, plasma, a nucleic acid-containing stabilized sample, a stabilized blood sample (e.g., blood in a Streck tube sample), urine, tears, wound fluid, CSF, bronchoalveolar lavage, or ascites.

A13. The method of any one of Embodiments A1-A12, wherein the sample is or comprises plasma.

A14. The method of any one of Embodiments A1-A13, wherein the sample is or comprises a blood sample.

A15. The method of any one of Embodiments A1-A14, wherein the blood sample is a whole blood (WB) sample.

A16. The method of any one of Embodiments A1-A15, wherein the blood sample is a red blood cell (RBC) sample.

A17. The method of any one of Embodiments A1-A16, wherein the blood sample is a white blood cell (WBC) sample.

A18. The method of any one of Embodiments A1-A17, wherein the blood sample is a frozen blood sample.

A19. The method of any one of Embodiments A1-A18, wherein the blood sample is a fresh blood sample.

A20. The method of any one of Embodiments A1-A19, wherein the sample has a volume in the range of 100 μL to 2 μL, 100 μL to 5 μL, 100 μL to 10 μL, 99 μL to 2 μL, 90 μL to 2 μL, 80 μL to 2 μL, 70 μL to 2 μL, 60 μL to 2 μL, 50 μL to 2 μL, 40 μL to 2 μL, 30 μL to 2 μL, 20 μL to 2 μL, 10 μL to 2 μL, or 5 μL to 2 μL.

A21. The method of any one of Embodiments A1-A20, wherein the sample has a volume of <100 μL, <50 μL, <30 μL, <10 μL, or <5 μL.

A22. The method of any one of Embodiments A1-A21, wherein the sample has a volume of at least 2 μL.

A23. The method of any one of Embodiments A1-A22, wherein the introducing step comprises absorbing the sample into the porous material.

A24. The method of any one of Embodiments A1-A23, wherein the introducing step comprises absorbing the sample into the porous material via a finger prick.

A25. The method of any one of Embodiments A1-A24, wherein the introducing step comprises dipping the porous material into the sample.

A26. The method of any one of Embodiments A1-A25, wherein the introducing step comprises pipetting a known volume of the sample into the porous material.

A27. The method of any one of Embodiments A1-A26, wherein the method comprises centrifuging the porous material containing the sample prior to the drying step.

A28. The method of any one of Embodiments A1-A27, wherein the centrifuging is at a speed of 500 g to 10,000 g.

A29. The method of any one of Embodiments A1-A28, wherein the centrifuging is for at least 1 to 15 minutes.

A30. The method of any one of Embodiments A1-A29, wherein the centrifuging is for no more than 15 minutes.

A31. The method of any one of Embodiments A1-A30, wherein the drying step, the extraction step, and the digesting step are conducted with said sample-containing porous material in said tube.

A32. The method of any one of Embodiments A1-A31, wherein the drying step comprises air drying the sample within the porous material, after which the sample is dried in the porous material.

A33. The method of any one of Embodiments A1-A32, wherein the drying step comprises centrifuging the sample within the porous material, after which the sample is dried in the porous material.

A34. The method of any one of Embodiments A1-A33, wherein the drying step comprises drying for a period of time to adhere the sample to the porous material.

A35. The method of any one of Embodiments A1-A34, wherein the drying step comprises air drying the sample within the porous material.

A36. The method of any one of Embodiments A1-A35, wherein the air drying is for at least 1, 2, 4, 6, 8, 10, 12, 16, 20 or 24 hours.

A37. The method of any one of Embodiments A1-A36, wherein the air drying is for 24 hours.

A38. The method of any one of Embodiments A1-A37, wherein the air drying is for at least 1 day.

A39. The method of any one of Embodiments A1-A38, wherein the drying step comprises centrifuging the sample within the porous material.

A40. The method of any one of Embodiments A1-A39, wherein the centrifuging is at a speed of 500 g to 10,000 g.

A41. The method of any one of Embodiments A1-A40, wherein the centrifuging is for at least 1 to 15 minutes.

A42. The method of any one of Embodiments A1-A41, wherein the centrifuging is for no more than 15 minutes.

A43. The method of any one of Embodiments A1-A42, wherein the drying step comprises vacuuming the sample within the porous material.

A44. The method of any one of Embodiments A1-A43, wherein the first, the second, the third, and the further extraction solution comprises an extracting agent comprising a salt, a mild detergent, a strong detergent, a chaotrope, a reducing agent, a thiol-containing reducing agent, an alkylating agent, an acid, an organic solvent, or an enzyme, or combinations thereof.

A45. The method of any one of Embodiments A1-A44, wherein the first extraction solution comprises an extracting agent comprising a salt, a mild detergent, a strong detergent, a chaotrope, a reducing agent, a thiol-containing reducing agent, an alkylating agent, an acid, an organic solvent, or an enzyme, or combinations thereof.

A46. The method of any one of Embodiments A1-A45, wherein the second extraction solution comprises an extracting agent comprising a salt, a mild detergent, a strong detergent, a chaotrope, a reducing agent, a thiol-containing reducing agent, an alkylating agent, an acid, an organic solvent, or an enzyme, or combinations thereof.

A47. The method of any one of Embodiments A1-A46, wherein the third extraction solution comprises an extracting agent comprising a salt, a mild detergent, a strong detergent, a chaotrope, a reducing agent, a thiol-containing reducing agent, an alkylating agent, an acid, an organic solvent, or an enzyme, or combinations thereof.

A48. The method of any one of Embodiments A1-A47, wherein the further extraction solution comprises an extracting agent comprising a salt, a mild detergent, a strong detergent, a chaotrope, a reducing agent, a thiol-containing reducing agent, an alkylating agent, an acid, an organic solvent, or an enzyme, or combinations thereof.

A49. The method of any one of Embodiments A1-A48, wherein the first, the second, the third, and the further extraction solution comprises a salt.

A50. The method of any one of Embodiments A1-A49, wherein the first, the second, the third, and the further extraction solution comprises a mild detergent.

A51. The method of any one of Embodiments A1-A50, wherein the first, the second, the third, and the further extraction solution comprises a strong detergent.

A52. The method of any one of Embodiments A1-A51, wherein the first, the second, the third, and the further extraction solution comprises a chaotrope.

A53. The method of any one of Embodiments A1-A52, wherein the first, the second, the third, and the further extraction solution comprises a reducing agent.

A54. The method of any one of Embodiments A1-A53, wherein the first, the second, the third, and the further extraction solution comprises a thiol-containing reducing agent.

A55. The method of any one of Embodiments A1-A54, wherein the first, the second, the third, and the further extraction solution comprises an alkylating agent.

A56. The method of any one of Embodiments A1-A55, wherein the salt is or comprises NaCl, LiCl, or Tris-HCl.

A57. The method of any one of Embodiments A1-A56, wherein the mild detergent is a non-ionic detergent, an ionic detergent, or a zwitterionic detergent.

A58. The method of any one of Embodiments A1-A57, wherein the mild detergent is or comprises: i) a non-ionic detergent comprising PBS or Tween; ii) an ionic detergent comprising sodium deoxycholate; or iii) a zwitterionic detergent comprising a sulfobetaine or an amidosulfobetaine.

A59. The method of any one of Embodiments A1-A58, wherein the strong detergent is or comprises CTAB, CHAPS, or SDS.

A60. The method of any one of Embodiments A1-A59, wherein the chaotrope is or comprises urea, thiourea, or guanidine.

A61. The method of any one of Embodiments A1-A60, wherein the reducing agent is or comprises a phosphine.

A62. The method of any one of Embodiments A1-A61, wherein the phosphine is or comprises TBP or TCEP.

A63. The method of any one of Embodiments A1-A62, wherein the thiol-containing reducing agent is or comprises betamercaptoethanol or dithiothreitol.

A64. The method of any one of Embodiments A1-A63, wherein the alkylating agent is or comprises iodoacetamide or acrylamide.

A65. The method of any one of Embodiments A1-A64, wherein the first extraction solution is a salt solution, a mild detergent-containing solution, or a salt and mild detergent-containing solution.

A66. The method of any one of Embodiments A1-A65, wherein the first extraction solution is a salt solution, a chaotrope-containing solution, or a salt and chaotrope-containing solution.

A67. The method of any one of Embodiments A1-A66, wherein the first extraction solution comprises NaCl, LiCl, Tris-HCl, PBS, or Tween, or combination thereof.

A68. The method of any one of Embodiments A1-A67, wherein the first extraction solution comprises NaCl, LiCl, Tris-HCl, PBS, Tween, CHAPS, SDS, urea, thiourea, or guanidine, or combination thereof.

A69. The method of any one of Embodiments A1-A68, wherein the first extraction solution comprises NaCl, LiCl, Tris-HCl, PBS, Tween, CHAPS, SDS, urea, thiourea, guanidine, TBP, TCEP, betamercaptoethanol, dithiothreitol, iodoacetamide, or acrylamide, or combination thereof.

A70. The method of any one of Embodiments A1-A69, wherein the first extraction solution and the second extraction solution are different.

A71. The method of any one of Embodiments A1-A70, wherein the first, the second, and the third extraction solutions are different.

A72. The method of any one of Embodiments A1-A71, wherein the first, the second, the third, and the further extraction solutions are different.

A73. The method of any one of Embodiments A1-A72, wherein selection of the first, the second, the third, and the further extraction solutions is to fractionate the proteins contained in the porous material via differential solubility.

A74. The method of any one of Embodiments A1-A73, wherein each sequential extraction solution selected is characterized as having greater solubility of the proteins remaining in the porous material.

A75. The method of any one of Embodiments A1-A74, wherein the first, the second, the third, and the further extraction solutions comprise identical extracting agents of increasing concentrations as progress from one extraction to the next extraction.

A76. The method of any one of Embodiments A1-A75, wherein the concentration of the salt in the first, the second, the third, or the further extraction solution, is 0.01-1 M.

A77. The method of any one of Embodiments A1-A76, wherein the concentration of the salt in the first, the second, the third, or the further extraction solution, is 0.5 M.

A78. The method of any one of Embodiments A1-A77, wherein the concentration of the salt in the first, the second, the third, or the further extraction solution, is 0.1 M.

A79. The method of any one of Embodiments A1-A78, wherein the salt is NaCl, LiCl, or Tris-HCl.

A80. The method of any one of Embodiments A1-A79, wherein the concentration of the NaCl in the first, the second, the third, or the further extraction solution, is 0.5 M.

A81. The method of any one of Embodiments A1-A80, wherein the concentration of the LiCl in the first, the second, the third, or the further extraction solution, is 0.5 M.

A82. The method of any one of Embodiments A1-A81, wherein the concentration of the Tris-HCl in the first, the second, the third, or the further extraction solution, is 0.1 M.

A83. The method of any one of Embodiments A1-A82, wherein the concentration of the chaotrope in the first, the second, the third, or the further extraction solution, is 0.01-8 M.

A84. The method of any one of Embodiments A1-A83, wherein the concentration of the chaotrope in the first, the second, the third, or the further extraction solution, is 0.1, 0.5, 1, 1.5, 2, 2.5, 3, 3.5, 4, 4.5, 5, 5.5, 6, 6.5, 7, 7.5, or 8 M.

A85. The method of any one of Embodiments A1-A84, wherein the concentration of the chaotrope in the first, the second, the third, or the further extraction solution, is 0.01-8 M.

A86. The method of any one of Embodiments A1-A85, wherein the chaotrope is urea, thiourea, or guanidine.

A87. The method of any one of Embodiments A1-A86, wherein the first, the second, the third, or the further extraction solution comprises urea, thiourea, or guanidine, or combinations thereof.

A88. The method of any one of Embodiments A1-A87, wherein the first, the second, the third, or the further extraction solution comprises urea at a concentration of 2 M.

A89. The method of any one of Embodiments A1-A88, wherein the first, the second, the third, or the further extraction solution comprises urea at a concentration of 5 M and thiourea at a concentration of 1 M.

A90. The method of any one of Embodiments A1-A89, wherein the first, the second, the third, or the further extraction solution comprises urea at a concentration of 6 M and thiourea at a concentration of 1.5 M.

A91. The method of any one of Embodiments A1-A90, wherein the first, the second, the third, or the further extraction solution comprises urea at a concentration of 7 M and thiourea at a concentration of 2 M.

A92. The method of any one of Embodiments A1-A91, wherein the separated first, second, third, or further, extraction solution comprises one or more proteins.

A93. The method of any one of Embodiments A1-A92, wherein the separated first extraction solution comprises the first set of proteins.

A94. The method of any one of Embodiments A1-A93, wherein the separated first extraction solution comprises albumin, hemoglobin, IgG, or one or more additional proteins compared to conventionally isolated serum or plasma.

A95. The method of any one of Embodiments A1-A94, wherein the separated second extraction solution comprises the second set of proteins.

A96. The method of any one of Embodiments A1-A95, wherein the separated third extraction solution comprises the third set of proteins.

A97. The method of any one of Embodiments A1-A96, wherein the separated further extraction solution comprises the further set of proteins.

A98. The method of any one of Embodiments A1-A97, wherein the first, the second, the third, or the further, extraction solution has a volume of less than 100 μL.

A99. The method of any one of Embodiments A1-A98, wherein the first, the second, the third, or the further, extraction solution has a volume of 95 μL or less.

A100. The method of any one of Embodiments A1-A99, wherein the first, the second, the third, or the further, extraction solution has a volume of 90 μL or less.

A101. The method of any one of Embodiments A1-A100, wherein the first, the second, the third, or the further, extraction solution has a volume of 1-5 times the volume of porous material.

A102. The method of any one of Embodiments A1-A101, wherein the first, the second, the third, or the further, extraction solution has a volume of 1, 2, 3, 4, or 5 times the volume of porous material.

A103. The method of any one of Embodiments A1-A102, wherein the first, the second, the third, or the further, extraction solution has a volume of 3 times the volume of porous material.

A104. The method of any one of Embodiments A1-A103, wherein the incubating in the first, the second, the third, or the further, extraction solution is conducted for a period of time long enough to extract one or more proteins contained within or adhered to the porous material.

A105. The method of Embodiment A104, wherein said incubating is conducted for 1 min-48 hour.

A106. The method of Embodiment A104, wherein said incubating is conducted for 1, 5, 10, 15, 20, 25, 30, 35, 40, 45, 50, 55 or 60 min.

A107. The method of Embodiment A104, wherein said incubating is conducted for 1-48 hours, 1-40 hours, 1-30 hours, 1-20 hours, 1-10 hours, 1-5 hours, or 1-40 hours.

A108. The method of any one of Embodiments A104-A107, wherein said incubating is conducted at ambient temperature.

A109. The method of any one of Embodiments A104-A107, wherein said incubating is conducted at a temperature elevated above ambient temperature or conducted at a temperature elevated below ambient temperature.

A110. The method of any one of Embodiments A104-A109, wherein said incubating is conducted with agitation.

A111. The method of any one of Embodiments A1-A110, wherein the incubating in the digestion solution is conducted for a period of time long enough to digest one or more proteins contained within or adhered to the porous material.

A112. The method of Embodiment A111, wherein said incubating is conducted for 1 min-48 hour.

A113. The method of Embodiment A111, wherein said incubating is conducted for 1, 5, 10, 15, 20, 25, 30, 35, 40, 45, 50, 55 or 60 min.

A114. The method of Embodiment A111, wherein said incubating is conducted for 1, 2, 3, 4, 5, 6, 7, 8, 9, 10, 11, 12, 13, 14, 15, 16, 17, 18, 19, 20, 21, 22, 23 or 24 hours.

A115. The method of any one of Embodiments A111-A114, wherein said incubating is conducted at ambient temperature, conducted at a temperature elevated above ambient temperature, or conducted at a temperature elevated below ambient temperature.

A116. The method of any one of Embodiments A111-A115, wherein said incubating is conducted with agitation.

A117. The method of any one of Embodiments A1-A116, wherein the separating comprises removing the first, the second, the third, or the further, incubated porous material from the first, the second, the third, or the further, extraction solution, respectively.

A118. The method of any one of Embodiments A1-A117, wherein the separating comprises removing the first, the second, the third, or the further, extraction solution from the first, the second, the third, or the further, incubated porous material, respectively.

A119. The method of any one of Embodiments A1-A118, wherein the separating of the first, the second, the third, or the further, extraction solution from the first, the second, the third, or the further, incubated porous material is via centrifugation, filtration, or a combination of centrifugation and filtration, respectively.

A120. The method of any one of Embodiments A117-A119, wherein the separating step is conducted via centrifugation.

A121. The method of any one of Embodiments A117-A119, wherein the separating step is conducted via filtration.

A122. The method of any one of Embodiments A117-A119, wherein the separating step is conducted via a combination of centrifugation and filtration.

A123. The method of any one of Embodiments 119-A122, wherein the centrifuging is at a speed of 500 g to 10,000 g.

A124. The method of any one of Embodiments A119-A123, wherein the centrifuging is for at least 1 minute.

A125. The method of any one of Embodiments A119-A124, wherein the centrifuging is for 1-15 minutes.

A126. The method of any one of Embodiments A119-A125, wherein the centrifuging is for no more than 15 minutes.

A127. The method of any one of Embodiments A1-A126, wherein the separating comprises removing the digestion incubated porous material from the digestion solution.

A128. The method of any one of Embodiments A1-A127, wherein the separating comprises removing the digestion solution from the digestion incubated porous material.

A129. The method of any one of Embodiments A1-A128, wherein the separating of the digestion solution from the digestion incubated porous material is via centrifugation, filtration, or a combination of centrifugation and filtration.

A130. The method of any one of Embodiments A127-129, wherein the separating step is conducted via centrifugation.

A131. The method of any one of Embodiments A127-A129, wherein the separating step is conducted via filtration.

A132. The method of any one of Embodiments A127-A129, wherein the separating step is conducted via a combination of centrifugation and filtration.

A133. The method of any one of Embodiments A129-A132, wherein the centrifuging is at a speed of 500 g to 10,000 g.

A134. The method of any one of Embodiments A129-A133, wherein the centrifuging is for at least 1 minute.

A135. The method of any one of Embodiments A129-A134, wherein the centrifuging is for 1-15 minutes.

A136. The method of any one of Embodiments A129-A135, wherein the centrifuging is for no more than 15 minutes.

A137. The method of any one of Embodiments A1-A136, wherein the method comprises washing the separated first, second, third, or further, extracted porous material.

A138. The method of any one of Embodiments A1-A137, wherein the method comprises washing the separated first extracted porous material.

A139. The method of any one of Embodiments A1-A138, wherein the method comprises washing the separated second extracted porous material.

A140. The method of any one of Embodiments A1-A139, wherein the method comprises washing the separated third extracted porous material.

A141. The method of any one of Embodiments A1-A140, wherein the method comprises washing the separated further extracted porous material.

A142. The method of any one of Embodiments A1-A141, wherein the separated first, second, third, or further extracted porous material is washed with a washing volume of the first, second, third, or further extraction solution, respectively.

A143. The method of any one of Embodiments A1-A142, wherein the separated first extracted porous material is washed with a washing volume of the first extraction solution.

A144. The method of any one of Embodiments A1-A143, wherein the separated second extracted porous material is washed with a washing volume of the first, the second, or a combination comprising the first and the second, extraction solution.

A145. The method of any one of Embodiments A1-A144, wherein the separated third extracted porous material is washed with a washing volume of the first, the second, the third, or a combination comprising the first, the second, or the third, extraction solution.

A146. The method of any one of Embodiments A1-A145, wherein the separated further extracted porous material is washed with a washing volume of the first, the second, the third, the further, or a combination comprising the first, the second, the third, or the further, extraction solution.

A147. The method of any one of Embodiments A1-A146, wherein the separated first extracted porous material is washed with an extraction solution different from the first extraction solution.

A148. The method of any one of Embodiments A1-A147, wherein selection of the different extraction solution is limited to solutions based on proteins remaining in the separated first extracted porous material have equivalent or less solubility in the different extraction solution than the first extraction solution.

A149. The method of any one of Embodiments A1-A148, wherein selection of the different extraction solution is based on the proteins remaining in the separated first extracted porous material having equivalent or less solubility in the different extraction solution than said first extraction solution.

A150. The method of any one of Embodiments A1-A149, wherein the washing volume of the first, the second, the third, or the further, extraction solution is 50 μL or more or is 100 μL or more.

A151. The method of any one of Embodiments A1-A150, wherein the washing volume of the first, the second, the third, or the further, extraction solution is between 5 times the volume of porous material and 3 mL.

A152. The method of any one of Embodiments A1-A151, wherein the washing volume of the first, the second, the third, or the further, extraction solution is 0.05-3 mL.

A153. The method of any one of Embodiments A1-A152, wherein the washing volume of the first, the second, the third, or the further, extraction solution is between 0.5-3 mL.

A154. The method of any one of Embodiments A1-A153, wherein the washing volume of the first, the second, the third, or the further, extraction solution is 0.5, 1, 1.5, 2, 2.5, or 3 mL.

A155. The method of any one of Embodiments A1-A154, wherein the washing of the separated first, second, third, or further extracted porous material is repeated 1, 2, or 3 times.

A156. The method of any one of Embodiments A1-A155, wherein the digestion solution comprises a reducing agent, an alkylating agent, a buffer, a detergent, or combinations thereof.

A157. The method of any one of Embodiments A1-A156, wherein the reducing agent is or comprises a phosphine.

A158. The method of any one of Embodiments A1-A157, wherein the phosphine is or comprises TBP or TCEP.

A159. The method of any one of Embodiments A1-A158, wherein the alkylating agent is or comprises iodoacetamide or acrylamide.

A160. The method of any one of Embodiments A1-A159, wherein the digestion solution further comprises a salt, a mild detergent, a strong detergent, a chaotrope, or a thiol-containing reducing agent, or combinations thereof.

A161. The method of any one of Embodiments A1-A160, wherein the salt is or comprises NaCl, LiCl, or Tris-HCl.

A162. The method of any one of Embodiments A1-A161, wherein the mild detergent is a non-ionic detergent, an ionic detergent, or a zwitterionic detergent.

A163. The method of any one of Embodiments A1-A162, wherein the mild detergent is or comprises: i) a non-ionic detergent comprising PBS or Tween; ii) an ionic detergent comprising sodium deoxycholate; or iii) a zwitterionic detergent comprising a sulfobetaine or an amidosulfobetaine.

A164. The method of any one of Embodiments A1-A163, wherein the strong detergent is or comprises CTAB, CHAPS, or SDS.

A165. The method of any one of Embodiments A1-A164, wherein the chaotrope is or comprises urea, thiourea, or guanidine.

A166. The method of any one of Embodiments A1-A165, wherein the thiol-containing reducing agent is or comprises betamercaptoethanol or dithiothreitol.

A167. The method of any one of Embodiments A1-A166, wherein the digestion solution further comprises a tryptic digestion solution.

A168. The method of any one of Embodiments A1-A167, wherein the tryptic digestion solution comprises triethylammonium bicarbonate, SDC, TCEP, chloroacetamide or combinations thereof.

A169. The method of any one of Embodiments A1-A168, wherein the digestion solution comprises triethylammonium bicarbonate, sodium deoxycholate (SDC), TCEP, or chloracetamide, or combinations thereof.

A170. The method of any one of Embodiments A1-A169, wherein the digestion solution comprises triethylammonium bicarbonate.

A171. The method of any one of Embodiments A1-A170, wherein the triethylammonium bicarbonate is present in the digestion solution at a concentration of 0.1 M.

A172. The method of any one of Embodiments A1-A171, wherein the digestion solution comprises sodium deoxycholate (SDC).

A173. The method of any one of Embodiments A1-A172, wherein the sodium deoxycholate (SDC) is present in the digestion solution at a concentration of 0.05% to 10% (w/v).

A174. The method of any one of Embodiments A1-A173, wherein the digestion solution comprises TCEP.

A175. The method of any one of Embodiments A1-A174, wherein the TCEP is present in the digestion solution at a concentration of 1 mM to 100 mM.

A176. The method of any one of Embodiments A1-A175, wherein the digestion solution comprises chloracetamide.

A177. The method of any one of Embodiments A1-A176, wherein the chloroacetamide is present in the digestion solution at a concentration of 5 mM to 100 mM.

A178. The method of any one of Embodiments A1-A177, wherein the digestion solution comprises a protease or a combination of proteases.

A179. The method of any one of Embodiments A1-A178, wherein the protease is trypsin or the combination of proteases comprises trypsin.

A180. The method of any one of Embodiments A1-A179, wherein the detecting step comprises detecting proteins in the separated first extraction solution.

A181. The method of any one of Embodiments A1-A180, wherein the detecting step comprises detecting proteins in the separated second extraction solution.

A182. The method of any one of Embodiments A1-A181, wherein the detecting step comprises detecting proteins in the separated third extraction solution.

A183. The method of any one of Embodiments A1-A182, wherein the detecting step comprises detecting proteins in the further extraction solution.

A184. The method of any one of Embodiments A1-A183, wherein the detecting of the first, second, third, or further sets of proteins from the first, second, third, or further extraction solutions, respectively, is via immunoassay.

A185. The method of any one of Embodiments A1-A184, wherein the detecting of the first, second, third, or further sets of proteins from the first, second, third, or further extraction solutions, respectively, is via Mass Spectrometry (MS).

A186. The method of any one of Embodiments A1-A185, wherein the detection via MS comprises subjecting said first, second, third, or further extraction solution to digestion prior to said detection via Mass Spectrometry (MS).

A187. The method of any one of Embodiments A1-A186, wherein prior to detecting the first, second, third, or further sets of proteins in the first, second, third, or further extraction solutions, respectively, the method further comprises incubating said first, second, third, or further extraction solution, in a digestion solution.

A188. The method of any one of Embodiments A1-A187, wherein the immunoassay is or comprises a Western blot.

A189. The method of any one of Embodiments A1-A188, wherein the immunoassay is or comprises an ELISA.

A190. The method of any one of Embodiments A1-A189, wherein said MS is LC-MS.

A191. The method of any one of Embodiments A1-A190, wherein said MS is selected reaction monitoring mass spectrometry (SRM-MS).

A192. The method of any one of Embodiments A1-A191, wherein said MS is data-dependent acquisition MS (DDA-MS).

A193. The method of any one of Embodiments A1-A192, wherein said MS is data-independent acquisition MS (DIA-MS).

A194. The method of any one of Embodiments A1-A193, wherein said MS is selected from the group consisting of matrix-assisted laser desorption/ionisation time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS; APCI-(MS)n; ion mobility spectrometry (IMS); inductively coupled plasma mass spectrometry (ICP-MS) atmospheric pressure photoionization mass spectrometry (APPI-MS), APPI-MS/MS; and APPI-(MS)n.

A195. The method of any one of Embodiments A1-A194, wherein the detecting step comprises a Luminex and Proximity Extension Assay.

A196. The method of any one of Embodiments A1-A195, wherein the Luminex and Proximity Extension Assay comprises Slow Offrate Modified Aptamer (SOMAmer) reagents.

A197. The method of any one of Embodiments A1-A196, wherein the porous material is a three-dimensional porous material.

A198. The method of any one of Embodiments A1-A197, wherein the three-dimensional porous material comprises a plastic.

A199. The method of any one of Embodiments A1-A198, wherein the three-dimensional porous material comprises a sponge.

A200. The method of any one of Embodiments A1-A199, wherein the three-dimensional porous material is a tip of a volumetric absorptive microsampling (VAMS) device.

A201. The method of any one of Embodiments A1-A200, wherein the porous material is a non-pre-loaded porous material.

A202. The method of any one of Embodiments A1-A201, wherein the porous material is not pre-loaded with protease inhibitors.

A203. The method of any one of Embodiments A1-A202, wherein the porous material is a pre-loaded porous material.

A204. The method of any one of Embodiments A1-A203, wherein the porous material is pre-loaded with a protease inhibitor.

A205. The method of any one of Embodiments A1-A204, wherein the porous material is pre-loaded with an anticoagulant.

A206. The method of any one of Embodiments A1-A205, wherein the anticoagulant is EDTA.

A207. The method of any one of Embodiments A1-A206, wherein the anticoagulant is heparin.

A208. The method of any one of Embodiments A1-A207, wherein the method further comprises placing the sample-containing porous material in a tube.

A209. The method of any one of Embodiments A1-A208, wherein the method further comprises placing the dried sample-containing porous material in a tube.

A210. The method of any one of Embodiments A1-A209, wherein the method uses a plurality of the porous materials.

A211. The method of any one of Embodiments A1-A210, wherein the sample is introduced into the plurality of the porous materials.

A212. The method of any one of Embodiments A1-A211, wherein the plurality of the porous materials comprises two or more porous materials.

A213. The method of any one of Embodiments A1-A212, wherein the plurality of the porous materials is two porous materials.

A214. The method of any one of Embodiments A1-A213, wherein the plurality of the porous materials is three porous materials.

A215. The method of any one of Embodiments A1-A214, wherein one or more proteins detected in the first, second, third, or further set of proteins is or comprises a non-membrane/soluble protein complex.

A216. The method of any one of Embodiments A1-A215, wherein one or more proteins detected in the separated digestion solution is or comprises a non-membrane/soluble protein complex.

A217. The method of any one of Embodiments A1-A216, wherein one or more proteins detected in the first, second, third, or further extraction solution following incubating in a digestion solution is or comprises a non-membrane/soluble protein complex.

A218. The method of any one of Embodiments A1-A217, wherein one or more proteins detected in the first, second, third, or further set of proteins is or comprises a cell-membrane associated protein.

A219. The method of any one of Embodiments A1-A218, wherein one or more proteins detected in the separated digestion solution is or comprises a cell-membrane associated protein.

A220. The method of any one of Embodiments A1-A219, wherein one or more proteins detected in the first, second, third, or further extraction solution following incubating in a digestion solution is or comprises a cell-membrane associated protein.

A221. The method of any one of Embodiments A218-A220, wherein the cell-membrane associated protein is a cell-membrane bound protein or a cell integral-membrane protein.

A222. The method of any one of Embodiments A218-A220, wherein the cell-membrane associated protein is a membrane protein complex.

A223. The method of any one of Embodiments A1-A222, wherein the method recovers 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or 2000 or more proteins compared to traditional microsampling.

A224. The method of any one of Embodiments A1-A223, wherein the method recovers a fraction of non-membrane/soluble protein complexes from a red blood cell (RBC) sample not obtained by traditional microsampling.

A225. The method of any one of Embodiments A1-A224, wherein the method recovers a fraction of non-membrane/soluble protein complexes from a plasma sample not obtained by traditional microsampling.

A226. The method of any one of Embodiments A1-A225, wherein the method recovers a fraction of non-membrane/soluble protein complexes from a whole blood sample not obtained by traditional microsampling.

A227. The method of any one of Embodiments A1-A226, wherein the method recovers a fraction of membrane protein complexes from a red blood cell (RBC) sample not obtained by traditional microsampling.

A228. The method of any one of Embodiments A1-A227, wherein the method recovers a fraction of membrane protein complexes from a plasma sample not obtained by traditional microsampling.

A229. The method of any one of Embodiments A1-A228, wherein the method recovers a fraction of membrane protein complexes from a whole blood sample not obtained by traditional microsampling.

A230. In an Embodiment, a method of producing a protein profile, comprising the methods of any one of Embodiments A1-A229.

A231. The method of Embodiment A230, wherein the protein profile is produced by immunoassay work flow.

A232. The method of Embodiment A230, wherein the protein profile is produced by a proteomics work flow.

A233. The method of Embodiment A230, wherein the protein profile is produced by a combination of an immunoassay work flow and a proteomics work flow.

A234. The method of any one of Embodiments A230-A233, wherein the method produces a protein profile of non-membrane/soluble protein complexes.

A235. The method of any one of Embodiments A230-A234, wherein the method produces a protein profile of cell-membrane associated proteins.

A236. In an Embodiment, a method of isolating one or more proteins, comprising the methods of any one of Embodiments A1-A235.

A237. In an Embodiment, a method of partitioning proteins in a sample by differential solubility, comprising the methods of any one of Embodiments A1-A235.

A238. In an Embodiment, a method for preparing sequential samples using an absorptive device, comprising the methods of any one of Embodiments A1-A235.

A239. In an Embodiment, a microsampling device, comprising:
a) a first porous material;
b) a second porous material; and
c) a tube.

A240. In an Embodiment, a microsampling device, comprising:
a) a first porous material,
b) a second porous material;
c) a third porous material; and
d) a tube.

A241. The device of Embodiment A239 or Embodiment A240, wherein the tube is an Eppendorf tube.

A242. In an Embodiment, a microsampling device, comprising:
a) a first porous material;
b) a second porous material; and
c) a shaft;
   wherein the first porous material is positioned at one end of the shaft and the second porous material is located at a position along the shaft so that the first porous material and the second porous material are separated and not in physical contact with each other.

A243. In an Embodiment, a microsampling device, comprising:
a) a first porous material;
b) a second porous material;
c) a third porous material; and
d) a shaft;
   wherein the first porous material is positioned at one end of the shaft and the second porous material and the third porous material are located at positions on the shaft so that the first porous material, the second porous material, and the third porous material are separated and not in physical contact with each other.

244. The device of any one of Embodiments A239-A243, wherein the microsampling device is a volumetric absorptive microsampling (VAMS) device.

A245. The device of any one of Embodiments A242-A244, wherein the first porous material and the second porous material are positioned on the shaft and separated from each other by a spacer such that said first porous material and said second porous material are separated and not in physical contact with each other.

A246. The device of any one of Embodiments A243-A245, wherein the first porous material, the second porous material, and the third porous material are positioned on the shaft and separated from each other by spacers such that said first porous material, said second porous material, and said third porous material are separated and not in physical contact with each other.

A247. The device of any one of Embodiments A239-A246, wherein the shaft is a threaded shaft.

A248. The device of any one of Embodiments A239-A247, wherein the shaft is a non-linear shaft.

A249. The device of any one of Embodiments A239-A248, wherein the shaft is a curved shaft.

A250. The device of any one of Embodiments A239-A249, wherein the shaft has a length of 10 mm to 50 mm.

A251. The device of any one of Embodiments A239-A250, wherein the shaft has a diameter of 0.5 mm to 3 mm.

A252. The device of any one of Embodiments A239-A251, wherein the shaft fits in the tube.

A253. The device of any one of Embodiments A239-A252, wherein the shaft fits in a tube having a volume of 5 mL, 2 mL, 1.5 mL, or 1 mL.

A254. The device of any one of Embodiments A239-A253, wherein the shaft fits in an Eppendorf tube.

A255. The device of any one of Embodiments A239-A254, wherein the shaft fits in an Eppendorf tube having a volume of 5 mL, 2 mL, 1.5 mL, or 1 mL.

A256. The device of any one of Embodiments A239-A255, wherein the shaft fits in an Eppendorf tube having a volume of 5 mL.

A257. The device of any one of Embodiments A239-A255, wherein the shaft fits in an Eppendorf tube having a volume of 2 mL.

A258. The device of any one of Embodiments A239-A255, wherein the shaft fits in an Eppendorf tube having a volume of 1.5 mL.

A259. The device of any one of Embodiments A239-A255, wherein the shaft fits in an Eppendorf tube having a volume of 1 mL.

A260. The device of any one of Embodiments A239-A259, wherein the shaft fits in a 96-well plate.

A261. The device of any one of Embodiments A239-A260, wherein the first porous material and the second porous material are equally spaced.

A262. The device of any one of Embodiments A240-A261, wherein the first porous material, the second porous material, and the third porous material are equally spaced.

A263. The device of any one of Embodiments A239-A262, wherein the first porous material and second porous material are pre-loaded with anticoagulant.

A264. The device of any one of Embodiments A239-A263, wherein the first porous material has an absorptive capacity in the range of 2.5 μL to 50 μL.

A265. The device of any one of Embodiments A239-A264, wherein the first porous material has an absorptive capacity of 2.5 μL, 5 μL, 10 μL, 20 μL, 30 μL, 40 μL, or 50 μL.

A266. The device of any one of Embodiments A239-A265, wherein the second porous material has an absorptive capacity in the range of 2.5 μL to 50 μL.

A267. The device of any one of Embodiments A239-A266, wherein the second porous material has an absorptive capacity of 2.5 μL, 5 μL, 10 μL, 20 μL, 30 μL, 40 μL, or 50 μL.

A268. The device of any one of Embodiments A240-A267, wherein the third porous material has an absorptive capacity in the range of 2.5 μL to 50 μL.

A269. The device of any one of Embodiments A240-A268, wherein the third porous material has an absorptive capacity of 2.5 μL, 5 L, 10 μL, 20 μL, 30 μL, 40 μL, or 50 μL.

A270. The device of any one of Embodiments A239-A269, wherein the first porous material and second porous material have the same absorptive capacity.

A271. The device of any one of Embodiments A239-A270, wherein the first porous material and second porous material have a different absorptive capacity.

A272. The device of any one of Embodiments A240-A270, wherein the first porous material, the second porous material, and the third porous material have the same absorptive capacity.

A273. The device of any one of Embodiments A240-A270, wherein the first porous material, the second porous material, and the third porous material have different absorptive capacities.

A274. The device of any one of Embodiments A240-A270, wherein the first porous material and the second porous material have the same absorptive capacity, and the third porous material has a different absorptive capacity relative to said first and second porous materials.

A275. A method of using the device of any one of Embodiments A239-A274 according to the method of any one of Embodiments A1-A238.

Further exemplary Embodiments are outlined in the following numbered statements. One or more than one (including for instance all) of the following exemplary Embodiments may comprise each of the other embodiments or parts thereof.

1. A method of fractionating a sample, comprising:
a) introducing a sample into a porous material;
b) optionally, centrifuging the porous material containing the sample;
c) drying the sample in the porous material;
d) extracting a first set molecules from the dried sample-containing porous material, comprising:
   1) incubating the dried sample-containing porous material in a first extraction solution;
   2) separating the first extraction solution from the porous material; and
   3) optionally, washing the porous material;
e) digesting proteins remaining in the porous material, comprising:
   1) incubating the porous material in a digestion solution; and
   2) separating the digestion solution from the porous material; and
f) optionally, detecting one or more molecules in the separated first extraction solution; and
g) detecting one or more peptides or proteins in the digestion solution.

2. The method of statement 1, wherein following extraction of the first set of molecules and prior to the digestion step the method further comprises:
A) extracting a second set of molecules from the first extracted porous material, comprising:
   1) incubating the first extracted porous material in a second extraction solution;
   2) separating the second extraction solution from the second incubated porous material; and
   h) optionally, washing the separated second extracted porous material; and
B) optionally, detecting the second set of molecules in the separated second extraction solution.

3. The method of statement 2, wherein prior to the digestion step the method further comprises sequentially extracting one or more further sets of molecules from the second extracted porous material comprising repeating the steps A)-B) with one or more further extraction solutions.

4. The method of any one of statements 1-3, wherein the first set of molecules is selected from the group consisting of proteins, nucleic acids, and glycans.

5. The method of any one of statements 2-4, wherein the second set of molecules is selected from the group consisting of proteins, nucleic acids, and glycans.

6. The method of any one of statements 3-5, wherein the one or more further sets of molecules is selected from the group consisting of proteins, nucleic acids, and glycans.

7. The method of any one of statements 4-6 wherein said proteins in the first, second, or one or more further sets of molecules, respectively, and/or the one or more peptides or proteins in the digestion solution are phosphoproteins or phosphopeptides.

8. The method of statement 7, comprising performing an enrichment step to enrich for phosphorylated proteins and/or peptides in the separated first, second, or one or more extraction solutions and/or the digestion solution.

9. The method of any one of statements 3-8, wherein the one or more further extraction solutions comprises an extracting agent comprising a salt, a mild detergent, a strong detergent, a chaotrope, a reducing agent, a thiol-containing reducing agent, or an alkylating agent, an acid, an organic solvent, or an enzyme, or combinations thereof.

10. The method of any one of statements 1-9, wherein the first extraction solution comprises an extracting agent comprising a salt, a mild detergent, a strong detergent, a chaotrope, a reducing agent, a thiol-containing reducing agent, or an alkylating agent, an acid, an organic solvent, or an enzyme, or combinations thereof.

11. The method of any one of statements 1-10, wherein the second extraction solution comprises an extracting agent comprising a salt, a mild detergent, a strong detergent, a chaotrope, a reducing agent, a thiol-containing reducing agent, or an alkylating agent, an acid, an organic solvent, or an enzyme, or combinations thereof.

12. The method of any one of statements 1-11, wherein the digestion solution comprises a reducing agent, an alkylating agent, a buffer, a detergent, or combinations thereof, and optionally, wherein the digestion solution further comprises a salt, a mild detergent, a strong detergent, a chaotrope, or a thiol-containing reducing agent, an acid, an organic solvent, or an enzyme, or combinations thereof.

13. The method of any one of statements 7-12, wherein the salt is or comprises NaCl, LiCl, CaCl₂), or Tris-HCl.

14. The method of any one of statements 7-13, wherein the mild detergent is a non-ionic detergent, an ionic detergent, or a zwitterionic detergent.

15. The method of any one of statements 7-14, wherein the mild detergent is or comprises: i) a non-ionic detergent comprising PBS or Tween; ii) an ionic detergent comprising sodium deoxycholate; or iii) a zwitterionic detergent comprising a sulfobetaine or an amidosulfobetaine.

16. The method of any one of statements 7-15, wherein the strong detergent is or comprises CTAB, CHAPS, or SDS.

17. The method of any one of statements 7-16, wherein the chaotrope is or comprises urea, thiourea, or guanidine.

18. The method of any one of statements 7-17, wherein the reducing agent is or comprises a phosphine.

19. The method of any one of statements 18, wherein the phosphine is or comprises TBP or TCEP.

20. The method of any one of statements 7-19, wherein the thiol-containing reducing agent is or comprises betamercaptoethanol or dithiothreitol.

21. The method of any one of statements 7-20, wherein the alkylating agent is or comprises iodoacetamide or acrylamide.

22. The method of any one of statements 7-21, wherein the acid is or comprises citric acid or trifluoroacetic acid.

23. The method of any one of statements 7-22, wherein the organic solvent is or comprises methanol.

24. The method of any one of statements 7-23, wherein the organic enzyme is or comprises benzonase.

25. The method of any one of statements 7-24, wherein the first, the second, the one or more further extraction solutions, and the digestion solutions are different.

26. The method of any one of statements 7-25, wherein selection of the first, the second, the one or more further extraction solutions is to fractionate the proteins contained in the porous material via differential solubility.

27. The method of any one of statements 7-26, wherein each sequential extraction solution selected is characterized as having greater solubility of the proteins remaining in the porous material.

28. The method of any one of statements 7-27, wherein the first, the second, the third, and the further extraction solutions comprise identical extracting agents of increasing concentrations as progress from one extraction to the next extraction.

29. The method of any one of statements 7-28, wherein the concentration of the salt in the first, the second, or one or more further extraction solutions, is 0.01-1 M.

30. The method of any one of statements 1-29, wherein the first, the second, or one or more further extraction solutions, comprises LiCl and Tris-HCl.

31. The method of statement 30, wherein the concentration of LiCl is 0.5 M, and the concentration of Tris-HCl is 0.1 M.

32. The method of any one of statements 7-31, wherein the concentration of the chaotrope in the first, the second, or one or more further extraction solutions, is 0.01-8 M.

33. The method of statement 32, wherein the concentration of the chaotrope in the first, the second, or one or more further extraction solutions, is 0.1, 0.5, 1, 1.5, 2, 2.5, 3, 3.5, 4, 4.5, 5, 5.5, 6, 6.5, 7, 7.5, or 8 M.

34. The method of any one of statements 7-33, wherein the first, the second, or one or more further extraction solutions, comprises urea at a concentration of 2, 5, 6, or 7M, and thiourea.

35. The method of any one of statements 1-34, wherein the digestion solution further comprises a tryptic digestion solution.

36. The method of any one of statements 1-35, wherein the digestion solution comprises triethylammonium bicarbonate, sodium deoxycholate (SDC), TCEP, or chloracetamide, or combinations thereof.

37. The method of statement 36, wherein the triethylammonium bicarbonate is present in the digestion solution at a concentration of 0.1 M.

38. The method of statements 36 or 37, wherein the sodium deoxycholate (SDC) is present in the digestion solution at a concentration of 0.05% to 10% (w/v).

39. The method of any one of statements 36-38, wherein the TCEP is present in the digestion solution at a concentration of 1 mM to 100 mM.

40. The method of any one of statements 36-38, wherein the chloroacetamide is present in the digestion solution at a concentration of 5 mM to 100 mM.

41. The method of any one of statements 1-40, wherein the digestion solution comprises a protease or a combination of proteases.

42. The method of any one of statements 1-41, wherein the protease is trypsin or the combination of proteases comprises trypsin.

43. The method of any one of statements 1-42, wherein the first, second, or one or more further extractions solutions comprises an endoglycosidase.

44. The method of statement 43, wherein the endoglycosidase is Peptide-N-Glycosidase F (PNGase F) and/or O-glycosidase.

45. The method of any one of statements 1-44, wherein the sample is or comprises a body fluid sample.

46. The method of any one of statements 1-45, wherein the sample comprises cells or tissue.

47. The method of any one of statements 1-46, wherein the sample comprises cells suspended in a liquid.

48. The method of any one of statements 1-47, wherein the sample comprises cultured cells suspended in a culture media.

49. The method of any one of statements 1-48, wherein the sample is or comprises blood, blood fractions, plasma, urine, tears, wound fluid, CSF, bronchoalveolar lavage, or ascites.

50. The method of any one of statements 1-49, wherein the sample is or comprises plasma.

51. The method of any one of statements 1-50, wherein the sample is or comprises a blood sample.

52. The method of statement 51, wherein the blood sample is: a whole blood (WB) sample, a red blood cell (RBC) sample, a white blood cell (WBC) sample, a frozen blood sample, or a fresh blood sample.

53. The method of any one of statements 1-52, wherein the sample has a volume in the range of 100 μL to 2 μL, 100 μL to 5 μL, 100 μL to 10 μL, 99 μL to 2 μL, 90 μL to 2 μL, 80 μL to 2 μL, 70 μL to 2 μL, 60 μL to 2 μL, 50 μL to 2 μL, 40 μL to 2 μL, 30 μL to 2 μL, 20 μL to 2 μL, 10 μL to 2 μL, or 5 μL to 2 μL.

54. The method of any one of statements 1-53, wherein the sample has a volume of <100 μL, <50 μL, <30 μL, <10 μL, or <5 μL.

55. The method of any one of statements 1-54, wherein the sample has a volume of at least 2 μL.

56. The method of any one of statements 1-55, wherein the introducing step comprises absorbing the sample into the porous material.

57. The method of any one of statements 1-56, wherein the introducing step comprises: absorbing the sample into the porous material via a finger prick, dipping the porous material into the sample, or pipetting a known volume of the sample into the porous material.

58. The method of any one of statements 1-57, wherein the method comprises centrifuging the porous material containing the sample prior to the drying step.

59. The method of any one of statements 1-58, comprising drying the sample in the porous material and wherein the drying step comprises drying for a period of time to adhere the sample to the porous material.

60. The method of statement 59, wherein the drying step comprises air drying the sample within the porous material.

61. The method of statement 59 or 60, wherein the drying step is for less than 30 minutes, preferably at least 5 minutes.

62. The method of statement 60, wherein the air drying is for at least 1, 2, 4, 6, 8, 10, 12, 16, 20 or 24 hours.

63. The method of statement 60, wherein the air drying is for at least 1 day.

64. The method of any one of statements 60-63, wherein the drying is in the presence of a desiccant.

65. The method of any one of statements 59-64, wherein the drying step comprises centrifuging the sample within the porous material.

66. The method of statement 58 or 65, wherein the centrifuging is at a speed of 500 g to 10,000 g.

67. The method of statement 66, wherein the centrifuging is for at least 1 to 15 minutes.

68. The method of any one of statements 65 or 66, wherein the centrifuging is for no more than 15 minutes.

69. The method of any one of statements 59-68, wherein the drying step comprises vacuuming the sample within the porous material.

70. The method of any one of statements 1-69, further comprising storing the sample-containing porous material for a period of time prior to the first extraction step.

71. The method of statement 70, wherein storing the sample-containing porous material immediately follows the drying step.

72. The method of statement 70 or 71, wherein the sample-containing porous material is stored frozen.

73. The method of statement 70 or 71, wherein the sample-containing porous material is stored at room temperature.

74. The method of any one of statements 1-73, wherein the drying step, the extraction step, and the digesting step are conducted with the sample-containing porous material in a tube.

75. The method of any one of statements 1-74, wherein the separated first extraction solution comprises albumin, hemoglobin, IgG, or one or more additional proteins compared to conventionally isolated serum or plasma.

76. The method of any one of statements 1-75, wherein the separated second extraction solution comprises the second set of proteins.

77. The method of any one of statements 1-76, wherein the separated one or more extraction solutions comprises a further set of proteins, respectively.

78. The method of any one of statements 1-77, wherein the first, the second, or one or more further, extraction solution has a volume of less than 100 μL, a volume less than 95 μL, a volume less than 90 μL, a volume of 1-5 times the volume of porous material, a volume of 1, 2, 3, 4, or 5 times the volume of porous material, preferably a volume of 3 times the volume of porous material.

79. The method of any one of statements 1-78, wherein the incubating in the first, the second, or one or more further extraction solutions is conducted for a period of time long enough to extract one or more proteins contained within or adhered to the porous material.

80. The method of statement 79, wherein said incubating is conducted for 1, 5, 10, 15, 20, 25, 30, 35, 40, 45, 50, 55, or 60 min.

81. The method of statement 79, wherein said incubating is conducted for 1-48 hours, 1-42 hours, 1-36 hours, 1-30 hours, 1-24 hours, 1-18 hours, 1-12 hours, or 1-6 hours.

82. The method of any one of statements 79-81, wherein said incubating is conducted at ambient temperature.

83. The method of any one of statements 79-81, wherein said, wherein said incubating is conducted at a temperature elevated above ambient temperature.

84. The method of any one of statements 1-83, wherein the incubating in the digestion solution is conducted for a period of time long enough to digest one or more proteins contained within or adhered to the porous material.

85. The method of statement 84, wherein said incubating is conducted for 1 min-48 hour.

86. The method of statement 84, wherein said incubating is conducted for 1, 5, 10, 15, 20, 25, 30, 35, 40, 45, 50, 55 or 60 min.

87. The method of statement 84, wherein said incubating is conducted for 1, 2, 3, 4, 5, 6, 7, 8, 9, 10, 11, 12, 13, 14, 15, 16, 17, 18, 19, 20, 21, 22, 23 or 24 hours.

88. The method of any one of statements 84-87, wherein said incubating is conducted at ambient temperature, conducted at a temperature elevated above ambient temperature, preferably at 37° C.

89. The method of any one of statements 79-88, wherein said incubating is conducted with agitation.

90. The method of any one of statements 1-89, wherein separating the first, the second, or the one or more further extraction solutions from the first, the second, the third, or the further, incubated porous material comprises removing the first, the second, the third, or the further, incubated porous material from the first, the second, or the one or more further extraction solutions, respectively.

91. The method of any one of statements 1-89, wherein separating the first, the second, or the one or more further extraction solutions from the first, the second, the third, or the further, incubated porous material comprises removing the first, the second, the third, or the one or more further extraction solutions from the first, the second, or the further, incubated porous material, respectively.

92. The method of any one of statements 1-91, wherein separating the digestion solution from the porous material comprises removing the digestion incubated porous material from the digestion solution.

93. The method of any one of statements 1-91, wherein separating the digestion solution from the porous material comprises removing the digestion solution from the digestion incubated porous material.

94 The method of any one of statements 90-93, wherein the separating is via centrifugation, filtration, or a combination of centrifugation and filtration, respectively.

95. The method of any one of statements 1-94, wherein the method comprises washing the separated first, second, or further, extracted porous material.

96. The method of statement 95, wherein the separated first, second, or further extracted porous material is washed with a washing volume of the first, second, or the one or more further extraction solutions, respectively, a volume of an extraction solution different from the first extraction solution, or in a volume of water.

97. The method of statement 96, wherein selection of the different extraction solution is limited to solutions based on proteins remaining in the separated extracted porous material that have equivalent or less solubility in the different extraction solution than the preceding extraction solution.

98. The method of statement 96 or 97, wherein the washing volume of the extraction solution is between 5 times the volume of porous material and 3 mL.

99. The method of statement 96 or 97, wherein the washing volume of the first, the second, the third, or the further, extraction solution is 0.05-3 mL.

100. The method of any one of statements 95-99, wherein the washing step is repeated 1, 2, or 3 times.

101. The method of any one of statements 1-100, comprising detecting one or more molecules in the separated first extraction solution, wherein said detecting comprises detecting proteins in the separated first extraction solution.

102. The method of any one of statements 2-101, comprising detecting one or more molecules in the separated second extraction solution wherein said detecting comprises detecting proteins in the separated second extraction solution.

103. The method of any one of statements 3-100, comprising detecting one or more molecules in the separated one or more further extraction solutions, wherein said detecting comprises detecting proteins in the separated one or more further extraction solution.

104. The method of any one of statements 101-103, wherein the detecting of proteins is via immunoassay.

105. The method of statement 104, wherein the immunoassay is or comprises a Western blot, or ELISA.

106. The method of statement 104, wherein the detecting step comprises a Luminex and Proximity Extension Assay.

107. The method of statement 106, wherein the Luminex and Proximity Extension Assay comprises Slow Offrate Modified Aptamer (SOMAmer) reagents.

108. The method of any one of statements 101-103, wherein the detecting of proteins is via Mass Spectrometry (MS).

109. The method of statement 108, wherein the detection via MS comprises subjecting the extraction solution to digestion prior to said detection via Mass Spectrometry (MS).

110. The method of any one of statements 1-100, comprising detecting one or more molecules in the separated first extraction solution, wherein said detecting comprises detecting glycans in the separated first extraction solution.

111. The method of any one of statements 2-101, or 104-109, comprising detecting one or more molecules in the second extraction solution wherein said detecting comprises detecting glycans.

112. The method of any one of statements 3-102, or 104-109, comprising detecting one or more molecules in the separated one or more further extraction solutions, wherein said detecting comprises detecting glycans in the separated one or more further extraction solution.

113. The method of any one of statements 110-112, wherein the detecting of glycans is via High Performance Liquid Chromatography (HPLC).

114. The method of any one of statements 1-113, wherein the detecting of one or more peptides or proteins in the digestion solution is via Mass Spectrometry (MS).

115. The method of any one of statements 108, 109 or 114, wherein said MS is LC-MS.

116. The method of any one of statements 108, 109 or 114, wherein said MS is selected reaction monitoring mass spectrometry (SRM-MS).

117. The method of any one of statements 108, 109 or 114, wherein said MS is data-dependent acquisition MS (DDA-MS).

118. The method of any one of statements 108, 109 or 114, wherein said MS is data-independent acquisition MS (DIA-MS).

119. The method of any one of statements 108, 109 or 114, wherein said MS is selected from the group consisting of matrix-assisted laser desorption/ionisation time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS; APCI-(MS)n; ion mobility spectrometry (IMS); inductively coupled plasma mass spectrometry (ICP-MS) atmospheric pressure photoionization mass spectrometry (APPI-MS), APPI-MS/MS; and APPI-(MS)n.

120. The method of any one of statements 1-119, wherein the porous material is a hydrophilic polymeric material.

121. The method of statement 120, wherein the porous material comprises a plastic.

122. The method of statement 120 or 121, wherein the porous material comprises a sponge.

123. The method of any one of statements 120-122, wherein the three-dimensional porous material is a tip of a volumetric absorptive microsampling (VAMS) device.

124. The method of any one of statements 1-123, wherein the porous material is not a pre-loaded porous material.

125. The method of any one of statements 1-123, wherein the porous material is pre-loaded with a protease inhibitor.

126. The method of any one of statements 1-123, wherein the porous material is pre-loaded with an enzyme, preferably a nuclease, more preferably benzonase.

127. The method of any one of statements 1-123, wherein the porous material is pre-loaded with a detergent, preferably SDS.

128. The method of any one of statements 1-123, wherein the porous material is pre-loaded with an anticoagulant.

129. The method of statement 128, wherein the anticoagulant is EDTA or heparin.

130. The method of any one of statements 1-129, wherein the method uses a plurality of the porous materials.

131. The method of statement 130, wherein the sample is introduced into the plurality of the porous materials.

132. The method of any one of statements 1-131, wherein one or more proteins detected in a first, second, or one or more further set of molecules is or comprises a non-membrane/soluble protein complex.

133. The method of any one of statements 1-132, wherein one or more proteins detected in the separated digestion solution is or comprises a non-membrane/soluble protein complex.

134. The method of any one of statements 1-133, wherein one or more proteins detected in a first, second, or one or more further set of molecules is or comprises a cell-membrane associated protein.

135. The method of any one of statements 1-134, wherein one or more proteins detected in the separated digestion solution is or comprises a cell-membrane associated protein.

136. The method of statement 134 or 135, wherein the cell-membrane associated protein is a cell-membrane bound protein or a cell integral-membrane protein.

137. The method of any one of statements 134-136, wherein the cell-membrane associated protein is a membrane protein complex.

138. The method of any one of statements 1-137, wherein the method recovers 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or 2000 or more proteins compared to traditional microsampling.

139. The method of any one of statements 1-138, wherein the method recovers from a red blood cell (RBC) sample, a plasma sample, or a whole blood sample, a fraction of non-membrane/soluble protein complexes not obtained by traditional microsampling, or a fraction of membrane protein complexes not obtained by traditional microsampling analysis.

140. The method of statement 1, wherein the porous material is a hydrophilic polymer tip of a volumetric absorptive microsampling (VAMS) device, wherein the first extraction solution comprises 100 mM-500 mM LiCl and 100 mM Tris, and wherein the method comprises washing the porous material at step d) 3) with a wash solution comprising 100 mM-500 mM LiCl and 100 mM Tris, and wherein the digestion solution comprises 1 μg Trypsin in a buffer comprising 1% sodium deoxycholate (SDC), 10 mM tris (2-carboxyethyl) phosphine (TCEP), 40 mM 2-Chloroacetamide (CLA), 100 mM and 100 mM triethylammonium bicarbonate buffer (TEAB). 141. The method of statement 1 or 2, wherein the porous material is a hydrophilic polymer tip of a volumetric absorptive microsampling (VAMS) device, wherein the first extraction solution comprises a PBS solution comprising a protease inhibitor, wherein the volume of the first extraction solution is ≤100 μL; wherein the method comprises washing the porous material at step d) 3) with a wash solution comprising 100 mM-500 mM LiCl and 100 mM Tris; wherein the digestion solution comprises 1 μg Trypsin in a buffer comprising 1% sodium deoxycholate (SDC), 10 mM tris (2-carboxyethyl) phosphine (TCEP), 40 mM 2-Chloroacetamide (CLA), 100 mM and 100 mM triethylammonium bicarbonate buffer (TEAB); and wherein the method comprises detecting one or more molecules in the separated first extraction solution, wherein the one or more molecules is one or more proteins and detection is via immunoassay.

142. The method of statement 141, wherein the second extraction solution comprises PNGase F in a buffer comprising 1% sodium deoxycholate (SDC), 10 mM tris (2-carboxyethyl) phosphine (TCEP), 40 mM 2-Chloroacetamide (CLA), 100 mM and 100 mM triethylammonium bicarbonate buffer (TEAB); and wherein the method comprises detecting the second set of molecules in the separated second extraction solution, wherein the one or more molecules is one or more glycans.

143. The method of statement 3, wherein the one or more further extraction solution comprises PNGase F in a buffer comprising 1% sodium deoxycholate (SDC), 10 mM tris (2-carboxyethyl) phosphine (TCEP), 40 mM 2-Chloroacetamide (CLA), 100 mM and 100 mM triethylammonium bicarbonate buffer (TEAB); and wherein the method comprises detecting a further set of molecules in the separated further extraction solution, wherein the one or more molecules is one or more glycans.

144. The method of statement 142 or 143, further comprising dividing the porous material prior to the digestion step e), and analysing a portion of the portion of the porous material for remaining glycans.

145. A method of producing a protein profile, comprising the methods of any one of statements 1-144.

146. The method of statement 145, wherein the protein profile is produced by immunoassay work flow.

147. The method of statement 145, wherein the protein profile is produced by a proteomics work flow.

148. The method of statement 145, wherein the protein profile is produced by a combination of an immunoassay work flow and a proteomics work flow.

149. The method of any one of statements 145-148, wherein the method produces a protein profile of non-membrane/soluble protein complexes.

150. The method of any one of statements 145-149, wherein the method produces a protein profile of cell-membrane associated proteins.

151. A method of isolating one or more proteins, comprising the methods of any one of statements 1-150.

152. A method of partitioning proteins in a sample by differential solubility, comprising the methods of any one of statements 1-150.

153. A method for preparing sequential samples using an absorptive device, comprising the methods of any one of statements 1-150.

154. A microsampling device, comprising:
a) a first porous material;
b) a second porous material; and
c) a tube.

155. A microsampling device, comprising:
a) a first porous material,
b) a second porous material;
c) a third porous material; and
d) a tube.

156. The device of statement 143 or 144, wherein the tube is an Eppendorf tube.

157. A microsampling device, comprising:
a) a first porous material;
b) a second porous material; and
c) a shaft;
   wherein the first porous material is positioned at one end of the shaft and the second porous material is located at a position along the shaft so that the first porous material and the second porous material are separated and not in physical contact with each other.

158. A microsampling device, comprising:
a) a first porous material;
b) a second porous material;
c) a third porous material; and
d) a shaft;
   wherein the first porous material is positioned at one end of the shaft and the second porous material and the third porous material are located at positions on the shaft so that the first porous material, the second porous material, and the third porous material are separated and not in physical contact with each other.

159. The device of any one of statements 154-158, wherein the microsampling device is a volumetric absorptive microsampling (VAMS) device.

160. The device of any one of statements 157-159, wherein the first porous material and the second porous material are positioned on the shaft and separated from each other by a spacer such that said first porous material and said second porous material are separated and not in physical contact with each other.

161. The device of any one of statements 158-159, wherein the first porous material, the second porous material, and the third porous material are positioned on the shaft and separated from each other by spacers such that said first porous material, said second porous material, and said third porous material are separated and not in physical contact with each other.

162. The device of any one of statements 157-161, wherein the shaft is a threaded shaft.

163. The device of any one of statements 157-162, wherein the shaft is a non-linear shaft.

164. The device of any one of statements 157-163, wherein the shaft has a length of 10 mm to 50 mm.

165. The device of any one of statements 157-164, wherein the shaft has a diameter of 0.5 mm to 3 mm.

166. The device of any one of statements 157-165, wherein the shaft fits in a tube having a volume of 5 mL, 2 mL, 1.5 mL, or 1 mL.

167. The device of statement 166, wherein the tube is an Eppendorf tube.

168. The device of any one of statements 157-167, wherein the shaft fits in a 96-well plate.

169. The device of any one of statements 154-168, wherein the porous materials are equally spaced.

170. The device of any one of statements 154-169, wherein the first porous material and second porous material are pre-loaded with anticoagulant.

171. The device of any one of statements 154-170, wherein the porous materials have an absorptive capacity in the range of 2.5 μL to 50 μL.

172. The device of any one of statements 154-170, wherein the porous materials have an absorptive capacity of 2.5 μL, 5 μL, 10 μL, 20 μL, 30 μL, 40 μL, or 50 μL.

173. The device of any one of statements 154-172, wherein at least two of the porous materials have the same absorptive capacity.

174. The device of any one of statements 154-172, wherein each of the porous materials and have a different absorptive capacity.

175. A method of using the device of any one of statements 154-174 according to the method of any one of statements 1-153.

176. A method of producing a protein profile comprising:
a) obtaining at least one protein profile produced according to one or more of statements 1-150 from a sample that has been obtained from a subject having a disease or disorder;
b) obtaining at least one protein profile produced according to one or more of statements 1 to 150 from a sample that has been obtained from at least one subject not having the disease or disorder;
c) comparing the protein profile of the subject having the disease or disorder to the protein profile of the at least one subject not having the disease or disorder, and
d) producing the disease protein profile from the comparison, wherein said produced disease protein profile comprises one or more proteins that have a different presence or level in the protein profile from the subject having the disease or disorder compared to the protein profile of the at least one subject not having the disease or disorder.

Throughout this application subheadings have been used for convenience of the reader, and are not intended, nor should be interpreted, as limiting the embodiments disclosed within the subheading to only apply to the disclosures contained in said subheading. Rather, all embodiments disclosed herein are equally applicable to any one or all embodiments in the instant application.

Throughout this application various publications have been referenced. The disclosures of these publications in their entireties are hereby incorporated by reference in this application in order to more fully describe the state of the art to which this invention pertains. Although the invention has been described with reference to the examples provided above, it should be understood that various modifications can be made without departing from the spirit of the invention.

What is claimed:

1. A method of fractionating a sample, comprising:
a) introducing a sample into a porous material:
b) optionally, centrifuging the porous material containing the sample:
c) drying the sample in the porous material:
d) extracting a first set molecules from the dried sample-containing porous material, comprising:
1) incubating the dried sample-containing porous material in a first extraction solution:
2) separating the first extraction solution from the porous material; and
3) optionally, washing the porous material:
e) digesting proteins remaining in the porous material, comprising:
1) incubating the porous material in a digestion solution; and
2) separating the digestion solution from the porous material;
f) detecting one or more molecules in the separated first extraction solution; and
g) detecting one or more peptides or proteins in the digestion solution.

2. The method of claim 1, wherein following extraction of the first set of molecules and prior to the digestion step the method further comprises:
A) extracting a second set of molecules from the first extracted porous material, comprising:
1) incubating the first extracted porous material in a second extraction solution:
2) separating the second extraction solution from the second incubated porous material; and
h) optionally, washing the separated second extracted porous material; and
B) optionally, detecting the second set of molecules in the separated second extraction solution.

3. The method of claim 2, wherein prior to the digestion step the method further comprises sequentially extracting one or more further sets of molecules from the second extracted porous material comprising repeating the steps A)-B) with one or more further extraction solutions.

4. The method of any one of claims 1-3, wherein the first set of molecules is selected from the group consisting of proteins, nucleic acids, and glycans.

5. The method of any one of claims 2-4, wherein the second set of molecules is selected from the group consisting of proteins, nucleic acids, and glycans.

6. The method of any one of claims 3-5, wherein the one or more further sets of molecules is selected from the group consisting of proteins, nucleic acids, and glycans.

7. The method of any one of claims 4-6 wherein said proteins in the first, second, or one or more further sets of molecules, respectively, and/or the one or more peptides or proteins in the digestion solution are phosphoproteins or phosphopeptides.

8. The method of claim 7, comprising performing an enrichment step to enrich for phosphorylated proteins and/or peptides in the separated first, second, or one or more extraction solutions and/or the digestion solution.

9. The method of any one of claims 3-8, wherein the one or more further extraction solutions comprises an extracting agent comprising a salt, a mild detergent, a strong detergent, a chaotrope, a reducing agent, a thiol-containing reducing agent, or an alkylating agent, an acid, an organic solvent, or an enzyme, or combinations thereof.

10. The method of any one of claims 1-9, wherein the first extraction solution comprises an extracting agent comprising a salt, a mild detergent, a strong detergent, a chaotrope, a reducing agent, a thiol-containing reducing agent, or an alkylating agent, an acid, an organic solvent, or an enzyme, or combinations thereof.

11. The method of any one of claims 1-10, wherein the second extraction solution comprises an extracting agent comprising a salt, a mild detergent, a strong detergent, a chaotrope, a reducing agent, a thiol-containing reducing agent, or an alkylating agent, an acid, an organic solvent, or an enzyme, or combinations thereof.

12. The method of any one of claims 1-11, wherein the digestion solution comprises a reducing agent, an alkylating agent, a buffer, a detergent, or combinations thereof, and optionally, wherein the digestion solution further comprises a salt, a mild detergent, a strong detergent, a chaotrope, or a thiol-containing reducing agent, an acid, an organic solvent, or an enzyme, or combinations thereof.

13. The method of any one of claims 9-12, wherein the salt is or comprises NaCl, LiCl, CaCl₂), or Tris-HCl.

14. The method of any one of claims 9-13, wherein the mild detergent is a non-ionic detergent, an ionic detergent, or a zwitterionic detergent.

15. The method of any one of claims 9-14, wherein the mild detergent is or comprises: i) a non-ionic detergent comprising PBS or Tween; ii) an ionic detergent comprising sodium deoxycholate; or iii) a zwitterionic detergent comprising a sulfobetaine or an amidosulfobetaine.

16. The method of any one of claims 9-15, wherein the strong detergent is or comprises CTAB, CHAPS, or SDS.

17. The method of any one of claims 9-16, wherein the chaotrope is or comprises urea, thiourea, or guanidine.

18. The method of any one of claims 9-17, wherein the reducing agent is or comprises a phosphine.

19. The method of any one of claim 18, wherein the phosphine is or comprises TBP or TCEP.

20. The method of any one of claims 9-19, wherein the thiol-containing reducing agent is or comprises betamercaptoethanol or dithiothreitol.

21. The method of any one of claims 9-20, wherein the alkylating agent is or comprises iodoacetamide or acrylamide.

22. The method of any one of claims 9-21, wherein the acid is or comprises citric acid or trifluoroacetic acid.

23. The method of any one of claims 9-22, wherein the organic solvent is or comprises methanol.

24. The method of any one of claims 9-23, wherein the organic enzyme is or comprises benzonase.

25. The method of any one of claims 7-24, wherein the first, the second, the one or more further extraction solutions, and the digestion solutions are different.

26. The method of any one of claims 7-25, wherein selection of the first, the second, the one or more further extraction solutions is to fractionate the proteins contained in the porous material via differential solubility.

27. The method of any one of claims 7-26, wherein each sequential extraction solution selected is characterized as having greater solubility of the proteins remaining in the porous material.

28. The method of any one of claims 7-27, wherein the first, the second, the third, and the further extraction solutions comprise identical extracting agents of increasing concentrations as progress from one extraction to the next extraction.

29. The method of any one of claims 7-28, wherein the concentration of the salt in the first, the second, or one or more further extraction solutions, is 0.01-1 M.

30. The method of any one of claims 1-29, wherein the first, the second, or one or more further extraction solutions, comprises LiCl and Tris-HCl.

31. The method of claim 30, wherein the concentration of LiCl is 0.5 M, and the concentration of Tris-HCl is 0.1 M.

32. The method of any one of claims 7-31, wherein the concentration of the chaotrope in the first, the second, or one or more further extraction solutions, is 0.01-8 M.

33. The method of claim 32, wherein the concentration of the chaotrope in the first, the second, or one or more further extraction solutions, is 0.1, 0.5, 1, 1.5, 2, 2.5, 3, 3.5, 4, 4.5, 5, 5.5, 6, 6.5, 7, 7.5, or 8 M.

34. The method of any one of claims 7-33, wherein the first, the second, or one or more further extraction solutions, comprises urea at a concentration of 2, 5, 6, or 7M, and thiourea.

35. The method of any one of claims 1-34, wherein the digestion solution further comprises a tryptic digestion solution.

36. The method of any one of claims 1-35, wherein the digestion solution comprises triethylammonium bicarbonate, sodium deoxycholate (SDC), TCEP, or chloracetamide, or combinations thereof.

37. The method of claim 36, wherein the triethylammonium bicarbonate is present in the digestion solution at a concentration of 0.1 M.

38. The method of claim 36 or 37, wherein the sodium deoxycholate (SDC) is present in the digestion solution at a concentration of 0.05% to 10% (w/v).

39. The method of any one of claims 36-38, wherein the TCEP is present in the digestion solution at a concentration of 1 mM to 100 mM.

40. The method of any one of claims 36-39, wherein the chloroacetamide is present in the digestion solution at a concentration of 5 mM to 100 mM.

41. The method of any one of claims 1-40, wherein the digestion solution comprises a protease or a combination of proteases.

42. The method of any one of claims 1-41, wherein the protease is trypsin or the combination of proteases comprises trypsin.

43. The method of any one of claims 1-42, wherein the first, second, or one or more further extractions solutions comprises an endoglycosidase.

44. The method of claim 43, wherein the endoglycosidase is Peptide-N-Glycosidase F (PNGase F) and/or O-glycosidase.

45. The method of any one of claims 1-44, wherein the sample is or comprises a body fluid sample.

46. The method of any one of claims 1-45, wherein the sample comprises cells or tissue.

47. The method of any one of claims 1-46, wherein the sample comprises cells suspended in a liquid.

48. The method of any one of claims 1-47, wherein the sample comprises cultured cells suspended in a culture media.

49. The method of any one of claims 1-48, wherein the sample is or comprises blood, blood fractions, plasma, urine, tears, wound fluid, CSF, bronchoalveolar lavage, or ascites.

50. The method of any one of claims 1-49, wherein the sample is or comprises plasma.

51. The method of any one of claims 1-50, wherein the sample is or comprises a blood sample.

52. The method of claim 51, wherein the blood sample is: a whole blood (WB) sample, a red blood cell (RBC) sample, a white blood cell (WBC) sample, a frozen blood sample, or a fresh blood sample.

53. The method of any one of claims 1-52, wherein the sample has a volume in the range of 100 μL to 2 μL, 100 μL to 5 μL, 100 μL to 10 μL, 99 μL to 2 μL, 90 μL to 2 μL, 80 μL to 2 μL, 70 μL to 2 μL, 60 μL to 2 μL, 50 μL to 2 μL, 40 μL to 2 μL, 30 μL to 2 μL, 20 μL to 2 μL, 10 μL to 2 μL, or 5 μL to 2 μL.

54. The method of any one of claims 1-53, wherein the sample has a volume of <100 μL, <50 μL, <30 μL, <10 μL, or <5 μL.

55. The method of any one of claims 1-54, wherein the sample has a volume of at least 2 μL.

56. The method of any one of claims 1-55, wherein the introducing step comprises absorbing the sample into the porous material.

57. The method of any one of claims 1-56, wherein the introducing step comprises: absorbing the sample into the porous material via a finger prick, dipping the porous material into the sample, or pipetting a known volume of the sample into the porous material.

58. The method of any one of claims 1-57, wherein the method comprises centrifuging the porous material containing the sample prior to the drying step.

59. The method of any one of claims 1-58, comprising drying the sample in the porous material and wherein the drying step comprises drying for a period of time to adhere the sample to the porous material.

60. The method of claim 59, wherein the drying step comprises air drying the sample within the porous material.

61. The method of claim 59 or 60, wherein the drying step is for less than 30 minutes, preferably at least 5 minutes.

62. The method of claim 60, wherein the air drying is for at least 1, 2, 4, 6, 8, 10, 12, 16, 20 or 24 hours.

63. The method of claim 60, wherein the air drying is for at least 1 day.

64. The method of any one of claims 60-63, wherein the drying is in the presence of a desiccant.

65. The method of any one of claims 59-64, wherein the drying step comprises centrifuging the sample within the porous material.

66. The method of claim 58 or 65, wherein the centrifuging is at a speed of 500 g to 10,000 g.

67. The method of claim 66, wherein the centrifuging is for at least 1 to 15 minutes.

68. The method of any one of claim 65 or 66, wherein the centrifuging is for no more than 15 minutes.

69. The method of any one of claims 59-68, wherein the drying step comprises vacuuming the sample within the porous material.

70. The method of any one of claims 1-69, further comprising storing the sample-containing porous material for a period of time prior to the first extraction step.

71. The method of claim 70, wherein storing the sample-containing porous material immediately follows the drying step.

72. The method of claim 70 or 71, wherein the sample-containing porous material is stored frozen.

73. The method of claim 70 or 71, wherein the sample-containing porous material is stored at room temperature.

74. The method of any one of claims 1-73, wherein the drying step, the extraction step, and the digesting step are conducted with the sample-containing porous material in a tube.

75. The method of any one of claims 1-74, wherein the separated first extraction solution comprises albumin, hemoglobin, IgG, or one or more additional proteins compared to conventionally isolated serum or plasma.

76. The method of any one of claims 1-75, wherein the separated second extraction solution comprises the second set of proteins.

77. The method of any one of claims 1-76, wherein the separated one or more extraction solutions comprises a further set of proteins, respectively.

78. The method of any one of claims 1-77, wherein the first, the second, or one or more further, extraction solution has a volume of less than 100 μL, a volume less than 95 μL, a volume less than 90 μL, a volume of 1-5 times the volume of porous material, a volume of 1, 2, 3, 4, or 5 times the volume of porous material, preferably a volume of 3 times the volume of porous material.

79. The method of any one of claims 1-78, wherein the incubating in the first, the second, or one or more further extraction solutions is conducted for a period of time long enough to extract one or more proteins contained within or adhered to the porous material.

80. The method of claim 79, wherein said incubating is conducted for 1, 5, 10, 15, 20, 25, 30, 35, 40, 45, 50, 55, or 60 min.

81. The method of claim 79, wherein said incubating is conducted for 1-48 hours, 1-42 hours, 1-36 hours, 1-30 hours, 1-24 hours, 1-18 hours, 1-12 hours, or 1-6 hours.

82. The method of any one of claims 79-81, wherein said incubating is conducted at ambient temperature.

83. The method of any one of claims 79-81, wherein said, wherein said incubating is conducted at a temperature elevated above ambient temperature.

84. The method of any one of claims 1-83, wherein the incubating in the digestion solution is conducted for a period of time long enough to digest one or more proteins contained within or adhered to the porous material.

85. The method of claim 84, wherein said incubating is conducted for 1 min-48 hour.

86. The method of claim 84, wherein said incubating is conducted for 1, 5, 10, 15, 20, 25, 30, 35, 40, 45, 50, 55 or 60 min.

87. The method of claim 84, wherein said incubating is conducted for 1, 2, 3, 4, 5, 6, 7, 8, 9, 10, 11, 12, 13, 14, 15, 16, 17, 18, 19, 20, 21, 22, 23 or 24 hours.

88. The method of any one of claims 84-87, wherein said incubating is conducted at ambient temperature, conducted at a temperature elevated above ambient temperature, preferably at 37° C.

89. The method of any one of claims 79-88, wherein said incubating is conducted with agitation.

90. The method of any one of claims 1-89, wherein separating the first, the second, or the one or more further extraction solutions from the first, the second, the third, or the further, incubated porous material comprises removing the first, the second, the third, or the further, incubated porous material from the first, the second, or the one or more further extraction solutions, respectively.

91. The method of any one of claims 1-89, wherein separating the first, the second, or the one or more further extraction solutions from the first, the second, the third, or the further, incubated porous material comprises removing the first, the second, the third, or the one or more further extraction solutions from the first, the second, or the further, incubated porous material, respectively.

92. The method of any one of claims 1-91, wherein separating the digestion solution from the porous material comprises removing the digestion incubated porous material from the digestion solution.

93. The method of any one of claims 1-91, wherein separating the digestion solution from the porous material comprises removing the digestion solution from the digestion incubated porous material.

94. The method of any one of claims 90-93, wherein the separating is via centrifugation, filtration, or a combination of centrifugation and filtration, respectively.

95. The method of any one of claims 1-94, wherein the method comprises washing the separated first, second, or further, extracted porous material.

96. The method of claim 95, wherein the separated first, second, or further extracted porous material is washed with a washing volume of the first, second, or the one or more further extraction solutions, respectively, a volume of an extraction solution different from the first extraction solution, or in a volume of water.

97. The method of claim 96, wherein selection of the different extraction solution is limited to solutions based on proteins remaining in the separated extracted porous material that have equivalent or less solubility in the different extraction solution than the preceding extraction solution.

98. The method of claim 96 or 97, wherein the washing volume of the extraction solution is between 5 times the volume of porous material and 3 mL.

99. The method of claim 96 or 97, wherein the washing volume of the first, the second, the third, or the further, extraction solution is 0.05-3 mL.

100. The method of any one of claims 95-99, wherein the washing step is repeated 1, 2, or 3 times.

101. The method of any one of claims 1-100, wherein said detecting one or more molecules in the separated first extraction solution comprises detecting proteins in the separated first extraction solution.

102. The method of any one of claims 2-101, comprising detecting one or more molecules in the separated second extraction solution wherein said detecting comprises detecting proteins in the separated second extraction solution.

103. The method of any one of claims 3-100, comprising detecting one or more molecules in the separated one or more further extraction solutions, wherein said detecting comprises detecting proteins in the separated one or more further extraction solution.

104. The method of any one of claims 101-103, wherein the detecting of proteins is via immunoassay.

105. The method of claim 104, wherein the immunoassay is or comprises a Western blot, or ELISA.

106. The method of claim 104, wherein the detecting step comprises a Luminex and Proximity Extension Assay.

107. The method of claim 106, wherein the Luminex and Proximity Extension Assay comprises Slow Offrate Modified Aptamer (SOMAmer) reagents.

108. The method of any one of claims 101-103, wherein the detecting of proteins is via Mass Spectrometry (MS).

109. The method of claim 108, wherein the detection via MS comprises subjecting the extraction solution to digestion prior to said detection via Mass Spectrometry (MS).

110. The method of any one of claims 1-100, wherein said detecting one or more molecules in the separated first extraction solution comprises detecting glycans in the separated first extraction solution.

111. The method of any one of claim 2-101, or 104-109, comprising detecting one or more molecules in the second extraction solution wherein said detecting comprises detecting glycans.

112. The method of any one of claim 3-102, or 104-109, comprising detecting one or more molecules in the separated one or more further extraction solutions, wherein said detecting comprises detecting glycans in the separated one or more further extraction solution.

113. The method of any one of claims 110-112, wherein the detecting of glycans is via High Performance Liquid Chromatography (HPLC).

114. The method of any one of claims 1-113, wherein the detecting of one or more peptides or proteins in the digestion solution is via Mass Spectrometry (MS).

115. The method of any one of claim 108, 109 or 114, wherein said MS is LC-MS.

116. The method of any one of claim 108, 109 or 114, wherein said MS is selected reaction monitoring mass spectrometry (SRM-MS).

117. The method of any one of claim 108, 109 or 114, wherein said MS is data-dependent acquisition MS (DDA-MS).

118. The method of any one of claim 108, 109 or 114, wherein said MS is data-independent acquisition MS (DIA-MS).

119. The method of any one of claim 108, 109 or 114, wherein said MS is selected from the group consisting of matrix-assisted laser desorption/ionisation time-of-flight (MALDI-TOF) MS: MALDI-TOF post-source-decay (PSD): MALDI-TOF/TOF: surface-enhanced laser desorption/ionization time-of-flight mass spectrometry (SELDI-TOF) MS: electrospray ionization mass spectrometry (ESI-MS): ESI-MS/MS: ESI-MS/(MS)n (n is an integer greater than zero); ESI 3D or linear (2D) ion trap MS: ESI triple quadrupole MS: ESI quadrupole orthogonal TOF (Q-TOF): ESI Fourier transform MS systems; desorption/ionization on silicon (DIOS): secondary ion mass spectrometry (SIMS): atmospheric pressure chemical ionization mass spectrometry (APCI-MS): APCI-MS: APCI-(MS)n: ion mobility spectrometry (IMS): inductively coupled plasma mass spectrometry (ICP-MS) atmospheric pressure photoionization mass spectrometry (APPI-MS), APPI-MS/MS; and APPI-(MS)n.

120. The method of any one of claims 1-119, wherein the porous material is a hydrophilic polymeric material.

121. The method of claim 120, wherein the porous material comprises a plastic.

122. The method of claim 120 or 121, wherein the porous material comprises a sponge.

123. The method of any one of claims 120-122, wherein the three-dimensional porous material is a tip of a volumetric absorptive microsampling (VAMS) device.

124. The method of any one of claims 1-123, wherein the porous material is not a pre-loaded porous material.

125. The method of any one of claims 1-123, wherein the porous material is pre-loaded with a protease inhibitor.

126. The method of any one of claims 1-123, wherein the porous material is pre-loaded with an enzyme, preferably a nuclease, more preferably benzonase.

127. The method of any one of claims 1-123, wherein the porous material is pre-loaded with a detergent, preferably SDS.

128. The method of any one of claims 1-123, wherein the porous material is pre-loaded with an anticoagulant.

129. The method of claim 128, wherein the anticoagulant is EDTA or heparin.

130. The method of any one of claims 1-129, wherein the method uses a plurality of the porous materials.

131. The method of claim 130, wherein the sample is introduced into the plurality of the porous materials.

132. The method of any one of claims 1-131, wherein one or more proteins detected in a first, second, or one or more further set of molecules is or comprises a non-membrane/soluble protein complex.

133. The method of any one of claims 1-132, wherein one or more proteins detected in the separated digestion solution is or comprises a non-membrane/soluble protein complex.

134. The method of any one of claims 1-133, wherein one or more proteins detected in a first, second, or one or more further set of molecules is or comprises a cell-membrane associated protein.

135. The method of any one of claims 1-134, wherein one or more proteins detected in the separated digestion solution is or comprises a cell-membrane associated protein.

136. The method of claim 134 or 135, wherein the cell-membrane associated protein is a cell-membrane bound protein or a cell integral-membrane protein.

137. The method of any one of claims 134-136, wherein the cell-membrane associated protein is a membrane protein complex.

138. The method of any one of claims 1-137, wherein the method recovers from a red blood cell (RBC) sample, a plasma sample, or a whole blood sample, a fraction of non-membrane/soluble protein complexes not obtained by traditional microsampling, or a fraction of membrane protein complexes not obtained by traditional microsampling analysis.

139. The method of claim 1, wherein the porous material is a hydrophilic polymer tip of a volumetric absorptive microsampling (VAMS) device, wherein the first extraction solution comprises 100 mM-500 mM LiCl and 100 mM Tris, and wherein the method comprises washing the porous material at step d) 3) with a wash solution comprising 100 mM-500 mM LiCl and 100 mM Tris, and wherein the digestion solution comprises 1 μg Trypsin in a buffer comprising 1% sodium deoxycholate (SDC), 10 mM tris (2-carboxyethyl) phosphine (TCEP), 40 mM 2-Chloroacetamide (CLA), 100 mM and 100 mM triethylammonium bicarbonate buffer (TEAB).

140. The method of claim 1 or 2, wherein the porous material is a hydrophilic polymer tip of a volumetric absorptive microsampling (VAMS) device, wherein the first extraction solution comprises a PBS solution comprising a protease inhibitor, wherein the volume of the first extraction solution is ≤100 μL; wherein the method comprises washing the porous material at step d) 3) with a wash solution comprising 100 mM-500 mM LiCl and 100 mM Tris: wherein the digestion solution comprises 1 μg Trypsin in a buffer comprising 1% sodium deoxycholate (SDC), 10 mM tris (2-carboxyethyl) phosphine (TCEP), 40 mM 2-Chloroacetamide (CLA), 100 mM and 100 mM triethylammonium bicarbonate buffer (TEAB); and wherein the one or more molecules in the separated first extraction solution is one or more proteins and detection is via immunoassay.

141. The method of claim 140, wherein the second extraction solution comprises PNGase F in a buffer comprising 1% sodium deoxycholate (SDC), 10 mM tris (2-carboxyethyl) phosphine (TCEP), 40 mM 2-Chloroacetamide (CLA), 100 mM and 100 mM triethylammonium bicarbonate buffer (TEAB); and wherein the method comprises detecting the second set of molecules in the separated second extraction solution, wherein the one or more molecules is one or more glycans.

142. The method of claim 3, wherein the one or more further extraction solution comprises PNGase F in a buffer comprising 1% sodium deoxycholate (SDC), 10 mM tris (2-carboxyethyl) phosphine (TCEP), 40 mM 2-Chloroacetamide (CLA), 100 mM and 100 mM triethylammonium bicarbonate buffer (TEAB); and wherein the method comprises detecting a further set of molecules in the separated further extraction solution, wherein the one or more molecules is one or more glycans.

143. The method of claim 141 or 142, further comprising dividing the porous material prior to the digestion step e), and analysing a portion of the portion of the porous material for remaining glycans.

144. A method of producing a protein profile, comprising the methods of any one of claims 1-143.

145. The method of claim 144, wherein the protein profile is produced by immunoassay work flow.

146. The method of claim 144, wherein the protein profile is produced by a proteomics work flow.

147. The method of claim 144, wherein the protein profile is produced by a combination of an immunoassay work flow and a proteomics work flow.

148. The method of any one of claims 144-147, wherein the method produces a protein profile of non-membrane/soluble protein complexes.

149. The method of any one of claims 144-148, wherein the method produces a protein profile of cell-membrane associated proteins.

150. A method of isolating one or more proteins, comprising the methods of any one of claims 1-149.

151. A method of partitioning proteins in a sample by differential solubility, comprising the methods of any one of claims 1-149.

152. A method for preparing sequential samples using an absorptive device, comprising the methods of any one of claims 1-149.

153. A microsampling device, comprising:
a) a first porous material;
b) a second porous material; and
c) a tube.

154. A microsampling device, comprising:
a) a first porous material,
b) a second porous material;
c) a third porous material; and
d) a tube.

155. The device of claim 142 or 143, wherein the tube is an Eppendorf tube.

156. A microsampling device, comprising:
a) a first porous material;
b) a second porous material; and
c) a shaft;
wherein the first porous material is positioned at one end of the shaft and the second porous material is located at a position along the shaft so that the first porous material and the second porous material are separated and not in physical contact with each other.

157. A microsampling device, comprising:
a) a first porous material:
b) a second porous material:
c) a third porous material; and
d) a shaft:
wherein the first porous material is positioned at one end of the shaft and the second porous material and the third porous material are located at positions on the shaft so that the first porous material, the second porous material, and the third porous material are separated and not in physical contact with each other.

158. The device of any one of claims 153-157, wherein the microsampling device is a volumetric absorptive microsampling (VAMS) device.

159. The device of any one of claims 156-158, wherein the first porous material and the second porous material are positioned on the shaft and separated from each other by a spacer such that said first porous material and said second porous material are separated and not in physical contact with each other.

160. The device of any one of claims 157-158, wherein the first porous material, the second porous material, and the third porous material are positioned on the shaft and separated from each other by spacers such that said first porous material, said second porous material, and said third porous material are separated and not in physical contact with each other.

161. The device of any one of claims 156-160, wherein the shaft is a threaded shaft.

162. The device of any one of claims 156-161, wherein the shaft is a non-linear shaft.

163. The device of any one of claims 156-162, wherein the shaft has a length of 10 mm to 50 mm.

164. The device of any one of claims 156-163, wherein the shaft has a diameter of 0.5 mm to 3 mm.

165. The device of any one of claims 156-164, wherein the shaft fits in a tube having a volume of 5 mL, 2 mL, 1.5 mL, or 1 mL.

166. The device of claim 165, wherein the tube is an Eppendorf tube.

167. The device of any one of claims 156-166, wherein the shaft fits in a 96-well plate.

168. The device of any one of claims 153-167, wherein the porous materials are equally spaced.

169. The device of any one of claims 153-168, wherein the first porous material and second porous material are pre-loaded with anticoagulant.

170. The device of any one of claims 153-169, wherein the porous materials have an absorptive capacity in the range of 2.5 μL to 50 μL.

171. The device of any one of claims 153-169, wherein the porous materials have an absorptive capacity of 2.5 μL, 5 μL, 10 μL, 20 UL, 30 μL, 40 μL, or 50 μL.

172. The device of any one of claims 153-171, wherein at least two of the porous materials have the same absorptive capacity.

173. The device of any one of claims 153-171, wherein each of the porous materials and have a different absorptive capacity.

174. A method of using the device of any one of claims 153-173 according to the method of any one of claims 1-152.

175. A method of producing a protein profile comprising:
a) obtaining at least one protein profile produced according to one or more of claims 1-149 from a sample that has been obtained from a subject having a disease or disorder;
b) obtaining at least one protein profile produced according to one or more of claims 1 to 149 from a sample that has been obtained from at least one subject not having the disease or disorder;
c) comparing the protein profile of the subject having the disease or disorder to the protein profile of the at least one subject not having the disease or disorder, and
d) producing the disease protein profile from the comparison, wherein said produced disease protein profile comprises one or more proteins that have a different presence or level in the protein profile from the subject having the disease or disorder compared to the protein profile of the at least one subject not having the disease or disorder.
